(19) United States
(12) Patent Application Publication
(10) Pub. No.: US 2026/0306509 A1
(43) Pub. Date: Oct. 8, 2026

(54) COMPOSITION, METHODS AND USES

(71) Applicant: 4E Therapeutics, Inc., Austin, TX (US)

(72) Inventors: Johan Patrik Ernfors, Solna (SE); Jie Su, Solna (SE); Mingdong Zhang, Solna (SE); Jussi Antero Kupari, Solna (SE)

(21) Appl. No.: 19/480,682

(22) PCT Filed: May 1, 2024
(86) PCT No.: PCT/US2024/027158

§ 371 Date: Oct. 31, 2025

(30) Foreign Application Priority Data
May 2, 2023 (GB) 2306456.1
Feb. 13, 2024 (GB) 2401979.6

(51) Int. Cl. A61K 31/56; A61K 31/137; A61K 31/167; A61K 31/202; A61K 31/216; A61K 31/415; A61K 31/444; A61K 31/485; A61K 45/06; A61P 29/00; G01N 33/68

(52) U.S. Cl. CPC A61K 31/56; A61K 31/137; A61K 31/167; A61K 31/202; A61K 31/216; A61K 31/415; A61K 31/444; A61K 31/485; A61K 45/06; A61P 29/00; G01N 33/68; G01N 2333/555

(57) ABSTRACT

The present disclosure relates to type I interferon inhibitors for use in treating or preventing pain associated with Rheumatoid Arthritis (RA) in a patient, related uses and kits, and methods for identifying a patient who has such pain and is in need of treatment with such an inhibitor.

### RELATED APPLICATIONS

This application claims priority to and benefit of United Kingdom Application No. 2306456.1, filed May 2, 2023, and United Kingdom Application No. 2401979.6, filed Feb. 13, 2024, the entire contents of each of which are incorporated herein by reference.

### FIELD OF THE DISCLOSURE

The present invention relates to type I interferon inhibitors for use in treating or preventing pain associated with Rheumatoid Arthritis (RA) in a patient, related uses and kits, and methods for identifying a patient who has such pain and is in need of treatment with such an inhibitor.

### BACKGROUND

Rheumatoid arthritis (RA) is a systemic autoimmune disease characterised by chronic inflammation and progressive deterioration of the joints, causing significant pain and stiffness. About 1% of the global population is estimated to develop RA. Pain associated with RA is highly debilitating and impacts quality of life in affected patients.

A breakdown of T-cell and/or B-cell tolerance starts the complex process of RA. This leads to activation and subsequent production of a range of antibodies that recognise self-proteins including disease-specific IgG autoantibodies directed towards modified IgG (rheumatoid factors), citrullinated or carbamylated proteins and collagen type II. Circulating autoantibodies activate the innate and adaptive immune system with the production of a range of inflammatory factors, including cytokines. Cytokines can be classified as circulating pro-inflammatory cytokines and/or inflammatory cytokines in the joints, but anti-inflammatory cytokines and natural cytokine antagonists are also involved.

RA involves increased levels of cytokines of different classes, with dynamic alterations through the disease process, whereby different combinations of cytokines display a hierarchical dominance. Examples of cytokines with altered systemic levels that have been associated with RA include pro-inflammatory cytokines interleukin (IL)-1, IL-32, IL-33, IL-36, tumor necrosis factor (TNF)-alpha, as well as an increase in joints of IL-1, TNF-alpha, IL-6, IL-15, IL-16, IL-17, IL-18, granulocyte macrophage-colony stimulating factor (GM-CSF) and various types of interferons (IFN). Some anti-inflammatory cytokines associated with RA include IL-10, IL-4, IL13, IL-20, IL-27, IL-35, IL-37, IL-38, as well as natural cytokine antagonists to the IL-1 receptor, soluble forms of IL-1 and TNF receptors, IL-18 binding protein and more (Alunno et al, 2017; Selim et al, 2017; Ridgley et al, 2018).

Pain is the most troublesome symptom of RA, and its cause is not well understood. People with RA can experience pain at rest and during normal activities and may display increased sensitivity to evoked pain in response to stimuli such as normal movement or gentle pressure on the joints. In addition, widespread pain and other evidence of central sensitisation are common and contribute to pain in people with RA (Heisler et al, 2020).

Pain associated with RA is thought to be complex with multifactorial causes including alterations in the immune cells as well as several areas of the nervous system, such as primary afferent sensitisation, spinal cord sensitisation as well as changes at supraspinal levels. Dysfunction of peripheral nerves, including increased excitability and ectopic activity of afferents are considered likely to contribute to pain. Such sensitisation could occur by pro-inflammatory factors present systemically and/or in the synovial fluid in RA, including TNF, IL-1, IL-6, IL-17, interferons and other cytokines, inflammatory lipids (e.g. PGE2), neuropeptides (CGRP, SP and others) and growth factors (e.g. NGF) (Cao et al, 2020).

Disease-modifying antirheumatic drugs (DMARDs) can be effective in leading to remission of inflammatory disease, but in many cases, pain persists even in the absence of inflammation (Vergne-Salle et al, 2020). DMARDs can reduce pain from the high levels associated with high disease activity but in reality, complete pain resolution is rare. It is estimated that chronic pain remains in about 10-25% of patients receiving DMARD treatment with successful inflammatory remission.

Consequently, RA patients often seek to treat the pain symptoms of RA using analgesics; however, the currently available analgesic compounds prescribed to people with RA do not offer an effective and/or long-term solution for preventing or treating pain associated with RA.

Therefore, persistent pain associated with RA remains a real problem.

The priority for many RA patients is the search for adequate analgesia. This lack of adequate pain relief in RA patients illustrates the great need of finding new analgesic strategies. In particular, there is a need for new analgesic strategies for pain relief during the weeks for the DMARD drugs to become effective, for patients with insufficient inflammatory disease control and for those with successful treatment but persistent pain. Identification of a specific cell type and molecular mechanisms of pain associated with RA would allow for more specific and effective analgesics.

### SUMMARY

Against this background, the present inventors have unexpectedly found that pain associated with RA is critically dependent on a specific type of peripheral nociceptor which can become sensitised through type I interferon signalling. The inventors have also found that, surprisingly, sensitisation and increased pain is reversible by blocking a specific type of cytokine signalling: type I interferon signalling. Consistently, inhibiting type I interferon interaction with its receptors or blocking signalling downstream of the type I interferon receptors can both prevent, and completely reverse, pain associated with RA. Notably, and also surprisingly, blocking type I interferon signalling did not robustly affect inflammatory disease activity which indicates that the pain is a mechanistically separate process from inflammatory disease. The inventors also show that the type I interferon signalling signature that exists in RA patients indicates that the type I interferon class is a key pain-causing cytokine.

Accordingly, in one aspect, the invention provides a type I interferon inhibitor for use in treating or preventing pain associated with Rheumatoid Arthritis in a patient.

In a related aspect, the invention provides a use of a type I interferon inhibitor in the manufacture of a medicament for treating or preventing pain associated with Rheumatoid Arthritis in a patient.

In a further related aspect, the invention provides a method of treating or preventing pain associated with Rheumatoid Arthritis in a patient, comprising the step of administering a type I interferon inhibitor to the patient.

The present invention is therefore a new and advantageous approach for treating or preventing pain associated with RA in a patient. As discussed further herein, the inventors' finding that pain can be treated or prevented by specifically targeting the type I interferon pathway is surprising.

### BRIEF DESCRIPTION OF THE DRAWINGS

Preferred, non-limiting examples which embody certain aspects of the invention will now be described, with reference to the following figures:

FIG. 1, comprising panels 1A-1I: Perturbation of primary sensory neuron types in antibody-induced arthritis.

FIG. 1A: Cartilage antibody (Cab) induced-arthritis mouse model.

FIG. 1B: Arthritis mice show transient joint inflammation (clinical score) from day 6 to day 23 after Cab injection in C57BL/6 N mice.

FIG. 1C: Mechanical allodynia is present as early as 4 hours and lasted until day 63 after Cab injection (n=6).

FIG. 1D: Illustration of DRG neuronal populations driven by different Cre and CreERT2 mice strains. Reflex responses percentage to blue light stimulation of those mice strains (crossed with R26-ChR2) under different stages of RA.

FIG. 1E: Uniform manifold approximation and projection (UMAP) shows the distribution of cell clusters (86,052 cells) from scRNA-seq of DRGs from control and RA mice. NonmyelSC, non-myelinating Schwann cell; MyelSC, myelinating Schwann cell; VSMC, vascular smooth muscle cell; EC, endothelial cell.

FIG. 1F: Heatmap represents the predicted similarity score of individual neurons to the neuronal types according to Usoskin et al (2015)'s annotation through machine learning classifier.

FIG. 1G: UMAP of neuronal clusters (6,200 cells) from scRNA-seq of DRGs from control and RA mice.

FIG. 1H: Mean of max prediction scores for predicted neurons within each cluster.

FIG. 1I: Perturbation in neuronal types in arthritis at different disease stages.

FIG. 2, comprising panels 2A-2J: IFN signalling in sensory neurons during arthritis.

FIG. 2A: Average score for the arthritis-induced co-regulated gene module in DRG neurons across different time points.

FIG. 2B: Heatmap of average expression of arthritis-induced module in DRG neurons across different time points of RA.

FIG. 2C: Dot plot of GO biological pathways analysis for module genes.

FIG. 2D: STRING network visualization of the co-regulated module.

FIG. 2E: Boxplots of gene module scores for individual neuronal clusters at different timepoints of arthritis.

FIG. 2F: Activity heatmap of four recurrent SCENIC regulons in individual neuronal types during arthritis.

FIG. 2G: Serum levels of IFNa at different timepoints of arthritis.

FIG. 2H: Pain-like behavioural tests of von Frey filaments (threshold) and 2 g von Frey filament (shaking numbers) show that Endo-b-N-acetylglucosaminidase (EndoS) treated Cab antibody failed to initiate pain.

FIG. 2I: Boxplot shows the co-regulated gene module scores for neurons from control, EndoS treated and IFNAR1 antibody blocked mice.

FIG. 2J: Expression heatmap of top differentially regulated genes at 0.25 days in DRG neurons of arthritis (left) and in mice injected with EndoS treated Cab or blocked by IFNAR1 antibody (right).

FIG. 3, comprising panels 3A-3G: Polymodal C-nociceptors are involved in pain associated with arthritis.

FIG. 3A: Skin-nerve recording. Representative C-type fiber recordings (CV<1.2 m/s) showing the activity during force ramp applications (10 s; 0 to 100 mN) from mice injected with saline (“C-type Control”; middle panel) or Cab (“C-type RA”; right panel), the insets show all the action potential waveforms elicit during the mechanical stimulus. This force ramp protocols were used to determine the mechanical threshold in each fiber.

FIG. 3B: Mechanical thresholds of mice injected with saline (“Control”) or Cab (“RA”). Dots represents individual values for each fiber, lines represent the mean and SD [unpaired t-test (*p<0.05)].

FIG. 3C: Mechanically induced firing frequencies of mice injected with saline (“Control”) or Cab (“RA”) during the force ramp application, thick lines represent mean number of action potentials in 1 second bins, shadowed regions are the SEM; (p<0.01 by two-way ANOVA with repeated measures for treatment effect; *p<0.05, †p<0.005 by unpaired t-test Vs Control).

FIG. 3D: Number of mechanically induced action potentials during the force ramp application [unpaired t-test (*p<0.05)].

FIG. 3E: Mechanical withdrawal threshold (von Frey filaments test), coping response (shaking numbers) to 2 g von Frey filament and acetone test without light or with subthreshold blue light (470 nm) in Gfra3-Cre^{ERT2}*R26-ChR2 (Gfra3-CHR2) mice (n=8).

FIG. 3F: Mechanical and cold sensitivity in Gfra3-Cre^{ERT2}*R26-ArchT (Gfra3-ArchT) mice before and after yellow light (566 nm) application (0.44 mWatt/mm², 45 min) (n=8).

FIG. 3G: Percentage of reflex reaction to different von Frey filaments without or with yellow light applied (0.44 mWatt/mm², 45 min): before (baseline) and after antibody injection (Early and late RA) (n=8). *** indicates p<0.001, ** indicates p<0.01 and * indicates p<0.05.

FIG. 4, comprising panels 4A-4F: Sustained interferon signalling drives pain associated with arthritis.

FIG. 4A: Perturbation in non-neuronal DRG cells at different disease stages of arthritis.

FIG. 4B: KEGG pathway of differentially expressed genes in endoneurial macrophages.

FIG. 4C: Quantitative PCR of INFa and IFNb expression in DRG.

FIG. 4D: Reversal of arthritis-induced pain by TYK2 inhibitor treatment. Five oral administrations of TYK2 inhibitor (15 mg/kg, twice daily, day13-day21) block Cab-induce mechanical hypersensitivity, while vehicle (EtOH:TPGs:PEG300=5:5:90) treatment has no effects (n=5-6).

FIG. 4E: Systemic administration of IFNAR1 mAb (1 mg, i.p.) has no effect on joint inflammation compared to Isotype group (mouse IgG).

FIG. 4F: IFNAR1 mAb one hour prior to Cab injection prevents mechanical allodynia and delivery on day 22 and day 45 reverses established pain associated with arthritis (n=5). *** indicate p<0.001, ** indicate p<0.01 and indicate p<0.05.

FIG. 5: Phosphorylation status of STAT1 (S-727), MNK1 (T-197/202) and eIF4E (S-209) in the dorsal root ganglion. Phospho-STAT1, MNK1 and eIF4E are increased in mice with cartilage antibody-induced arthritis. Numbers refer to different animals. Cont, control mice; RA-d33, samples from animals 33 days after induced arthritis; RA-d33 anti-IFNR animals with neutralization of IFNAR1 (interferon alpha/beta receptor subunit 1) on day 33 after induced arthritis. 2 h, 12 h, 33 h refers to time after arthritis was induced.

FIG. 6, comprising panels 6A-6B: Mechanical and cold hypersensitivity in in cartilage antibody-induced arthritis mouse.

FIG. 6A: Mechanical allodynia started from 4 hours after antibody injection and prolonged till day 63 in C57BL/6N mice; significant decreased withdrawal threshold at 4 hours, day 12, day 30 and day 63 compared to control mice by von Frey up-down test (n=6). Increased shaking numbers of acetone test and 2 g von Frey filament is found both in early and late phases. No differences in latency of Hargreaves test or Pinprick test are detected between Cab and control groups.

FIG. 6B: Characterization of mice strains with immunohistochemistry by comparing the reporter and neuronal cluster makers. NF200, Neurofilament 200; CGRP, Calcitonin-gene related peptide; IB4, Isolectin B4; TH, Tyrosine hydroxylase. Scale bars=100 μm and 20 μm for the inset.

FIG. 7, comprising panels 7A-7E: Single-cell RNA sequencing in DRGs of antibody-injected mice.

FIG. 7A: Illustration of sampling and workflow for scRNA-seq of cells in DRGs from antibody-induced arthritis mice.

FIG. 7B: Violin plots represent sample metrics after initial quality control for scRNA-seq data.

FIG. 7C: Dot plot represents top 3 marker genes for each major cell type of sequenced cells in DRGs.

FIG. 7D: Dot plot represents top 3 marker genes for each neuronal cluster of DRG neurons.

FIG. 7E: UMAP represents DRG neuronal cluster distribution and the same distribution with randomly sampled (25%) dataset, where the cells are intermingled from control and RA samples.

FIG. 8, comprising panels 8A-8K: Sc-RNA seq dataset of neuronal cells and immune cells in DRGs of RA.

FIG. 8A: UMAP represents the distribution of cell clusters (86,052 cells) from different time points of RA and control DRG samples.

FIG. 8B: Stacked barplot of the cell type composition (percentage) at different stages of RA.

FIG. 8C: UMAP of the distribution of subclusters of immune cells.

FIG. 8D: Stacked barplot of the composition of immune cells (percentage) at different stages of RA.

FIG. 8E: CCL2 (MCP-1) and CCL4 (MIP-1β) in serum from arthritis animals.

FIG. 8F: Feature plot represent Ccl2 and Ccl4 expression in control and RA 12 hr single cell data set (9500 cells each).

FIG. 8G: Stacked barplot of the neuron type composition (percentage) at different stages of arthritis.

FIG. 8H: Heatmap of the regulon activity across DRG neuron types and timepoints by SCENIC analysis.

FIG. 8I: UMAP of the distribution of sequenced cells from control DRG samples, EndoS treated Cab antibody arthritis samples and Cab antibody arthritis samples treated with IFNAR1 antibody.

FIG. 8J: UMAP of the distribution of cell clusters from DRG samples in (I).

FIG. 8K: Stacked barplot represents the composition (percentage) of subclusters of immune cells in (3).

FIG. 9: Gene expression of cytokine receptors in DRG neuronal types. In the upper panel, dot plot represents gene expression of receptors of cytokines (TNF-alpha receptors, CCRs, CXCRs, CSF receptors, interleukin receptors, Fc-gamma receptors and type I interferon receptors IFNAR1 and IFNAR2) for each neuronal cluster of DRG neurons; in the lower panel, dot plot represents gene expression of cytokine receptors in PEP1 cluster at different time points of arthritis.

FIG. 10, comprising panels 10A-10D: TrkA positive populations of sensory neurons contribute to pain associated with arthritis.

FIG. 10A: Representative C-type fibers recordings during static force application from mice injected with saline (“C-type Control”; upper panel) or Cab (“C-type RA”; lower panel), the insets show all the action potential waveforms elicit during the mechanical stimulus.

FIG. 10B: Average number of mechanically induced action potentials during the static indentation (p=0.05 by two-way ANOVA with repeated measures for treatment effect; *p<0.05 by unpaired t-test Vs Control).

FIG. 10C: In Wnt1-Cre*R26-ChR2 (Wnt1-CHR2) mice, with photo-stimulation of blue light, normal mice show lower withdrawal threshold for von Frey up-down test and increased shaking numbers in 2 g von Frey filament and acetone tests (reflex subthreshold: 12.7×10⁻³ mWatt/mm², coping subthreshold: 18.5×10⁻³ mWatt/mm²); while combination of blue led (reflex subthreshold: 11.7×10⁻³ mWatt/mm², coping subthreshold: 14.5 mWatt/mm²) further enhanced hypersensitivity to mechanical and cold stimuli in both early and late phases of RA mice (n=6-7). In Mrgprd-Cre*R26-ChR2 (MrgprD-CHR2) mice, only decreased withdrawal threshold is found in von Frey test combined with subthreshold of blue light in normal mice; no differences are detected between the groups without vs. with light. In TrkA-Cre^{ERT2}*R26-ChR2 (TrkA-CHR2) mice, together with blue light (reflex subthreshold: 12.6×10⁻³ mWatt/mm²), there is reduced withdrawal threshold compared to without light in the early and late RA phases; combination of blue light (coping subthreshold: 31.6×10⁻³ mWatt/mm²) only can enhance the shaking numbers in 2 g von Frey filament and Acetone test in early phase. No effects are shown in Sst-Cre*R26-ChR2 (SSt-CHR2) or Vglut3-Cre*R26-ChR2 (Vglut3-CHR2) strains with the gain-of-function by blue led in normal or RA mice.

FIG. 10D: In normal TrkA-Cre^{ERT2}*R26-ArchT (TrkA-ArchT) mice, exposure to yellow LED light (0.44 mWatt/mm², 30 min) causes less percentage of reflex reaction to 1.4 g and 2.0 g von Frey filaments; in antibody-induced arthritis mice, inhibition of TrkA population by stimulation of yellow light (0.44 mWatt/mm², 30 min) total reverses mechanical and cold allodynia as well as mechanical copping in both early and late RA phases (n=7-8). t test *** indicates p<0.001, ** indicates p<0.01 and * indicates p<0.05.

FIG. 11, comprising panels 11A-11C: PEP1 population involved in normal mechanical and cold sensitisation.

FIG. 11A: Gfra3⁺ neurons (Tom) in DRGs of Gfra3-Cre^{ERT2}*R26-Tom (Gfra3-Tom) mice, DAPI (blue) as nuclear counter staining. Scale bar=100 μm.

FIG. 11B: Percentage of reflex responses to blue light in Gfra3-Cre^{ERT2}*R26-ChR2 (Gfra3-CHR2) mice, with left-shifted response curve after antibody injection (n=8).

FIG. 11C: Combination of blue light (subthreshold of reflex: 30.2×10⁻³ mWatt/mm²; subthreshold of copping: 39.6×10⁻³ mWatt/mm²) can increase mechanical and cold sensitivity with lower mechanical threshold and more copping episode to 2 g von Frey filament and acetone tests, but has no effects on thermal sensitivity in Gfra3-CHR2 mice (n=25); After inhibition by yellow led (0.44 mWatt/mm², 45 min), Gfra3-Cre^{ERT2}*R26-ArchT (Gfra3-ArchT) mice only show higher withdrawal threshold compared to von Frey test before exposure to yellow light (n=16). t test *** indicates p<0.001 and ** indicates p<0.01.

FIG. 12: TYK2 inhibitor deucravacitinib reserved pain in chronic arthritis mice. Oral administration of TYK2 inhibitor deucravacitinib (15 mg/kg, twice daily, 7 times) reversed joint pain, dexterity and limb function; the analgesic effect of deucravacitinib was washed out at 24 h after the last injection (n=10). ***p<0.001.

FIG. 13: Systemic inhibition of MNK fully blocked RA-induced pain. (A) On day 48 after antibody injection, i.p. injection of MNK1/2 inhibitor, eFT508 (Tomivosertib/HY-100022, 1 mg/kg, in DMSO:PEG300:Tween-80:Saline of 5:40:5:50), totally reversed joint pain (shaking number in squeeze test) as well as mechanical hypersensitivity in antibody-induced arthritis mice (n=10). (B) Oral administration of 4ET-03-053 (MNK1/2 inhibitor, 1 mg/kg, in PEG300:Saline of 50:50, on day 51) into arthritis mice. Joint pain (squeeze test) was measured 1 h and 24 h after 4ET-03-053 delivery (n=6). *p<0.05, ***p<0.001.

FIG. 14: Local inhibition of MNK attenuated type I IFN-induced mechanical hypersensitivity. In veh+IFN mice, intraplantar (i.pl.) administration of IFNA3 (300 U/10 μl) induced paw mechanical hypersensitivity responses with lower withdrawal threshold (allodynia) and more shaking numbers (coping). Pre-treatment of MNK inhibitor, eFT508 (2 mg/kg, MSO:PEG300:Saline of 10:40:50) delayed onset of IFN-induced mechanical allodynia to 24 h as well as quicker reversal. No changes of mechanical sensitivity in BSA (0.1%, control) injected mice (n=5).

FIG. 15: eIF4E inhibitor reversed chronic RA pain. Systemic injection of eIF4E/eIF4G interaction inhibitor, 4EGI-1 (1 mg/kg, in DMSO:PEG300:Tween-80:Saline of 5:40:5:50), totally reversed joint as well as mechanical allodynia and coping in arthritis mice (n=5) on day 56 after antibody injection, and the effect was washed out in 24 hours. **p<0.01.

FIG. 16: MNK inhibitor blocked IFN-α-caused DRG neuron hyperexcitability. (A) Representative traces of action potential firing in the control (BSA, n=9 cells) and IFN-α (n=11 cells) groups. Number of spikes was significantly higher in the IFN-α group at each ramp intensity. In graph on right, for each ramp current, the left bar is control values and the right bar is IFN values. (B) Representative traces of action potential firing in the IFN+vehicle (n=13 cells) and IFN+MNK1/2 inhibitor (n=15 cells), increased firing by type I interferon was reversed by the MNK1/2 inhibitor pre-habituation (eFT508, 10 uM, 1 h). In graph on right, for each ramp current, the left bar is IFN+veh. values and the right bar is IFN+MNK1/2 inhib. values. (C) Small and medium sized DRG neurons were sampled for patch-clamp electrophysiology experiments. The resting membrane potential was similar across the groups, with no significant effect of IFN-α treatment. Group differences were assessed using two-way ANOVA followed by Fisher's LSD test. *p<0.05.

FIG. 17: Type I IFN protein level elevated in DRGs from RA patients with joint pain. Western blot presented IFNalpha protein as well as phospho-STAT (S727) levels were increased in donor DRGs from patients with rheumatoid arthritis (n=4) and joint pain as compared to healthy donor tissue without rheumatoid arthritis and pain (n=6). *p<0.05.

### DETAILED DESCRIPTION

RA is a complex autoimmune disease in which many cytokines and immune cells play a role. The cause of pain associated with RA is not well understood and many factors were thought to be involved. For example, any of a long list of numerous pro-inflammatory cytokines associated with RA, inflammatory lipid mediators, neuropeptides and NGF cause sensory sensitisation and pain when injected into experimental animals. These include for example, TNF-alpha, IL-1b, IL-6, IL-8, IL-12, IL-15, IL-17, IL-18, IL-23, prostaglandin E2 (PGE2), NGF (nerve growth factor), GM-CSF, CGRP, SP, PGE2 (Cunha et al, 2000; Poole et al, 1995; Amann et al 1996; Iyengar et al, 2017; Kim et al, 2011; Lee et al, 2020; Ji et al, 2021; Barragan-Iglesias et al, 2020; Achuthan et al, 2016; Raoof et al, 2018). In Ridgley et al. (2018) the cytokines of the IL-23/Th17 axis, and IL-8 were associated with the progression of joint pain. Inhibitors targeting multiple cytokine pathways (e.g. GM-CSF, G-CSF, IL-3, IL-4, IL-6, IL-7, IL-9, IL-10, IL-11, IL-12, IL-13, IL-15, IL-17, IL-22, IL-23, IFN) namely JAK1/JAK2 inhibitors, have been shown to alleviate pain associated with RA (Simon et al, 2021). In addition, inflammatory mediators are also thought to contribute to pain associated with RA.

The invention described herein specifically inhibits the type I interferon cytokine class to alleviate pain associated by RA. Such type I inhibitors do not necessarily have an effect on the inflammatory component of RA. The inventors' findings are unexpected and surprising because it was previously thought that pain associated with RA was caused by a variety of interacting cytokines; and it was not known that specifically inhibiting type I interferons could alleviate pain associated with RA.

Up until the present invention, pain onset and progression in RA was thought to be associated with a multitude of different cytokines, and the critical molecules and their relative contribution to pain were unknown. Others have unsuccessfully tried to treat pain by targeting other cytokine types—for example, adalimumab (an antibody which specifically inhibits the tumour necrosis factor (TNF) cytokine class) was unsuccessful in treating pain associated with RA.

Those skilled in the art will be familiar with type I interferon. Interferons are a family of potent immunostimulatory cytokines that are broadly divided into three subtypes: type I interferons (alpha, beta, epsilon, kappa and omega), type II interferon (gamma) and type III interferon (lambda).

Accordingly, by type I interferon, we include one or more selected from the group comprising: Interferon-alpha; Interferon-beta; Interferon-epsilon; Interferon-kappa; and Interferon-omega.

Interferon-alpha is the most abundant and best characterised, and exists in 13 distinct although homologous subtypes, excluding pseudogenes. Different genes can be active in different cells and under different conditions. In some embodiment, the interferon-alpha is one or more selected from the list comprising: IFNA1, IFNA2, IFNA4, IFNA5, IFNA6, IFNA7, IFNA8, IFNA10, IFNA13, IFNA14, IFNA16, IFNA17, and IFNA21. In one embodiment, the type I interferon is Interferon-alpha.

In contrast, the interferon-beta is only one gene (IFNB1). In another embodiment, the type I interferon is Interferon-beta.

Type I interferon production is tightly regulated such that levels are practically undetectable in healthy individuals. However, during pro-inflammatory states, type I interferon can be rapidly produced in large quantities. Especially notable in their propensity to secrete type I interferon are plasmacytoid dendritic cells (pDCs), which abundantly express intracellular pattern recognition receptors such as toll-like receptor (TLR)-7 and TLR-9. On ligation of a type I interferon receptor, type I interferons exert their effects on intracellular signalling proteins which could include but are not limited to IRF7, IRF9, STAT1 and STAT2, JAK1, TYK2, AKT, MAPK, NFkB. In turn, these induce the upregulated expression of a stereotypical set of genes, known as interferon-stimulated genes (ISGs). ISGs are upregulated in a subset of patients with RA. ISGs could include but are not limited to IFIT1, BST2, IFITM3, B2M, OASL or EPSTI1, HERC5, IFI44L, ISG15, LY6E, MX1, MX2, RSAD2 or IFI27, IFI44, IFI44L, IFI6, RSAD2, or other genes activated by type I interferon intracellular signalling.

The effects of type I interferons are pro-inflammatory and include dendritic cell maturation and activation, Th1 and Th17 polarisation, reduced regulatory T cells (T_{reg}) function and increased B-cell activation and subsequent antibody production.

The type I interferons Interferon-alpha and Interferon-beta have previously been discussed in relation to the initiation and/or progression of RA disease, albeit along with a long list of other cytokines and inflammatory factors (Castaneda-Delgado et al, 2017; Ridgley et al, 2018 and van der Pouw Kraan, 2007). It is immediately apparent that—in all of those studies—it was unclear what role (if any) type I interferons actually played in RA pathogenesis.

As discussed herein and in the accompanying Examples, the present invention now surprisingly identifies a critical role for type I interferon in pain associated with RA, and that inhibition of type I Interferon can be used to treat or prevent such pain.

By “inhibitor”, we include the meaning of a substance, such as small molecule or biologic, which reduces or prevents one or more activity of its target, such as a particular reactant, catalyst, or enzyme. As will be appreciated, the present invention relates to an inhibitor of type I interferon, and therefore reduces or prevents one or more activity of type I interferon. Such activities may include pro-inflammatory effects, dendritic cell maturation, dendritic cell activation, Th1 and Th17 polarisation, reduction of T_{reg} function, increase in B-cell activation, increase in antibody production, activation of type I interferon receptors, activation of type I interferon stimulated intracellular signalling, expression of one or more type I interferon-stimulated gene, and/or the expression of one or more type I interferon-repressed gene.

In some embodiments the type I interferon inhibitor specifically targets the type I interferon pathway or specifically inhibits type I interferon. By “specifically” in this context we include the meaning that the type I interferon inhibitor reduces or prevents one or more activity of type I interferon to a substantially (or significantly) higher level than it reduces or prevents one or more activity of another type of molecule, such as another cytokine. Accordingly, in the present case, the dominant function of the inhibitor is to reduce or prevent one or more activity of type I interferon.

By “patient”, we include a patient experiencing pain associated with RA, and a patient capable of developing pain associated with RA.

As will be appreciated, RA is a systemic autoimmune disease characterised by chronic inflammation and progressive deterioration of the joints. The disease progression can be divided into: early stage (joint pain stiffness, swelling and tenderness); moderate stage (inflammation damages the cartilage of the joint bones, patient has reduced mobility and range of motion); severe stage (further increase in impact on mobility and motion, development of joint deformities, formation of rheumatoid nodules); and end stage (symptoms become much more chronic and severe, possible inability to manage day-to-day tasks, need of assistive devices).

In a preferred embodiment, the patient is a human, or an animal (such as a fish, bird, reptile, amphibian, or mammal). Mammals include but are not limited to primates (including humans), cows, sheep, goats, horses, dogs, cats, mink, rabbits, guinea pigs, hamsters, ferrets, rats, mice, or bovine, ovine, equine, canine, feline, rodent or mu rifle species.

Various delivery systems are known and can be used to administer a type I interferon inhibitor to the patient, e.g., encapsulation in various way (such as liposomes, microparticles, microcapsules), delivery via small molecules or proteins, delivery via gene vectors (such as viruses), gene therapy (DNA or RNA). Methods of administration include, but are not limited to, intradermal, transdermal, intramuscular, intraperitoneal, intravenous, subcutaneous, intranasal, epidural and oral routes. The type I interferon inhibitor may be administered by any convenient route, for example by infusion or bolus injection, by absorption through epithelial or mucocutaneous linings (e.g. oral mucosa, rectal and intestinal mucosa, etc.) and may be administered together with other biologically active agents. The type I interferon inhibitor can be also delivered in a vesicle, in particular a liposome. Administration can be systemic or local. Those skilled in the art would be capable of selecting an appropriate route of administration, for the particular patient and type I interferon inhibitor therapy. The substance may be administered at a therapeutic dose to the patient.

By “pain associated with RA”, we include the meaning of at least one type of pain associated with and/or caused by RA, such as those discussed herein.

Pain may be associated with the onset of RA. The onset of RA can be measured, for example, using the Disease Activity Score (DAS) which indicates the severity of RA disease activity at a given moment in time. For example, DAS28 is a measure of disease activity RA, the number 28 referring to the 28 joints that are examined in this assessment.

Pain associated with RA can be identified, determined, assessed, and/or quantified using recognised tests and scales. Those skilled in the art will be capable of selecting the appropriate method—for example, by:
(i) using questionnaires, such as the “Visual analogue pain scale (VAS)” or “Patient pain VAS” to assess as ‘experienced pain’; and/or
(ii) clinical examination and quantification of joint tenderness and/or swelling (swollen joint count and tender joint count, of a possible 28); and/or
(iii) measuring the distance (mm) on the 10-cm line between the “no pain” anchor and the patient's mark, providing a range of scores from 0-100 (wherein a higher score indicates greater pain intensity). Based on the distribution of pain VAS scores as none, mild, moderate, or severe, use the following cut points on the pain VAS have been recommended: no pain (0-4 mm), mild pain (5-44 mm), moderate pain (45-74 mm), and severe pain (75-100 mm); and/or
(iv) the standardised “Quantitative sensory testing (QST)” (German Research Network for Neuropathic Pain (DFNS)) to assess neuropathic pain.

It will be appreciated that pain associated with an alternative diagnosis (i.e. pain not associated with and/or caused by RA but instead associated with other diseases, such as psoriatic arthritis, acute viral polyarthritis, polyarticular gout, calcium pyrophosphate deposition disease, systemic lupus erythematosus (SLE)) can be excluded by other tests, such as radiography (of the hands, wrists, feet), Magnetic resonance imaging (MRI), serologic studies for infections, synovial fluid analysis.

By “treating pain associated with RA”, we include the meaning of reducing the severity of at least one type of pain associated with RA in a patient. The term includes reducing or preventing the progression of the pain or the worsening of the pain. The term also includes reducing or delaying the positive prognosis of pain associated with RA.

By “preventing pain associated with RA”, we include the meaning of inhibiting the manifestation of at least one type of pain associated with RA, i.e., the subject may be free of pain or may have reduced or no pain upon administration of a substance such as a type I interferon inhibitor.

In an embodiment the pain is dysfunctional pain, such as inflammatory joint pain.

By “dysfunctional pain” we include the meaning of a special type of inflammatory pain, which can be and/or experienced in the skin, muscle, tendons, bones, and joints through the communications between immune and other non-neuronal cells to sensory neurons. Dysfunctional pain includes a dysfunction of peripheral nerves, such as an increased excitability and/or ectopic activity of afferents. It can occur independently from peripheral stimuli of nociceptors and can be caused by central sensitisation or other central mechanisms. Dysfunctional pain is caused by a disturbance of normal sensory nociceptive function resulting in spontaneous or stimulus induced chronic pain. In contrast, nociceptive pain is the body's ‘normal’ defence against harmful or potential harmful stimuli.

Peripheral sensitisation could also promote subsequent amplification of signalling in central circuits at all levels of the ascending neural pathway, a process that is referred to as central sensitisation. Such central sensitisation in the spinal cord has been ascribed to an enlargement of the receptive field through temporal and spatial summation of repetitive nociceptive input, increased pain sensitivity caused by enhanced neurotransmission through N-methyl-D-aspartate (NMDA) receptors resulting in synaptic changes as well as activation of spinal microglia and a secondary spinal cord inflammation through local cytokine release. It has been suggested that pain associated with RA involves alterations in the brain (supraspinal). Pain is continuously modulated through a descending pathway which integrates input on mood, stress, sleep and more which affects pain perception. The executing region of the descending modulation of pain is the rostral ventromedial medulla, which integrates incoming information and determines how much pain signalling is allowed to travel through the spinal cord to the brain (Cao et al, 2020).

Peripheral sensitisation may be due to articular inflammation. This pain sensitivity may indicate sensitisation of peripheral or central nociceptive pathways and contributes to the clinical pain reported by people with RA (Joharatnam et al, 2015).

By “joint”, we include the meaning of the anatomical area of the patient at which two or more skeletal components are connected. For example, the metacarpophalangeal joint is those between the first and second phalanges, proximal interphalangeal joint is where the finger bones meet the hand bones, elbow joint is the connection between the humerus, the ulna, and the radius bones. The joint may include the bones themselves, cartilage, ligaments, articular capsule, synovial membrane, bursae and/or synovial fluid. The joint may be a fibrous joint, a cartilaginous joint, synovial joint, an ankle joint, a condylar joint (such as those found in the wrist and at the base of the index finger), an elbow joint, a glenohumeral joint, a humeral (shoulder) joint, a sacroiliac joint, a hip joint, a knee joint, temporomandibular joint.

By “inflammatory joint pain”, we include the meaning that pro-inflammatory factors (e.g. cytokines, lipid mediators, peptides, growth factors) present systemically (e.g. in the serum) and/or locally at the site of pain, such as the joint (e.g. the synovial joint), leading to pain, swelling and tenderness. Inflammatory pain can be assessed by identifying clinical inflammation.

By “clinical inflammation”, we include the meaning that the levels of inflammation can be detected using standard clinical measures and markers of inflammation. Those skilled in the art will be able to select appropriate measures, for example erythrocyte sedimentation rate (ESR or“sed” rate), swollen/tenderjoin count, hand tenderness, acute phase reactants (APRs) such as C-reactive protein (CRP), ferritin, plasma fibrinogen and platelet count. Those skilled in the art will be able to select appropriate markers of inflammation, for example proinflammatory cytokines present systemically and/or locally at the site of pain, such as TNF, IL-6, IL1, GM-CSF, IL17, IL20, IL23, IL24. Changes and statistical tests used to compare levels of cytokines would be known to the skilled person.

In an embodiment, the pain is not inflammatory pain. By “not inflammatory pain”, we include the meaning that the pain is present in the absence of clinical inflammation. Non-inflammatory pain is pain that is not associated with measures of inflammation, and is common in patients with RA. In this case, the patient experiences pain associated with RA despite there being an apparent absence (or a “healthy” level) of pro-inflammatory factors. Without being bound by theory, the inventors believe that there remains a level of subclinical inflammation in such circumstances.

In one embodiment the pain is not neuropathic pain or neuroplastic pain.

In an alternative embodiment, the pain is neuropathic pain or neuroplastic pain. Neuropathic pain may occur in isolation or in combination with other forms of pain, such as inflammatory pain. In some embodiments, where type I interferon inhibitors may be particularly useful, the neuropathic pain occurs in combination with inflammatory pain.

By “neuropathic pain”, we include the meaning that pain that is caused by lesion, disease, and/or damage to the nervous system. This can include the peripheral nervous system (PNS) and/or the central nervous system (CNS). In some embodiments, the neuropathic pain may be peripheral neuropathic pain, central neuropathic pain, or mixed (peripheral and central) neuropathic pain. Appropriate tests for determining the presence of pain will be well known to those skilled in the art. For example, using questionnaires (such as VAS), quantitative sensory testing etc. Appropriate tests for determining whether the pain is neuropathic pain will be well known to those skilled in the art. For example, clinicians may look for an underlying lesion to the CNS or PNS or an inciting cause consistent with the development of neuropathic pain. Magnetic resonant imaging (MRI), Quantitative sensory testing (QST), sudomotor assessments, or skin biopsies may also be used.

By “neuroplastic pain”, we include the meaning that pain symptoms are caused by learned neural pathways in the brain that are not due to ongoing structural damage or disease in the body. Neuroplastic pain does not involve any damage or alterations in incoming sensory system. Neuroplastic changes in brain structure and function can be a consequence of chronic pain and may also be involved in the maintenance of pain symptoms. Appropriate tests for determining the presence of pain will be well known to those skilled in the art. For example, clinical examination to determine the presence of pain that is not proportional to injury, burning and tingling ongoing/spontaneous pain, and/or neurological examination to determine the presence of nerve injury or lesions/damage in spinal cord or brain.

Regardless of the origin of the pain and the mechanisms involved (discussed herein), the pain “experience” can be perceived as the same by the patient. For example, non-inflammatory pain processes, such as those involved in some neuropathic pain, may produce the same pain symptoms as those produced by inflammatory pain processes.

In an embodiment, the pain is chronic pain. By “chronic pain”, we include the meaning of pain that extends beyond the expected period of healing. Chronic pain is contrasted with acute pain, which is much shorter lasting. Chronic pain may be nociceptive and/or neuropathic chronic pain. Chronic pain often persists irrespective of RA treatment.

Preferably, “chronic pain” is pain that is present for twelve weeks or more, three months or more, thirteen weeks or more, fourteen weeks or more, fifteen weeks or more, sixteen weeks or more, four months or more, five months or more, six months or more, seven months or more, eight months or more, nine months or more, ten months or more, eleven months or more, twelve months or more, one year or more, thirteen months or more, fourteen months or more, fifteen months or more, sixteen months or more, seventeen months or more, eighteen months or more, two years or more, three years or more, four years or more, five years or more, ten years or more, or for the remainder of the patient's lifetime.

By “pain that is present for”, we include the meaning that the pain may persist for, recur and/or progress over the time period.

By “pain may persist for the time period”, we include the meaning that the pain is continuous pain, such that at all time points assessed, the pain may be present at the same, at a lower, or at a higher intensity than at the start. For example, when tested over multiple time points, there may be no statistically significant changes in the recognised pain tests and scales discussed herein. Therefore, the pain neither improves nor worsens, yet remains over time.

By “pain may recur over the time period”, we include the meaning that, when tested over multiple time points, although there is an initial statistically significant improvement in at least one of the recognised pain tests and scales; however, at a later stage, the improvement is reversed. Therefore, during the twelve weeks or more, three months or more, thirteen weeks or more, fourteen weeks or more, fifteen weeks or more, sixteen weeks or more, four months or more, five months or more, or six months or more, seven months or more, eight months or more, nine months or more, ten months or more, eleven months or more, twelve months or more, one year or more, thirteen months or more, fourteen months or more, fifteen months or more, sixteen months or more, seventeen months or more, eighteen months or more, two years or more, three years or more, four years or more, five years or more, ten years or more, the remainder of the patient's lifetime, the pain may be absent at one or more of the time points assessed but recurs.

By “pain may progress over the time period”, we include the meaning that, when tested over multiple time points, there is a statistically significant improvement in at least one of the recognised pain tests and scales discussed herein. Therefore, the pain improves (i.e. reduces) over time.

In yet another embodiment, the pain is one or more selected from the group consisting of: pain hypersensitivity; allodynia; hyperalgesia; arthralgia.

By “pain hypersensitivity”, we include the meaning that the patient experiences an increased sensitivity to pain, that is, an abnormal painful reaction. In a patient with pain hypersensitivity, the pain threshold (the point at which something uncomfortable or unpleasant causes pain) can be lower compared to when pain hypersensitivity is absent in that patient or another patient. Different types of pain hypersensitivity can include hyperalgesia and allodynia. Hyperalgesia and allodynia can be caused by neural and non-neural mechanisms (for example skin, joints) and can be experienced in focal areas, discrete areas, or as a more diffuse, body-wide form.

By “allodynia”, we include the meaning of a pain response due to a stimulus that does not normally provoke pain. In allodynia, the patient will experience pain in situations where a stimulus normally does not elicit pain (i.e. a non-painful stimulus can be experienced as painful). Different types of allodynia include mechanical/tactile allodynia, static mechanical allodynia, dynamic mechanical allodynia, thermal (hot or cold) allodynia, movement allodynia (i.e. pain is triggered by normal movement of the joints or muscles). A skilled person would be aware of suitable methods for determining allodynia, for example using a cotton swab or a brush for assessing dynamic mechanical allodynia, a pressure algometer and standardised monofilaments, Quantitative sensory testing (QST), a thermal tester, or the Von Frey Test used in RA patients or animal models of RA.

By “hyperalgesia”, we include the meaning of an exaggerated or inappropriate response to a painful stimulus. In hyperalgesia, the patient can experience pain in situations where pain is “normal”, but the intensity of pain may be experienced as more severe compared to when hyperalgesia is absent in that patient or another patient. The patient with hyperalgesia may respond faster to a painful stimulus compared to when hyperalgesia is absent in that patient or another patient. The pain experienced from a painful stimulus by a patient with hyperalgesia may last longer compared to the pain experienced from the same painful stimulus by that patient when hyperalgesia is absent or by another patient. Hyperalgesia may be primary hyperalgesia, secondary hyperalgesia, referred hyperalgesia, visceral hyperalgesia or a combination thereof. A skilled person would be aware of suitable methods for determining hyperalgesia; for example, one could apply a painful stimulus to a patient and rate the experienced pain, Quantitative sensory testing (QST), or a pressure algometer and weighted pinprick stimuli.

In these forms of increased sensitivity, neurons may have increased excitability and/or may produce action potentials without any stimuli (ongoing or spontaneous pain) or increased number of action potentials as compared to normal neurons following stimuli (pain hypersensitivity). The increased sensitivity can be caused by lower threshold for firing, hypopolarisation, larger and/or longer receptor potential amplitudes, more efficient generation of action potentials from receptor potentials. In some situations, the increased sensitivity is caused by ectopic activity of sensory neurons, such as sleeping or silent nociceptors which could be considered to be ‘unsilenced’, ‘woken up’.

By “arthralgia”, we include the meaning that the pain is joint pain. Such joint pain can be present in the presence or in the absence of disease inflammation, for example during or after remission of RA, or in RA patients where the inflammatory disease is well controlled. Preferably, the arthralgia associated with RA to be prevented or treated is not one that is present in the prodromal stages of RA. A skilled person would be aware of suitable methods for determining arthralgia, for example by the use of questionnaires and performing physical examination of the patient.

As will be appreciated, and as described herein, the pain may be associated with and/or caused by:
systemic inflammation; and/or
local inflammation; and/or
clinical inflammation.

By “systemic inflammation”, we include the meaning that the inflammation may be present anywhere in the body as a whole. Inflammatory factors (e.g. cytokines, lipid mediators, peptides, growth factors) may be present in the blood cells and/or serum.

By “local inflammation”, we include the meaning that the inflammation may be present only in a localised part of the body. Inflammatory factors (e.g. cytokines, lipid mediators, peptides, growth factors) may be present in a specific organ, joint, or other location of the body and absent in another location of the body. The inflammatory factors may be different or overlapping with those present in systemic inflammation.

By “caused by systemic inflammation; and/or local inflammation; and/or clinical inflammation”, we include the meaning that pain is a direct consequence of the systemic inflammation; and/or local inflammation; and/or the clinical inflammation.

By “associated with systemic inflammation; and/or local inflammation; and/or clinical inflammation”, we include the meaning that the pain is not necessarily caused by the systemic inflammation; and/or local inflammation; and/or the clinical inflammation, but that the pain exists concurrently with such inflammatory states.

In an embodiment, the pain is not associated with and/or is not caused by Rheumatoid Arthritis inflammatory disease activity.

By “Rheumatoid Arthritis inflammatory disease activity”, we include the meaning that the RA patient is in the active RA inflammatory disease state. By “the active RA inflammatory disease state”, we include the meaning that the inflammation reaches the level of clinical inflammation, which can be assessed as described herein. For example, by Clinical Disease Activity Index (CDAI), by Routine Assessment of Patient Index Data 3 (RAPID3), by DAS28 score. Inflammatory disease activity may be classified as low activity, moderate activity, or high activity, as known in the art. For example, a DAS28 score greater than or equal to 2.6 and less than 3.1 indicates low activity; a score greater than or equal to 3.1 and less than 5.1 indicates moderate activity and a score a score greater than or equal to 5.1 indicates high activity.

A patient can be in an inactive RA inflammatory disease state before, during or after being considered as “having RA”. A patient is considered as having RA when diagnosed as such, including differential diagnosis of other inflammatory polyarthritis diagnoses.

A patient in an inactive RA inflammatory disease state before being considered as “having RA” may be experiencing any of the pain described herein as well have circulating antibodies to citrullinated peptides (ACPA)/cyclic citrullinated peptide (CCP). In a preferred embodiment, the inactive RA inflammatory disease state is not present before the patient has RA.

A patient in an inactive RA inflammatory disease state during being considered as “having RA” may be able to control inflammatory disease using DMARDs; however, upon stopping using DMARDs (i) the patient would fall back into an active RA inflammatory state, or (ii) the patient will remain in an inactive RA inflammatory disease state (i.e. RA remission). By “remission” we include the meaning of a decrease in or disappearance of inflammatory signs and/or symptoms. Those skilled in the art will be capable of selecting the appropriate method of determining remission, for example a DAS28 score of less than 2.6 indicated remission.

By “not caused by Rheumatoid Arthritis inflammatory disease activity”, we include the meaning that pain is not direct consequence of the RA inflammatory disease activity (also called the active RA inflammatory disease state). Therefore, removal or reduction of this RA inflammatory disease activity should not alleviate or stop the pain.

By “pain not associated with by Rheumatoid Arthritis inflammatory disease activity”, we include the meaning that the pain does not necessarily happen concurrently with RA inflammatory disease activity. Therefore, the pain may be present in the absence of RA inflammatory disease activity (also called the inactive RA inflammatory disease state).

In another embodiment, the pain is at an affected joint and/or opposite parts of an affected joint and/or cephalic parts of an affected joint and/or caudal parts of an affected joint.

By “affected joint”, we include the meaning of the joint where there is inflammation, swelling, tenderness, and/or where the onset of pain was first experienced.

By “opposite parts of an affected joint”, we include the meaning of the opposite parts of an affected joint in relation to the left-right axis.

By “cephalic parts of an affected joint” and “caudal parts of an affected joint”, we include the meaning of the cephalic (top) and caudal (bottom) parts of an affected joint in relation to cephalo-caudal axis. For example, if the affected joint is the right knee joint, an opposite part of the affected joint could be the left knee joint, a cephalic part of the affected joint could be any of the shoulder joints, a caudal part of the affected joint could be any of the ankle joints.

In an alternative or additional embodiment, the pain is extra-articular pain. By “extra-articular pain”, we include the meaning that the pain is not joint pain. Such extra-articular pain, unpleasantness or itch may be experienced as coming from the skin of the patient.

In an embodiment, the type I interferon inhibitor does not prevent or treat an inflammatory disease with increased type I interferon signalling.

By “an inflammatory disease with increased type I interferon signalling”, we include any of RA, polyarthritis, Aicardi-Goutieres syndrome (AGS)1, systemic lupus erythematosus (SLE), Crohn's disease, Psoriasis, Psoriatic arthritis, osteoarthritis, dermatomyositis, primary Sjögren syndrome, systemic sclerosis and type I interferonopathies and fibromyalgia.

In one embodiment, the pain is present along with disease inflammation.

By “present along with disease inflammation”, we include the meaning that pain can be present in a patient at the same time as the patient being in the active RA inflammatory disease state. The patient may or may not be receiving treatment, such as DMARDs. In the case that the patient is receiving DMARD treatment and is still in an active RA inflammatory disease state, it will be considered that the patient does not have a complete control of inflammation.

In an alternative embodiment, the pain is present in the absence of disease inflammation.

The patient may have gone from being in an active RA inflammatory disease state to an inactive RA inflammatory disease state because of treatments, such as DMARDs. In the case that the patient is receiving a DMARD treatment and is no longer in active RA inflammatory disease state, it will be considered that the patient has control of inflammation.

In a preferred embodiment, the pain is not present before disease inflammation. By “before disease inflammation”, we include the meaning of the prodromal stage of RA. Patients presenting with both pain (such as arthralgia) and circulating antibodies to citrullinated peptides (ACPA)/cyclic citrullinated peptide (CCP) may be considered to be in the prodromal stage of RA.

In one embodiment, the pain is present during or after the remission of disease inflammation.

By “during the remission of disease inflammation”, we include the meaning that pain can be present in a patient who has a decrease of inflammatory signs and/or symptoms. The patient may or may not be still in an active RA inflammatory disease state. The decrease of inflammatory signs and/or symptoms may be caused by the treatments, such as DMARDs. During remission of disease inflammation, the RA patient may still be reliant on such treatments to enter and remain in an inactive RA inflammatory disease state.

By “after the remission of disease inflammation”, we include the meaning that pain can be present in a patient where inflammatory signs and/or symptoms are no longer present/measurable. The patient may be still in an inactive RA inflammatory disease state. The disappearance of inflammatory signs and/or symptoms may be caused by the treatments, such as DMARDs. After remission of disease inflammation, the RA patient may still no longer be reliant on such treatments to remain in an inactive RA inflammatory disease state.

As will be appreciated, and as described herein the patient may have received, or may be receiving pain treatment, but the pain may persist and/or may recur and/or may progress.

Pain treatments are known in the art and may include nonsteroidal anti-inflammatory drugs (NSAIDs), such as celecoxib, diclofenac, etoricoxib, ibuprofen, naproxen; steroidal drugs such as corticosteroid, glucocorticoid (e.g. prednisolone); acetaminophen (also known as paracetamol); weak opioids, such as codeine, dextropropoxyphene, tramadol; antidepressants, such as tricyclic antidepressant; an anticonvulsant; or a combination thereof. Where the pain persists, recurs or progresses, the pain treatment may be considered ineffective, sub-optimal and/or failed.

NSAIDS have been estimated to relieve symptoms in only about fifteen out of one hundred people. Steroids and weak opioids can help to reduce pain and swelling in the affected joints; however, extended usage can lead to severe side-effects and thus, they are predominantly used in the short-term management of RA pain (McWilliams et al, 2022; Day et al, 2019). Paracetamol only has a weak anti-inflammatory effect, and has been shown to relieve pain associated with RA less even effectively than NSAIDs do (Bullock et al, 2018; Evidence review G Analgesics, NICE guidelines, 2018).

By “the patient may have received pain treatment”, we include the meaning that at the time of performing assessment of pain as described herein, the patient is no longer receiving pain treatment.

By “the patient may be receiving pain treatment”, we include the meaning that at the time of performing assessment of pain as described herein, the patient is still receiving pain treatment.

In one embodiment, the pain is associated with and/or caused by increased type I interferon signalling in the patient.

Changes (increase or reductions) of type I interferon signalling can be detected in the serum and at different locations in the patient (such as in the tissue or cells surrounding sensory neurons and nerves). Depending on the location of the sample (for example blood, synovium or sensory neuron, nerve or skin samples), the markers of increased/reduced type I interferon signalling can differ. Consequently, methods of determining whether type I interferon signalling is different compared to a healthy control will be adapted depending on the type of sample obtained from a patient.

By “the pain is associated with type I interferon signalling in the patient”, we include the meaning that the pain is not necessarily caused by increased type I interferon signalling in the patient, but that the pain may happen concurrently to the increased type I interferon signalling in the patient.

By “the pain is caused by increased type I interferon signalling in the patient”, we include the meaning that pain is a direct consequence of increased type I interferon signalling in the patient. Therefore, decrease of increased type I interferon signalling in the patient may be able to alleviate or stop the pain.

Preferably, the increased type I interferon signalling comprises:
increased type I interferon intracellular signalling in the patient
increased levels of type I interferons in the patient;
increased activation of type I interferon receptors in the patient;
increased expression of one or more type I interferon-stimulated gene in the patient; and/or
reduced expression of one or more type I interferon-repressed gene in the patient.

As will be appreciated, a patient may have increased levels of type I interferon without increased type I interferon intracellular signalling, for example due to cell intrinsic negative feedback mechanisms which can lead to a maintenance or reduction in signalling.

Alternatively, a patient may have increased levels of type I interferon and/or increased activation of type I interferon receptors without increased expression of one or more interferon-stimulated genes, for example due to cell intrinsic inhibitors of interferon-induced transcription (which may or may not affect signalling).

By “increased” signalling, levels, activation, expression and by “reduced” expression, we include the meaning that the signalling, levels, activation or expression is statistically significantly increased or reduced compared to baseline, for example in patients where pain is not present or in healthy controls. In some embodiments, the p value is less than 0.05, less than 0.04, less than 0.03, is less than 0.02, is less than 0.01, is less than 0.001, is less than 0.0001.

A person skilled in the art would be able to select the appropriate assay for measuring type I interferon intracellular signalling in the patient.

A person skilled in the art would be able to select the appropriate assay for measuring levels of type I interferon in the patient. Methods might include but are not limited to quantitative PCR (qPCR), single cell or bulk RNA-sequencing, hybridisation-based methods, or enzyme-linked immunosorbent assay (ELISA), sodium dodecyl sulfate-polyacrylamide gel electrophoresis (SDS-PAGE), mass spectrometry, reporter cell assays. For example, transcript levels of any of the Interferon-alpha genes listed herein could be assessed.

A person skilled in the art would be able to select the appropriate assay for measuring activation of type I interferon receptors. Methods might include but are not limited to quantitative PCR (qPCR), single cell or bulk RNA-sequencing, hybridisation-based methods, or enzyme-linked immunosorbent assay (ELISA), sodium dodecyl sulfate-polyacrylamide gel electrophoresis (SDS-PAGE), mass spectrometry, phosphorylation/dephosphorylation states. For example, phosphorylation of the intracellular domains of IFNAR1, IFNAR2, TYK2, MNK1, MNK2, and/or eukaryotic translation initiation factor 4E (eIF4E) could be assessed.

A person skilled in the art would be able to select the appropriate assay for measuring increased expression of one or more type I interferon-stimulated gene or reduced expression of one or more type I interferon-repressed gene. Methods might include but are not limited to quantitative PCR (qPCR), single cell or bulk RNA-sequencing, hybridisation-based methods, or enzyme-linked immunosorbent assay (ELISA), sodium dodecyl sulfate-polyacrylamide gel electrophoresis (SDS-PAGE), mass spectrometry, reporter cell assays. For example, transcript levels of any of the interferon-stimulated genes listed herein could be assessed.

As will be appreciated, the level of significant fold change in transcript levels measured by, for example, qPCR, may depend on the specific transcript being measured and the specific household transcript used for standardisation. In some embodiments, the value of fold change is at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 6 fold, at least 7 fold, at least 8 fold, at least 9 fold, at least 10 fold, at least 20 fold, at least 30 fold, at least 40 fold, at least 50 fold.

In some embodiments, samples may be obtained systemically, such as blood samples (e.g. whole blood cells, peripherical blood mononuclear cells) or serum. In an additional and/or alternative embodiments, samples may be obtained locally, such as within the dorsal root ganglion (DRG), synovium, nerve, skin, or muscle. Anatomically, a DRG emerges from the dorsal root of the spinal nerves. They carry sensory messages from various receptors (i.e., pain and temperature) at the periphery towards the central nervous system for a response. The role of DRG in chronic pain has been well established; however, up until now, it was not known that which specific cell type were affected in pain associated with RA.

The increase or reduction can occur over time and may be measured by obtaining samples periodically. In one embodiment, the increase or reduction could be a transient increase, with signalling, levels, activation or expression returning to baseline after a few hours. In another embodiment, the increase could be a persistent increase or reduction, with signalling, levels, activation or expression remaining increased or reduced over days, weeks or months. In some embodiment, the systemic increase in type I interferon signalling is transient. In another embodiment, the local increase in in type I interferon signalling is persistent.

Suitable type I interferons are selected from the group comprising: Interferon-alpha; Interferon-beta.

In another embodiment, the pain is associated with increased number and/or activity of one or more active sensory neuron in the patient; preferably increased number and/or activity of one or more nociceptors of the patient.

By “sensory neurons”, we include the meaning of afferent neurons, specific cell type of the nervous system that converts stimuli into action or graded potentials or graded potentials. In vertebrates, the cell bodies of the sensory neurons are located in the dorsal ganglia of the spinal cord, jugular ganglion and trigeminal ganglion. The sensory information travels from the ganglia to the brain via the spinal cord or brain stem.

As will be appreciated, a specific type of sensory neuron is the nociceptor (also called pain receptor). Nociceptors respond to damage, or threat of damage, to body tissues, leading (often but not always) to pain perception. The brain creates the sensation of pain to direct attention to the body part, so the threat can be mitigated; this process is called nociception.

Nociceptors can be found in internal (such as nociceptor in muscles, joints, bladder, visceral organs, gastro-intestinal tract) or external (such as cutaneous nociceptors, nociceptors in the cornea, mucosa) and can detect different kinds of noxious stimuli.

Noxious stimuli are detected at the peripheral terminal of the mature nociceptor and transduced into electrical energy. When this energy reaches a threshold value, an action potential is induced and driven towards the CNS. This in turn leads to pain perception. The sensory specificity of nociceptors is established by the high threshold only to particular features of stimuli. Only when the high threshold has been reached by either chemical, thermal, or mechanical environments are the nociceptors triggered.

The majority of nociceptors are classified by the type of modality they respond to. For example, some nociceptors may respond to one modality and are referred to as unimodal nociceptors. Other nociceptors may respond to more than one of these modalities and are consequently designated polymodal nociceptors, such as C-polymodal nociceptors. Other nociceptors respond to none of these modalities (although they may respond to stimulation under conditions of inflammation of the surrounding tissue) and are referred to as sleeping or silent nociceptors. Alternatively, nociceptors can be classified by the axon conveying the pain signal and fall into either the A-fiber nociceptors (such as Aδ nociceptors and Aβ nociceptors), which conduct above 5 m/s, or into the C-fiber nociceptors, which conduct at velocities of generally less than 2 m/s. In one embodiment, the sensory neurons are C-polymodal nociceptors and/or C-fiber nociceptors and/or A-fiber nociceptors.

By “active sensory neuron”, we include the meaning that the sensory neuron generates one or more electrical current resulting in one ore more graded potential and/or one or more action potential. A person skilled in the art would be able to select the appropriate assay for assessing whether a sensory neuron is active, for example by using electrophysiology techniques.

It will be appreciated that a relatively small increase in the number of active sensory neurons is sufficient to result in pain and/or an increase in pain—for example, activation or addition of one or a few sensory neurons (out of the approximately 10,000 in each ganglion) is sufficient to result in pain and/or an increase in pain.

In one embodiment, the increase in the number of active sensory neurons is an increase (compared to inactive sensory neurons) in one or more ganglion of: at least 0.0001%, at least 0.0002%, at least 0.0005%, at least 0.001%, at least 0.002%, at least 0.005%, at least 0.01%, at least 0.02%, at least 0.05%, at least 0.1%, at least 0.2%, at least 0.5%, at least 1%, at least 2%, at least 5%, at least 10%.

In another embodiment, the increase in the number of active sensory neurons is an increase in one or more sensory ganglion of: at least 100%, at least 125%, at least 150%, at least 175%, at least 200%, at least 225%, at least 250%, at least 275%, at least 300%, at least 325%, at least 350%, at least 375%, at least 400%, at least 425%, at least 450%, at least 475%, at least 500%, at least 525%, at least 550%, at least 575%, at least 600%.

By “increase in activity of one or more sensory neuron”, we include the meaning that the sensory neuron may have an increased number of action potentials (also known as nerve impulses) and/or larger receptor potential amplitudes and/or longer receptor potential amplitudes. This can occur during chronic pain. This increase in the number of action potentials and/or larger receptor potential leads to an increase in the intensity of pain. A person skilled in the art would be able to select the appropriate assay for measuring the activity of sensory neurons, for example by using microneurography techniques.

In one embodiment, the sensory neurons of the patient are tropomyosin receptor kinase A (TrkA)-expressing sensory neurons; preferably (TrkA)-expressing nociceptors.

TrkA may also be known as tyrosine kinase A, high affinity nerve growth factor (NGF) receptor, neurotrophic tyrosine kinase receptor (NTKR) type 1, or TRK1-transforming tyrosine kinase protein; TRK; TRK1; TRKA; Irk-A; p140-TrkA. In humans, TrkA is encoded by the NTRK1 gene. This kinase is a membrane-bound receptor that, upon neurotrophin binding, autophosphorylates and activates intracellular signalling via the MAPK pathway. In some embodiments, the TrkA-expressing sensory neurons are located in the DRG.

In additional or alternative embodiment, the sensory neurons of the patient are GDNF family receptor alpha 3 (GFRa3)-expressing sensory neurons; preferably GFRa3-expressing nociceptors.

GFRa3 may also be known as GFRA3 or GFR-alpha 3. In humans, GFRa3 is encoded by the GDNFR3 gene. This protein is a glycosylphosphatidylinositol (GPI)-linked cell surface receptor and a member of the glial cell line-derived neurotrophic factor (GDNF) receptor family. It forms a signalling receptor complex with RET tyrosine kinase receptor and binds the ligand, artemin (also known as enovin or neublastin). In some embodiments, the GFRa3-expressing sensory neurons are located in the DRG.

In additional or alternative embodiment, the sensory neurons of the patient are transient receptor potential cation channel subfamily V member 1 (TrpV1)-expressing sensory neurons; preferably TrpV1-expressing nociceptors.

TrpV1 may also be known as transient receptor potential vanilloid subfamily-1 receptor, vanilloid receptor, capsaicin receptor, VR1. In humans, TrpV1 is encoded by the TRPV1 gene. Four transcript variants encoding the same protein, but with different 5′ UTR sequence, have been described for this gene. The protein encoded by this gene is a receptor for capsaicin and is a non-selective cation channel that is structurally related to members of the TRP family of ion channels. TrpV1 is a polymodal channel sensitive to different physical and chemical stimuli, including heat, low pH and mechanical stimuli and can be activated by different ligands (such as vanilloids, capsaicinoids, resiniferanoids, cannabinoids, ginsenosides). In sensory neurons, the TrpV1 channel may interact with the TRPA1 ion channel to mediate the detection of noxious stimuli. In some embodiments, the TrpV1-expressing sensory neurons are located in the DRG.

In additional or alternative embodiment, the sensory neurons of the patient are Calcitonin gene related peptide (CGRP)-expressing sensory neurons; preferably CGRP-expressing nociceptors.

CGRP may also be known as Calca. In humans, CGRP is encoded by the Calca gene, also called CT, KC, PCT, CALC1, CGRP1, CGRP-I, CGRP-alpha. Calcitonin gene-related peptide is a member of the calcitonin family of peptides and is secreted and stored in the nervous system. In humans, the peptide exists in two forms: CGRP alpha (also called alpha-CGRP or CGRP I), and CGRP beta (also called beta-CGRP or CGRP II). CGRP may bind to calcitonin receptor-like receptor (CALCRL) and a receptor activity-modifying protein (RAMP1). In some embodiments, the CGRP-expressing sensory neurons are located in the DRG. In some embodiments, CGRP-expressing sensory neurons release the CGRP protein which can affect inflammation by causing vasodilation and increasing vascular permeability leading to leakage from vessels (ie plasma protein extravasation) and swelling. CGRP can also activate immune cells, such as mast cells. This leads to a phenomenon is known as neurogenic inflammation.

By “expressing”, we include the meaning that the relevant gene is transcribed and optionally translated by the cell and is shuttled to the correct location in the cell, such as the cell surface or is secreted from the cell.

A person skilled in the art would be able to determine whether a sensory neuron is a TrkA, a GFRa3, a TrpV1 and/or a CGRP-expressing sensory neuron, using methods known in the art. For example, in situ hybridisation, RNA sequencing, immunostaining, gene reporter strains, optogenetics.

### Type I Interferon Inhibitors

In one embodiment, the type I interferon inhibitor reduces type I interferon signalling in the patient.

As will be appreciated, when administered to the patient, the type I interferon inhibitor may reverse the increased type I interferon signalling. Changes in type I interferon signalling can be detected using the method discussed herein.

In some embodiments, the type I interferon inhibitor:
prevents or reduces type I interferon intracellular signalling in the patient;
prevents or reduces levels of type I interferons in the patient;
prevents or reduces activation of type I interferon receptors in the patient;
prevents or reduces expression of one or more type I interferon-stimulated gene in the patient; and/or
induces and/or increases expression of one or more type I interferon-repressed gene in the patient.

A person skilled in the art would be able to assess the ability of type I interferon inhibitor to reduce type I interferon signalling in the patient through in vitro or in vivo assays. For in vitro assays, the type I interferon inhibitor could be applied to cells. In an embodiment, an in vitro method for screening type I interferon inhibitors suitable for use in treating or preventing pain associated with Rheumatoid Arthritis in a patient may comprise: i) applying the inhibitor to a sensory neuron with increased type I interferon signalling; and ii) measuring the reduction in type I interferon signalling.

For in vivo assays, the type I interferon inhibitor could be administered to a patient. Methods for measuring type I interferon intracellular signalling, levels of type I interferons, activation of type I interferon receptors, expression of type I interferon-stimulated genes, expression of type I interferon-repressed genes are discussed herein.

By “the type I interferon inhibitor prevents”, we include the meaning that the type I interferon inhibitor may prevent (i) the increased type I interferon intracellular signalling in the patient, (ii) the increased levels of type I interferons in the patient, (iii) the increased activation of type I interferon receptors in the patient and/or (iv) the increased expression of one or more type I interferon-stimulated gene in the patient.

By “the type I interferon inhibitor reduces”, we include the meaning the type I interferon inhibitor may reduce (i) the increased type I interferon intracellular signalling in the patient, (ii) the increased levels of type I interferons in the patient, (iii) the increased activation of type I interferon receptors in the patient and/or (iv) the increased expression of one or more type I interferon-stimulated gene in the patient.

By “the type I interferon inhibitor induces expression of one or more type I interferon-repressed gene in the patient”, we include the meaning the type I interferon inhibitor may ‘turn on’ or ‘de-repress’ the expression of one or more type I interferon-repressed gene in the patient.

By “the type I interferon inhibitor increases expression of one or more type I interferon-repressed gene in the patient”, we include the meaning type I interferon inhibitor may increase the reduced expression of one or more type I interferon-repressed gene in the patient.

Preferably, the type I interferon inhibitor is selected from the group comprising: an interferon alpha/beta receptor alpha chain (IFNAR1) inhibitor, an interferon alpha/beta receptor subunit 2 (IFNAR2) inhibitor, a tyrosine kinase 2 (TYK2) inhibitor, a type I interferon neutraliser, a MAP kinase-interacting serine/threonine-protein kinase (“MNK”) inhibitor (such as a MNK1 and/or MNK2 inhibitor), a eukaryotic translation initiation factor 4E (eIF4E) inhibitor.

By “type I interferon neutraliser”, we include the meaning that the agent will be able to neutralise, counteract, inhibit or block one or more type I interferon.

Suitable type I interferon inhibitors may be selected from the group comprising: a small molecule, a biologic (such as an antibody, an antibody mimetic, a decoy receptor, a receptor body, a vaccine).

By “small molecule”, we include the meaning of an organic compound with low molecular weight. By “biologic”, we include the meaning of a biological agent such as proteins and/or oligo- or polypeptides, enzymes, antibodies, antibody parts thereof, vaccines, antibody mimetics, or combinations thereof. Such antibodies may be monoclonal antibodies and can be produced using methods known in the art. Other polyclonal or chimeric antibody preparations may also be used.

By “antibody mimetic”, we include the meaning of an agent mimicking an antibody (for example, an affibody or other substance).

By “antibody parts thereof”, we include the meaning of an antibody fragment (for example, a nanobody).

As will be appreciated, neutralisation may arise by antibodies, antibody parts thereof (such as rontalizumab, sifalimumab, or S95021) or interferon decoy receptors binding to type I interferons (such as interferon-alpha and/or interferon-beta) to block interferon-receptor binding. Neutralisation may also arise by using a vaccine-based approach (such as the interferon-alpha kinoid (IFN-K)) to induce of active immunity (endogenous antibodies) against type I interferon (such as interferon-alpha and/or interferon-beta).

### IFNAR1 Inhibitors

In an embodiment, the type I interferon receptor is composed of the IFNAR1 and IFNAR2 subunits.

IFNAR1 may also be known as CRF2-1, IFN-R-1, IFN-alpha/beta receptor 1, IFN alpha/beta receptor 1, alpha-type antiviral protein, beta-type antiviral protein, cytokine receptor class-II member 1, cytokine receptor family 2 member 1, interferon (alpha, beta and omega) receptor 1, interferon receptor 1, interferon-alpha/beta receptor alpha chain, interferon-beta receptor 1, type I interferon receptor 1. In humans, IFNAR1 is encoded by the IFNAR1 gene. This receptor is membrane protein that forms one of the two chains of a receptor for interferon alpha and interferon beta.

IFNAR2 may also be known as IFN-alpha/beta receptor 2, IFN alpha/beta receptor subunit 2, human interferon alpha/beta receptor, interferon (alpha, beta and omega) receptor 2, interferon alpha binding protein, interferon receptor, interferon-alpha/beta receptor beta chain, type I interferon receptor 2. In humans, IFNAR2 is encoded by the IFNAR2 gene. This receptor is membrane protein that forms the other of the two chains of a receptor for interferon alpha and interferon beta.

Without being bound by theory, IFNAR1 inhibitors and IFNAR2 inhibitors may have an extracellular inhibiting action (such as by inhibiting binding of the receptor to type 1 interferons) and/or an intracellular inhibiting action (such as by inhibiting activated IFNAR1 or IFNAR2 downstream signalling).

In some embodiments the type I interferon inhibitor may inhibit both IFNAR1 and IFNAR2. In some embodiments, the IFNAR2 inhibitor is an IFNAR2a inhibitor, IFNAR2b inhibitor and/or IFNAR2c inhibitor.

Examples of IFNAR1 inhibitors include: anifrolumab (AstraZeneca), which is a human monoclonal antibody to IFNAR1 (DrugBank Accession Number: DB11976); MAR1-5A3 (InVivoMAb anti mouse IFNAR1, BioXCell), a monoclonal antibody which reacts with mouse IFNAR1.

### TYK2 Inhibitors

As will be appreciated, in humans TYK2 is encoded by the TYK2 gene. This protein is a member of the tyrosine kinase family and associates with the IFNAR receptors. Upon binding, TYK2 may phosphorylate the receptor units to activate downstream signalling.

Without being bound by theory, TYK2 inhibitors may work by inhibiting association with and/or phosphorylation of IFNAR receptors or in any other way to inhibit downstream signalling of type I interferon activated TYK2.

Examples of TYK2 inhibitors include: the allosteric tyrosine kinase 2 (TYK2) inhibitor deucravacitinib (also called Sotyktu)(Bristol Myers Squibb); brepocitinib and PF-06826647 (also called ropsacitinib) (Pfizer Inc); NDI-034858, NDI-031232, NDI-031301, NDI-031407 (Nimbus therapeutics); ESK-001 (Alumis Inc); VTX-958 (Ventyx Biosciences Inc); ICP-488 (InnoCare Pharma Ltd); GLPG3667 (Galapagos Inc); the TYK2 inhibitors described in WO-2022175745 and WO-2022136914 (Sudo Biosciences); the TYK2 inhibitors described in WO-2022104206, WO-2022011338 and WO-2022011337 (Neuron23); the TYK2 inhibitors described in WO-2013125543 and WO-2013146963 (Takeda); the TYK2 inhibitors described in WO-2019178079 and WO-2010010190 (AbbVie Inc); the TYK2 inhibitors described in EP2634185B1, WO-2015032423, WO-2018073438, WO-2021204762, WO-2020074461 (Sareum Ltd).

### MNK Inhibitors

As will be appreciated, in humans MNK1 and MNK2 are encoded by the MKNK1 and MKNK2 genes. These proteins are a member of the serine-threonine kinase family, and MNK activation associates with activation of the IFNAR receptors. Upon receptor binding, TYK2 may phosphorylate the receptor units to activate downstream signalling which leads to activation of MNK kinase.

Without being bound by theory, MNK inhibitors may work by inhibiting MNK activity on downstream substrates or in any other way to inhibit downstream signalling of type I interferon.

Those in the art will be aware of MNK inhibitors and/or be capable of identifying such inhibitors.

Examples of MNK inhibitors include those described in the following documents, all of which inhibitors and related disclosures are hereby incorporated by reference:
MNK inhibitors as described in WO2019079369, WO2015200481, WO2018152117, WO2017075412, WO2017075394, WO2017087808, WO2020086713, US2019152988, US2019330216, US2017266185, US2018085368, US2018085368, US2018228803, U.S. Ser. No. 11/083,727, US2019275039, US2017266185 (eFFECTOR Therapeutics Inc);
MNK inhibitors as described in WO2023278686, WO2023014943, and WO2022006331 (4E Therapeutics);
MNK inhibitors as described in WO2013174744, WO2012163942, WO2014118226, WO2014048894, WO2013174735, WO2015181104, WO2015181063, WO2018134335, WO2017081003, WO2014118229, WO2014118135, WO2014076162, WO2014044691, WO2015104254, WO2015004024, WO2015074986, WO2014128093, WO2014048869, WO2013034570, WO2012156367, WO2016102427, WO2016096721, WO2018134148 (Bayer Schering Pharma AG);
MNK inhibitors as described in WO2017085483 and WO2017085484 as well as CN108495855, EP3377501 and IP2018534314 (LifeArc);
MNK inhibitors as described in CN116425750 (Nuowosida Pharmaceutical Co., Ltd);
MNK inhibitors such as ETC-7114 and ETC-206, the latter also called tinodasertib available at MedChem Express, and related compounds as described in WO2013147711, WO2014088519 WO2019013703, WO2015050505 (Agency For Science Technology & Research Bioprocessing Technology Institute);
MNK inhibitors as described in CN110903286 (Shenyang Pharmaceutical University);
MNK inhibitors as described in WO2007147874 (Swedish Orphan Biovitrum AB);
MNK inhibitors as described in WO2007115822, WO2006136402 WO2008006547, WO2007059905 (Evotec (Goettingen) AG);
MNK inhibitors as described in WO2013087581, WO2013034570, WO2012163942, WO2012175591, WO2013041634, WO2013144189 (Bayer Intellectual Property GmbH);
MNK inhibitors as described in WO2015169677, WO2015091156, WO2006066937, WO2014206922, WO2011104340, WO2015082324, WO2010023181, WO2011104338, WO2013149909, WO2011104334, WO2006066937, US2015361055 (Boehringer Ingelheim Pharma GmbH & Co KG);
MNK inhibitor as described in CN103417545 (Taicang Shengzhou Biotechnology Co., Ltd);
MNK inhibitors as described in WO2020155842 (Novostar Pharmaceuticals Ltd);
MNK inhibitors as described in WO2020115072 (Instituto Ramon y Cajal de Investigacion Sanitaria:);
MNK inhibitors as described in US2018244654, WO2017075367 (Northwestern University):.
MNK inhibitors as described in WO2020233716, CN111978317, CN111978318 (Shanghai Daoshang Biology Tech Co., Ltd.);
MNK inhibitors as described in WO2020108619 (Shanghai De Novo Pharmatech Co Ltd);
MNK inhibitors as described in WO2017165908 and WO2023093700 (South Australian Health and Medical Research Institute and Ocean University of China);
MNK inhibitors as described in WO2017068064 (Ryvu Therapeutics);
MNK inhibitors as described in WO2010055072 (Friedrich Miescher Institute for Biomedical Research);
MNK inhibitors as described in WO2018228275 (Beijing InnoCare Pharma Tech Co Ltd);
MNK inhibitors as described in WO2021127589 (The Children's Hospital of Philadelphia);
MNK inhibitors as described in WO2016081589 and WO2021127438 (University of Maryland);
MNK inhibitors as described in WO2016128465 (Basilea Pharmaceutica Ltd, Allschwil);
MNK inhibitors as described in WO2023168381 (University of Maryland);
MNK inhibitors as described in WO2021005183 (Centro de Investigacion Biomedica en Red and Institut Quimic de Sarria and Vail d'Hebron Institut de Recerca);
MNK inhibitors as described in US2020197400 (New York University);
MNK inhibitors as described in WO2022237682 (Jumbo Drug Bank Co Ltd); and
MNK inhibitors as described in WO2022038563 (Hepagene Therapeutics (HK) Ltd).

In a preferred embodiment, the type I interferon inhibitor is an MNK inhibitor (such as an inhibitor of MNK1 and/or MNK2). Preferably, the MNK inhibitor is a small molecule, or is an antibody or part thereof. Preferably, the MNK inhibitor is selected from the group consisting of: eFT508 (also called Tomivosertib); 4ET-03-053; BAY1143269; ETC-1907206 (also called ETC-206 (AUM 001) and tinodasertib), and derivatives of the above compounds.

The formula of eFT508 (manufacturer MedChemExpress, Selleck Chemicals) (which is described in WO2015200481 and WO2020086713) is:

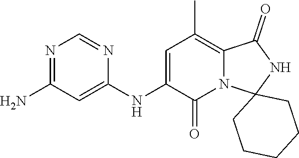 [SMILES: Cc1cc(Nc2cc(N)ncn2)c(=O)n2c1C(=O)NC21CCCCC1]

The formula of 4ET-03-053 (which are described in WO2023278686) is:

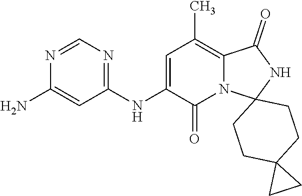 [SMILES: Cc1cc(Nc2cc(N)ncn2)c(=O)n2c1C(=O)NC21CCC2(CC2)CC1]

Additional MNK inhibitors described in WO2023278686 are:

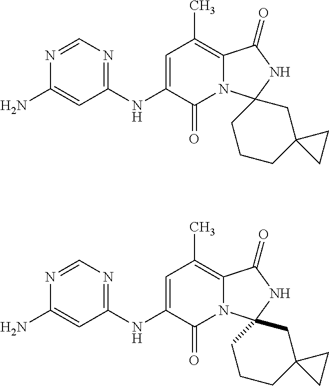

Further preferred MNK inhibitors include the following formula, and derivatives thereof (which are described in WO2023278686):

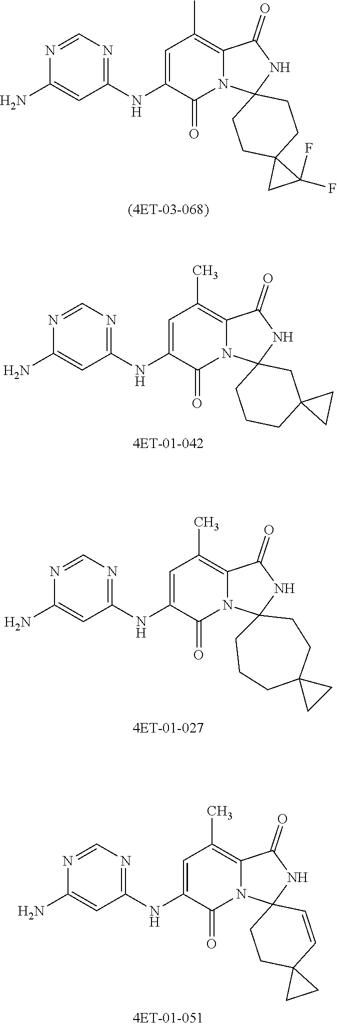 [SMILES: Cc1cc(Nc2cc(N)ncn2)c(=O)n2c1C(=O)NC21CCC2(CC1)CC2(F)F ; Cc1cc(Nc2cc(N)ncn2)c(=O)n2c1C(=O)NC21CCCC2(CC2)C1 ; Cc1cc(Nc2cc(N)ncn2)c(=O)n2c1C(=O)NC21CCCC2(CC2)CC1 ; Cc1cc(Nc2cc(N)ncn2)c(=O)n2c1C(=O)NC21C=CC2(CC2)CC1]

Further preferred MNK inhibitors include the following formula of 4ET-01-21, and derivatives thereof (which are described in WO2022006331):

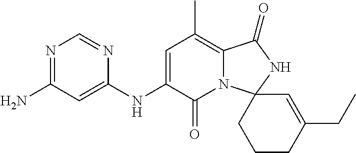 [SMILES: CCC1=CC2(CCC1)NC(=O)c1c(C)cc(Nc3cc(N)ncn3)c(=O)n12]

The formula of BAY-1143269 (which is compound 806820 in WO2013034570) is:

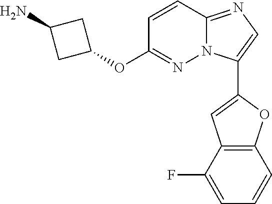 [SMILES: N[C@H]1C[C@H](Oc2ccc3ncc(-c4cc5c(F)cccc5o4)n3n2)C1]

The formula of ETC-1907206 (also called tinodasertib, AUM001, or ETC206, and described in WO2013147711) is:

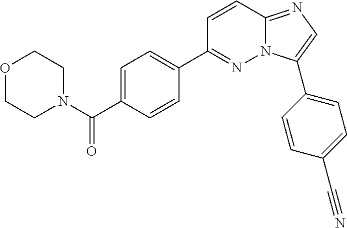 [SMILES: N#Cc1ccc(-c2cnc3ccc(-c4ccc(C(=O)N5CCOCC5)cc4)nn23)cc1]

In some embodiments, a MNK inhibitor is a MNK inhibitor described in Li, Q. et al., Discovery of D25, a Potent and Selective MNK Inhibitor for Sepsis-Associated Acute Spleen Injury, J. Med. Chem. 2024, 67, 4, 3167-89, which is incorporated herein by reference. For example, in some embodiments, a MNK inhibitor is compound D25 from Li et al.:

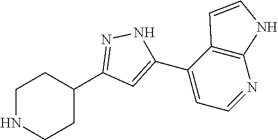 [SMILES: c1cc(-c2cc(C3CCNCC3)n[nH]2)c2cc[nH]c2n1]

In some embodiments, a MNK inhibitor is compound 7g, compound 18, or phorbazole C from Li et al.:

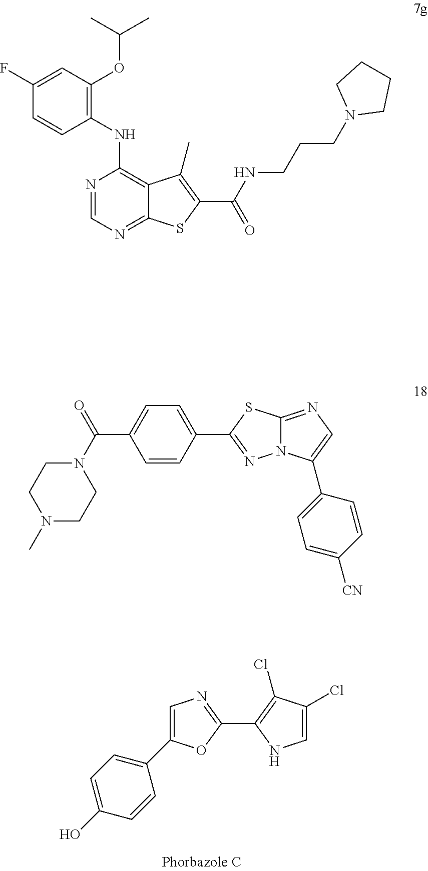 [SMILES: Cc1c(C(=O)NCCCN2CCCC2)sc2ncnc(Nc3ccc(F)cc3OC(C)C)c12 ; CN1CCN(C(=O)c2ccc(-c3nn4c(-c5ccc(C#N)cc5)cnc4s3)cc2)CC1 ; Oc1ccc(-c2cnc(-c3[nH]cc(Cl)c3Cl)o2)cc1]

In some embodiments, a MNK inhibitor is DS12881479, described in Matsui, Y., et al., A novel inhibitor stabilizes the inactive conformation of MAPK-interacting kinase 1, Acta Cryst. 2018, F74, 156-60:

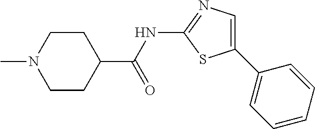 [SMILES: CN1CCC(C(=O)Nc2ncc(-c3ccccc3)s2)CC1]

### MNK Inhibitors of Structure I and Structure II

In one aspect, a MNK inhibitor is a compound of Structure (I):

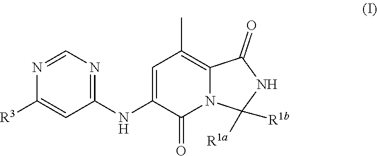

or a pharmaceutically acceptable salt thereof, wherein each of R^{1a}, R^{1b}, and R³ are as defined herein.

In another aspect, a MNK inhibitor is a compound of Structure (II):

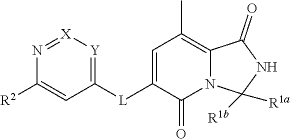

or a pharmaceutically acceptable salt thereof, wherein, R^{1a}, R^{1b}, R², X, Y, and L are as defined herein.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

The following definitions apply to Structure (I) and Structure (II) and subgenera thereof:

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Nitro” refers to the —NO₂ radical.

“Oxo” refers to the ═O substituent.

“Thiol” refers to the —SH substituent.

“Thioxo” refers to the ═S substituent.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching, but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂-C₈ alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching, but does not include carbon atoms belonging to any substituents. Unless stated otherwise specifically in the specification, an alkenyl group is optionally substituted.

The term “alkynyl” refers to unsaturated straight or branched hydrocarbon radical, having 2 to 12 carbon atoms (C₂-C₁₂ alkynyl), two to nine carbon atoms (C₂-C₉ alkynyl), or two to six carbon atoms (C₂-C₆ alkynyl), or any value within these ranges, and having at least one carbon-carbon triple bond. Examples of alkynyl groups may be selected from the group consisting of ethynyl, propargyl, but-1-ynyl, but-2-ynyl and the like. The number of carbons referred to relates to the carbon backbone and carbon branching, but does not include carbon atoms belonging to any substituents. Unless stated otherwise specifically in the specification, an alkynyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Aminyl” refers to a radical of the formula —NRₐR_{b}, where Rₐ is H or C₁-C₆ alkyl and R_{b} is C₁-C₆ alkyl as defined above. The C₁-C₆ alkyl portion of an aminyl group is optionally substituted unless stated otherwise.

“Aminylalkylcycloalkyl” refers to a radical of the formula —RₐR_{b}NR_{c}R_{d} where Rₐ is cycloalkyl as defined herein, R_{b} is C₁-C₆ alkyl, R_{c} is H or C₁-C₆ alkyl and R_{d} is C₁-C₆ alkyl as defined above. The cycloalkyl and each C₁-C₆ alkyl portion of an aminylalkylcycloalkyl group are optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of n-electrons (for example, alternating double and single bonds) that is even but not a multiple of 4 (i.e., 4n+2 n-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, and pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene.

“Aryl” as used herein, includes a fused ring system that includes non-aromatic moieties. For example, in some embodiments, aryl may have one of the following structures:

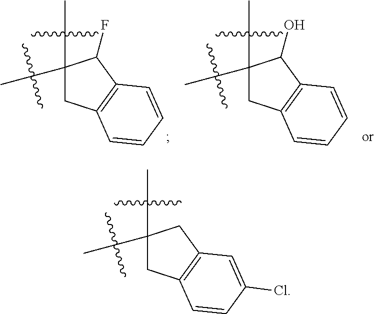 [SMILES: *C1(*)Cc2ccccc2C1F ; *C1(*)Cc2ccccc2C1O ; *C1(*)Cc2ccc(Cl)cc2C1]

Unless stated otherwise specifically in the specification, an aryl group is optionally substituted.

The term “arylalkyl” or “aralkyl” refers to the group -alkyl-aryl, where the alkyl and aryl groups are as defined herein. Aralkyl groups of the present disclosure are optionally substituted. Examples of arylalkyl groups include, for example, benzyl, 1-phenylethyl, 2-phenylethyl, 3-phenylpropyl, 2-phenylpropyl, fluorenylmethyl and the like.

“Cyanoalkyl” refers to an alkyl group comprising at least one cyano substituent. The —CN substituent may be on a primary, secondary or tertiary carbon. Unless stated otherwise specifically in the specification, a cyanoalkyl group is optionally substituted.

“Carbocyclic” or “carbocycle” refers to a ring system, wherein each of the ring atoms are carbon.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Alkylcycloalkyl” refers to a radical group of the formula —RₐR_{b} where Rₐ is a cycloalkyl group and R_{b} is an alkyl group as defined above. Unless otherwise stated specifically in the specification, an alkylcycloalkyl group is optionally substituted.

“Fused” refers to any ring structure described herein which is fused to another ring structure.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Halocycloalkyl” refers to a cycloalkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a halocycloalkyl group is optionally substituted.

“Haloalkylcycloalkyl” refers to a radical group of the formula —RₐR_{b} where Rₐ is a cycloalkyl group and R_{b} is a haloalkyl group as defined above. Unless otherwise stated specifically in the specification, a haloalkylcycloalkyl group is optionally substituted.

“Halocycloalkylalkyl” refers to a radical group of the formula —RₐR_{b} where Rₐ is an alkyl group and R_{b} is a halocycloalkyl group as defined above. Unless otherwise stated specifically in the specification, a halocycloalkylalkyl group is optionally substituted.

“Heterocyclylcycloalkyl” refers to a radical group of the formula —RₐR_{b} where Rₐ is a cycloalkyl group and R_{b} is a heterocyclyl group as defined herein. Unless otherwise stated specifically in the specification, a heterocyclylcycloalkyl group is optionally substituted.

“Hydroxylalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxylalkyl group is optionally substituted.

“Heterocyclyl,” “heterocyclic,” or “heterocycle” refer to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen and sulfur. Unless stated otherwise specifically in the specification, the heterocyclyl radical is partially or fully saturated and is a monocyclic, bicyclic, tricyclic or tetracyclic ring system, which may include fused, spirocyclic, and/or bridged ring systems. Nitrogen, carbon, and sulfur atoms in a heterocyclyl radical are optionally oxidized, and nitrogen atoms may be optionally quaternized. Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-I (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl. “Heterocyclyl” as used herein, includes a fused ring system that comprises additional non-heterocyclyl components. For example, in some embodiments, heterocyclyl may have one of the following structures:

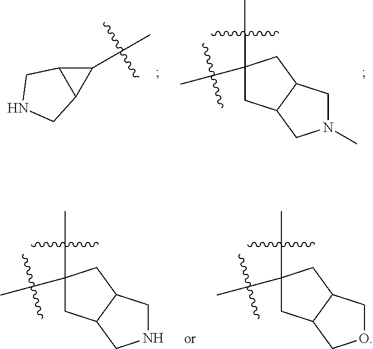 [SMILES: *C1C2CNCC12 ; *C1(*)CC2CN(C)CC2C1 ; *C1(*)CC2CNCC2C1 ; *C1(*)CC2COCC2C1]

Unless stated otherwise specifically in the specification, a heterocyclyl group is optionally substituted.

“Haloheterocyclyl” refers to a heterocyclyl group comprising at least one halo substituent. The halo substituent may be on a primary, secondary or tertiary carbon. Unless stated otherwise specifically in the specification, a haloheterocyclyl group is optionally substituted.

“Haloheterocyclylalkyl” refers to a radical group of the formula —RₐR_{b} where Rₐ is an alkyl group and R_{b} is a haloheterocyclyl group as defined herein. Unless otherwise stated specifically in the specification, a haloheterocyclylalkyl group is optionally substituted.

“Heterocyclylalkyl” refers to a radical group of the formula —RₐR_{b} where Rₐ is an alkyl group and R_{b} is a heterocyclyl group as defined herein. Unless otherwise stated specifically in the specification, a heterocyclylalkyl group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen and sulfur, and at least one aromatic ring. Heteroaryl radicals may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). “Heteroaryl” as used herein, includes a fused ring system where the heteroatom (e.g., oxygen, sulfur, nitrogen, etc.) is not part of the aryl moiety. For example, in some embodiments, heteroaryl may have the following structure:

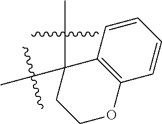 [SMILES: *C1(*)CCOc2ccccc21]

Unless stated otherwise specifically in the specification, a heteroaryl group is optionally substituted.

Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., two R groups taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purpose of the present disclosure fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

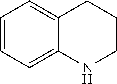

is, for the purposes of the present disclosure, considered a heterocyclic unit. 6,7-Dihydro-5H-cyclopentapyrimidine having the formula:

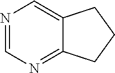

is, for the purposes of the present disclosure, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

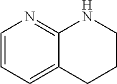

is, for the purposes of the present disclosure, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups are so indicated.

The compounds of the disclosure (i.e., compounds of Structure (I) or (II)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) or (II) are a mixture of enantiomers or diastereomers. In other embodiments, the compounds of Structure (I) or (II) are substantially one enantiomer or diastereomer.

A “tautomer” refers to a proton shift from one atom of a molecule to another atom of the same molecule. Embodiments thus include tautomers of the disclosed compounds.

In some embodiments, a MNK inhibitor has the following Structure (Ia):

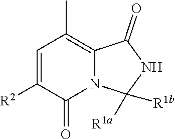

or a pharmaceutically acceptable salt thereof, wherein:
R^{1a} and R^{1b} are each independently alkyl. In some embodiments, R^{1a} and R^{1b} are the same. In certain embodiments, R^{1a} and R^{1b} are different. R^{1a} or R^{1b} may be alkyl groups, such as a methyl, ethyl, propyl, isopropyl, or tert-butyl group. The R^{1a} or R^{1b} substituent groups may be the same alkyl group, or different alkyl groups. For example, R^{1a} may be a methyl group, while R^{1b} may be an ethyl group. By way of another example, R^{1a} may be an isopropyl group, while R^{1b} may be a tert-butyl group. Any alkyl group combinations of substituents R^{1a} or R^{1b} may be used.

In some embodiments, R^{1a} and R^{1b} joint to form a cyclic moiety. In certain embodiments, the compound has the following Structure (Ib):

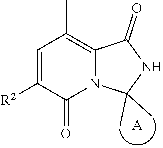

or a pharmaceutically acceptable salt thereof, wherein: R^{1a} and R^{1b} may join together to form ring A.

The Structure (Ib), substituents R^{1a} or R^{1b} may together form a cyclic compound indicated as cyclic moiety A. For example, the cyclic moiety A of the Structure (Ib) may include a five-membered ring. The cyclic moiety A of the Structure (Ib) may be a non-substituted cyclic compound. For instance, the cyclic moiety A may be a non-substituted five-membered ring such as a cyclopentane. Further, the cyclic moiety A of the Structure (Ib) may have one or more alkyl substitutions. For example, the alkyl substitutions on the cyclic moiety A may include methyl, ethyl, propyl, isopropyl, cyclopropyl, or tert-butyl group. Substituted positions may be 2-, 3—, 4-, or 5-position of the cyclopentane. The degree of the substitutions may include mono-, di-, tri-, or tetra-substitutions. For instance, the cyclic moiety A may be 2,2,5,5-tetramethylcyclopentane. Synthetic routes may be used to install different substitution patterns on the cyclopentane ring. For example, the cyclic moiety A may be 3,3,4,4-tetramethylcyclopenta ne.

Additionally, the cyclic moiety A may have a fused ring. A part of the cyclic moiety A may include a fused benzene ring. For example, the cyclic moiety A may include the fused benzene ring with a cyclopentyl or cyclohexyl ring. For instance, the synthetic route to prepare the benzene fused cyclohexyl compound may involve the use of 1-tetralone. Further, the cyclic moiety A may include a fused cyclopentyl or cyclohexyl ring with other cyclic structures.

The cyclic moiety A may include a six-membered ring. The cyclic moiety A may be non-substituted cyclic moiety. For example, the cyclic moiety A may be a non-substituted six-membered ring such as a cyclohexane. Further, the cyclic moiety A may have one or more alkyl substitutions. For example, the alkyl substitutions on the cyclic moiety A may include methyl, ethyl, propyl, isopropyl, cyclopropyl, or tert-butyl group. The cyclic moiety A may have one or more heteroatom-containing substituents, such as alcohols, sulfonamides, or carboxylic acids. Substituted positions may be 2-, 3-, 4-, 5-, or 6-position of the cyclohexane. The degree of the substitutions may include mono-, di-, tri-, or tetra-substitutions. For instance, the cyclic moiety A may be 3,5-dimethylcyclohexane. Synthetic routes may be used to install different substitution patterns on the cyclohexane ring. For example, the cyclic moiety A may be 2,3,4,5,6-pentamethylcyclohexane.

The cyclic moiety A may include a heterocyclic compound. The heterocyclic compound is a cyclic compound that has atoms of at least two different elements such as a carbon and an oxygen atom. For example, the cyclic moiety A may be tetrahydropyran. The tetrahydropyran includes one oxygen atom and five carbon atoms in a six-membered ring. The heterocyclic compound may further be substituted with alkyl substituents or functional groups on various positions with various degrees of substitutions. It is noted that, while some of the structures shown in the present disclosure include an oxygen atom in a cyclic compound, such a structure is merely provided for illustrative purposes. Synthetic routes may be used to install different heteroatoms in the cyclic compounds. For example, the cyclic moiety A of the structure (Ib) may include piperidine (a nitrogen atom), phosphinate (a phosphorus atom), silinane (a silicon atom), or thiane (a sulfur atom).

The cyclic moiety A may be unsaturated. Unsaturated cyclic compounds may include aromatic cyclic compounds such as a benzene, pyridine, diazine, oxazine, dioxine, or thiazine. Alternatively, the cyclic moiety A may be saturated.

The cyclic moiety A may have one or more functional group substitutions. For example, the functional groups may include a hydroxyl, amine, amide, carboxylic acid, ether, or sulfonamide. Thus, the cyclic moiety A may include 4-hydroxyl cyclohexane, 4-carboxylic acid cyclohexane, 4-methoxyl cyclohexane, or 4-alkylsulfonamide cyclohexane. Substituted positions may be 2-, 3-, 4-, 5-, or 6-position of the cyclohexane. The degree of the substitutions may include mono-, di-, tri-, tetra-, or penta-substitutions. One or more functional groups may be installed on a heterocyclic compound with various substitution positions and degree.

The substituent R² of the structures (Ia) and (Ib) may include a nitrogen containing functional group. For example, the nitrogen containing functional group of the substituent R² may include amides, amidine, amines, amine oxides, azo, carbamates, carbodiimides, enamines, aromatic heterocycles, non-aromatic heterocycles, hydrazones, hydroxamic acids, imides, imines, nitriles, sulfonamide, or urea. For example, the aromatic heterocycles may include pyrrole, imidazole, pyrazole, thiazole, pyridine, pyridazine, pyrimidine, pyrazine, or triazine. The nitrogen containing functional group of substituent R² may be unsubstituted or substituted. For instance, a pyridazine may be substituted with an amine group at 3 position as shown in 4ET-004-006 hereinafter. In another instance, a pyridazine may be substituted with an amide containing a cyclopropyl ring at 3 position as shown in 4ET-004-003 hereinafter. The degree and location of substitution on the nitrogen containing functional group may differ. The nitrogen containing functional group of the substituent R² may be attached via an alkyl chain represented by —CₙH₂ₙ— where n is between zero and five. In this regard, the nitrogen containing functional groups of the substituent R² and the backbone structures (Ia) and (Ib) are separated by n carbon atoms.

Substituent R² of the structures (Ia) and (Ib) may include an aromatic heterocycle. For instance, in some embodiments, substituent R² may include 4-aminopyrimidinyl moiety. In some specific embodiments, the compound is a compound of Structure (Ic):

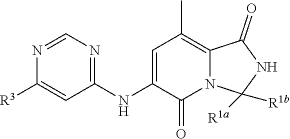

or pharmaceutically acceptable salt thereof, wherein: R³ may include an amine.

In some embodiments, the amine is a primary amine. In some embodiments, R³ is —NH₂.

In some embodiments, R³ may include a secondary amine. When the amine is a secondary amine, R³ may further include a functional group at one end. For example, the functional group may include a hydroxyl, sulfonamide, carboxylic acid, ester, amine, amide, morpholine, piperazine, or thiomorpholine. The secondary amine and the functional group may be attached via an alkyl chain represented by —CₙH₂ₙ— where n is between one and five. Thus, the secondary amine of the substituent R³ and the functional group may be separated by n carbon atoms. For example, the secondary amine attached to a hydroxyl group separated by carbons atoms forms an aminoalcohol (HO—C₂H₄NH—), which is shown as examples 4ET-02-001, 4ET-03-004, 4ET-03-007, and 4ET-03-011 hereinafter. By way of another example, the secondary amine attached to a sulfonamide group separated by two carbon atoms forms amino sulfonamide (CH₃SO₂NHC₂H₄NH—), which is shown as examples 4ET-02-004, 4ET-03-012, 4ET-03-013, and 4ET-03-014 hereinafter.

The amine of substituent R³ may include a tertiary amine. The tertiary amine of the substituent R³ may be cyclic. The cyclic tertiary amine of the substituent R³ may be a part of saturated five-membered ring or six-membered ring. For example, the cyclic tertiary amine of the substituent R³ in a saturated five-membered ring may be pyrrolidine, imidazolidine, or pyrazolidine. The cyclic tertiary amine of the substituent R³ in a saturated six-membered ring may be piperidine or piperazine.

The tertiary amine may further include a functional group at one end. For example, the functional group may include a hydroxyl, sulfonamide, carboxylic acid, ester, amide, amine, morpholine, piperazine, or thiomorpholine. The tertiary amine and the functional group may be attached via an alkyl chain represented by —CₙH₂ₙ— where n is between one and five. Thus, the tertiary amine of the substituent R³ and the functional group may be separated by n carbon atoms.

The tertiary amine of the substituent R³ may be cyclic. The cyclic tertiary amine of the substituent R³ may be a part of unsaturated five-membered ring or six-membered ring. For example, the cyclic tertiary amine of the substituent R³ in an unsaturated five-membered ring may be pyrazole, imidazole, or oxazole. The cyclic tertiary amine of the substituent R³ in an unsaturated six-membered ring may be pyridine, diazine, triazine, or oxazine.

The amine of substituent R³ may also include an amide group. The amide group of substituent R³ may further include a functional group at one end. For example, the functional group may include a hydroxyl, sulfonamide, carboxylic acid, ester, amine, amide, morpholine, piperazine, or thiomorpholine.

The amide of the substituent R³ and the functional group may be attached via an alkyl chain represented by —CₙH₂ₙ— where n is between zero and five. Thus, the amide of the substituent R³ and the functional group may be separated by n carbon atoms. For example, the amide attached to morpholine group by one methylene forms morpholine amide, which is shown as examples 4ET-02-007, 4ET-03-027, and 4ET-03-028 hereinafter. The amide attached to morpholine group by two methylenes forms morpholine amide, which is shown as example 4ET-02-031 hereinafter. The amide of the substituent R³ may also be directly attached to one of the functional groups.

The amide of the substituent R³ may be directly attached to a cyclic structure. For example, the amide of the substituent R³ may be directly attached to cyclopropane. In this case, there is no carbon atom between the amide and cyclopropane. Structures with the amide group directly attached to cyclopropane as a part of the substituent R³ include 4ET-02-003, 4ET-02-009, 4ET-02-010, 4ET-02-011, 4ET-02-012, 4ET-02-016, 4ET-03-002, 4ET-03-009, 4ET-03-017, 4ET-03-019, 4ET-03-020, 4ET-03-023, 4ET-03-026, 4ET-03-034, and 4ET-04-003 hereinafter. Cyclopropanes may be unsubstituted or substituted with one or more functional groups. For instance, the substituted cyclopropanes may include fluorine, hydroxyl, hydroxylmethylene, alkyl, carboxylic acid, amine, aminomethylene, ester, ether, amide, sulfonamide, morpholine, piperazine, or thiomorpholine group attached to a cyclopropane ring. The substituted position on the cyclopropane where the functional group is attached may be the 1-, 2-, or 3-position. The functional group attached to the cyclopropane may have an additional alkyl chain (—CₙH₂ₙ—) between the functional group and the cyclopropane where n is between zero and 5. When n is equal to zero, there is no methylene between the functional group and the cyclopropane. Thus, the functional group may be directly attached to the cyclopropane on the 1-, 2-, or 3-position. Similarly, when n is equal to one, there is one methylene between the functional group and the cyclopropane. In this case, the functional group is one carbon away from the cyclopropane, which gives an extra degree of freedom to the structure. Structures with the amide group directly attached to substituted cyclopropane as a part of the substituent R³ include 4ET-02-009, 4ET-02-010, 4ET-02-011, 4ET-02-012, 4ET-02-016, 4ET-03-019, 4ET-03-020,4ET-03-023, 4ET-03-026, and 4ET-03-034 hereinafter.

The amide of the substituent R³ may be directly attached to cyclobutane. In this case, there is no carbon atom between the amide and cyclobutane. The cyclobutane may further have a functional group. For instance, the functional group may include hydroxyl, alkyl, carboxylic acid, amine, ester, ether, amide, sulfonamide, morpholine, piperazine, or thiomorpholine. The substituted position on the cyclobutane where the functional group is attached may be the 1-, 2-, 3-, or 4-position. The functional group may have an additional alkyl chain (CₙH₂ₙ) between the functional group and the cyclobutane where n is between zero and 5.

The cyclic structure that is attached to the amide via an alkyl chain or directly may include at least one heteroatom to form a heterocyclic compound. The heterocyclic compound may include a three-membered ring with one heteroatom or a four-membered ring with one heteroatom. For example, the three-membered ring with one heteroatom may include aziridines or ethylene oxide. By way of another example, the four-membered ring with one heteroatom may include azetidine or oxetane. Azetidine directly attached to the amide is shown for example in 4ET-02-017 hereinafter. As described above, functional groups may be attached to the heterocyclic compound. In the case of ethylene oxide (epoxide), Sharpless epoxidation may be used to generate chiral epoxides.

While the examples herein only have a monosubstitution on the cyclic structure, such a configuration is merely provided for illustrative purposes. Embodiments of the present disclosure include disubstituted cyclic structures as well. For example, a total of two amine groups may be attached to the cyclopropane; a first amine group may be attached to 1-position of cyclopropane, while a second amine group is attached to 2-position of cyclopropane.

The amide of the substituent R³ may be a reverse amide. Instead of a nitrogen atom of the amide of the substituent R³ being directly attached to the structure (Ic), a carbon atom of the amide of the substituent R³ may be attached to the structure (Ic). The reverse amide attached to the structure (Ic) is shown for example in 4ET-03-024 hereinafter. Embodiments of the present disclosure described above including the amide in the substituent R³ may also be replaced with a reverse amide. For instance, the amide group of examples such as 4ET-02-003, 4ET-02-009, 4ET-02-010, 4ET-02-011, 4ET-02-012, 4ET-02-016, 4ET-03-002, 4ET-03-009, 4ET-03-017, 4ET-03-019, 4ET-03-020, 4ET-03-023, 4ET-03-026, 4ET-03-034, 4ET-04-003, 4ET-02-007, 4ET-03-027, 4ET-03-028, and 4ET-02-031 may be replaced with a reverse amide.

The structure (Ic) may be equipped with an amide analog of the substituent R³. For example, a thioamide group may be used instead of the amide group shown in 4ET-02-013 hereinafter. Similar to the amide substituent, the thioamide group may be replaced with a reverse thioamide. In this regard, instead of a nitrogen atom of the thioamide of the substituent R³ being directly attached to the structure (Ic), a carbon atom of the thioamide of the substituent R³ may be attached to the structure (Ic).

Additionally, other amide analogs of the substituent R³ may be used for the structure (Ic). For example, a urea group may be used instead of the amide group shown in 4ET-02-015 hereinafter. By way of another example, a thiourea group may be used instead of the amide group. An amide, a reverse amide, a thioamide, a reverse thioamide, a urea, and a thiourea as a part of the substituent R³ are interchangeable in the structure (Ic).

In some MNK inhibitors of the present disclosure, the 4-aminopyrimidine moiety in structure (Ic) may be modified. The pyrimidine moiety and the parent structure as shown in the structure (Ia) or (Ib) are connected via the amine linker (NH—) in the structure (Ic). The amine linker may be extended. For example, the amine linker may include additional alkyl chain (—CₙH₂ₙ—) between the amine and pyrimidine moiety where n is between one and five. For instance, one extra carbon atom (n=1) may be added, such that the amine linker and pyrimidine are one carbon away from the parent structure, as shown in 4ET-04-004, which gives the structure (Ic) more structural flexibility via an extra degree of freedom. One carbon extension, which is an insertion of a methylene unit, between the amine and the pyrimidine moiety provides a benzylpyrimidine moiety. By way of another example, the amine linker may include additional alkyl chain (—CₙH₂ₙ—) between the amine and the parent structure shown as the structure (Ia) or (Ib) where n is between one and five. For instance, one extra carbon atom (n=1) may be added, as shown in 4ET-04-015, such that the amine linker and the parent structure shown as the structure (Ia) or (Ib) are one carbon away from the parent structure. One carbon extension, which is an insertion of a methylene unit, between the amine and the structure (Ia) or (Ib) provides a methylaminopyrimidine moiety. In this regard, methylene units may be added both sides of the amine linker of the structure (Ic). Amine linker extension with extra methylene units may be used in conjunction with any of the other variations of structures (Ia), (Ib), and (Ic) disclosed herein.

Additionally, the pyrimidine moiety in the structure (Ic) may be modified to substitute a different unsaturated six-membered ring with two nitrogen atoms isomer, such as 1,2-diazine (pyridazine) or 1,4-diazine (pyrazine). For example, 1,2-diazine (pyridazine) may be used instead of 1,3-diazine (pyrimidine) in the structure (Ic) shown in example 4ET-04-003 and 4ET-04-006 hereinafter. These modifications may be used in conjunction with any of the other variations of structures (Ia), (Ib), and (Ic) disclosed herein.

Pyrimidine in the structure (Ic) may be replaced with a five-membered heterocyclic compound. Pyrimidine is a six-membered heterocyclic compound with two nitrogen atoms. In general, five-membered heterocyclic compounds have different chemical and physical properties than the six-membered heterocyclic compounds. Some MNK inhibitors of the present disclosure may take advantage of such differences between five- and six-membered heterocyclic compounds. For example, the five-membered heterocyclic compound may include nitrogen and sulfur atoms. For instance, the five-membered heterocyclic compound with N and S may include thiazole as shown in example 4ET-04-001 hereinafter. By way of another example, the five-membered heterocyclic compound with S may include thiophene. The five-membered heterocyclic compound may include nitrogen and oxygen atoms. For instance, the five-membered heterocyclic compound with N and O may include oxazole or isoxazole. Yet in another example, the five-membered heterocyclic compound may include two nitrogen atoms. For instance, the five-membered heterocyclic compound with two nitrogen atoms may include imidazole or pyrazole. These modifications may be used on conjunction with any of the other variations of structures (Ia), (Ib), and (Ic) disclosed herein.

As examples of how various modifications disclosed herein may be used in combination with one another, the amine linker with additional carbon atom may be attached to a pyridazine moiety and the pyridazine moiety may be connected to the pyridone scaffold with an amine or sulfonamide. By way of another example, the amine linker with additional carbon atom may be attached to a pyridazine moiety and the pyridazine moiety may be directly connected to an amino group.

In some MNK inhibitors of the present disclosure, the 4-aminopyrimidine moiety and a parent structure, for example, a pyridone moiety in structure (Ic) may be attached via other nitrogen containing linkers. The pyrimidine moiety and the parent structure as shown in the structure (Ia) or (Ib) are connected via the amine linker (NH—) in the structure (Ic). Embodiments of the present disclosure may be configured to install an amide group between the 4-aminopyrimidine moiety and the parent structure. This can be synthesized by using an amide containing starting material in Buchwald-Hartwig amination. For example, the resulting MNK inhibitor may include an amide as shown in example 4ET-04-013 or a reverse amide as shown in example 4ET-04-014 hereinbelow between the 4-aminopyrimidine moiety and the parent structure.

Further, embodiments of the present disclosure may be configured to install a sulfonamide group between the 4-aminopyrimidine moiety and the parent structure. This can be synthesized by using a sulfonamide containing starting material in Buchwald-Hartwig amination. Another approach involves the use of a sulfonyl chloride reagent or intermediate. For example, the resulting MNK inhibitor may include a sulfonamide as shown in examples 4ET-04-010 and 4ET-04-011 or a reverse sulfonamide as shown in example 4ET-04-012 hereinbelow between the 4-aminopyrimidine moiety and the parent structure.

Additionally, embodiments of the present disclosure may be configured to install an ether group between the 4-aminopyrimidine moiety and the parent structure. This can be synthesized by using an alcohol containing starting material in Buchwald-Hartwig amination. Another approach involves using an alcohol containing starting material in an Ullmann-type coupling reaction.

The substituents R^{1a} or R^{1b} of the structure (Ic) may be alkyl groups, as discussed hereinabove in the structure (Ia). Alternatively, the substituents R^{1a} or R^{1b} of the structure (Ic) may together form a cyclic compound indicated as a ring structure A below. The detailed discussion of the ring structure A of the structure (Ib) may also apply to the structure (Id):

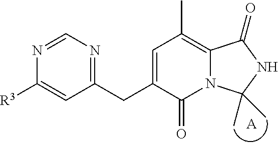

or pharmaceutically acceptable salt thereof, wherein: R³ may include an amine.

One embodiment provides an MNK inhibitor having the following Structure (II):

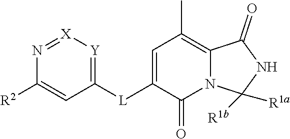

or a pharmaceutically acceptable salt thereof, wherein:
R^{1a} is C₁-C₆ alkyl or aryl;
R^{1b} is C₁-C₆ alkyl or aryl,
   or R^{1a} and R^{1b}, together with the carbon to which they are both attached, join to form cycloalkyl, cycloalkenyl, heterocyclyl, aryl, or heteroaryl;
R² is —NHR^{3a}, —NHC(═O)R^{3b}, —NHC(═S)R^{3b}, or —C(═O)R^{3c};
R^{3a} is hydrogen, C₁-C₆ alkyl, or C₃-C₆ cycloalkyl, each of which is optionally substituted with one or more substituents selected from the group consisting of hydroxyl, C₃-C₆ cycloalkyl, —NHS(O)₂CH₃, heterocyclyl, —C(═O)OH, —C(═O)N(R^{3d})R^{3d}, or —N(R^{3d})R^{3d};
R^{3b} is C₁-C₆ alkyl, C₃-C₆ cycloalkyl, or heterocyclyl each of which is optionally substituted with one or more substituents selected from the group consisting of hydroxyl, halo, C₁-C₆ alkyl, C₃-C₆ cycloalkyl, —NHS(O)₂CH₃, —N(R^{3d})R^{3d}, heterocyclyl, —C(═O)OH, —C(═O)N(R^{3d})R^{3d}, —NHC(═O)CH₃, —CH₂C(═O)OH,
R^{3c} is N(Rid)Rid or heterocyclyl;
R^{3d} is, at each occurrence, independently hydrogen, C₁-C₆ alkyl, or C₃-C₆ cycloalkyl;
L is —NH— or —CH₂NH—; and
x is N and Y is CH or X is CH and Y is N

In some embodiments, when R^{1a} and R^{1b} are both —CH₃ or when R^{1a} and R^{1b} join to form a 5- or 6-membered cycloalkyl or heterocyclyl, then R² does not have the following structure:

### —NH₂ or

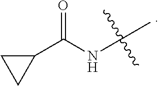 [SMILES: *NC(=O)C1CC1 ; "."]

In some embodiments, R^{1a} is C₁-C₆ alkyl. In some embodiments, R^{1a} is methyl. In certain embodiments, R^{1a} is aryl. In certain embodiments, R^{1a} is phenyl.

In certain specific embodiments, R^{1b} is C₁-C₆ alkyl. In some embodiments, R^{1b} is methyl. In some embodiments, R^{1a} and R^{1b}, together with the carbon to which they are both attached, join to form cycloalkyl. In more specific embodiments, the cycloalkyl is cyclopentyl or cyclohexyl. In some embodiments, R^{1a} and R^{1b}, together with the carbon to which they are both attached, join to form cycloalkenyl. In some embodiments, the cycloalkenyl is cyclopentenyl, cyclohexenyl, or cycloheptenyl. In certain specific embodiments, R^{1a} and R^{1b}, together with the carbon to which they are both attached, join to form heterocyclyl. In some specific embodiments, R^{1a} and R^{1b}, together with the carbon to which they are both attached, join to form aryl. In some embodiments, R^{1a} and R^{1b}, together with the carbon to which they are both attached, join to form heteroaryl.

In more specific embodiments, the compound has one of the following structures:

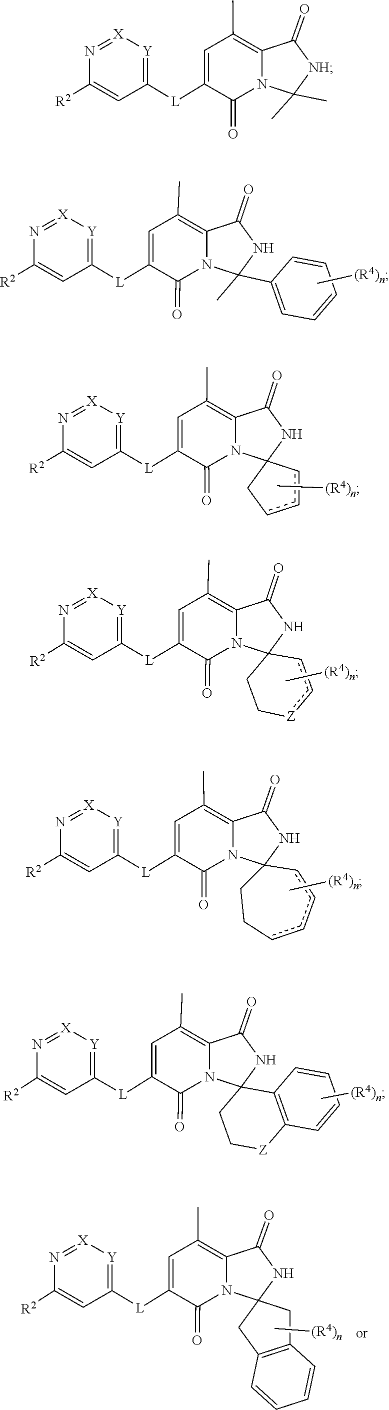

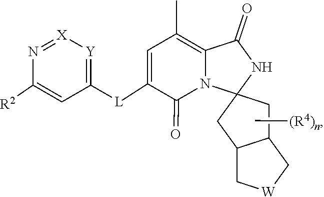

or a pharmaceutically acceptable salt thereof, wherein
[symbol] indicates a double or single bond;
R⁴ is, at each occurrence, independently C₁-C₆ alkyl, C₃-C₆ cycloalkyl, halo, haloalkyl, hydroxyl, —NHS(O)₂CH₃, or —C(O)OH,
   or two R⁴, together with the carbon to which they are both attached, join to form a cycloalkyl;
W is N or O;
Z is C or O; and
n is 0, 1, 2, 3, or 4.

In some embodiments, n is 0, 1, or 2. In some more specific embodiments, only one location depicted with [symbol] is a double bond and the rest are single bonds. In some embodiments, the compound has the following structure:

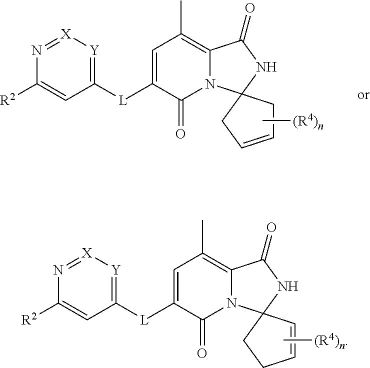

In some more specific embodiments, the compound has the following structure:

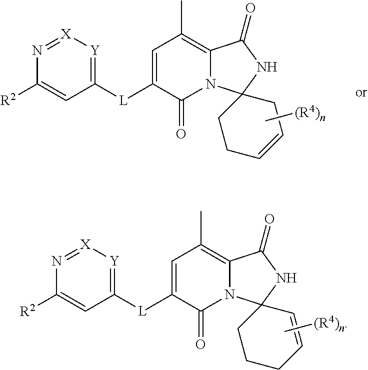

In some embodiments, the compound has one of the following structures:

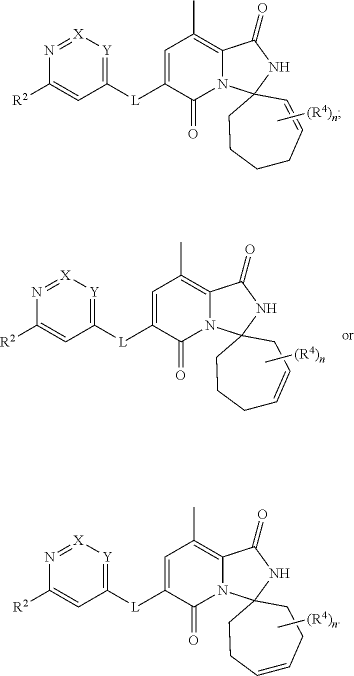

In more specific embodiments, R² is —NHR^{3a}. In more specific embodiments. R² has one of the following structures:

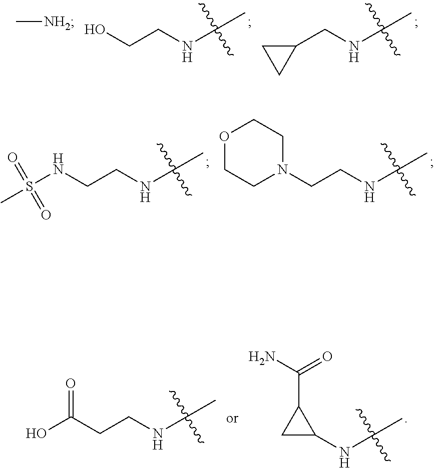

In some embodiments, R² is —NHC(═O)R^{3b}. In more specific embodiments, R² has one of the following structures:

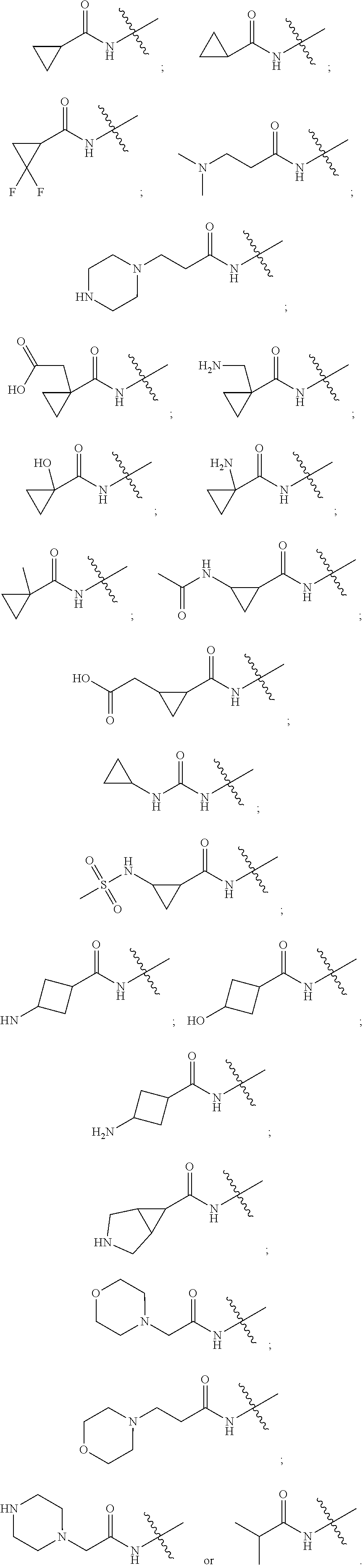

In certain embodiments, R² is —NHC(═S)R^{3b}. In certain embodiments, R² has the following structure:

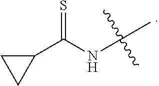 [SMILES: *NC(=S)C1CC1 ; "."]

In certain embodiments, R² is —C(═O)R^{3c}. In some embodiments, R² has one of the following structures:

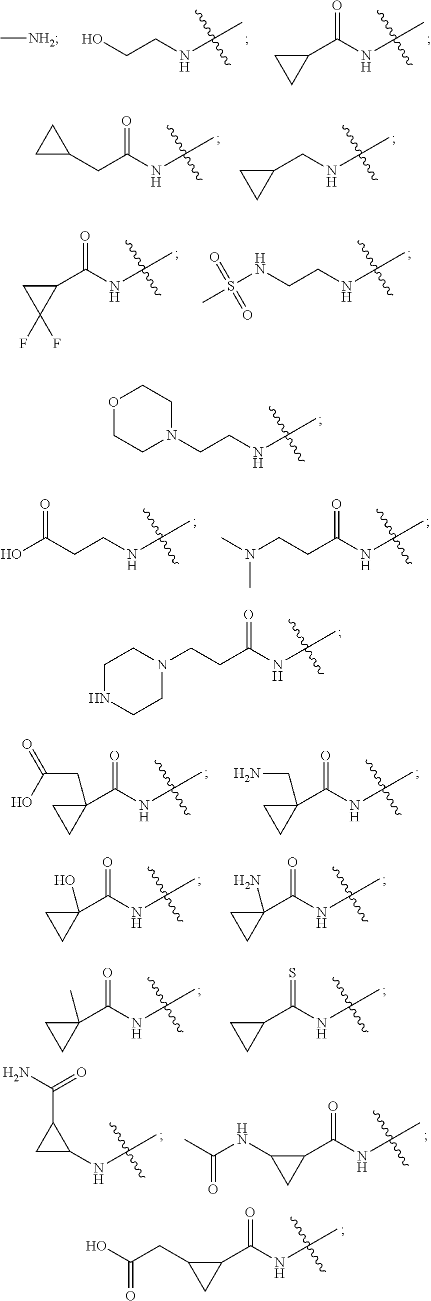

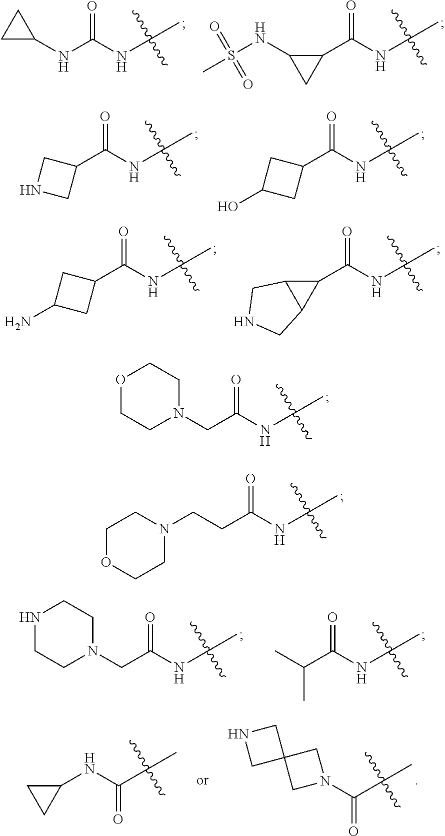

In certain specific embodiments, Rz has one of the following structures:

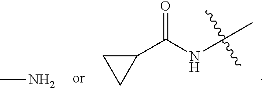

In some embodiments, X is CH and Y is N. In certain embodiments, X is N and Y is CH. In some embodiments, L is —NH—. In more embodiments, L is —CH₂NH—.

In some embodiments, R^{3a} is a branched C₁-C₆ alkyl. In some embodiments, R^{3a} is iso-propyl.

In various different embodiments, the compound has one of the structures set forth in Table 1 below, or a pharmaceutically acceptable salt thereof. Compounds in Table 1 were prepared as described in WO 2022006331 and/or methods known in the art.

TABLE 1

| No. | Structure |
|---|---|
| 4ET-01-001 | 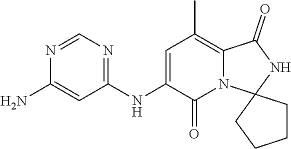 [SMILES: Cc1cc(Nc2cc(N)ncn2)c(=O)n2c1C(=O)NC21CCCC1] |
| 4ET-01-002 | 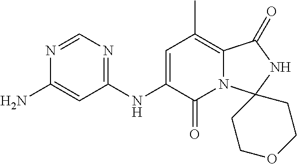 [SMILES: Cc1cc(Nc2cc(N)ncn2)c(=O)n2c1C(=O)NC21CCOCC1] |
| 4ET-01-003 | 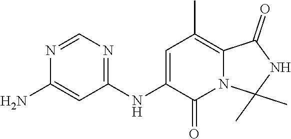 [SMILES: Cc1cc(Nc2cc(N)ncn2)c(=O)n2c1C(=O)NC2(C)C] |
| 4ET-01-004 | 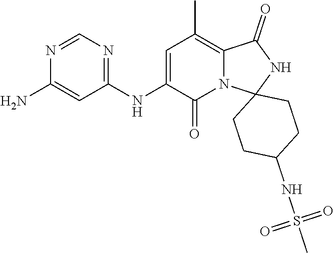 [SMILES: Cc1cc(Nc2cc(N)ncn2)c(=O)n2c1C(=O)NC21CCC(NS(C)(=O)=O)CC1] |
| 4ET-01-005 | 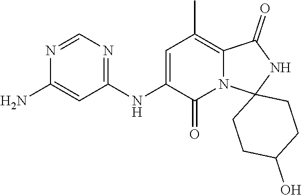 [SMILES: Cc1cc(Nc2cc(N)ncn2)c(=O)n2c1C(=O)NC21CCC(O)CC1] |
| 4ET-01-010A | 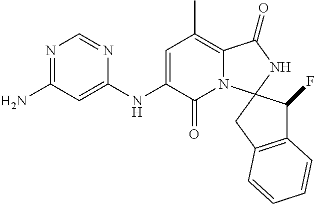 [SMILES: Cc1cc(Nc2cc(N)ncn2)c(=O)n2c1C(=O)NC21Cc2ccccc2[C@@H]1F] |
| 4ET-01-010B | 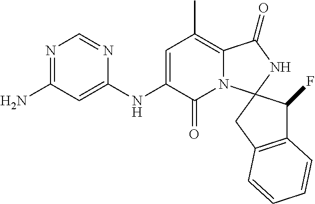 [SMILES: Cc1cc(Nc2cc(N)ncn2)c(=O)n2c1C(=O)NC21Cc2ccccc2[C@@H]1F] |
| 4ET-01-014A | 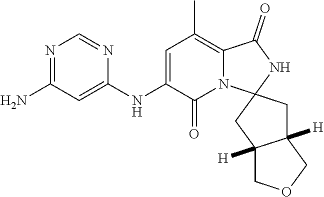 [SMILES: Cc1cc(Nc2cc(N)ncn2)c(=O)n2c1C(=O)NC21C[C@H]2COC[C@H]2C1] |
| 4ET-01-014B | 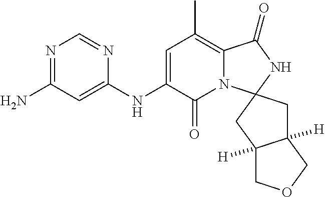 [SMILES: Cc1cc(Nc2cc(N)ncn2)c(=O)n2c1C(=O)NC21C[C@H]2COC[C@H]2C1] |
| 4ET-01-021 | 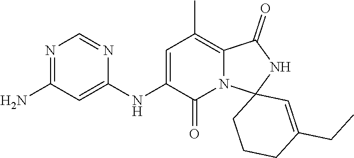 [SMILES: CCC1=CC2(CCC1)NC(=O)c1c(C)cc(Nc3cc(N)ncn3)c(=O)n12] |
| 4ET-01-058 | 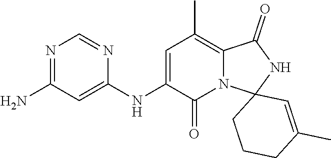 [SMILES: CC1=CC2(CCC1)NC(=O)c1c(C)cc(Nc3cc(N)ncn3)c(=O)n12] |
| 4ET-02-001 | 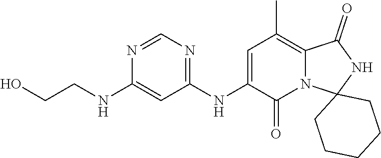 [SMILES: Cc1cc(Nc2cc(NCCO)ncn2)c(=O)n2c1C(=O)NC21CCCCC1] |
| 4ET-02-002 | 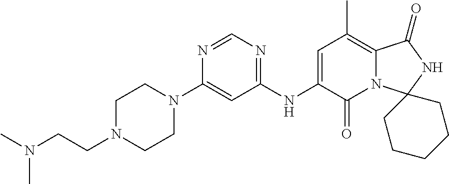 [SMILES: Cc1cc(Nc2cc(N3CCN(CCN(C)C)CC3)ncn2)c(=O)n2c1C(=O)NC21CCCCC1] |
| 4ET-02-003 | 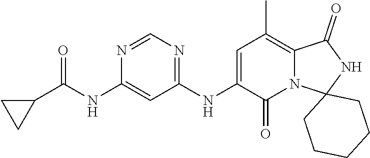 [SMILES: Cc1cc(Nc2cc(NC(=O)C3CC3)ncn2)c(=O)n2c1C(=O)NC21CCCCC1] |
| 4ET-02-004 | 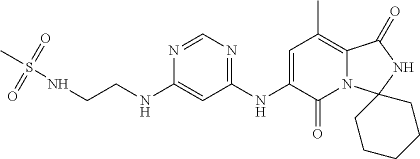 [SMILES: Cc1cc(Nc2cc(NCCNS(C)(=O)=O)ncn2)c(=O)n2c1C(=O)NC21CCCCC1] |
| 4ET-02-005 | 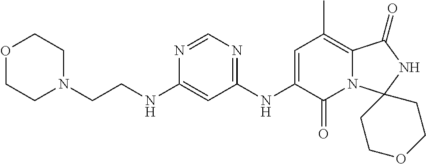 [SMILES: Cc1cc(Nc2cc(NCCN3CCOCC3)ncn2)c(=O)n2c1C(=O)NC21CCOCC1] |
| 4ET-02-006 | 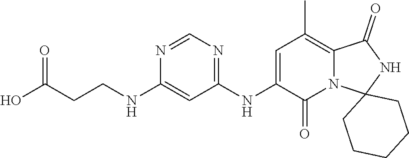 [SMILES: Cc1cc(Nc2cc(NCCC(=O)O)ncn2)c(=O)n2c1C(=O)NC21CCCCC1] |
| 4ET-02-007 | 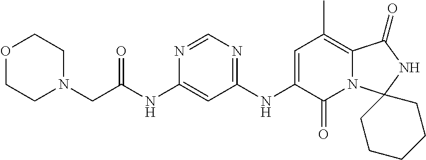 [SMILES: Cc1cc(Nc2cc(NC(=O)CN3CCOCC3)ncn2)c(=O)n2c1C(=O)NC21CCCCC1] |
| 4ET-02-008 | 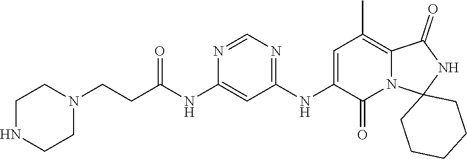 [SMILES: Cc1cc(Nc2cc(NC(=O)CCN3CCNCC3)ncn2)c(=O)n2c1C(=O)NC21CCCCC1] |
| 4ET-02-009 | 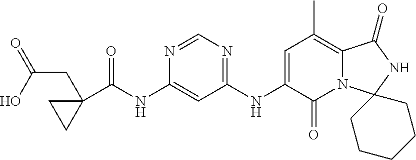 [SMILES: Cc1cc(Nc2cc(NC(=O)C3(CC(=O)O)CC3)ncn2)c(=O)n2c1C(=O)NC21CCCCC1] |
| 4ET-02-010 | 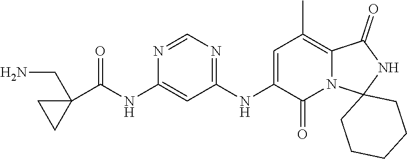 [SMILES: Cc1cc(Nc2cc(NC(=O)C3(CN)CC3)ncn2)c(=O)n2c1C(=O)NC21CCCCC1] |
| 4ET-02-011 | 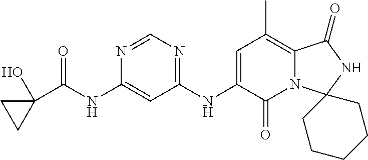 [SMILES: Cc1cc(Nc2cc(NC(=O)C3(O)CC3)ncn2)c(=O)n2c1C(=O)NC21CCCCC1] |
| 4ET-02-012 | 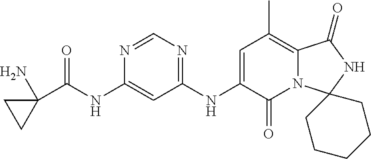 [SMILES: Cc1cc(Nc2cc(NC(=O)C3(N)CC3)ncn2)c(=O)n2c1C(=O)NC21CCCCC1] |
| 4ET-02-013 | 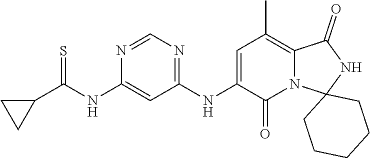 [SMILES: Cc1cc(Nc2cc(NC(=S)C3CC3)ncn2)c(=O)n2c1C(=O)NC21CCCCC1] |
| 4ET-02-014 | 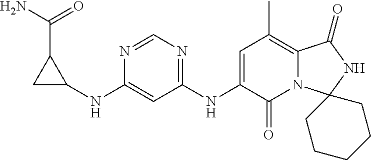 [SMILES: Cc1cc(Nc2cc(NC3CC3C(N)=O)ncn2)c(=O)n2c1C(=O)NC21CCCCC1] |
| 4ET-02-015 | 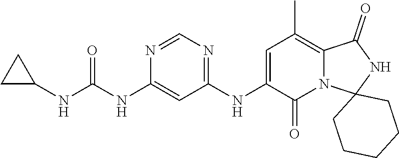 [SMILES: Cc1cc(Nc2cc(NC(=O)NC3CC3)ncn2)c(=O)n2c1C(=O)NC21CCCCC1] |
| 4ET-02-016 | 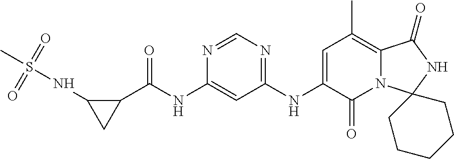 [SMILES: Cc1cc(Nc2cc(NC(=O)C3CC3NS(C)(=O)=O)ncn2)c(=O)n2c1C(=O)NC21CCCCC1] |
| 4ET-02-017 | 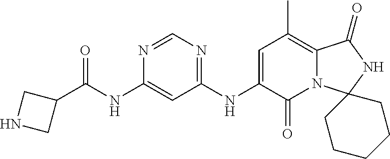 [SMILES: Cc1cc(Nc2cc(NC(=O)C3CNC3)ncn2)c(=O)n2c1C(=O)NC21CCCCC1] |
| 4ET-02-018 | 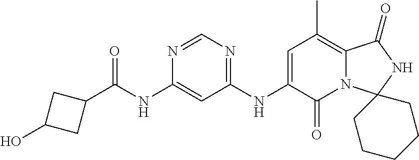 [SMILES: Cc1cc(Nc2cc(NC(=O)C3CC(O)C3)ncn2)c(=O)n2c1C(=O)NC21CCCCC1] |
| 4ET-02-019 | 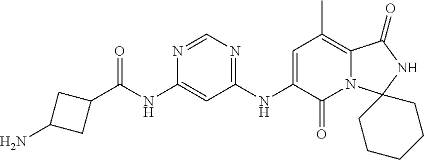 [SMILES: Cc1cc(Nc2cc(NC(=O)C3CC(N)C3)ncn2)c(=O)n2c1C(=O)NC21CCCCC1] |
| 4ET-02-020 | 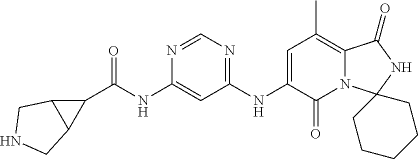 [SMILES: Cc1cc(Nc2cc(NC(=O)C3C4CNCC43)ncn2)c(=O)n2c1C(=O)NC21CCCCC1] |
| 4ET-02-021 | 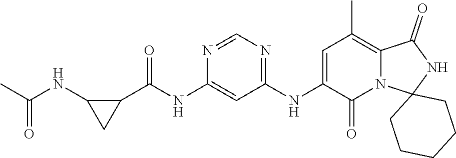 [SMILES: CC(=O)NC1CC1C(=O)Nc1cc(Nc2cc(C)c3n(c2=O)C2(CCCCC2)NC3=O)ncn1] |
| 4ET-02-022 | 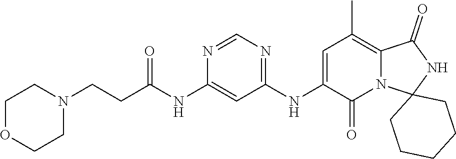 [SMILES: Cc1cc(Nc2cc(NC(=O)CCN3CCOCC3)ncn2)c(=O)n2c1C(=O)NC21CCCCC1] |
| 4ET-02-023 | 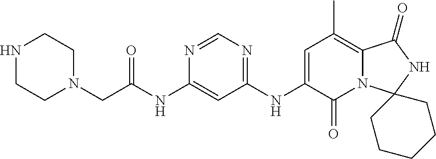 [SMILES: Cc1cc(Nc2cc(NC(=O)CN3CCNCC3)ncn2)c(=O)n2c1C(=O)NC21CCCCC1] |
| 4ET-03-001 | 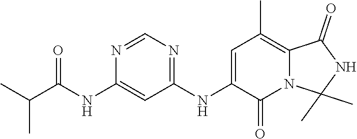 [SMILES: Cc1cc(Nc2cc(NC(=O)C(C)C)ncn2)c(=O)n2c1C(=O)NC2(C)C] |
| 4ET-03-002 | 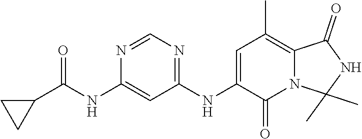 [SMILES: Cc1cc(Nc2cc(NC(=O)C3CC3)ncn2)c(=O)n2c1C(=O)NC2(C)C] |
| 4ET-03-003 | 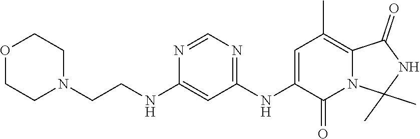 [SMILES: Cc1cc(Nc2cc(NCCN3CCOCC3)ncn2)c(=O)n2c1C(=O)NC2(C)C] |
| 4ET-03-004 | 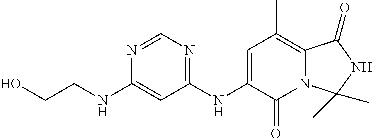 [SMILES: Cc1cc(Nc2cc(NCCO)ncn2)c(=O)n2c1C(=O)NC2(C)C] |
| 4ET-03-005 | 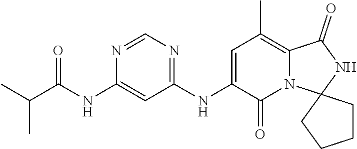 [SMILES: Cc1cc(Nc2cc(NC(=O)C(C)C)ncn2)c(=O)n2c1C(=O)NC21CCCC1] |
| 4ET-03-006 | 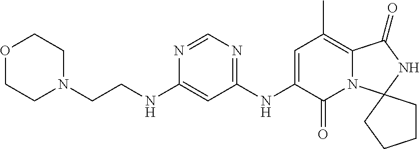 [SMILES: Cc1cc(Nc2cc(NCCN3CCOCC3)ncn2)c(=O)n2c1C(=O)NC21CCCC1] |
| 4ET-03-007 | 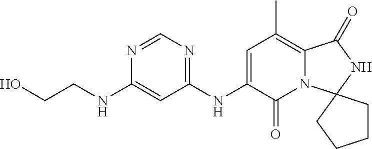 [SMILES: Cc1cc(Nc2cc(NCCO)ncn2)c(=O)n2c1C(=O)NC21CCCC1] |
| 4ET-03-008 | 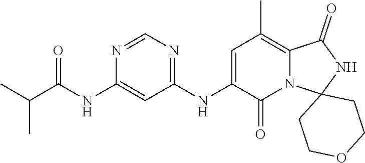 [SMILES: Cc1cc(Nc2cc(NC(=O)C(C)C)ncn2)c(=O)n2c1C(=O)NC21CCOCC1] |
| 4ET-03-009 | 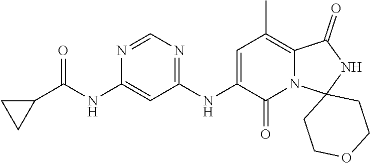 [SMILES: Cc1cc(Nc2cc(NC(=O)C3CC3)ncn2)c(=O)n2c1C(=O)NC21CCOCC1] |
| 4ET-03-010 | 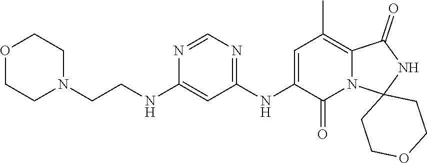 [SMILES: Cc1cc(Nc2cc(NCCN3CCOCC3)ncn2)c(=O)n2c1C(=O)NC21CCOCC1] |
| 4ET-03-011 | 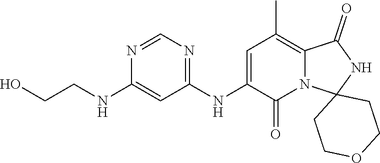 [SMILES: Cc1cc(Nc2cc(NCCO)ncn2)c(=O)n2c1C(=O)NC21CCOCC1] |
| 4ET-03-012 | 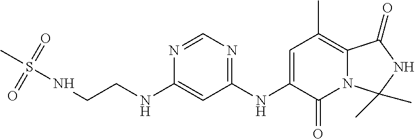 |
| 4ET-03-013 | 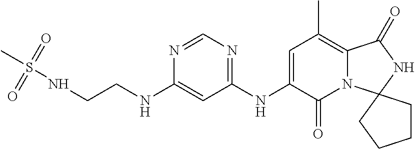 [SMILES: Cc1cc(Nc2cc(NCCNS(C)(=O)=O)ncn2)c(=O)n2c1C(=O)NC21CCCC1] |
| 4ET-03-014 | 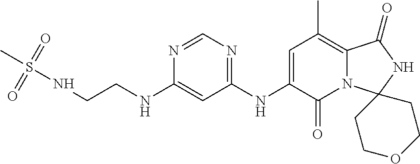 [SMILES: Cc1cc(Nc2cc(NCCNS(C)(=O)=O)ncn2)c(=O)n2c1C(=O)NC21CCOCC1] |
| 4ET-03-015 | 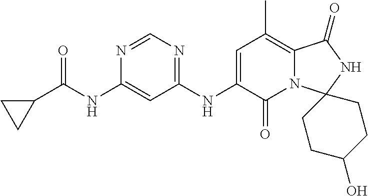 [SMILES: Cc1cc(Nc2cc(NC(=O)C3CC3)ncn2)c(=O)n2c1C(=O)NC21CCC(O)CC1] |
| 4ET-03-016 | 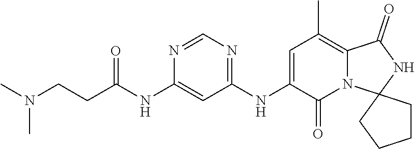 [SMILES: Cc1cc(Nc2cc(NC(=O)CCN(C)C)ncn2)c(=O)n2c1C(=O)NC21CCCC1] |
| 4ET-03-017 | 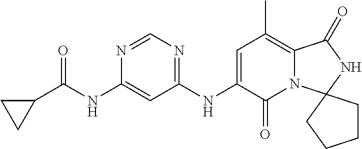 [SMILES: Cc1cc(Nc2cc(NC(=O)C3CC3)ncn2)c(=O)n2c1C(=O)NC21CCCC1] |
| 4ET-03-018 | 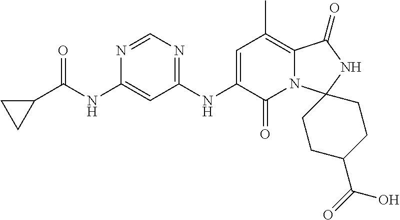 [SMILES: Cc1cc(Nc2cc(NC(=O)C3CC3)ncn2)c(=O)n2c1C(=O)NC21CCC(C(=O)O)CC1] |
| 4ET-03-019 | 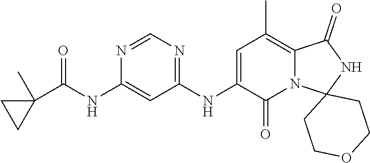 [SMILES: Cc1cc(Nc2cc(NC(=O)C3(C)CC3)ncn2)c(=O)n2c1C(=O)NC21CCOCC1] |
| 4ET-03-020 | 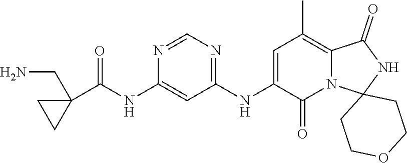 [SMILES: Cc1cc(Nc2cc(NC(=O)C3(CN)CC3)ncn2)c(=O)n2c1C(=O)NC21CCOCC1] |
| 4ET-03-021 | 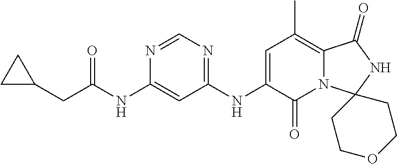 [SMILES: Cc1cc(Nc2cc(NC(=O)CC3CC3)ncn2)c(=O)n2c1C(=O)NC21CCOCC1] |
| 4ET-03-022 | 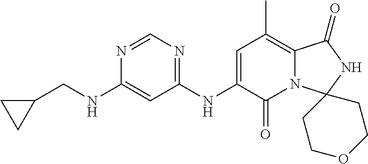 [SMILES: Cc1cc(Nc2cc(NCC3CC3)ncn2)c(=O)n2c1C(=O)NC21CCOCC1] |
| 4ET-03-023 | 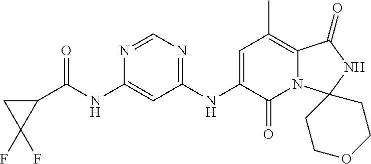 [SMILES: Cc1cc(Nc2cc(NC(=O)C3CC3(F)F)ncn2)c(=O)n2c1C(=O)NC21CCOCC1] |
| 4ET-03-024 | 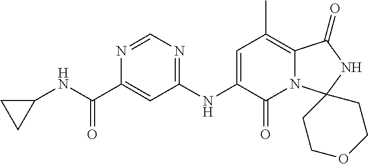 [SMILES: Cc1cc(Nc2cc(C(=O)NC3CC3)ncn2)c(=O)n2c1C(=O)NC21CCOCC1] |
| 4ET-03-025 | 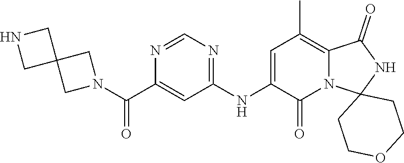 [SMILES: Cc1cc(Nc2cc(C(=O)N3CC4(CNC4)C3)ncn2)c(=O)n2c1C(=O)NC21CCOCC1] |
| 4ET-03-026 | 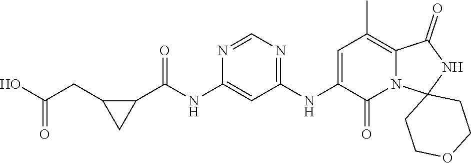 [SMILES: Cc1cc(Nc2cc(NC(=O)C3CC3CC(=O)O)ncn2)c(=O)n2c1C(=O)NC21CCOCC1] |
| 4ET-03-027 | 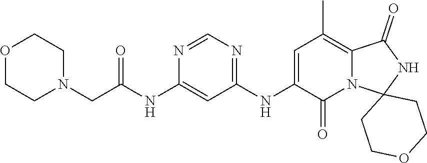 [SMILES: Cc1cc(Nc2cc(NC(=O)CN3CCOCC3)ncn2)c(=O)n2c1C(=O)NC21CCOCC1] |
| 4ET-03-028 | 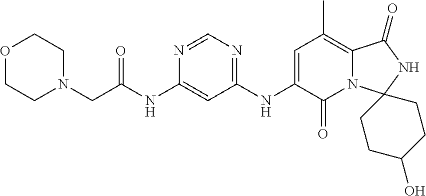 [SMILES: Cc1cc(Nc2cc(NC(=O)CN3CCOCC3)ncn2)c(=O)n2c1C(=O)NC21CCC(O)CC1] |
| 4ET-03-029 | 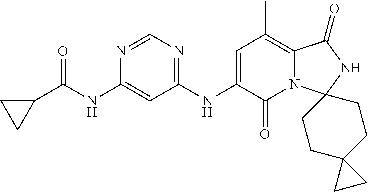 [SMILES: Cc1cc(Nc2cc(NC(=O)C3CC3)ncn2)c(=O)n2c1C(=O)NC21CCC2(CC2)CC1] |
| 4ET-03-030 | 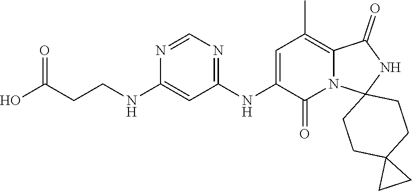 [SMILES: Cc1cc(Nc2cc(NCCC(=O)O)ncn2)c(=O)n2c1C(=O)NC21CCC2(CC2)CC1] |
| 4ET-03-031 | 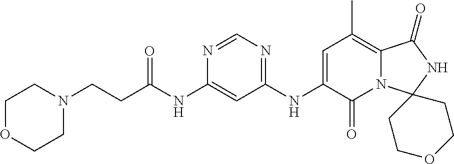 [SMILES: Cc1cc(Nc2cc(NC(=O)CCN3CCOCC3)ncn2)c(=O)n2c1C(=O)NC21CCOCC1] |
| 4ET-03-032 | 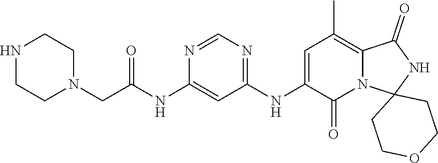 [SMILES: Cc1cc(Nc2cc(NC(=O)CN3CCNCC3)ncn2)c(=O)n2c1C(=O)NC21CCOCC1] |
| 4ET-03-033 | 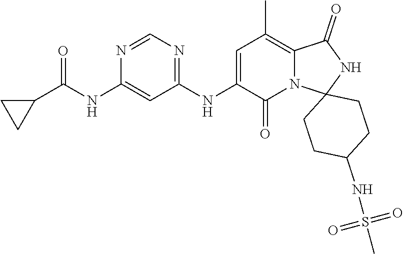 [SMILES: Cc1cc(Nc2cc(NC(=O)C3CC3)ncn2)c(=O)n2c1C(=O)NC21CCC(NS(C)(=O)=O)CC1] |
| 4ET-03-034 | 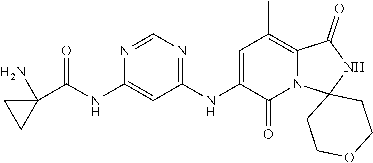 [SMILES: Cc1cc(Nc2cc(NC(=O)C3(N)CC3)ncn2)c(=O)n2c1C(=O)NC21CCOCC1] |
| 4ET-03-035 | 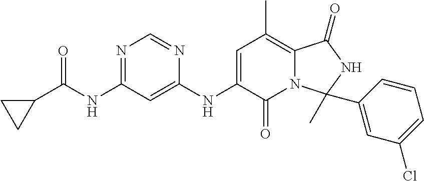 [SMILES: Cc1cc(Nc2cc(NC(=O)C3CC3)ncn2)c(=O)n2c1C(=O)NC2(C)c1cccc(Cl)c1] |
| 4ET-03-036 | 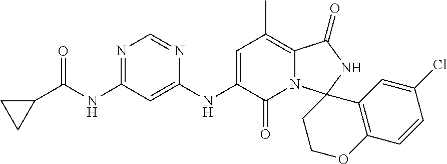 [SMILES: Cc1cc(Nc2cc(NC(=O)C3CC3)ncn2)c(=O)n2c1C(=O)NC21CCOc2ccc(Cl)cc21] |
| 4ET-03-039 | 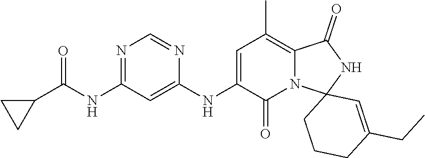 [SMILES: CCC1=CC2(CCC1)NC(=O)c1c(C)cc(Nc3cc(NC(=O)C4CC4)ncn3)c(=O)n12] |
| 4ET-03-050A | 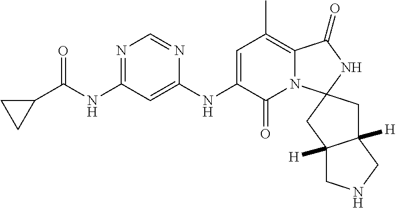 [SMILES: Cc1cc(Nc2cc(NC(=O)C3CC3)ncn2)c(=O)n2c1C(=O)NC21C[C@H]2CNC[C@H]2C1] |
| 4ET-03-050B | 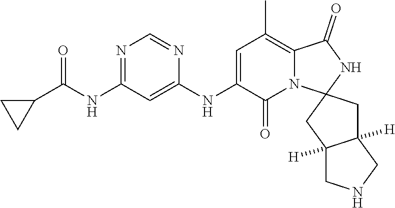 [SMILES: Cc1cc(Nc2cc(NC(=O)C3CC3)ncn2)c(=O)n2c1C(=O)NC21C[C@H]2CNC[C@H]2C1] |
| 4ET-03-052A | 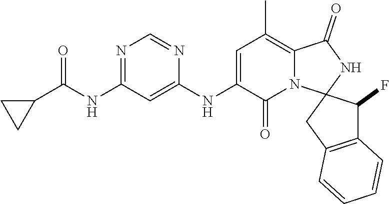 [SMILES: Cc1cc(Nc2cc(NC(=O)C3CC3)ncn2)c(=O)n2c1C(=O)NC21Cc2ccccc2[C@@H]1F] |
| 4ET-03-052B | 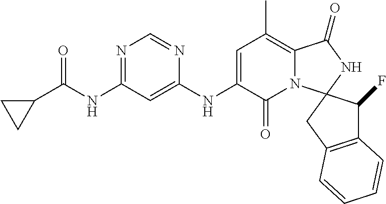 [SMILES: Cc1cc(Nc2cc(NC(=O)C3CC3)ncn2)c(=O)n2c1C(=O)NC21Cc2ccccc2[C@@H]1F] |
| 4ET-03-054 | 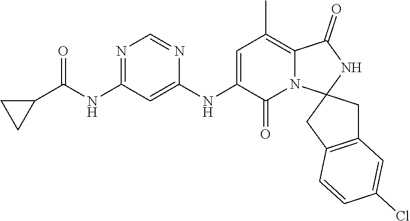 [SMILES: Cc1cc(Nc2cc(NC(=O)C3CC3)ncn2)c(=O)n2c1C(=O)NC21Cc2ccc(Cl)cc2C1] |
| 4ET-03-063 | 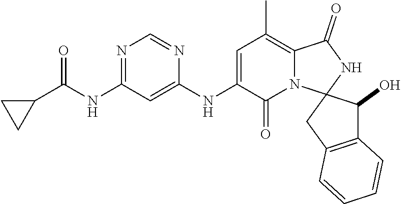 [SMILES: Cc1cc(Nc2cc(NC(=O)C3CC3)ncn2)c(=O)n2c1C(=O)NC21Cc2ccccc2[C@@H]1O] |
| 4ET-03-066 | 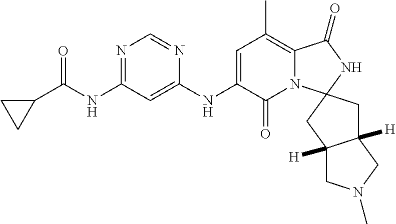 [SMILES: Cc1cc(Nc2cc(NC(=O)C3CC3)ncn2)c(=O)n2c1C(=O)NC21C[C@H]2CN(C)C[C@H]2C1] |
| 4ET-04-003 | 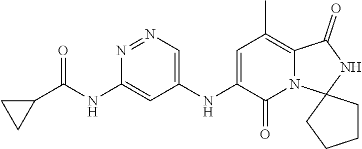 [SMILES: Cc1cc(Nc2cnnc(NC(=O)C3CC3)c2)c(=O)n2c1C(=O)NC21CCCC1] |
| 4ET-04-004 | 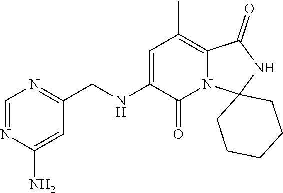 [SMILES: Cc1cc(NCc2cc(N)ncn2)c(=O)n2c1C(=O)NC21CCCCC1] |
| 4ET-04-006 | 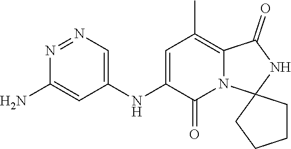 [SMILES: Cc1cc(Nc2cnnc(N)c2)c(=O)n2c1C(=O)NC21CCCC1] |
| 4ET-04-010 | 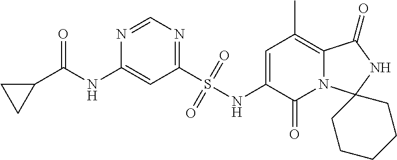 [SMILES: Cc1cc(NS(=O)(=O)c2cc(NC(=O)C3CC3)ncn2)c(=O)n2c1C(=O)NC21CCCCC1] |
| 4ET-04-011 | 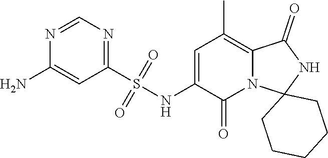 [SMILES: Cc1cc(NS(=O)(=O)c2cc(N)ncn2)c(=O)n2c1C(=O)NC21CCCCC1] |
| 4ET-04-012 | 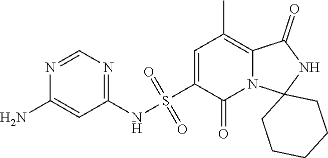 [SMILES: Cc1cc(S(=O)(=O)Nc2cc(N)ncn2)c(=O)n2c1C(=O)NC21CCCCC1] |
| 4ET-04-013 | 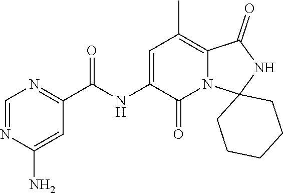 [SMILES: Cc1cc(NC(=O)c2cc(N)ncn2)c(=O)n2c1C(=O)NC21CCCCC1] |
| 4ET-04-014 | 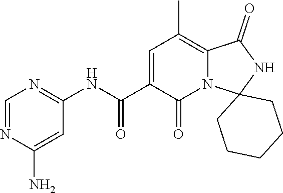 [SMILES: Cc1cc(C(=O)Nc2cc(N)ncn2)c(=O)n2c1C(=O)NC21CCCCC1] |
| 4ET-04-015 | 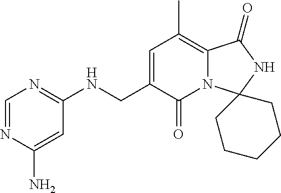 [SMILES: Cc1cc(CNc2cc(N)ncn2)c(=O)n2c1C(=O)NC21CCCCC1] |
| 4ET-04-023 | 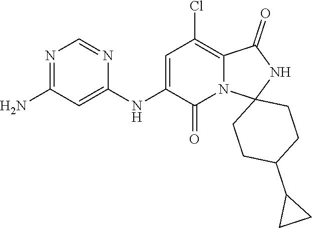 [SMILES: Nc1cc(Nc2cc(Cl)c3n(c2=O)C2(CCC(C4CC4)CC2)NC3=O)ncn1] |
| 1 | 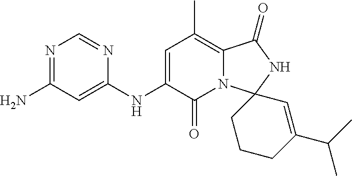 [SMILES: Cc1cc(Nc2cc(N)ncn2)c(=O)n2c1C(=O)NC21C=C(C(C)C)CCC1] |
| 2 | 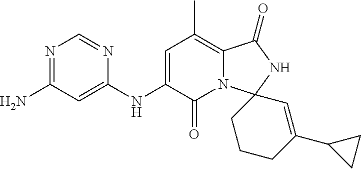 [SMILES: Cc1cc(Nc2cc(N)ncn2)c(=O)n2c1C(=O)NC21C=C(C2CC2)CCC1] |
| 3 | 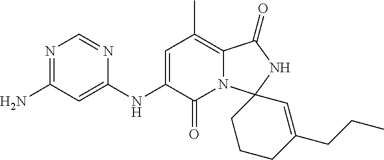 [SMILES: CCCC1=CC2(CCC1)NC(=O)c1c(C)cc(Nc3cc(N)ncn3)c(=O)n12] |
| 4 | 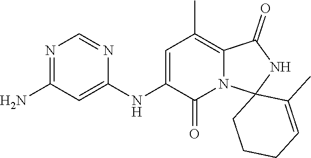 [SMILES: CC1=CCCCC12NC(=O)c1c(C)cc(Nc3cc(N)ncn3)c(=O)n12] |
| 5 | 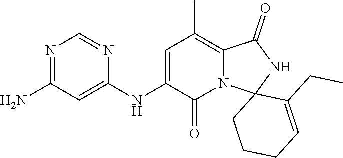 [SMILES: CCC1=CCCCC12NC(=O)c1c(C)cc(Nc3cc(N)ncn3)c(=O)n12] |
| 6 | 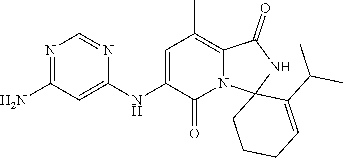 [SMILES: Cc1cc(Nc2cc(N)ncn2)c(=O)n2c1C(=O)NC21CCCC=C1C(C)C] |
| 7 | 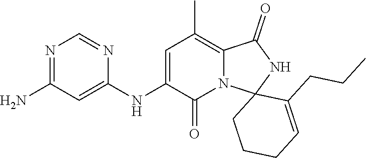 [SMILES: CCCC1=CCCCC12NC(=O)c1c(C)cc(Nc3cc(N)ncn3)c(=O)n12] |
| 8 | 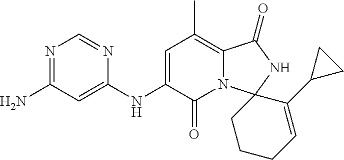 [SMILES: Cc1cc(Nc2cc(N)ncn2)c(=O)n2c1C(=O)NC21CCCC=C1C1CC1] |
| 9 | 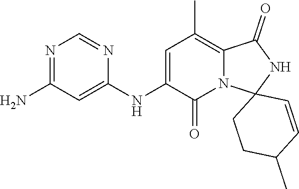 [SMILES: Cc1cc(Nc2cc(N)ncn2)c(=O)n2c1C(=O)NC21C=CC(C)CC1] |
| 10 | 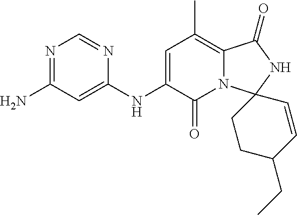 [SMILES: CCC1C=CC2(CC1)NC(=O)c1c(C)cc(Nc3cc(N)ncn3)c(=O)n12] |
| 11 | 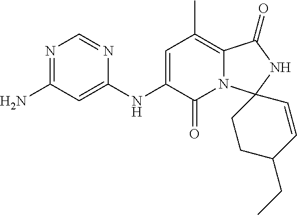 [SMILES: CCC1C=CC2(CC1)NC(=O)c1c(C)cc(Nc3cc(N)ncn3)c(=O)n12] |
| 12 | 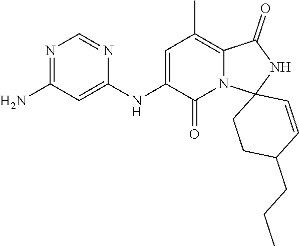 [SMILES: CCCC1C=CC2(CC1)NC(=O)c1c(C)cc(Nc3cc(N)ncn3)c(=O)n12] |
| 13 | 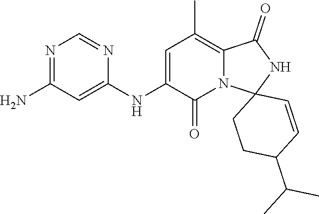 [SMILES: Cc1cc(Nc2cc(N)ncn2)c(=O)n2c1C(=O)NC21C=CC(C(C)C)CC1] |
| 14 | 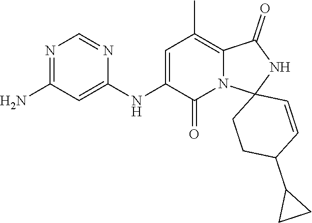 [SMILES: Cc1cc(Nc2cc(N)ncn2)c(=O)n2c1C(=O)NC21C=CC(C2CC2)CC1] |
| 15 | 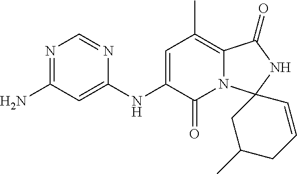 [SMILES: Cc1cc(Nc2cc(N)ncn2)c(=O)n2c1C(=O)NC21C=CCC(C)C1] |
| 16 | 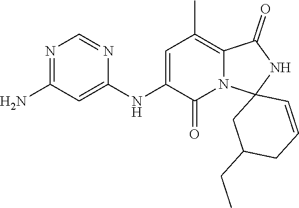 [SMILES: CCC1CC=CC2(C1)NC(=O)c1c(C)cc(Nc3cc(N)ncn3)c(=O)n12] |
| 17 | 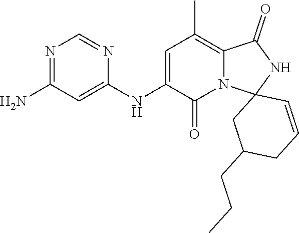 [SMILES: CCCC1CC=CC2(C1)NC(=O)c1c(C)cc(Nc3cc(N)ncn3)c(=O)n12] |
| 18 | 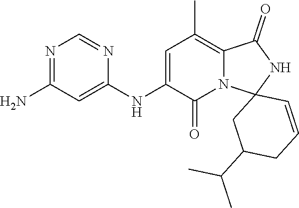 [SMILES: Cc1cc(Nc2cc(N)ncn2)c(=O)n2c1C(=O)NC21C=CCC(C(C)C)C1] |
| 19 | 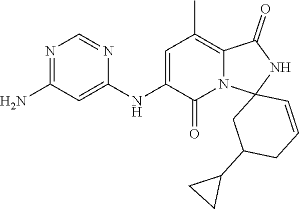 [SMILES: Cc1cc(Nc2cc(N)ncn2)c(=O)n2c1C(=O)NC21C=CCC(C2CC2)C1] |
| 20 | 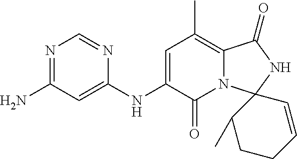 [SMILES: Cc1cc(Nc2cc(N)ncn2)c(=O)n2c1C(=O)NC21C=CCCC1C] |
| 21 | 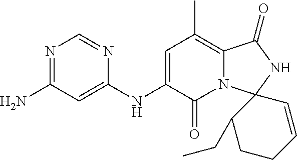 [SMILES: CCC1CCC=CC12NC(=O)c1c(C)cc(Nc3cc(N)ncn3)c(=O)n12] |
| 22 | 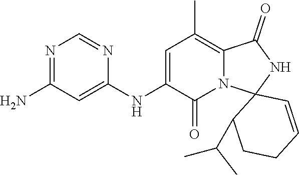 [SMILES: Cc1cc(Nc2cc(N)ncn2)c(=O)n2c1C(=O)NC21C=CCCC1C(C)C] |
| 23 | 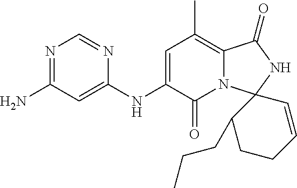 [SMILES: CCCC1CCC=CC12NC(=O)c1c(C)cc(Nc3cc(N)ncn3)c(=O)n12] |
| 24 | 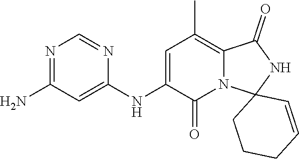 [SMILES: Cc1cc(Nc2cc(N)ncn2)c(=O)n2c1C(=O)NC21C=CCCC1] |
| 25 | 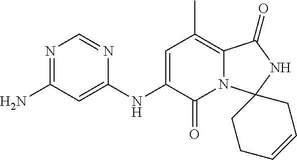 [SMILES: Cc1cc(Nc2cc(N)ncn2)c(=O)n2c1C(=O)NC21CC=CCC1] |
| 26 | 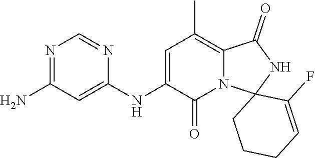 [SMILES: Cc1cc(Nc2cc(N)ncn2)c(=O)n2c1C(=O)NC21CCCC=C1F] |
| 27 | 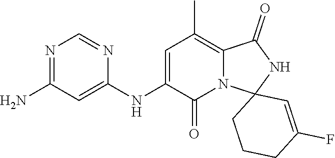 [SMILES: Cc1cc(Nc2cc(N)ncn2)c(=O)n2c1C(=O)NC21C=C(F)CCC1] |
| 28 | 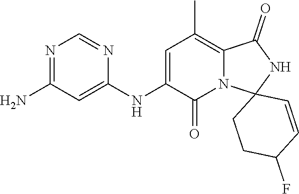 [SMILES: Cc1cc(Nc2cc(N)ncn2)c(=O)n2c1C(=O)NC21C=CC(F)CC1] |
| 29 | 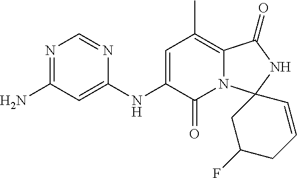 [SMILES: Cc1cc(Nc2cc(N)ncn2)c(=O)n2c1C(=O)NC21C=CCC(F)C1] |
| 30 | 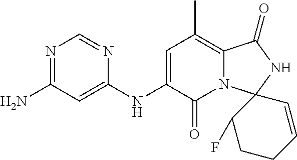 [SMILES: Cc1cc(Nc2cc(N)ncn2)c(=O)n2c1C(=O)NC21C=CCCC1F] |
| 31 | 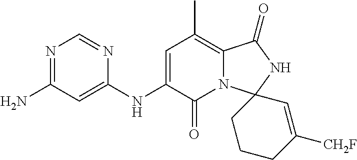 |
| 32 | 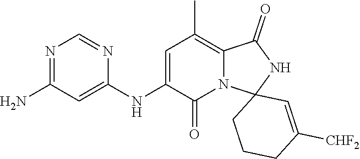 [SMILES: Cc1cc(Nc2cc(N)ncn2)c(=O)n2c1C(=O)NC21C=C(C(F)F)CCC1] |
| 33 | 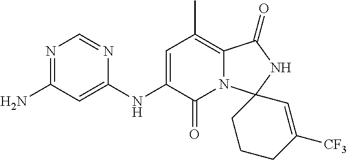 [SMILES: Cc1cc(Nc2cc(N)ncn2)c(=O)n2c1C(=O)NC21C=C(C(F)(F)F)CCC1] |
| 34 | 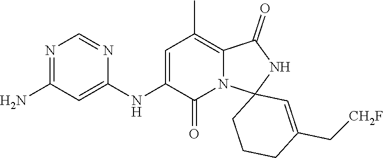 |
| 35 | 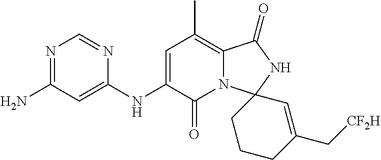 [SMILES: Cc1cc(Nc2cc(N)ncn2)c(=O)n2c1C(=O)NC21C=C(CC(F)F)CCC1] |
| 36 | 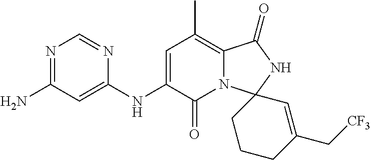 [SMILES: Cc1cc(Nc2cc(N)ncn2)c(=O)n2c1C(=O)NC21C=C(CC(F)(F)F)CCC1] |
| 37 | 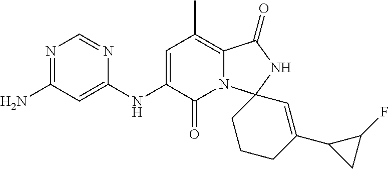 [SMILES: Cc1cc(Nc2cc(N)ncn2)c(=O)n2c1C(=O)NC21C=C(C2CC2F)CCC1] |
| 38 | 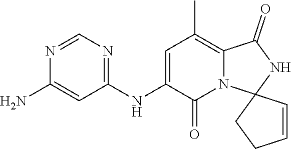 [SMILES: Cc1cc(Nc2cc(N)ncn2)c(=O)n2c1C(=O)NC21C=CCC1] |
| 39 | 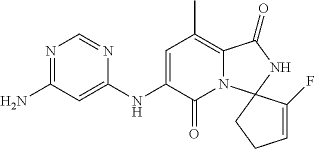 [SMILES: Cc1cc(Nc2cc(N)ncn2)c(=O)n2c1C(=O)NC21CCC=C1F] |
| 40 | 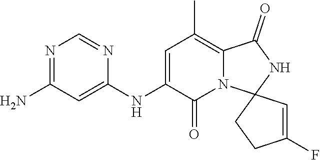 [SMILES: Cc1cc(Nc2cc(N)ncn2)c(=O)n2c1C(=O)NC21C=C(F)CC1] |
| 41 | 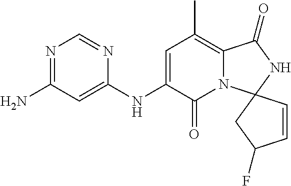 [SMILES: Cc1cc(Nc2cc(N)ncn2)c(=O)n2c1C(=O)NC21C=CC(F)C1] |
| 42 | 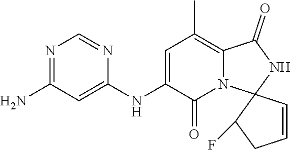 [SMILES: Cc1cc(Nc2cc(N)ncn2)c(=O)n2c1C(=O)NC21C=CCC1F] |
| 43 | 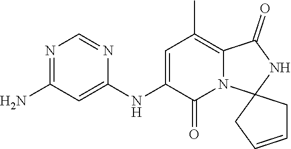 [SMILES: Cc1cc(Nc2cc(N)ncn2)c(=O)n2c1C(=O)NC21CC=CC1] |
| 44 | 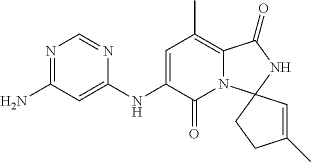 [SMILES: CC1=CC2(CC1)NC(=O)c1c(C)cc(Nc3cc(N)ncn3)c(=O)n12] |
| 45 | 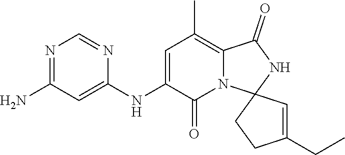 [SMILES: CCC1=CC2(CC1)NC(=O)c1c(C)cc(Nc3cc(N)ncn3)c(=O)n12] |
| 46 | 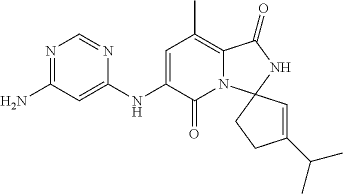 [SMILES: Cc1cc(Nc2cc(N)ncn2)c(=O)n2c1C(=O)NC21C=C(C(C)C)CC1] |
| 47 | 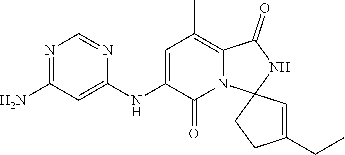 [SMILES: CCC1=CC2(CC1)NC(=O)c1c(C)cc(Nc3cc(N)ncn3)c(=O)n12] |
| 48 | 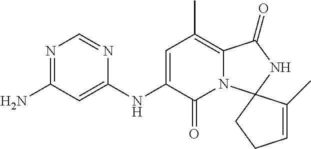 [SMILES: CC1=CCCC12NC(=O)c1c(C)cc(Nc3cc(N)ncn3)c(=O)n12] |
| 49 | 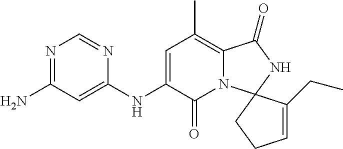 [SMILES: CCC1=CCCC12NC(=O)c1c(C)cc(Nc3cc(N)ncn3)c(=O)n12] |
| 50 | 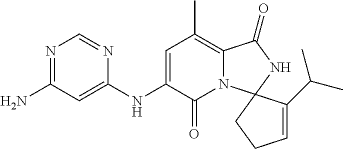 [SMILES: Cc1cc(Nc2cc(N)ncn2)c(=O)n2c1C(=O)NC21CCC=C1C(C)C] |
| 51 | 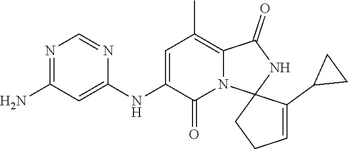 [SMILES: Cc1cc(Nc2cc(N)ncn2)c(=O)n2c1C(=O)NC21CCC=C1C1CC1] |
| 52 | 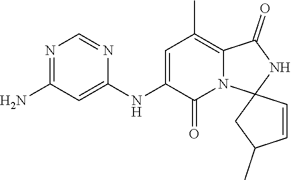 [SMILES: Cc1cc(Nc2cc(N)ncn2)c(=O)n2c1C(=O)NC21C=CC(C)C1] |
| 53 | 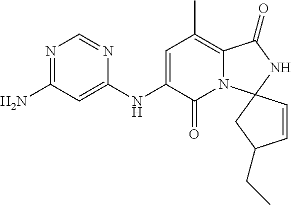 [SMILES: CCC1C=CC2(C1)NC(=O)c1c(C)cc(Nc3cc(N)ncn3)c(=O)n12] |
| 54 | 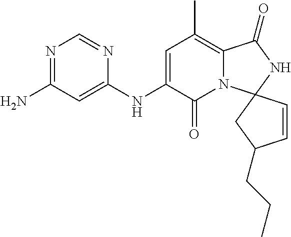 [SMILES: CCCC1C=CC2(C1)NC(=O)c1c(C)cc(Nc3cc(N)ncn3)c(=O)n12] |
| 55 | 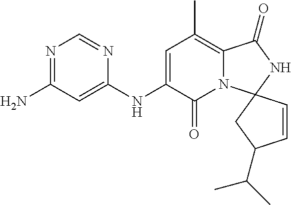 [SMILES: Cc1cc(Nc2cc(N)ncn2)c(=O)n2c1C(=O)NC21C=CC(C(C)C)C1] |
| 56 | 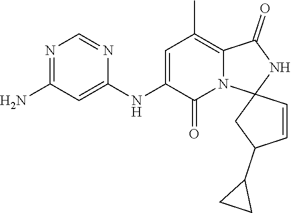 [SMILES: Cc1cc(Nc2cc(N)ncn2)c(=O)n2c1C(=O)NC21C=CC(C2CC2)C1] |
| 57 | 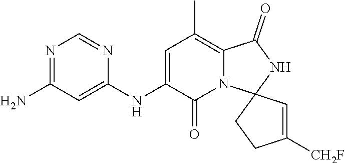 |
| 58 | 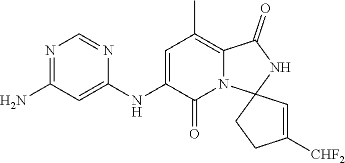 [SMILES: Cc1cc(Nc2cc(N)ncn2)c(=O)n2c1C(=O)NC21C=C(C(F)F)CC1] |
| 59 | 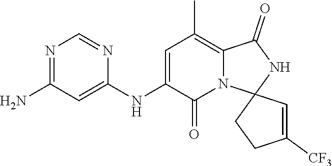 [SMILES: Cc1cc(Nc2cc(N)ncn2)c(=O)n2c1C(=O)NC21C=C(C(F)(F)F)CC1] |
| 60 | 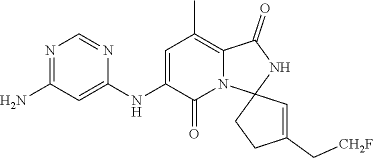 |
| 61 | 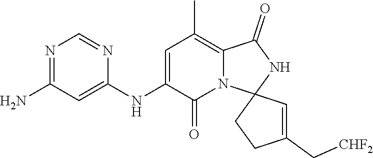 [SMILES: Cc1cc(Nc2cc(N)ncn2)c(=O)n2c1C(=O)NC21C=C(CC(F)F)CC1] |
| 62 | 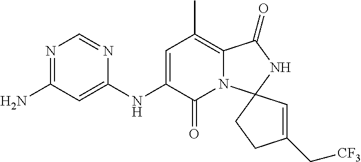 [SMILES: Cc1cc(Nc2cc(N)ncn2)c(=O)n2c1C(=O)NC21C=C(CC(F)(F)F)CC1] |
| 63 | 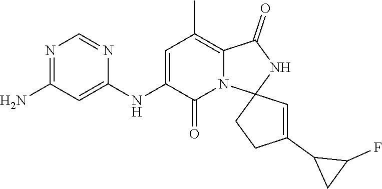 [SMILES: Cc1cc(Nc2cc(N)ncn2)c(=O)n2c1C(=O)NC21C=C(C2CC2F)CC1] |
| 64 | 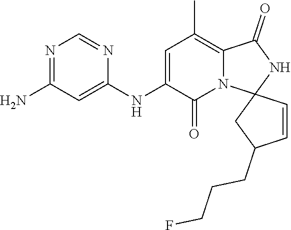 [SMILES: Cc1cc(Nc2cc(N)ncn2)c(=O)n2c1C(=O)NC21C=CC(CCCF)C1] |
| 65 | 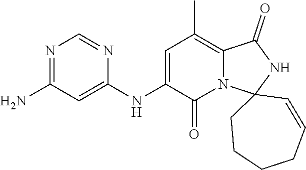 [SMILES: Cc1cc(Nc2cc(N)ncn2)c(=O)n2c1C(=O)NC21C=CCCCC1] |
| 66 | 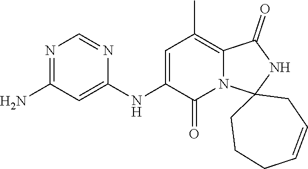 [SMILES: Cc1cc(Nc2cc(N)ncn2)c(=O)n2c1C(=O)NC21CC=CCCC1] |
| 67 | 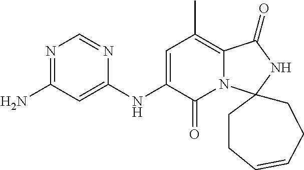 [SMILES: Cc1cc(Nc2cc(N)ncn2)c(=O)n2c1C(=O)NC21CCC=CCC1] |
| 68 | 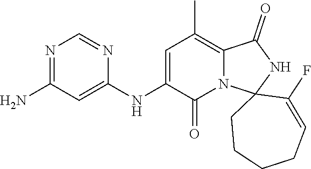 [SMILES: Cc1cc(Nc2cc(N)ncn2)c(=O)n2c1C(=O)NC21CCCCC=C1F] |
| 69 | 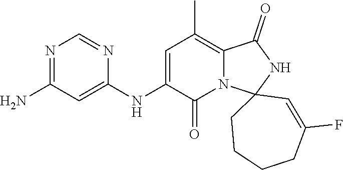 [SMILES: Cc1cc(Nc2cc(N)ncn2)c(=O)n2c1C(=O)NC21C=C(F)CCCC1] |
| 70 | 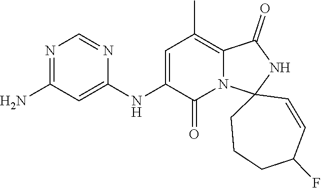 [SMILES: Cc1cc(Nc2cc(N)ncn2)c(=O)n2c1C(=O)NC21C=CC(F)CCC1] |
| 71 | 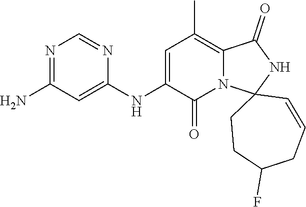 [SMILES: Cc1cc(Nc2cc(N)ncn2)c(=O)n2c1C(=O)NC21C=CCC(F)CC1] |
| 72 | 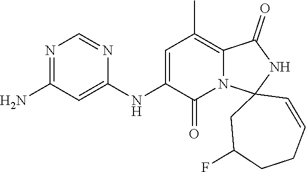 [SMILES: Cc1cc(Nc2cc(N)ncn2)c(=O)n2c1C(=O)NC21C=CCCC(F)C1] |
| 73 | 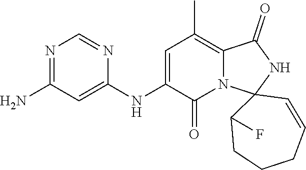 |
| 74 | 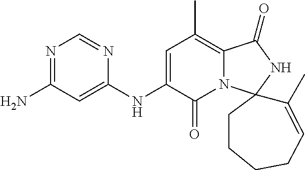 [SMILES: CC1=CCCCCC12NC(=O)c1c(C)cc(Nc3cc(N)ncn3)c(=O)n12] |
| 75 | 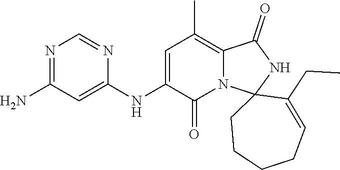 [SMILES: CCC1=CCCCCC12NC(=O)c1c(C)cc(Nc3cc(N)ncn3)c(=O)n12] |
| 76 | 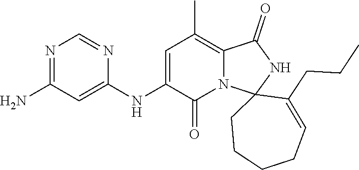 [SMILES: CCCC1=CCCCCC12NC(=O)c1c(C)cc(Nc3cc(N)ncn3)c(=O)n12] |
| 77 | 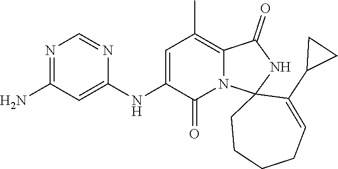 [SMILES: Cc1cc(Nc2cc(N)ncn2)c(=O)n2c1C(=O)NC21CCCCC=C1C1CC1] |
| 78 | 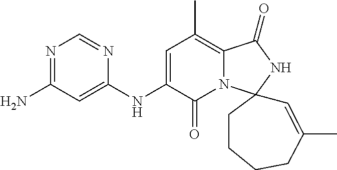 [SMILES: CC1=CC2(CCCC1)NC(=O)c1c(C)cc(Nc3cc(N)ncn3)c(=O)n12] |
| 79 | 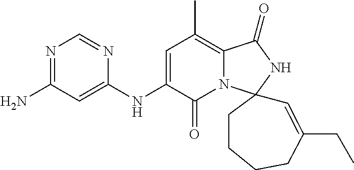 [SMILES: CCC1=CC2(CCCC1)NC(=O)c1c(C)cc(Nc3cc(N)ncn3)c(=O)n12] |
| 80 | 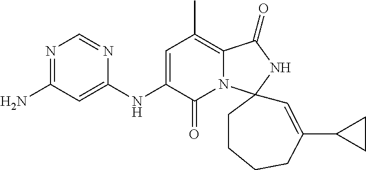 [SMILES: Cc1cc(Nc2cc(N)ncn2)c(=O)n2c1C(=O)NC21C=C(C2CC2)CCCC1] |
| 81 | 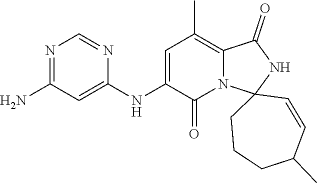 [SMILES: Cc1cc(Nc2cc(N)ncn2)c(=O)n2c1C(=O)NC21C=CC(C)CCC1] |
| 82 | 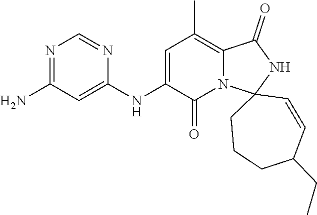 [SMILES: CCC1C=CC2(CCC1)NC(=O)c1c(C)cc(Nc3cc(N)ncn3)c(=O)n12] |
| 83 | 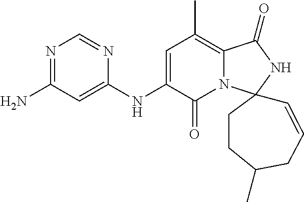 [SMILES: Cc1cc(Nc2cc(N)ncn2)c(=O)n2c1C(=O)NC21C=CCC(C)CC1] |
| 84 | 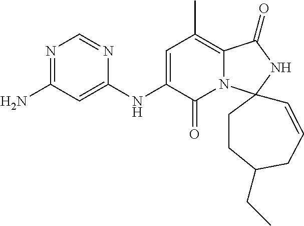 [SMILES: CCC1CC=CC2(CC1)NC(=O)c1c(C)cc(Nc3cc(N)ncn3)c(=O)n12] |
| 85 | 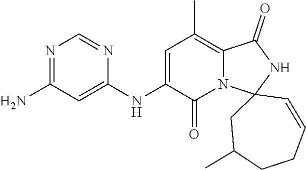 [SMILES: Cc1cc(Nc2cc(N)ncn2)c(=O)n2c1C(=O)NC21C=CCCC(C)C1] |
| 86 | 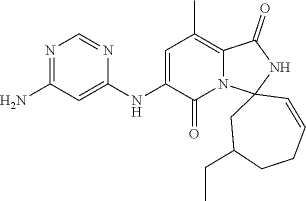 [SMILES: CCC1CCC=CC2(C1)NC(=O)c1c(C)cc(Nc3cc(N)ncn3)c(=O)n12] |
| 87 | 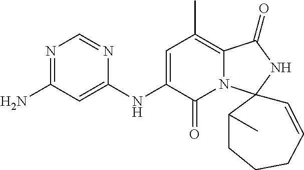 |
| 88 | 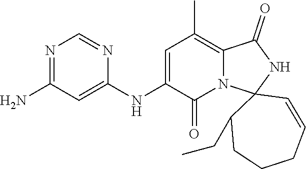 [SMILES: CCC1CCCC=CC12NC(=O)c1c(C)cc(Nc3cc(N)ncn3)c(=O)n12] |
| 89 | 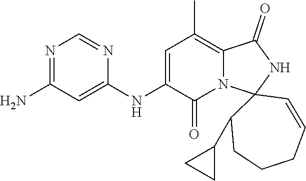 [SMILES: Cc1cc(Nc2cc(N)ncn2)c(=O)n2c1C(=O)NC21C=CCCCC1C1CC1] |
| 90 | 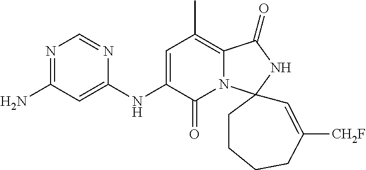 |
| 91 | 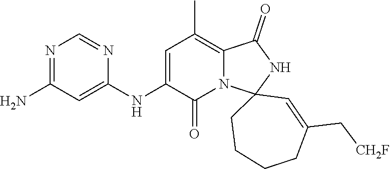 |
| 92 | 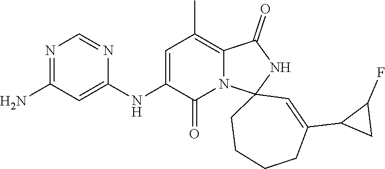 [SMILES: Cc1cc(Nc2cc(N)ncn2)c(=O)n2c1C(=O)NC21C=C(C2CC2F)CCCC1] |

### MNK Inhibitors of Formula (I′) and Formula (IA):

In some embodiments, a MNK inhibitor is a compound of Formula (I′):

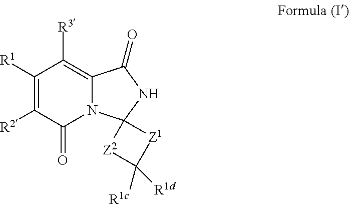

or a pharmaceutically acceptable salt thereof, wherein:
R¹ is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl:
R^{2′} is selected from the group consisting of

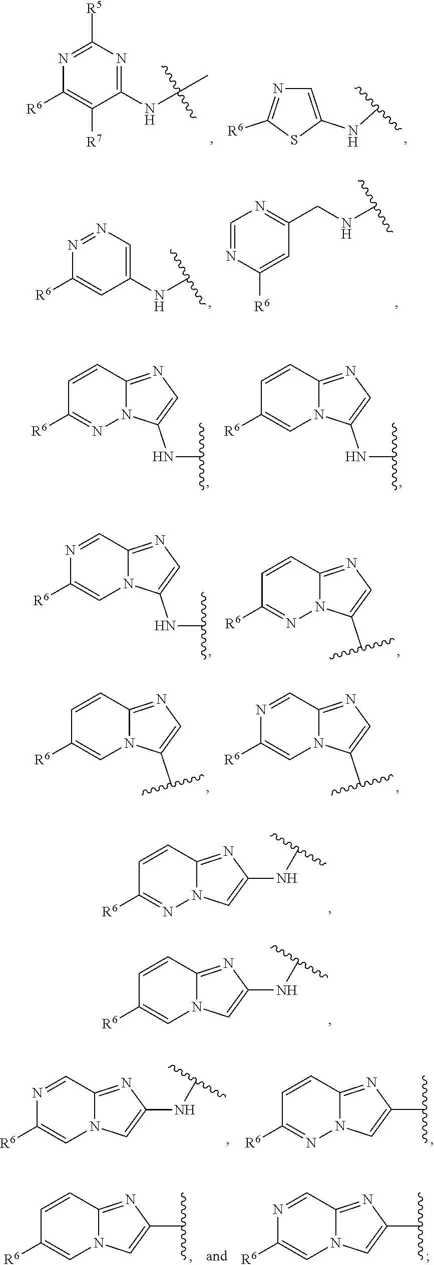

R^{3′} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{1c} and R^{1d} are taken together to form a 3- to 7-membered ring having 0-2 heteroatoms selected from the group consisting of N, O and S, wherein the 3- to 7-membered ring may be further optionally substituted with one or more substituents selected from the group consisting of halo, oxo, C₁₋₆ alkyl, R⁸, and —C(═O)OR⁹;
Z¹ and Z² are each independently a direct bond or —{C(R^{4a})(R^{4b})}ₚ—Y¹—; wherein p is 0, 1, 2, 3, 4, or 5, Y¹ is a direct bond, —O—, or —N(R⁸)—;
R^{4a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl); or two R^{4a} attached to two adjacent carbons to form a direct bond;
R^{4b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R⁵ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxy:
R⁶ is selected from the group consisting of hydrogen, NH₂, NHR^{6a}, NHCH₂CH₂OH, NHCH₂CH₂NHSO₂Me, C₁₋₆ alkoxyl, C₃₋₇ branched alkoxy, and hydroxy;
R^{6a} is selected from the group consisting of —(CO)C₁₋₆ alkyl, —(CO)C₃₋₇ branched alkyl, —(CO)C₁₋₆ hydroxyalkyl,

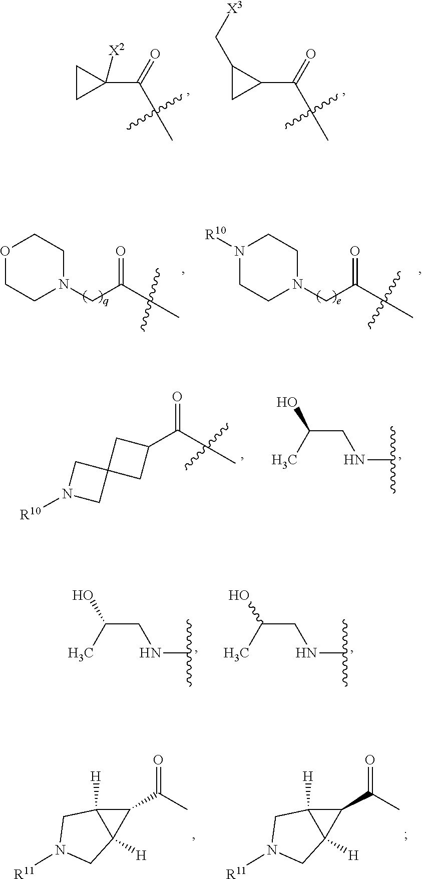

q is 1, 2, 3, 4, 5, or 6;
e is 1, 2, 3, 4, 5, or 6;
X² is selected from the group consisting of hydrogen, halogen, C₁₋₆alkyl, C₃₋₇ branched alkyl, C₁₋₆haloalkyl, C₃₋₇ branched haloalkyl, hydroxy, C₁₋₆hydroxyalkyl, C₃₋₇ branched hydroxyalkyl, C₁₋₆ alkoxy, C₃₋₇ branched alkoxy, C₁₋₆haloalkoxy, C₃₋₇ branched haloalkoxy, NH₂, NH(C₁₋₆ alkyl), N(C₁₋₆ alkyl)₂, C₁₋₅(COOH), C₁₋₅(NHSO₂Me);
X³ is selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ hydroxyalkyl, C₃₋₇ branched hydroxyalkyl, C₁₋₆ alkoxy, C₃₋₇ branched alkoxy, C₁₋₆ haloalkoxy, C₃₋₇ branched haloalkoxy, NH₂, NH(C₁₋₆ alkyl), N(C₁₋₆ alkyl)₂, COOH, C₁₋₅(COOH), NHSO₂Me, C₁₋₅(NHSO₂Me);
R⁷ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxyl;
R⁸ is selected from the group consisting of C₁₋₆ alkyl, C₁₋₆ haloalkyl, C₃₋₇ branched haloalkyl, C₁₋₆ hydroxyalkyl, C₃₋₇ branched hydroxyalkyl, C₁₋₆ alkoxyl, C₃₋₇ branched alkoxy, CO(C₁₋₆alkyl), CO(C₃₋₇ branched alkyl), SO₂(C₁₋₆alkyl), and SO₂(C₃₋₇ branched alkyl);
R⁹ is selected from the group consisting of hydrogen, C₁₋₆ alkyl, and aralkyl.

In more specific embodiments, a MNK inhibitor is a pyridine-1,5-dione of formula (IA):

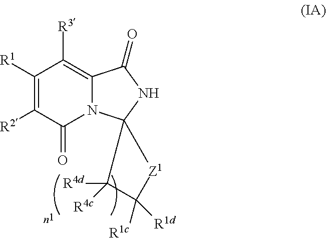

or a pharmaceutically acceptable salt thereof, wherein:
Z¹ is selected from the groups consisting of

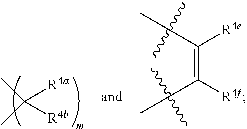

R¹ is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{2′} is selected from the group consisting of

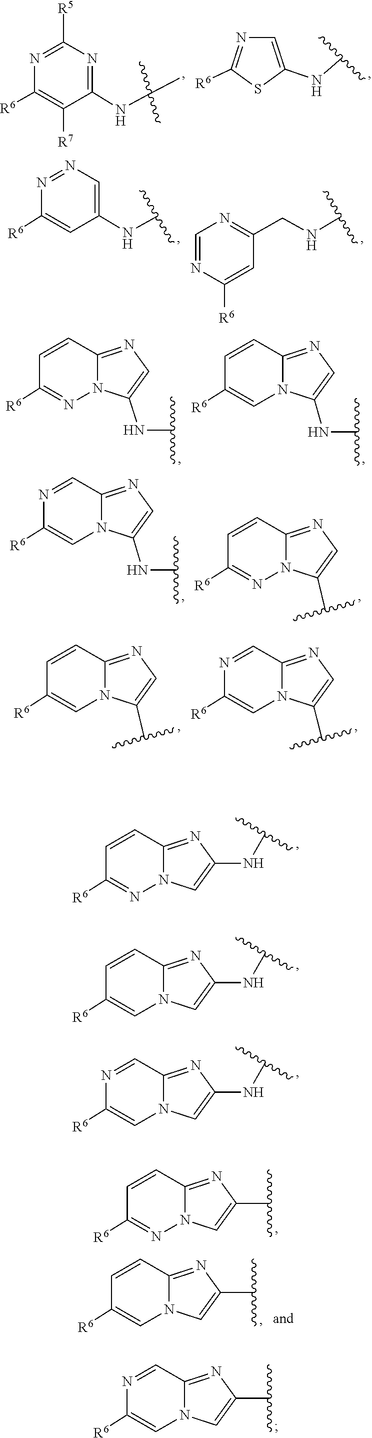

R^{3′} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{4a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R^{4b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl)
R^{4c} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl)
R^{4d} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R^{4e} is hydrogen, halogen, C₁₋₆ alkyl, C₃₋₇ branched alkyl, C₁₋₆ haloalkyl, and C₃₋₇ branched haloalkyl;
R^{4f} is hydrogen, halogen, C₁₋₆ alkyl, C₃₋₇ branched alkyl, C₁₋₆ haloalkyl, and C₃₋₇ branched haloalkyl;
R^{1c} and R^{1d} are taken together to form an optionally substituted 3 to 7 membered ring that optionally contains an X¹ group;
X¹ is selected from the group consisting of CF₂, CHCO₂R¹², O, NH, NR⁸, and SO₂;
m is 0, 1, or 2;
n¹ is 1, 2, or 3;
R⁵ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxy;
R⁶ is selected from the group consisting of hydrogen, NH₂, NHR^{6a}, NHCH₂CH₂OH, NHCH₂CH₂NHSO₂Me, C₁₋₆ alkoxyl, C₃₋₇ branched alkoxy, and hydroxy;
R^{6a} is selected from the group consisting of —(CO)C₁₋₆ alkyl, —(CO)C₃₋₇ branched alkyl, (CO)C₁₋₆ hydroxyalkyl,

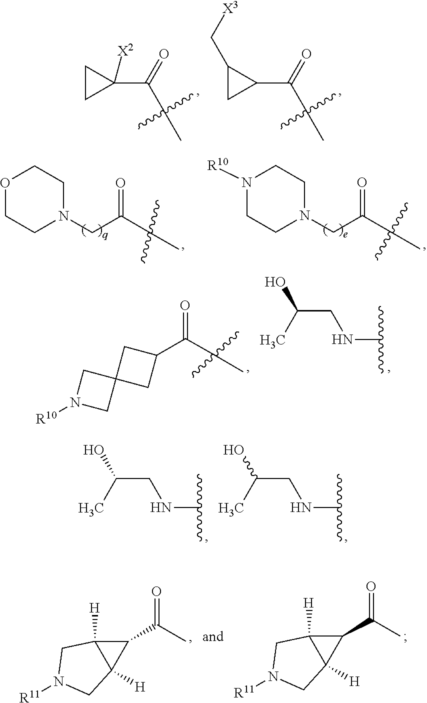

q is 1, 2, 3, 4, 5, or 6;
e is 1, 2, 3, 4, 5, or 6;
X² is selected from the group consisting of hydrogen, halogen, C₁₋₆alkyl, C₃₋₇ branched alkyl, C₁₋₆haloalkyl, C₃₋₇ branched haloalkyl, hydroxy, C₁₋₆hydroxyalkyl, C₃₋₇ branched hydroxyalkyl, C₁₋₆alkoxy, C₃₋₇ branched alkoxy, C₁₋₆haloalkoxy, C₃₋₇ branched haloalkoxy, NH₂, NH(C₁₋₆alkyl), N(C₁₋₆alkyl)₂, C₁₋₅(COOH), C₁₋₆(NHSO₂Me);
X³ is selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ hydroxyalkyl, C₃₋₇ branched hydroxyalkyl, C₁₋₅ alkoxy, C₃₋₇ branched alkoxy, C₁₋₆ haloalkoxy, C₃₋₇ branched haloalkoxy, NH₂, NH(C₁₋₆ alkyl), N(C₁₋₆ alkyl)₂, COOH, C₁₋₅(COOH), NHSO₂Me, C₁₋₅(NHSO₂Me);
R⁷ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxy;
R⁸ is selected from the group consisting of C₁₋₆ alkyl, C₁₋₆ haloalkyl, C₃₋₇ branched haloalkyl, C₁₋₆ hydroxyalkyl, C₃₋₇ branched hydroxyalkyl, C₁₋₆ alkoxyl, C₃₋₇ branched alkoxy, CO(C₁₋₆alkyl), CO(C₃₋₇ branched alkyl), SO₂(C₁₋₆alkyl), and SO₂(C₃₋₇ branched alkyl);
R¹⁰ is selected from the group consisting of hydrogen, C₁₋₆ alkyl, C₁₋₆ haloalkyl, C₃₋₇ branched haloalkyl, C₁₋₆ hydroxyalkyl, C₁₋₆ alkoxyl, C₃₋₇ branched alkoxy, CO(C₁₋₆ alkyl), CO(C₃₋₇ branched alkyl), SO₂(C₁₋₆alkyl), and SO₂(C₃₋₇ branched alkyl);
R¹¹ is selected from the group consisting of hydrogen and C₁₋₆ alkyl;
R¹² is selected from the group consisting of hydrogen and C₁₋₆ alkyl.

The following definitions apply to Formula (I′) and Formula (IA) and subgenera thereof:
As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example, 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, unless otherwise noted, “hydroxyalkyl” whether used alone or as part of a substituent group refers to straight and branched carbon chains having 1 to 20 carbon atoms or any number within this range, for example, 1 to 6 carbon atoms or 1 to 4 carbon atoms that also contains a hydroxyl substituent. Designated numbers of carbon atoms (e.g. C₁₋₆) shall refer independently to the number of carbon atoms in an alkyl moiety or to the alkyl portion of a larger alkyl-containing substituent. Non-limiting examples of hydroxyalkyl groups include hydroxymethyl, hydroxyethyl, hydroxy-n-propyl, hydroxy-iso-propyl, hydroxy-n-butyl, hydroxy-sec-butyl, hydroxy-iso-butyl and the like. Hydroxyalkyl groups can be optionally substituted. In substituent groups with multiple alkyl groups such as (C₂₋₆hydroxyalkyl)2amino, the hydroxyalkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or Spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present disclosure are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocyclyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., two R groups taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present disclosure fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

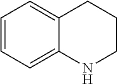

is, for the purposes of the present disclosure, considered a heterocyclic unit. 6,7-Dihydro-5H-cyclopentapyrimidine having the formula:

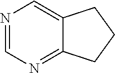

is, for the purposes of the present disclosure, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

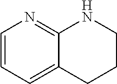

is, for the purposes of the present disclosure, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR^{x}, —SR^{x}, —N(R^{x})₂, —NR^{x}C(O)R^{x}, —SO₂R^{x}, —SO₂OR^{x}, —SO₂N(R^{x})₂, —C(O)R^{x}, —C(O)OR^{x}, —C(O)N(R^{x})₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R^{x}; wherein R^{x}, at each occurrence, independently is hydrogen, —OR^{x+1}, —SR^{x+1}, —C(O)R^{x+1}, —C(O)OR^{x+1}, —C(O)N(R^{x+1})₂, —SO₂R^{x+1}, —S(O)₂OR^{x+1}, —N(R^{x+1})₂, —NR^{x+1}C(O)R^{x+1}, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R^{x}units taken together with the atom(s) to which they are bound form an optionally substituted carbocycle or heterocycle wherein said carbocycle or heterocycle has 3 to 7 ring atoms; wherein R^{x+1}, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆cycloalkyl), aryl, heterocycle, or heteroaryl, or two R^{x+1} units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR^{x+2}; for example, —OH, —OCH₃, —OCH₂CH₃, —OCH₂CH₂CH₃;
ii) —C(O)R^{x+2}; for example, —COCH₃, —COCH₂CH₃, —COCH₂CH₂CH₃;
iii) —C(O)OR^{x+2}; for example, —CO₂CH₃, —CO₂CH₂CH₃, —CO₂CH₂CH₂CH₃;
iv) —C(O)N(R^{x+2})₂; for example, —CONH₂, —CONHCH₃, —CON(CH₃)₂;
v) —N(R^{x+2})₂; for example, —NH₂, —NHCH₃, —N(CH₃)₂, —NH(CH₂CH₃);
vi) halogen, —F, —Cl, —Br, and —I;
vii) —CHₑX_{g} wherein X is halogen, m is from 0 to 2, e+g=3; for example, —CH₂F, —CHF₂, —CF₃, —CCl₃, or —CBr₃;
viii) —SO₂R^{x+2}; for example, —SO₂H; —SO₂CH₃; —SO₂C₆H₅;
ix) C₁-C₆ linear, branched, or cyclic alkyl;
x) Cyano
xi) Nitro;
xii) N(R^{x+2})C(O)R^{x+2};
xiii) Oxo (═O);
xiv) Heterocycle; and
xv) Heteroaryl.
   wherein each R^{x+2} is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R^{x+2} units can be taken together to form a ring comprising 3-7 ring atoms. In certain aspects, each R^{x+2} is independently hydrogen, C₁-C₆ linear or branched alkyl optionally substituted with halogen or C₃-C₆ cycloalkyl or C₃-C₆ cycloalkyl.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

In some embodiments, a MNK inhibitor of the present disclosure is a pyridine-1,5-dione having the formula (I′):

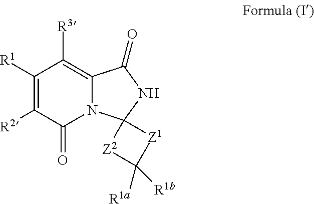

or a pharmaceutically acceptable salt thereof, wherein: PGP-249,01
R¹ is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{2′} is selected from the group consisting of

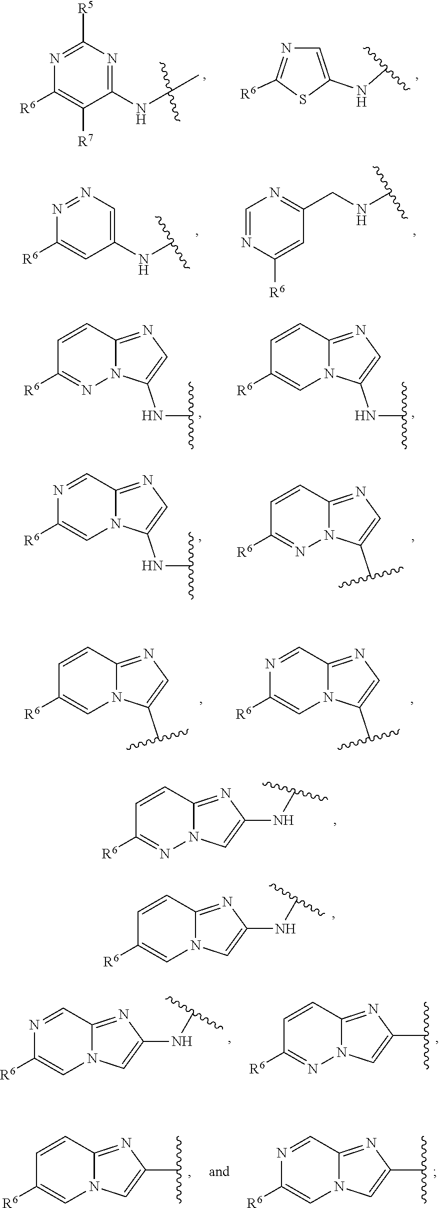

R^{3′} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{1c} and R^{1d} are taken together to form a 3- to 7-membered ring having 0-2 heteroatoms selected from the group consisting of N, O and S, wherein the 3- to 7-membered ring may be further optionally substituted with one or more substituents selected from the group consisting of halo, oxo, C₁₋₆ alkyl, R⁸, and —C(═O)OR⁹;
Z¹ and Z² are each independently a direct bond or —{C(R^{4a})(R^{4b})}ₚ—Y¹—; wherein p is 0, 1, 2, 3, 4, or 5, Y¹ is a direct bond, —O—, or —N(R⁸)—;
R^{4a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl); or two R^{4a} attached to two adjacent carbons to form a direct bond;
R^{4b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R⁵ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxy;
R⁶ is selected from the group consisting of hydrogen, NH₂, NHR^{6a}, NHCH₂CH₂OH, NHCH₂CH₂NHSO₂Me, C₁₋₆ alkoxyl, C₃₋₇ branched alkoxy, and hydroxy;
R^{6a} is selected from the group consisting of —(CO)C₁₋₆ alkyl, —(CO)C₃₋₇ branched alkyl, —(CO)C₁₋₆ hydroxyalkyl,

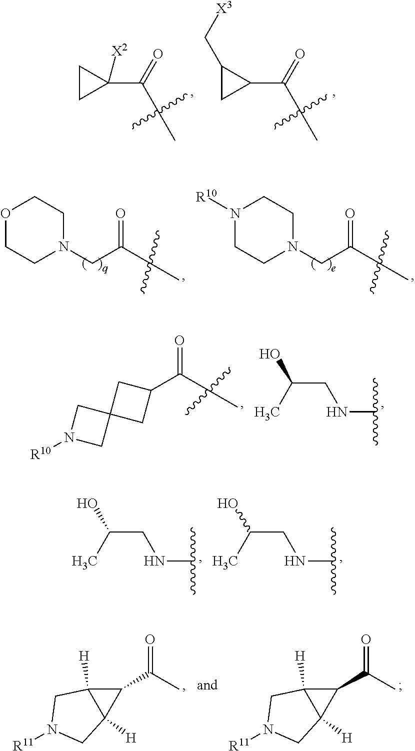

q is 1, 2, 3, 4, 5, or 6;
e is 1, 2, 3, 4, 5, or 6;
X² is selected from the group consisting of hydrogen, halogen, C₁₋₆alkyl, C₃₋₇ branched alkyl, C₁₋₆haloalkyl, C₃₋₇ branched haloalkyl, hydroxy, C₁₋₆hydroxyalkyl, C₃₋₇ branched hydroxyalkyl, C₁₋₆ alkoxy, C₃₋₇ branched alkoxy, C₁₋₆haloalkoxy, C₃₋₇ branched haloalkoxy, NH₂, NH(C₁₋₆ alkyl), N(C₁₋₆ alkyl)2, C₁₋₅(COOH), C₁₋₅(NHSO₂Me);
X³ is selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ hydroxyalkyl, C₃₋₇ branched hydroxyalkyl, C₁₋₆ alkoxy, C₃₋₇ branched alkoxy, C₁₋₆ haloalkoxy, C₃₋₇ branched haloalkoxy, NH₂, NH(C₁₋₅ alkyl), N(C₁₋₆ alkyl)₂, COOH, C₁₋₅(COOH), NHSO₂Me, C₁₋₅(NHSO₂Me);
R⁷ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxyl;
R⁸ is selected from the group consisting of C₁₋₆ alkyl, C₁₋₆ haloalkyl, C₃₋₇ branched haloalkyl, C₁₋₆ hydroxyalkyl, C₃₋₇ branched hydroxyalkyl, C₁₋₆ alkoxyl, C₃₋₇ branched alkoxy, CO(C₁₋₆alkyl), CO(C₃₋₇ branched alkyl), SO₂(C₁₋₆alkyl), and SO₂(C₃₋₇ branched alkyl);
R⁹ is selected from the group consisting of hydrogen, C₁₋₆ alkyl, and aralkyl.

In more specific embodiments, the compound exhibiting MNK inhibition has the following structure, represented by Formula (IA):

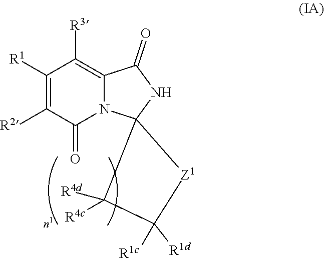

or a pharmaceutically acceptable salt thereof, wherein:
Z¹ is selected from the groups consisting of,

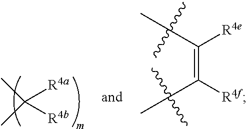

R¹ is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{2′} is selected from the group consisting of

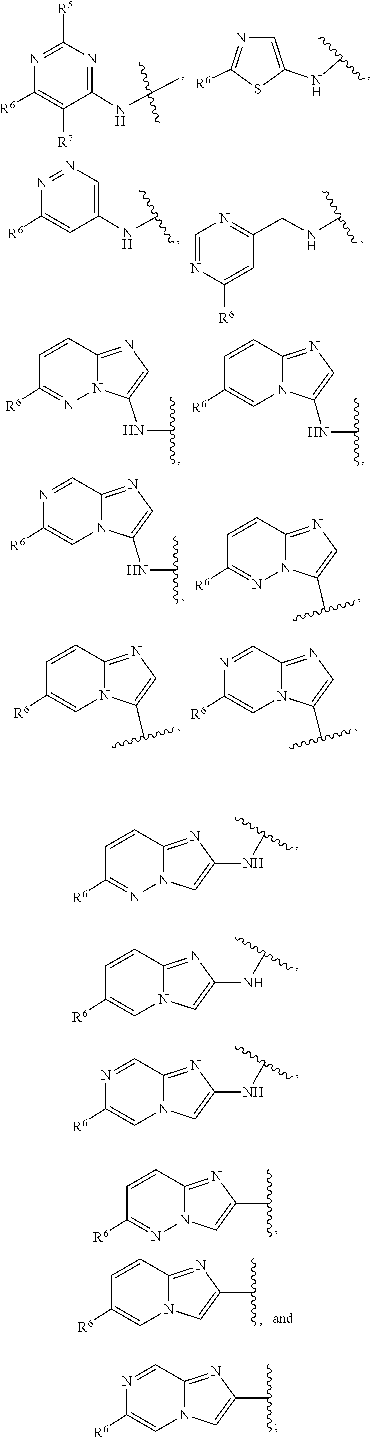

R^{3′} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{4a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R^{4b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇cycloalkyl), NHSO₂(C₁₋₆alkyl), NHSO₂(C₃₋₇ branched alkyl), and NHSO₂(C₃₋₇ cycloalkyl);
R^{4c} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R^{4d} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R^{4e} is hydrogen, halogen, C₁₋₆ alkyl, C₃₋₇ branched alkyl, C₁₋₆ haloalkyl, and C_{3*-7} branched haloalkyl;
R^{4f} is hydrogen, halogen, C₁₋₆ alkyl, C₃₋₇ branched alkyl, C₁₋₆ haloalkyl, and C₃₋₇ branched haloalkyl;
R^{1c} and R^{1d} are taken together to form an optionally substituted 3 to 7 membered ring that optionally contains an X¹ group forming a part of the ring;
X¹ is selected from the group consisting of —C(F)₂—, —CH(CO₂R¹²)—, —O—, —NH—, —N(R⁸)—, and —S(═O)₂—;
m is 0, 1, or 2;
n¹ is 1, 2, or 3;
R⁵ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxy;
R⁶ is selected from the group consisting of hydrogen, NH₂, NHR^{6a}, NHCH₂CH₂OH, NHCH₂CH₂NHSO₂Me, C₁₋₆ alkyl, C₃₋₇ branched alkyl, C₁₋₆ alkoxyl, C₃₋₇ branched alkoxy, and hydroxy;
R^{6a} is selected from the group consisting of —(CO)C₁₋₆ alkyl, —(CO)C₃₋₇ branched alkyl, —(CO)C₁₋₆ hydroxyalkyl,

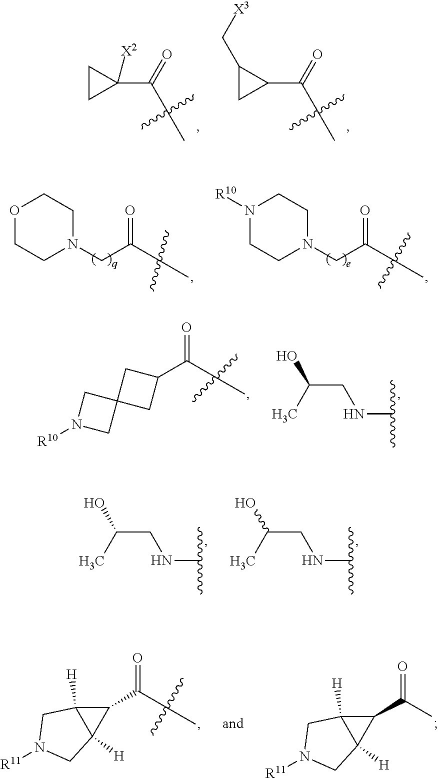

q is 1, 2, 3, 4, 5, or 6;
e is 1, 2, 3, 4, 5, or 6;
X² is selected from the group consisting of hydrogen, halogen, C₁₋₆alkyl, C₃₋₇ branched alkyl, C₁₋₆ haloalkyl, C₃₋₇ branched haloalkyl, hydroxy, C₁₋₆hydroxyalkyl, C₃₋₇ branched hydroxyalkyl, C₁₋₆ alkoxy, C₃₋₇ branched alkoxy, C₁₋₆haloalkoxy, C₃₋₇ branched haloalkoxy, NH₂, NH(C₁₋₆ alkyl), N(C₁₋₆ alkyl)2, C₁₋₅(COOH), C₁₋₆(NHSO₂Me);
X³ is selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ hydroxyalkyl, C₃₋₇ branched hydroxyalkyl, C₁₋₅ alkoxy, C₃₋₇ branched alkoxy. C₁₋₆ haloalkoxy, C₃₋₇ branched haloalkoxy. NH₂, NH(C₁₋₆ alkyl), N(C₁₋₆ alkyl)₂, COOH, C₁₋₅(COOH), NHSO₂Me, C₁₋₅(NHSO₂Me);
R⁷ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxy;
R⁸ is selected from the group consisting of C₁₋₆ alkyl, C₁₋₆ haloalkyl, C₃₋₇ branched haloalkyl, C₁₋₆ hydroxyalkyl, C₃₋₇ branched hydroxyalkyl, C₁₋₆ alkoxyl, C₃₋₇ branched alkoxy, CO(C₁₋₆alkyl), CO(C₃₋₇ branched alkyl), SO₂(C₁₋₆alkyl), and SO₂(C₃₋₇ branched alkyl);
R¹⁰ is selected from the group consisting of hydrogen. C₁₋₆ alkyl, C₁₋₆ haloalkyl, C₃₋₇ branched haloalkyl, C₁₋₆ hydroxyalkyl, C₁₋₆ alkoxyl, C₃₋₇ branched alkoxy, CO(C₁₋₆alkyl), CO(C₃₋₇ branched alkyl), SO₂(C₁₋₆alkyl), and SO₂(C₃₋₇ branched alkyl).
R¹¹ is selected from the group consisting of hydrogen and C₁₋₆ alkyl;
R¹² is selected from the group consisting of hydrogen and C₁₋₆ alkyl.

In more specific embodiments, the compounds of the present disclosure include compounds having formula (IIA):

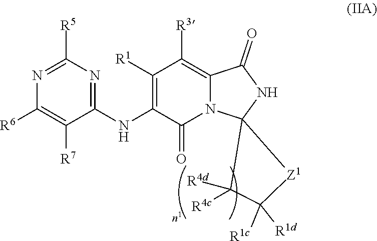

or a pharmaceutically acceptable salt thereof. R^{1c}, R^{1d}, R¹, R^{3′}, R^{4d}, R^{4c}, n¹, Z¹, R⁵, R⁶ and R⁷ are as defined herein.

In more specific embodiments, the compounds of the present disclosure include compounds having formula (III):

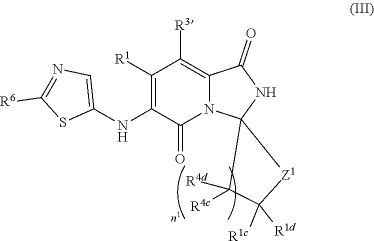

or a pharmaceutically acceptable salt thereof. R^{1c}, R^{1d}, R¹, R^{3′}, R^{4d}, R^{4c}, n¹, Z¹, and R⁶ are as defined herein.

In more specific embodiments, the compounds of the present disclosure include compounds having formula (IV):

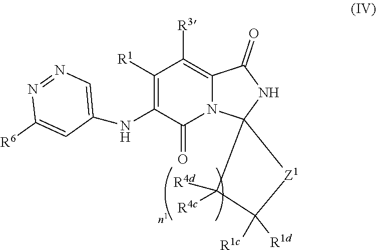

or a pharmaceutically acceptable salt thereof. R^{1c}, R^{1d}, R¹, R^{3′}, R^{4d}, R^{4c}, n¹, Z¹, and R⁶ are as defined herein.

In more specific embodiments, the compounds of the present disclosure include compounds having formula (V):

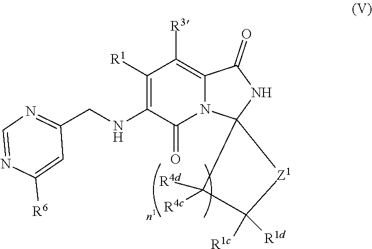

or a pharmaceutically acceptable salt thereof. R^{1c}, R^{1d}, R¹, R^{3′}, R^{4d}, R^{4c}, n¹, Z¹, and R⁶ are as defined herein.

In more specific embodiments, the compounds of the present disclosure include compounds having formula (VI):

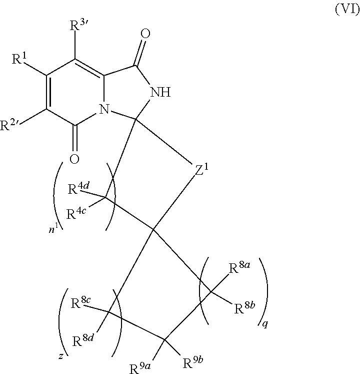

or a pharmaceutically acceptable salt thereof, wherein
R¹, R^{2′}, R^{3′}, R^{4d}, R^{4c}, n¹, and Z¹ are as defined herein;
R^{8a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8c} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8d} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{9a} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, and C₃₋₇ branched alkoxy;
R^{9b} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, and C₃₋₇ branched alkoxy;
R^{9a} and R^{9b} are taken together to form an optionally substituted 3 to 7-membered ring;
q is 1, 2, or 3; and
z is 0, 1, or 2.

In more specific embodiments, the compounds of the present disclosure include compounds having formula (VII):

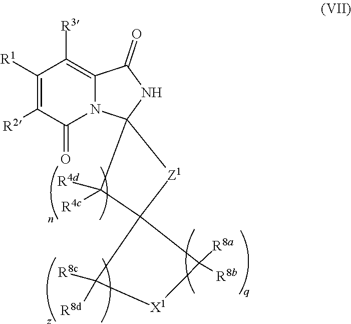

or a pharmaceutically acceptable salt thereof, wherein
R¹, R^{2′}, R^{3′}, R^{4d}, R^{4c}, Z¹, X¹ and n¹ are as defined herein;
R^{8a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8c} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8d} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
q is 1, 2, or 3; and
z is 0, 1, or 2.

In more specific embodiments, the compounds of the present disclosure include compounds having formula (VIII):

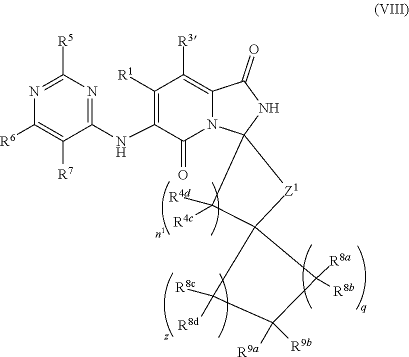

or a pharmaceutically acceptable salt thereof, wherein
R¹, R^{3′}, R^{4d}, R^{4c}, Z¹, R⁵, R⁶, R⁷, and n¹ are as defined herein;
R^{8a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8c} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8d} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{9a} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{9b} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
q is 1, 2, or 3; and
z is 0, 1, or 2.

In more specific embodiments, the compounds of the present disclosure include compounds having formula (IX):

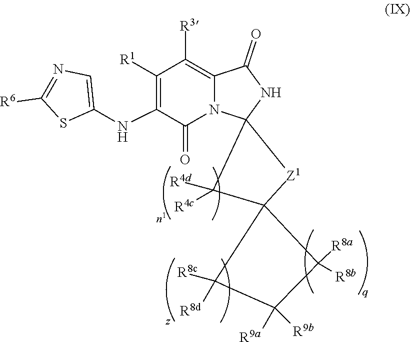

or a pharmaceutically acceptable salt thereof. R¹, R^{3′}, R^{4d}, R^{4c}, Z¹, R⁶, R^{8a}, R^{8b}, R^{8c}, R^{8d}, n¹ and z are as defined herein.

In more specific embodiments, the compounds of the present disclosure include compounds having formula (X):

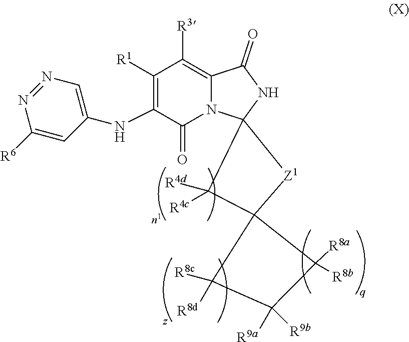

or a pharmaceutically acceptable salt thereof. R¹, R^{3′}, R^{4d}, R^{4c}, Z¹, R⁶, R^{8a}, R^{8b}, R^{8c}, R^{8d}, R^{9a}, R^{9b}, n¹ and z are as defined herein.

In more specific embodiments, the compounds of the present disclosure include compounds having formula (XI):

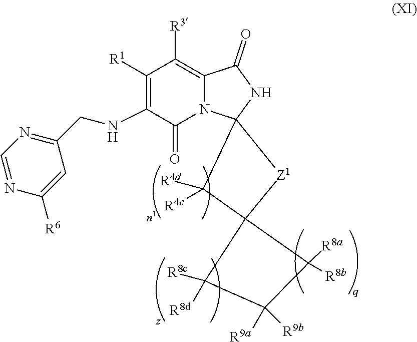

or a pharmaceutically acceptable salt thereof. R¹, R^{3′}, R^{4d}, R^{4c}, Z¹, R⁶, R^{8a}, R^{8b}, R^{8c}, R^{8d}, R^{9a}, R^{9b}, n¹ and z are as defined herein.

In more specific embodiments, the compounds of the present disclosure include compounds having formula (XII):

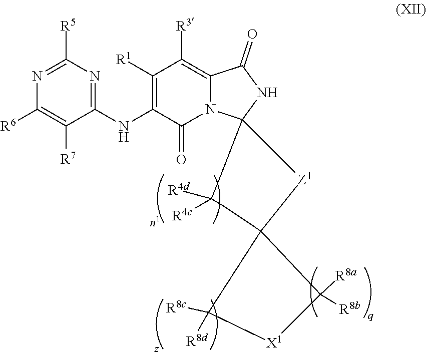

or a pharmaceutically acceptable salt thereof, wherein
R¹, R^{3′}, R^{4d}, R^{4c}, Z¹, R⁵, R⁶, R⁷, X¹, and n¹ are as defined herein;
R⁸ is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8c} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
R^{8d} is at each occurrence independently selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, hydroxy, C₁₋₆ alkoxyl, C₃₋₇ branched alkoxy, NHCO(C₁₋₆alkyl), NHCO(C₃₋₇ branched alkyl), NHSO₂(C₁₋₆alkyl), and NHSO₂(C₃₋₇ branched alkyl);
q is 1, 2, or 3; and
z is 0, 1, or 2.

In more specific embodiments, the compounds of the present disclosure include compounds having formula (XIII):

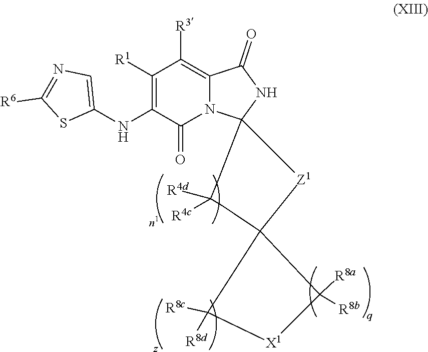

(or a pharmaceutically acceptable salt thereof. R¹, R^{3′}, R^{4d}, R^{4c}, Z¹, R⁶, R^{8a}, R^{8b}, R^{8c}, R^{8d}, X¹, n¹ and z are as defined herein.

In more specific embodiments, the compounds of the present disclosure include compounds having formula (XIV):

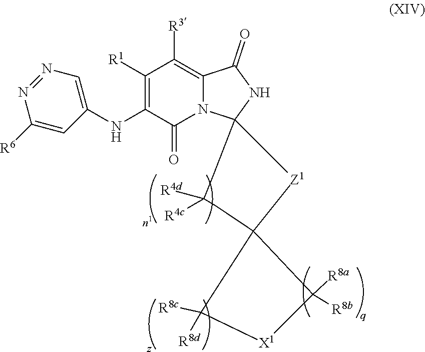

or a pharmaceutically acceptable salt thereof. R¹, R^{3′}, R^{4d}, R^{4c}, Z¹, R⁶, R^{8a}, R^{8b}, R^{8c}, R^{8d}, X¹, n¹ and z are as defined herein.

In more specific embodiments, the compounds of the present disclosure include compounds having formula (XV):

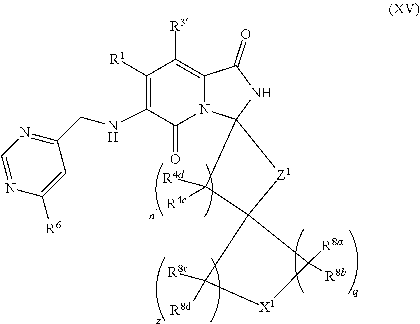

or a pharmaceutically acceptable salt thereof. R¹, R^{3′}, R^{4d}, R^{4c}, Z¹, R⁶, R^{8a}, R^{8b}, R^{8c}, R^{8d}, X¹, n¹ and z are as defined herein.

In more specific embodiments, the compounds of the present disclosure include compounds having formula (XVI):

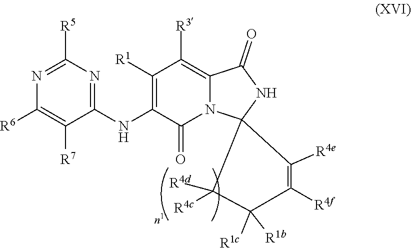

or a pharmaceutically acceptable salt thereof. R^{1c}, R^{1d}, R¹, R^{3′}, R^{4d}, R^{4c}, R^{4e}, R^{4f}, R⁵, R⁶, R⁷ and n¹ are as defined herein.

The compounds of the present disclosure include compounds having formula (XV):

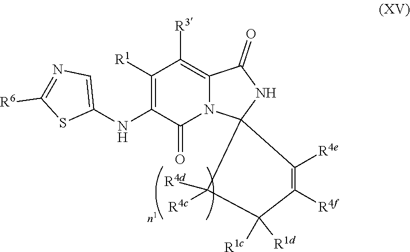

or a pharmaceutically acceptable salt thereof. R^{1c}, R^{1d}, R¹, R^{3′}, R^{4d}, R^{4c}, R^{4e}, R^{4f}, R⁶, and n¹ are as defined herein.

In more specific embodiments, the compounds of the present disclosure include compounds having formula (XVI):

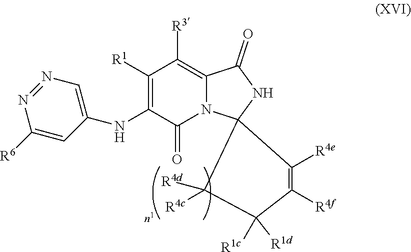

or a pharmaceutically acceptable salt thereof. R^{1c}, R^{1d}, R¹, R^{3′}, R^{4d}, R^{4c}, R^{4e}, R^{4f}, R⁶, and n¹ are as defined herein.

In more specific embodiments, the compounds of the present disclosure include compounds having formula ((VII):

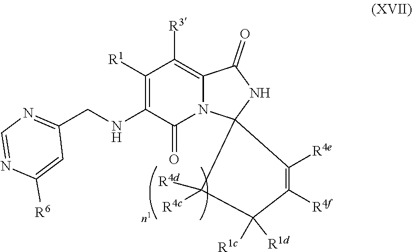

or a pharmaceutically acceptable salt thereof. R^{1c}, R^{1d}, R¹, R^{3′}, R^{4d}, R^{4c}, R^{4e}, R^{4f}, R⁶, and n¹ are as defined herein.

In more specific embodiments, the compounds of the present disclosure include compounds having formula (XVIII):

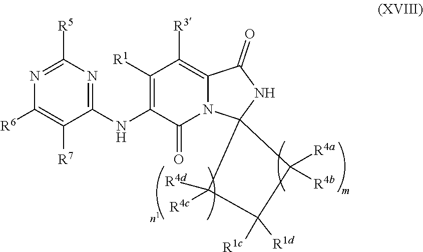

or a pharmaceutically acceptable salt thereof. R^{1c}, R^{1d}, R¹, R^{3′}, R^{4a}, R^{4b}, R^{4d}, R^{4c}, R⁵, R⁶, R⁷, m and n¹ are as defined herein.

In more specific embodiments, the compounds of the present disclosure include compounds having formula (XIX):

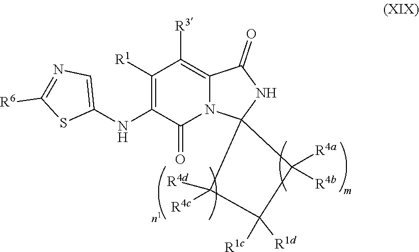

or a pharmaceutically acceptable salt thereof. R^{1c}, R^{1d}, R¹, R^{3′}, R^{4d}, R^{4c}, R^{4a}, R^{4b}, R⁶, m and n¹ are as defined herein.

In more specific embodiments, the compounds of the present disclosure include compounds having formula (XX):

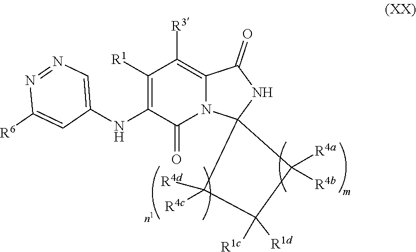

or a pharmaceutically acceptable salt thereof. R^{1c}, R^{1d}, R¹, R^{3′}, R^{4d}, R^{4c}, R^{4a}, R^{4b}, R⁶, m and n¹ are as defined herein.

In more specific embodiments, the compounds of the present disclosure include compounds having formula (XX):

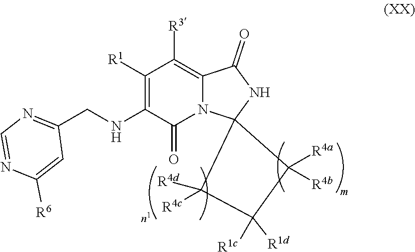

or a pharmaceutically acceptable salt thereof. R^{1c}, R^{1d}, R¹, R^{3′}, R^{4d}, R^{4c}, R^{4a}, R^{4b}, R⁶, m and n¹ are as defined herein.

In more specific embodiments, the compounds of the present disclosure include compounds having formula (XXI):

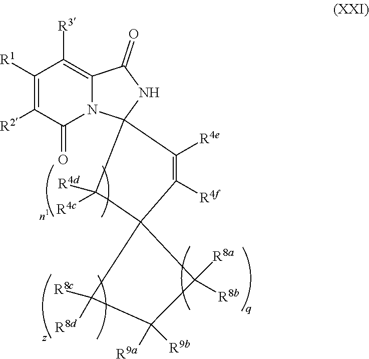

or a pharmaceutically acceptable salt thereof. R¹, R^{2′}, R^{3′}, R^{4d}, R^{4c}, R^{4e}, R^{4f}, R^{8a}, R^{8b}, R^{8c}, R^{8d}, R^{9a}, R^{9b}, n¹, q and z are as defined herein.

In more specific embodiments, the compounds of the present disclosure include compounds having formula (XXII):

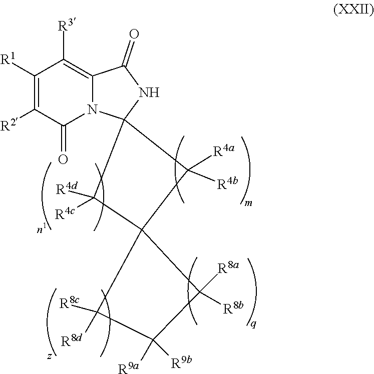

or a pharmaceutically acceptable salt thereof. R¹, R^{2′}, R^{3′}, R^{4d}, R^{4c}, R^{4e}, R^{4f}, R^{8a}, R^{8b}, R^{8c}, R^{8d}, R^{9a}, R^{9b}, m, n¹, q and z are as defined herein.

In more specific embodiments, the compounds of the present disclosure include compounds having formula (XXIII):

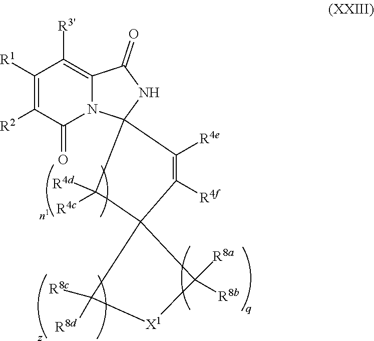

or a pharmaceutically acceptable salt thereof. R¹, R^{2′}, R^{3′}, R^{4d}, R^{4c}, R^{4e}, R^{4f}, R^{8a}, R^{8b}, R^{8c}, R^{8d}, X¹, n¹, q and z are as defined herein.

In more specific embodiments, the compounds of the present disclosure include compounds having formula (XXIV):

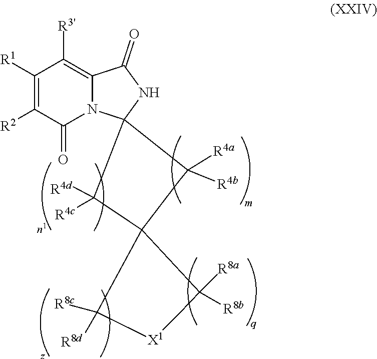

or a pharmaceutically acceptable salt thereof. R¹, R^{2′}, R^{3′}, R^{4d}, R^{4c}, R^{4e}, R^{4f}, R^{8a}, R^{8b}, R^{8c}, R^{8d}, X¹, m, n¹, q and z are as defined herein.

In more specific embodiments, the compounds of the present disclosure include compounds having formula (XXV):

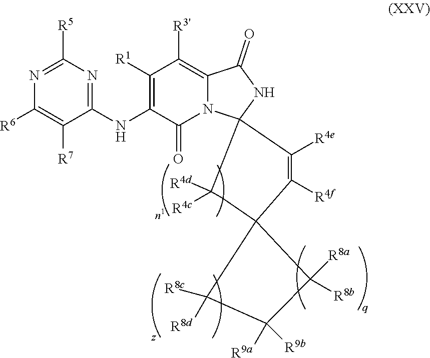

or a pharmaceutically acceptable salt thereof. R¹, R^{3′}, R^{4d}, R^{4c}, R^{4e}, R^{4f}, R⁵, R⁶, R⁷, R^{8a}, R^{8b}, R^{8c}, R^{8d}, R^{9a}, R^{9b}, n¹, q and z are as defined herein.

In more specific embodiments, the compounds of the present disclosure include compounds having formula (XXVI):

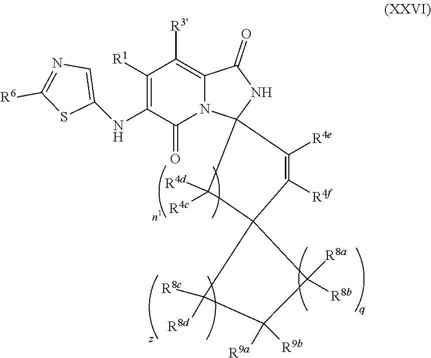

or a pharmaceutically acceptable salt thereof. R¹, R^{3′}, R^{4d}, R^{4c}, R^{4e}, R^{4f}, R⁶, R^{8a}, R^{8b}, R^{8c}, R^{8d}, R^{9a}, R^{9b}, n¹, q and z are as defined herein.

In more specific embodiments, the compounds of the present disclosure include compounds having formula (X)(VII):

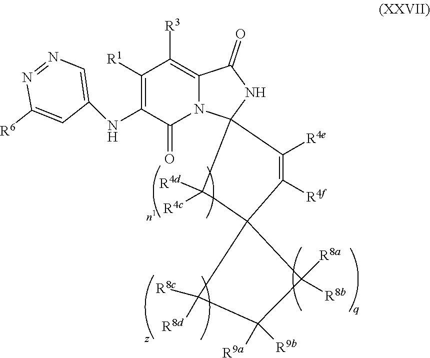

or a pharmaceutically acceptable salt thereof. R¹, R^{3′}, R^{4d}, R^{4c}, R^{4e}, R^{4f}, R⁶, R^{8a}, R^{8b}, R^{8c}, R^{8d}, R^{9a}, R^{9b}, n¹, q and z are as defined herein.

In more specific embodiments, the compounds of the present disclosure include compounds having formula (X(VIII):

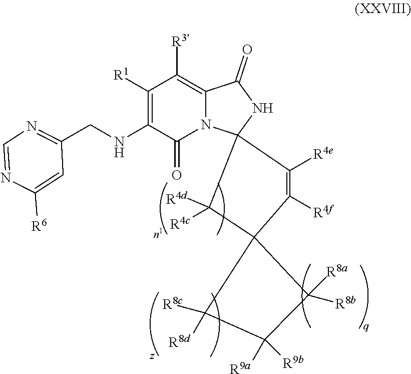

or a pharmaceutically acceptable salt thereof. R¹, R^{3′}, R^{4d}, R^{4c}, R^{4e}, R^{4f}, R⁶, R^{8a}, R^{8b}, R^{8c}, R^{8d}, R^{9a}, R^{9b}, n¹, q and z are as defined herein.

In more specific embodiments, the compounds of the present disclosure include compounds having formula (XXIX):

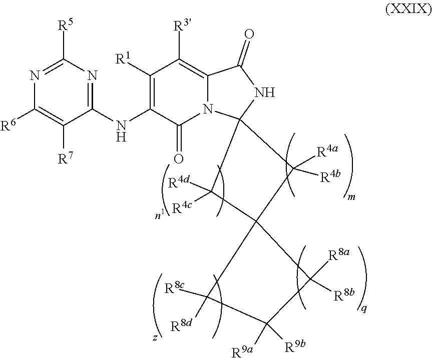

or a pharmaceutically acceptable salt thereof. R¹, R^{3′}, R^{4a}, R^{4b}, R^{4d}, R^{4c}, R⁵, R⁶, R⁷, R^{8a}, R^{8b}, R^{8c}, R^{8d}, R^{9a}, R^{9b}, m, n¹, q and z are as defined herein.

In more specific embodiments, the compounds of the present disclosure include compounds having formula (XXX):

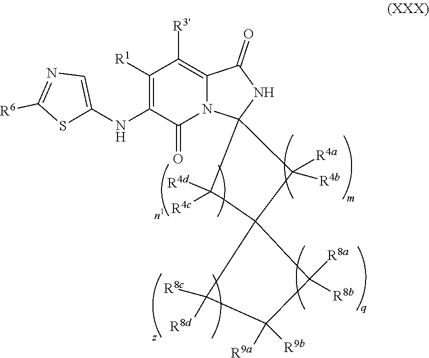

or a pharmaceutically acceptablesalt thereof. R¹, R^{3′}, R^{4a}, R^{4b}, R^{4d}, R^{4c}, R⁶, R^{8a}, R^{8b}, R^{8c}, R^{8d}, R^{9a}, R^{9b}, m, n¹, q and z are as defined herein.

In more specific embodiments, the compounds of the present disclosure include compounds having formula (XX(I):

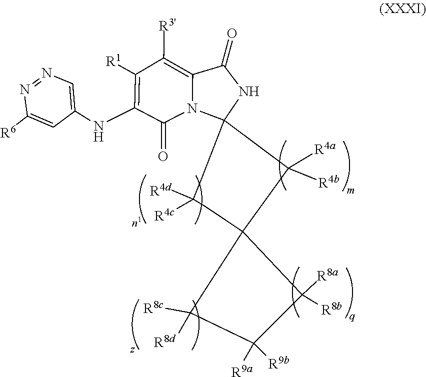

or a pharmaceutically acceptable salt thereof. R¹, R^{3′}, R^{4a}, R^{4b}, R^{4d}, R^{4c}, R⁶, R^{8a}, R^{8b}, R^{8c}, R^{8d}, R^{9a}, R^{9b}, m, n¹, q and z are as defined herein.

In more specific embodiments, the compounds of the present disclosure include compounds having formula (XXXII):

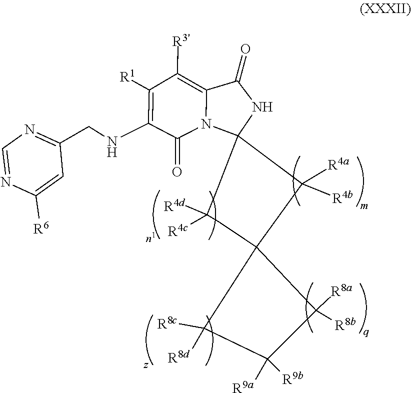

or a pharmaceutically acceptable salt thereof. R¹, R^{3′}, R^{4a}, R^{4b}, R^{4d}, R^{4c}, R⁶, R^{8a}, R^{8b}, R^{8c}, R^{8d}, R^{9a}, R^{9b}, m, n¹, q and z are as defined herein.

In more specific embodiments, the compounds of the present disclosure include compounds having formula (XXXIII):

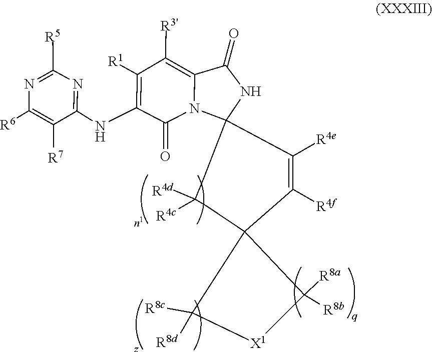

or a pharmaceutically acceptable salt thereof. R¹, R^{3′}, R^{4d}, R^{4c}, R^{4e}, R^{4f}, R⁵, R⁶, R⁷, R^{8a}, R^{8b}, R^{8c}, R^{8d}, X¹, n¹, q and z are as defined herein.

In more specific embodiments, the compounds of the present disclosure include compounds having formula (XXXIV):

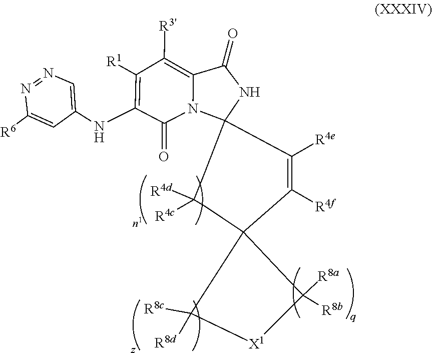

or a pharmaceutically acceptable salt thereof. R¹, R^{3′}, R^{4d}, R^{4c}, R^{4e}, R^{4f}, R⁶, R^{8a}, R^{8b}, R^{8c}, R^{8d}, X¹, n¹, q and z are as defined herein.

In more specific embodiments, the compounds of the present disclosure include compounds having formula (X)O(V):

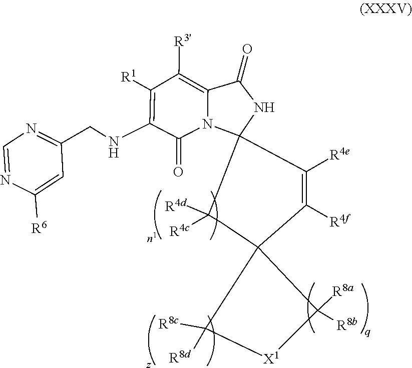

or a pharmaceutically acceptable salt thereof. R¹, R^{3′}, R^{4d}, R^{4c}, R^{4e}, R^{4f}, R⁶, R^{8a}, R^{8b}, R^{8c}, R^{8d}, X¹, n¹, q and z are as defined herein.

In more specific embodiments, the compounds of the present disclosure include compounds having formula (OO(VI):

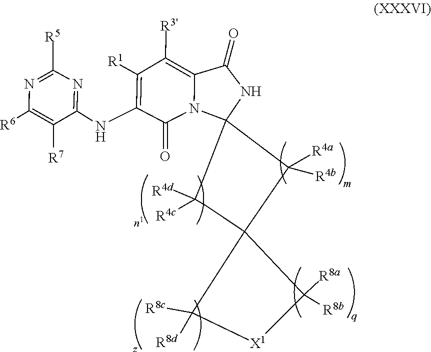

or a pharmaceutically acceptable salt thereof. R¹, R^{3′}, R^{4d}, R^{4c}, R^{4a}, R^{4b}, R⁵, R⁶, R⁷, R^{8a}, R^{8b}, R^{8c}, R^{8d}, X¹, m, n¹, q and z are as defined herein.

In more specific embodiments, the compounds of the present disclosure include compounds having formula (X)O(VII):

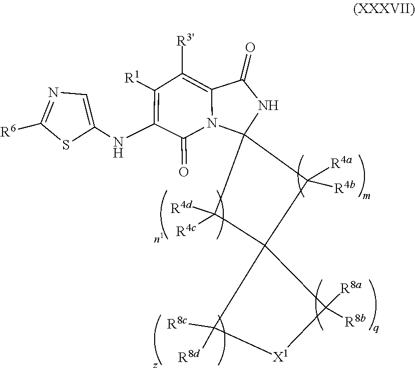

or a pharmaceutically acceptable salt thereof. R¹, R^{3′}, R^{4d}, R^{4c}, R^{4a}, R^{4b}, R⁶, R^{8a}, R^{8b}, R^{8c}, R^{8d}, X¹, m, n¹, q and z are as defined herein.

In more specific embodiments, the compounds of the present disclosure include compounds having formula (XX(VIII):

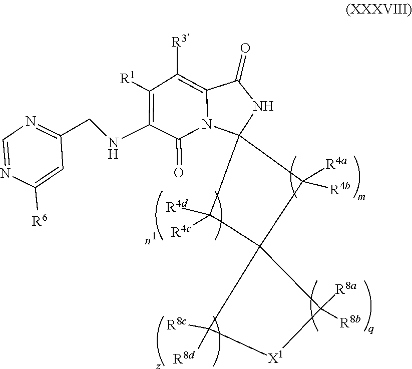

or a pharmaceutically acceptable salt thereof. R¹, R^{3′}, R^{4d}, R^{4c}, R^{4a}, R^{4b}, R⁶, R^{8a}, R^{8b}, R^{8c}, R^{8d}, X¹, m, n¹, q and z are as defined herein.

In more specific embodiments, the compounds of the present disclosure include compounds having formula (XX(VIIII) through (LI):

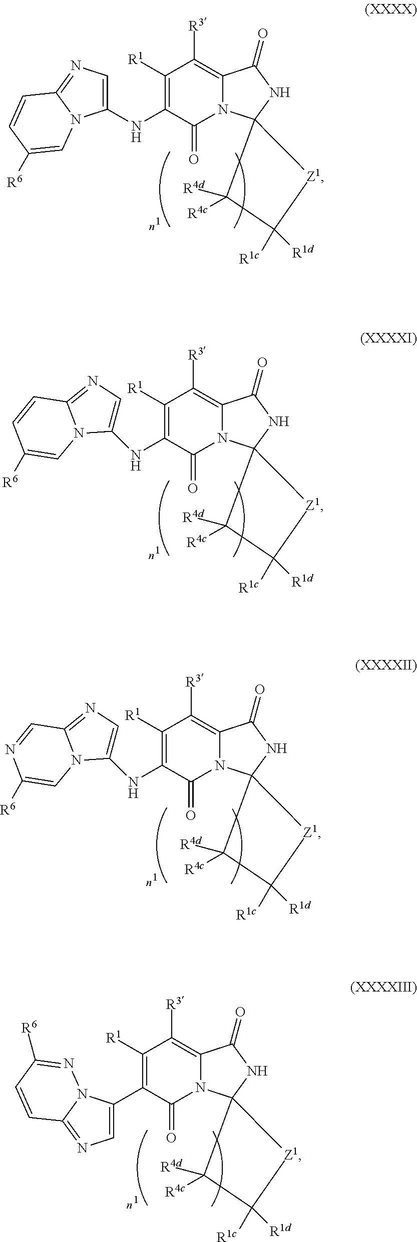

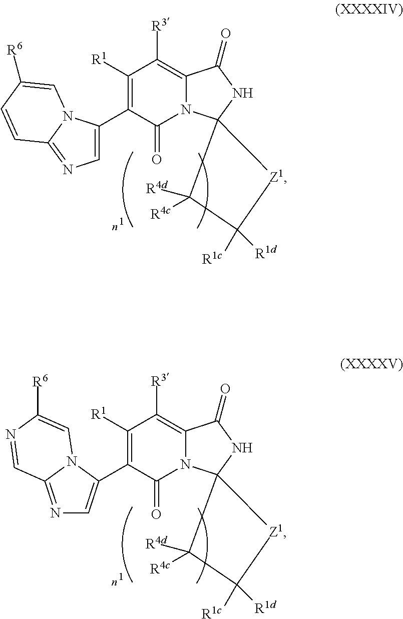

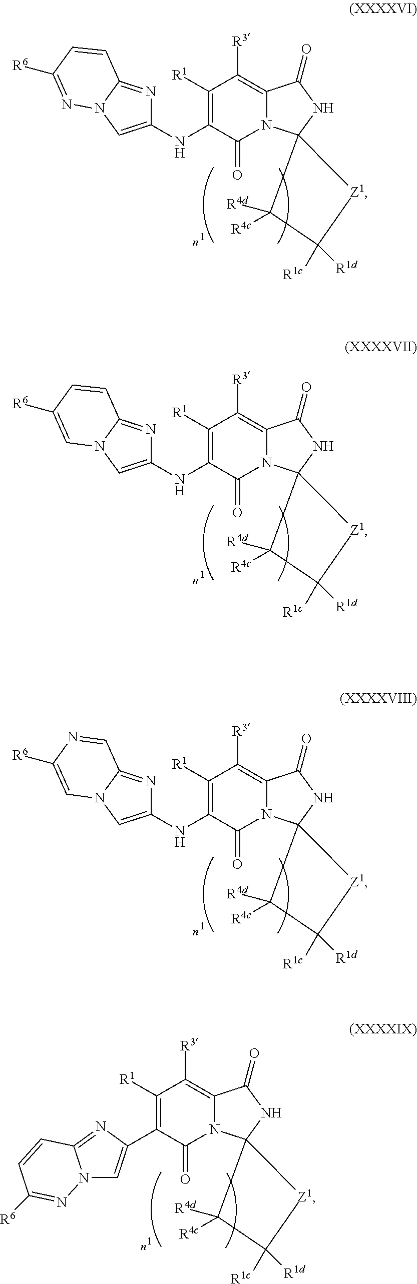

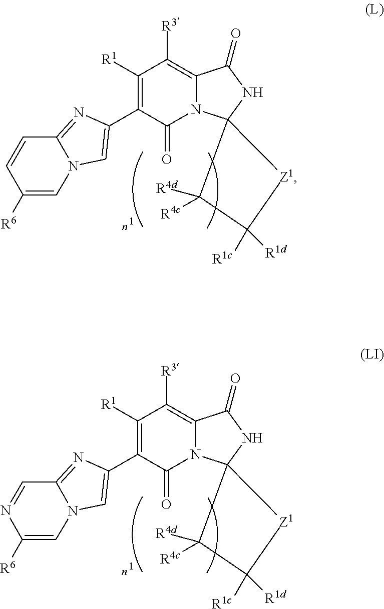

or a pharmaceutically acceptable salt thereof.

In some embodiments, Z¹ is

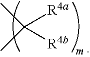

In some embodiments, Z¹ is

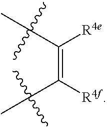

In some embodiments, R¹ is hydrogen. In some embodiments, R¹ is halogen. In some embodiments, R¹ is C₁₋₆ alkyl. In some embodiments, R1 is C₃₋₇ branched alkyl. In some embodiments, R1 is C₁₋₆ haloalkyl. In some embodiments, R1 is C₃₋₇ branched haloalkyl. In some embodiments, R1 is C₁₋₆ hydroxyalkyl. In some embodiments, R1 is C₃₋₇ branched hydroxyalkyl. In some embodiments, R1 is cyano. In some embodiments, R¹ is C₁₋₆ alkoxyl. In some embodiments, R¹ is C₃₋₇ branched alkoxy. In some embodiments, R1 is hydroxy. In some embodiments, R1 is C₃₋₆ cycloalkyl. In some embodiments, R¹ is C₃₋₆ cycloalkyl that is substituted 1 substituent selected from the groups consisting of halogen, C₁₋₆ alkyl, C₁₋₆ haloalkyl, and C₁₋₆ hydroxyalkyl. In some embodiments, R¹ is C₃₋₆ cycloalkyl that is substituted 2 substituents selected from the groups consisting of halogen, C₁₋₆ alkyl, C₁₋₆ haloalkyl, and C₁₋₆ hydroxyalkyl. In some embodiments, R1 is C₃₋₆ cycloalkyl that is substituted 3 substituents selected from the groups consisting of halogen, C₁₋₆ alkyl, C₁₋₆ haloalkyl, and C₁₋₆ hydroxyalkyl.

In some embodiments, R^{2′} is

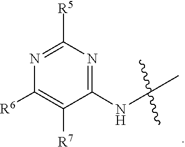

In some embodiments, R^{2′} is

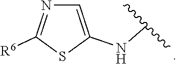

In some embodiments, R^{2′} is

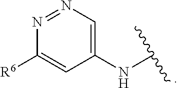

In some embodiments, R^{2′} is

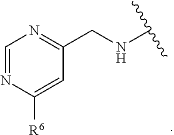

In some embodiments, R^{2′} is

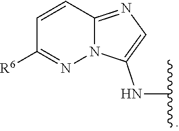

In some embodiments, R^{2′} is

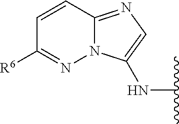

In some embodiments, R^{2′} is

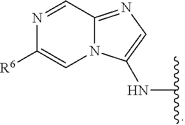

In some embodiments, R^{2′} is

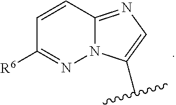

In some embodiments, R^{2′} is

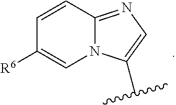

In some embodiments, R^{2′} is

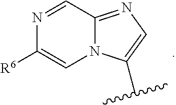

In some embodiments, R^{2′} is

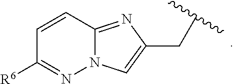

In some embodiments, R^{2′} is

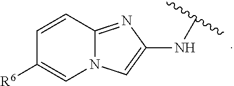

In some embodiments, R^{2′} is

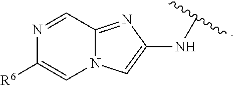

In some embodiments, R^{2′} is

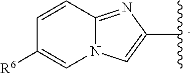

In some embodiments, R^{2′} is

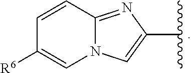

In some embodiments, R^{2′} is

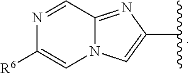

In some embodiments, R^{3′} is hydrogen. In some embodiments, R^{3′} is halogen. In some embodiments, R^{3′} is C₁₋₆ alkyl. In some embodiments, R^{3′} is C₃₋₇ branched alkyl. In some embodiments, R^{3′} is C₁₋₆ haloalkyl. In some embodiments, R^{3′} is C₃₋₇ branched haloalkyl. In some embodiments, R^{3′} is C₁₋₆ hydroxyalkyl. In some embodiments, R^{3′} is C₃₋₇ branched hydroxyalkyl. In some embodiments, R^{3′} is cyano. In some embodiments, R^{3′} is C₁₋₆ alkoxyl. In some embodiments, R^{3′} is C₃₋₇ branched alkoxy. In some embodiments, R^{3′} is hydroxy. In some embodiments, R^{3′} is C₃₋₆ cycloalkyl. In some embodiments, R^{3′} is C₃₋₆ cycloakyl that is substituted with 1 substituent selected from the groups consisting of halogen, C₁₋₆ alkyl, C₁₋₆ haloalkyl, and C₁₋₆ hydroxyalkyl. In some embodiments, R^{3′} is C₃₋₆ cycloakyl that is substituted with 2 substituent selected from the groups consisting of halogen, C₁₋₆ alkyl, C₁₋₆ haloalkyl, and C₁₋₆ hydroxyalkyl. In some embodiments, R^{3′} is C₃₋₆ cycloakyl that is substituted with 3 substituent selected from the groups consisting of halogen, C₁₋₆ alkyl, C₁₋₆ haloalkyl, and C₁₋₆ hydroxyalkyl.

In some embodiments, R^{4a} is hydrogen. In some embodiments, R^{4a} is halogen. In some embodiments, R^{4a} is C₁₋₆ alkyl. In some embodiments, R^{4a} is C₃₋₇ branched alkyl. In some embodiments, R^{4a} is C₁₋₆ haloalkyl. In some embodiments, R^{4a} is C₃₋₇ branched haloalkyl. In some embodiments, R^{4a} is hydroxy. In some embodiments, R^{4a} is C₁₋₆ alkoxyl. In some embodiments, R^{4a} is C₃₋₇ branched alkoxy. In some embodiments, R⁴⁸ is NHCO(C₁₋₆alkyl). In some embodiments, R^{4a} is NHCO(C₃₋₇ branched alkyl). In some embodiments, R^{4a} is NHCO(C₃₋₇ cycloalkyl). In some embodiments, R^{4a} is NHSO₂(C₁₋₆alkyl). In some embodiments, R^{4a} is NHSO₂(C₃₋₇ branched alkyl). In some embodiments, R^{4a} is NHSO₂(C₃₋₇ cycloalkyl).

In some embodiments, R^{4b} is hydrogen. In some embodiments, R^{4b} is halogen. In some embodiments, R^{4b} is C₁₋₆ alkyl. In some embodiments, R^{4b} is C₃₋₇ branched alkyl. In some embodiments, R^{4b} is C₁₋₆ haloalkyl. In some embodiments, R^{4b} is C₃₋₇ branched haloalkyl. In some embodiments, R^{4b} is hydroxy. In some embodiments, R^{4b} is C₁₋₆ alkoxyl. In some embodiments, R^{4b} is C₃₋₇ branched alkoxy. In some embodiments, R^{4b} is NHCO(C₁₋₆alkyl). In some embodiments, R^{4b} is NHCO(C₃₋₇ branched alkyl). In some embodiments, R^{4b} is NHCO(C₃₋₇ cycloalkyl). In some embodiments, R^{4b} is NHSO₂(C₁₋₆alkyl). In some embodiments, R^{4b} is NHSO₂(C₃₋₇ branched alkyl). In some embodiments, Rob is NHSO₂(C₃₋₇ cycloalkyl).

In some embodiments, Roc is hydrogen. In some embodiments, Roc is halogen. In some embodiments, Roc is C₁₋₆ alkyl. In some embodiments, R^{4C} is C₃₋₇ branched alkyl. In some embodiments, R^{4c} is C₁₋₆ haloalkyl. In some embodiments, Roc is C₃₋₇ branched haloalkyl. In some embodiments, Roc is hydroxy. In some embodiments, Roc is C₁₋₆ alkoxyl. In some embodiments, Roc is C₃₋₇ branched alkoxy. In some embodiments, Roo is NHCO(C₁₋₆alkyl). In some embodiments, Roc is NHCO(C₃₋₇ branched alkyl). In some embodiments, Roc is NHCO(C₃₋₇ cycloalkyl). In some embodiments, Roc is NHSO₂(C₁₋₆alkyl). In some embodiments, Roc is NHSO₂(C₃₋₇ branched alkyl). In some embodiments, Roc is NHSO₂(C₃₋₇ cycloalkyl).

In some embodiments, R^{4d} is hydrogen. In some embodiments, R^{4d} is halogen. In some embodiments, Rod is C₁₋₆ alkyl. In some embodiments, R^{4d} is C₃₋₇ branched alkyl. In some embodiments, R^{4d} is C₁₋₆ haloalkyl. In some embodiments, Rod is C₃₋₇ branched haloalkyl. In some embodiments, Rod is hydroxy. In some embodiments, R^{4d} is C₁₋₆ alkoxyl. In some embodiments, R^{4d} is C₃₋₇ branched alkoxy. In some embodiments, Rod is NHCO(C₁₋₆alkyl). In some embodiments, R^{4d} is NHCO(C₃₋₇ branched alkyl). In some embodiments, Rod is NHCO(C₃₋₇ cycloalkyl). In some embodiments, Rod is NHSO₂(C₁₋₆alkyl). In some embodiments, R^{4d} is NHSO₂(C₃₋₇ branched alkyl). In some embodiments, Rod is NHSO₂(C₃₋₇ cycloalkyl).

In some embodiments, R^{4e} is hydrogen. In some embodiments, R^{4e} is halogen. In some embodiments, R^{4e} is C₁₋₆ alkyl. In some embodiments, R^{4e} is C₃₋₇ branched alkyl. In some embodiments, R^{4e} is C₁₋₆ haloalkyl. In some embodiments, R^{4e} is C₃₋₇ branched haloalkyl.

In some embodiments, R^{4f} is hydrogen. In some embodiments, R^{4f} is halogen. In some embodiments, R^{4f} is C₁₋₆ alkyl. In some embodiments, R^{4f} is C₃₋₇ branched alkyl. In some embodiments, R^{4f} is C₁₋₆ haloalkyl. In some embodiments, R^{4f} is C₃₋₇ branched haloalkyl.

In some embodiments, R^{1c} and R^{1d} are taken together to form an optionally substituted 3 membered ring. In some embodiments, R^{1c} and R^{1d} are taken together to form an optionally substituted 4 membered ring. In some embodiments, R^{1c} and R^{1d} are taken together to form an optionally substituted 5 membered ring. In some embodiments, R^{1c} and R^{1d} are taken together to form an optionally substituted 6 membered ring. In some embodiments, R^{1c} and R^{1d} are taken together to form an optionally substituted 7 membered ring. In some embodiments, R^{1c} and R^{1d} are taken together to form an optionally substituted 3 membered ring that contains an X¹ group. In some embodiments, R^{1c} and R^{1d} are taken together to form an optionally substituted 4 membered ring that contains an X¹ group. In some embodiments, R^{1c} and R^{1d} are taken together to form an optionally substituted 5 membered ring that contains an X¹ group. In some embodiments, R^{1c} and R^{1d} are taken together to form an optionally substituted 6 membered ring that contains an X¹ group. In some embodiments, R^{1c} and R^{1d} are taken together to form an optionally substituted 7 membered ring that contains an X¹ group.

In some embodiments, X¹ is CF₂. In some embodiments, X¹ is CHCO₂R¹². In some embodiments, X¹ is O. In some embodiments, X¹ is NH. In some embodiments, X¹ is NR⁸. In some embodiments, X¹ is SO₂.

In some embodiments, m is 0. In some embodiments, m is 1. In some embodiments, m is 2.

In some embodiments, n¹ is 1. In some embodiments, n¹ is 2. In some embodiments, n¹ is 3.

In some embodiments, R⁵ is hydrogen. In some embodiments, R⁵ is halogen. In some embodiments, R⁵ is C₁₋₆ alkyl. In some embodiments, R⁵ is C₃₋₇ branched alkyl. In some embodiments, R⁵ is C₁₋₆ haloalkyl. In some embodiments, R⁵ is C₃₋₇ branched haloalkyl. In some embodiments, R⁵ is C₁₋₆ alkoxyl. In some embodiments, R⁵ is C₃₋₇ branched alkoxy. In some embodiments, R⁵ is hydroxy.

In some embodiments, R⁶ is hydrogen. In some embodiments, R⁶ is NH₂. In some embodiments, R⁶ is NHR⁶a. In some embodiments, R⁶ is NHCH₂CH₂OH. In some embodiments, R⁶ is NHCH₂CH₂NHSO₂Me. In some embodiments, R⁶ is C₁₋₆ alkoxyl. In some embodiments, R⁶ is C₃₋₇ branched alkoxy. In some embodiments, R⁶ is hydroxy.

In some embodiments, R^{6a} is —(CO)C₁₋₆ alkyl. In some embodiments, R^{6a} is —(CO)C₃₋₇ branched alkyl. In some embodiments, R^{6a} is —(CO)C₁₋₆ hydroxyalkyl. In some embodiments, R^{6a} is

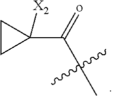

In some embodiments, R^{6a} is

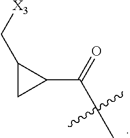

some embodiments, R^{6a} is

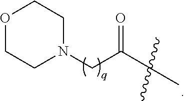

In some embodiments, R^{6a} is

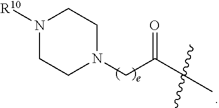

In some embodiments, R^{6a} is

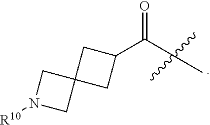

In some embodiments, R^{6a} is

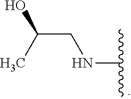 [SMILES: *NC[C@@H](C)O]

In some embodiments, R^{6a} is

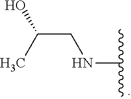 [SMILES: *NC[C@H](C)O]

In some embodiments, R^{6a} is

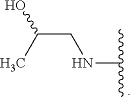 [SMILES: *NCC(C)O]

In some embodiments, R^{6a} is

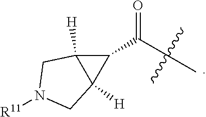

In some embodiments, R^{6a} is

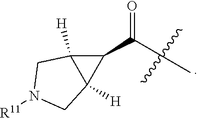

In some embodiments, q is 1. In some embodiments, q is 2. In some embodiments, q is 3. In some embodiments, q is 4. In some embodiments, q is 5. In some embodiments, q is 6.

In some embodiments, e is 1. In some embodiments, e is 2. In some embodiments, e is 3. In some embodiments, e is 4. In some embodiments, e is 5. In some embodiments, e is 6.

In some embodiments, X² is hydrogen. In some embodiments, X² is halogen. In some embodiments, X² is C₁₋₆alkyl. In some embodiments, X² is C₃₋₇ branched alkyl. In some embodiments, X² is C₁-6haloalkyl. In some embodiments, X² is C₃₋₇ branched haloalkyl. In some embodiments, X² is hydroxy. In some embodiments, X² is C₁-6hydroxyalkyl. In some embodiments, X² is C₃₋₇ branched hydroxyalkyl. In some embodiments, X² is C₁₋₆alkoxy. In some embodiments, X² is C₃₋₇ branched alkoxy. In some embodiments, X² is C₁-6haloalkoxy. In some embodiments, X² is C₃₋₇ branched haloalkoxy. In some embodiments, X² is NH₂. In some embodiments, X² is NH(C₁₋₆alkyl). In some embodiments, X² is N(C₁₋₆alkyl)₂. In some embodiments, X² is C₁₋₅(COOH). In some embodiments, X² is C₁₋₆(NHSO₂Me).

In some embodiments, X³ is hydrogen. In some embodiments, X³ is halogen. In some embodiments, X³ is C₁₋₆ alkyl. In some embodiments, X³ is C₃₋₇ branched alkyl. In some embodiments, X³ is C₁₋₆ haloalkyl. In some embodiments, X³ is C₃₋₇ branched haloalkyl. In some embodiments, X³ is hydroxy. In some embodiments, X³ is C₁₋₆ hydroxyalkyl. In some embodiments, X³ is C₃₋₇ branched hydroxyalkyl. In some embodiments, X³ is C₁₋₆ alkoxy. In some embodiments, X³ is C₃₋₇ branched alkoxy. In some embodiments, X³ is C₁₋₅ haloalkoxy. In some embodiments, X³ is C₃₋₇ branched haloalkoxy. In some embodiments, X³ is NH₂. In some embodiments, X³ is NH(C₁₋₆ alkyl). In some embodiments, X³ is N(C₁₋₆ alkyl)₂. In some embodiments, X³ is COOH. In some embodiments, X³ is C₁₋₅(COOH). In some embodiments, X³ is NHSO₂Me. In some embodiments, X³ is C₁₋₅(NHSO₂Me).

In some embodiments, R⁷ is hydrogen. In some embodiments, R⁷ is halogen. In some embodiments, R⁷ is C₁₋₆ alkyl. In some embodiments, R⁷ is C₃₋₇ branched alkyl. In some embodiments, R⁷ is C₁₋₆ haloalkyl. In some embodiments, R⁷ is C₃₋₇ branched haloalkyl. In some embodiments, R⁷ is C₁₋₆ alkoxyl. In some embodiments, R⁷ is C₃₋₇ branched alkoxy. In some embodiments, R⁷ is hydroxy.

In some embodiments, R⁸ is C₁₋₆ alkyl. In some embodiments, R⁸ is C₁₋₆ haloalkyl. In some embodiments, R⁸ is C₃₋₇ branched haloalkyl. In some embodiments, R⁸ is C₁₋₆ hydroxyalkyl. In some embodiments, R⁸ is C₃₋₇ branched hydroxyalkyl. In some embodiments, R⁸ is C₁₋₆ alkoxyl. In some embodiments, R⁸ is C₃₋₇ branched alkoxy. In some embodiments, R⁸ is CO(C₁₋₆alkyl). In some embodiments, R⁸ is CO(C₃₋₇ branched alkyl). In some embodiments, R⁸ is SO₂(C₁₋₆alkyl). In some embodiments, R⁸ is SO₂(C₃₋₇ branched alkyl).

In some embodiments, R⁸ is hydrogen. In some embodiments, R^{8a} is halogen. In some embodiments, R⁸ is C₁₋₆ alkyl. In some embodiments, R⁸ is C₃₋₇ branched alkyl. In some embodiments, R⁸ is C₁₋₆ haloalkyl. In some embodiments, R⁸ is C₃₋₇ branched haloalkyl. In some embodiments, R^{8a} is C₁₋₆ hydroxyalkyl. In some embodiments, R⁸ is C₃₋₇ branched hydroxyalkyl. In some embodiments, R^{8a} is hydroxy. In some embodiments, R^{8a} is C₁₋₆ alkoxyl. In some embodiments, R⁸ is C₃₋₇ branched alkoxy. In some embodiments, R⁸ is NHCO(C₁₋₆alkyl). In some embodiments, R⁸ is NHCO(C₃₋₇ branched alkyl). In some embodiments, R⁸ is NHSO₂(C₁₋₆alkyl). In some embodiments, R⁸ is NHSO₂(C₃₋₇ branched alkyl).

In some embodiments, R^{8b} is hydrogen. In some embodiments, R^{8b} is halogen. In some embodiments, R^{8b} is C₁₋₆ alkyl. In some embodiments, R^{8b} is C₃₋₇ branched alkyl. In some embodiments, R^{8b} is C₁₋₆ haloalkyl. In some embodiments, R^{8b} is C₃₋₇ branched haloalkyl. In some embodiments, R^{8b} is C₁₋₆ hydroxyalkyl. In some embodiments, R^{8b} is C₃₋₇ branched hydroxyalkyl. In some embodiments, R^{8b} is hydroxy. In some embodiments, R^{8b} is C₁₋₆ alkoxyl. In some embodiments, R^{8b} is C₃₋₇ branched alkoxy. In some embodiments, R^{8b} is NHCO(C₁₋₆alkyl). In some embodiments, R^{8b} is NHCO(C₃₋₇ branched alkyl). In some embodiments, R^{8b} is NHSO₂(C₁₋₆alkyl). In some embodiments, R^{8′} is NHSO₂(C₃₋₇ branched alkyl).

In some embodiments, R^{8c} is hydrogen. In some embodiments, R^{8c} is halogen. In some embodiments, R^{8c} is C₁₋₆ alkyl. In some embodiments, R^{8c} is C₃₋₇ branched alkyl. In some embodiments, R^{8c} is C₁₋₆ haloalkyl. In some embodiments, R^{8c} is C₃₋₇ branched haloalkyl. In some embodiments, R^{8c} is C₁₋₆ hydroxyalkyl. In some embodiments, R^{8c} is C₃₋₇ branched hydroxyalkyl. In some embodiments, R^{8c} is hydroxy.

In some embodiments, R^{8c} is C₁₋₆ alkoxyl. In some embodiments, R^{8c} is C₃₋₇ branched alkoxy. In some embodiments, R^{8c} is NHCO(C₁₋₆alkyl). In some embodiments, R^{8c} is NHCO(C₃₋₇ branched alkyl). In some embodiments, R^{8c} is NHSO₂(C₁₋₆alkyl). In some embodiments, R^{8c} is NHSO₂(C₃₋₇ branched alkyl).

In some embodiments, R^{8d} is hydrogen. In some embodiments, R^{8d} is halogen. In some embodiments, R⁸¹ is C₁₋₆ alkyl. In some embodiments, R^{8d} is C₃₋₇ branched alkyl, In some embodiments, R^{8d} is C₁₋₆ haloalkyl. In some embodiments, R⁸¹ is C₃₋₇ branched haloalkyl. In some embodiments, R^{8d} is C₁₋₆ hydroxyalkyl. In some embodiments, R^{8d} is C₃₋₇ branched hydroxyalkyl. In some embodiments, R^{8d} is hydroxy. In some embodiments, R^{8d} is C₁₋₆ alkoxyl. In some embodiments, R^{8d} is C₃₋₇ branched alkoxy. In some embodiments, R^{8d} is NHCO(C₁₋₆alkyl). In some embodiments, R^{8d} is NHCO(C₃₋₇ branched alkyl). In some embodiments, R^{8d} is NHSO₂(C₁₋₆alkyl). In some embodiments, R^{8d} is NHSO₂(C₃₋₇ branched alkyl).

In some embodiments, R^{9a} is hydrogen. In some embodiments, R^{9a} is halogen. In some embodiments, R^{9a} is C₁₋₆ alkyl. In some embodiments, R^{9a} is C₃₋₇ branched alkyl. In some embodiments, R^{9a} is C₁₋₆ haloalkyl. In some embodiments, R^{9a} is C₃₋₇ branched haloalkyl. In some embodiments, R^{9a} is C₁₋₆ hydroxyalkyl. In some embodiments, R^{9a} is C₃₋₇ branched hydroxyalkyl. In some embodiments, R^{9a} is hydroxy. In some embodiments, R^{9a} is C₁₋₆ alkoxyl. In some embodiments, R^{9a} is C₃₋₇ branched alkoxy.

In some embodiments, R^{9b} is hydrogen. In some embodiments, R^{9b} is halogen. In some embodiments, R^{9b} is C₁₋₆ alkyl. In some embodiments, R^{9b} is C₃₋₇ branched alkyl. In some embodiments, R^{9b} is C₁₋₆ haloalkyl. In some embodiments, R^{9b} is C₃₋₇ branched haloalkyl. In some embodiments, R^{9b} is C₁₋₆ hydroxyalkyl. In some embodiments, R^{9b} is C₃₋₇ branched hydroxyalkyl. In some embodiments, R^{9b} is hydroxy. In some embodiments, R^{9b} is C₁₋₆ alkoxyl. In some embodiments, R^{9b} is C₃₋₇ branched alkoxy.

In some embodiments, R^{9a} and R^{9b} are taken together to form a 3 membered ring. In some embodiments, R^{9a} and R^{9b} are taken together to form a 4 membered ring. In some embodiments, R^{9a} and R^{9b} are taken together to form a 5 membered ring. In some embodiments, R^{9a} and R^{9b} are taken together to form a 6 membered ring. In some embodiments, R^{9a} and R^{9b} are taken together to form a 7 membered ring. In some embodiments, R^{9a} and R^{9b} are taken together to form an optionally substituted 3 membered ring. In some embodiments, R^{9a} and R^{9b} are taken together to form an optionally substituted 4 membered ring. In some embodiments, R^{9a} and R^{9b} are taken together to form an optionally substituted 5 membered ring. In some embodiments, R^{9a} and R^{9b} are taken together to form an optionally substituted 6 membered ring. In some embodiments, R^{9a} and R^{9b} are taken together to form an optionally substituted 7 membered ring.

In some embodiments, q is 1. In some embodiments, q is 2. In some embodiments, q is 3.

In some embodiments, z is 0. In some embodiments, z is 1. In some embodiments, z is 2.

In some embodiments, R¹⁰ is hydrogen. In some embodiments, R¹⁰ is C₁₋₆ alkyl. In some embodiments, R¹⁰ is C₁₋₆ haloalkyl. In some embodiments, R¹⁰ is C₃₋₇ branched haloalkyl. In some embodiments, R¹⁰ is C₁₋₆ hydroxyalkyl. In some embodiments, R¹⁰ is C₁₋₆ alkoxyl. In some embodiments, R¹⁰ is C₃₋₇ branched alkoxy. In some embodiments, R¹⁰ is CO(C₁₋₆alkyl). In some embodiments, R¹⁰ is CO(C₁₋₇ branched alkyl). In some embodiments, R¹⁰ is SO₂(C₁₋₆alkyl). In some embodiments, R¹⁰ is SO₂(C₃₋₇ branched alkyl).

In some embodiments, R¹¹ is hydrogen. In some embodiments, R¹¹ is C₁₋₆ alkyl.

In some embodiments, R¹² is hydrogen. In some embodiments, R¹² is C₁₋₆ alkyl.

In some embodiments the compounds of Formula (IA), (I′) or substructures exclude N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropanecarboxamide; and/or 3-((6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)amino)propanoic acid.

MNK inhibitors of the present disclosure include compounds having the formula (LII) or a pharmaceutically acceptable salt form thereof:

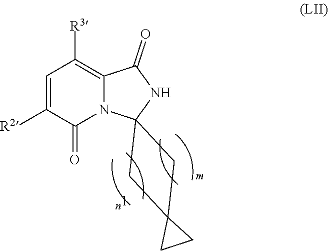

Wherein m, n¹, R^{3′} and R² are as defined herein. PGP-326,01

Examples of R^{2′}, R^{3′}, m and n¹, without limitation, are set forth in Table 2.

TABLE 2

| Entry | R^{2′} | R^{3′} | n¹ | m |
|---|---|---|---|---|
| 1 | 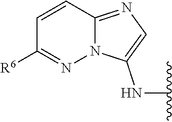 | CH₃ | 1 | 1 |
| 2 | 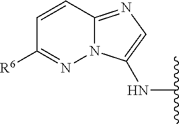 | CH₃ | 2 | 1 |
| 3 | 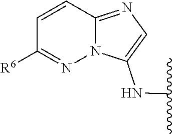 | CH₃ | 3 | 1 |
| 4 | 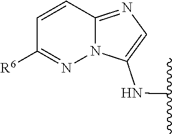 | CH₃ | 2 | 2 |
| 5 | 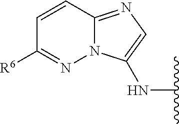 | CH₃ | 3 | 2 |
| 6 | 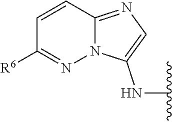 | Cl | 4 | 1 |
| 7 | 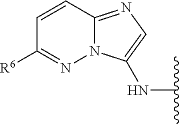 | Cl | 1 | 1 |
| 8 | 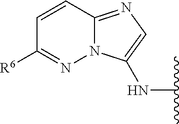 | Cl | 2 | 1 |
| 9 | 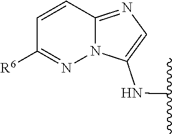 | Cl | 3 | 1 |
| 10 | 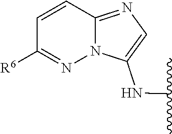 | Cl | 2 | 2 |
| 11 | 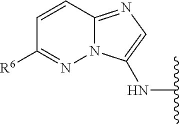 | Cl | 3 | 2 |
| 12 | 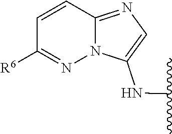 | CH₃ | 4 | 1 |
| 13 | 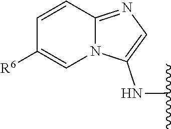 | CH₃ | 1 | 1 |
| 14 | 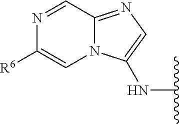 | CH₃ | 2 | 1 |
| 15 | 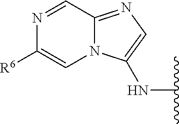 | CH₃ | 3 | 1 |
| 16 | 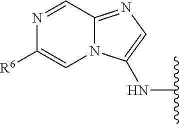 | CH₃ | 2 | 2 |
| 17 | 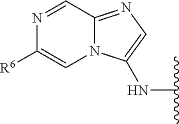 | CH₃ | 3 | 2 |
| 18 | 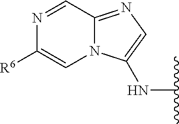 | CH₃ | 4 | 1 |
| 19 | 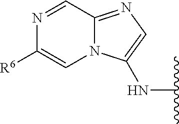 | Cl | 1 | 1 |
| 20 | 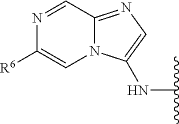 | Cl | 2 | 1 |
| 21 | 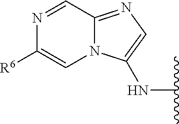 | Cl | 3 | 1 |
| 22 | 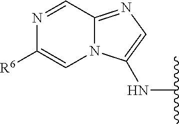 | Cl | 2 | 2 |
| 23 | 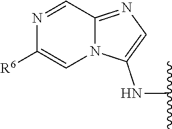 | Cl | 3 | 2 |
| 24 | 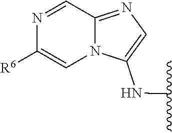 | Cl | 4 | 1 |
| 25 | 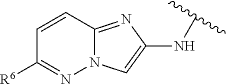 | CH₃ | 1 | 1 |
| 26 | 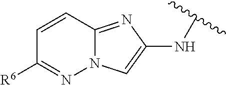 | CH₃ | 2 | 1 |
| 27 | 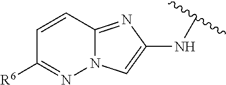 | CH₃ | 3 | 1 |
| 28 | 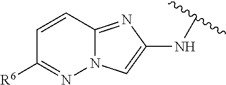 | CH₃ | 2 | 2 |
| 29 | 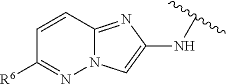 | CH₃ | 3 | 2 |
| 30 | 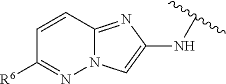 | CH₃ | 4 | 1 |
| 31 | 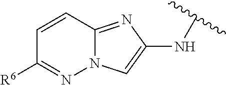 | Cl | 1 | 1 |
| 32 | 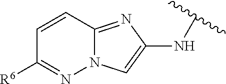 | Cl | 2 | 1 |
| 33 | 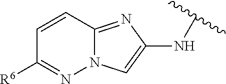 | Cl | 3 | 1 |
| 34 | 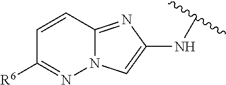 | Cl | 2 | 2 |
| 35 | 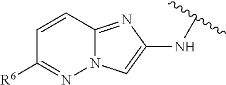 | Cl | 3 | 2 |
| 36 | 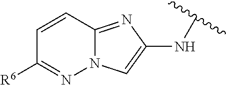 | Cl | 4 | 1 |
| 37 | 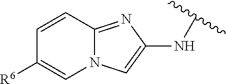 | CH₃ | 1 | 1 |
| 38 | 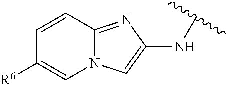 | CH₃ | 2 | 1 |
| 39 | 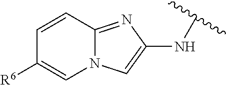 | CH₃ | 3 | 1 |
| 40 | 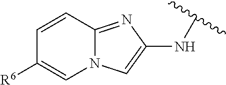 | CH₃ | 2 | 2 |
| 41 | 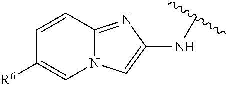 | CH₃ | 3 | 2 |
| 42 | 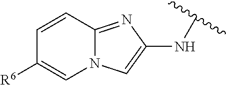 | CH₃ | 4 | 1 |
| 43 | 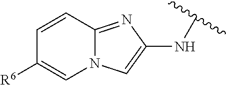 | Cl | 1 | 1 |
| 44 | 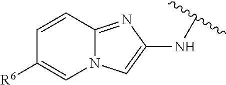 | Cl | 2 | 1 |
| 45 | 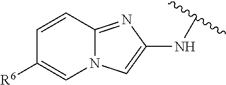 | Cl | 3 | 1 |
| 46 | 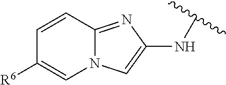 | Cl | 2 | 2 |
| 47 | 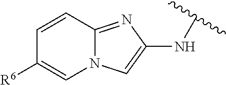 | Cl | 3 | 2 |
| 48 | 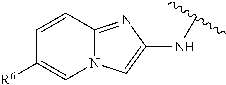 | Cl | 4 | 1 |
| 49 | 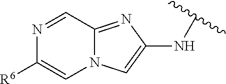 | CH₃ | 1 | 1 |
| 50 | 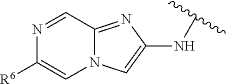 | CH₃ | 2 | 1 |
| 51 | 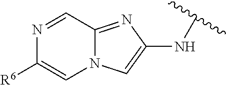 | CH₃ | 3 | 1 |
| 52 | 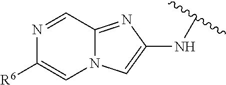 | CH₃ | 2 | 2 |
| 53 | 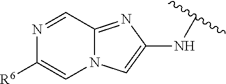 | CH₃ | 3 | 2 |
| 54 | 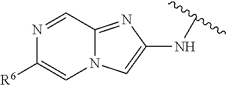 | CH₃ | 4 | 1 |
| 55 | 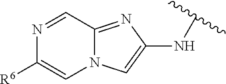 | Cl | 1 | 1 |
| 56 | 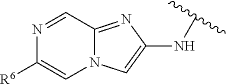 | Cl | 2 | 1 |
| 57 | 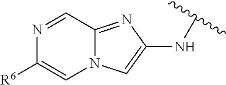 | Cl | 3 | 1 |
| 58 | 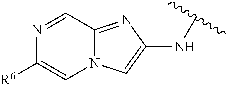 | Cl | 2 | 2 |
| 59 | 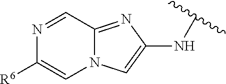 | Cl | 3 | 2 |
| 60 | 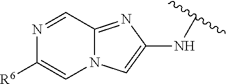 | Cl | 4 | 1 |
| 61 | 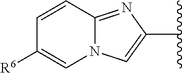 | CH₃ | 1 | 1 |
| 62 | 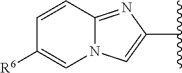 | CH₃ | 2 | 1 |
| 63 | 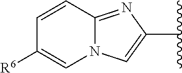 | CH₃ | 3 | 1 |
| 64 | 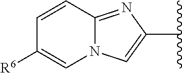 | CH₃ | 2 | 2 |
| 65 | 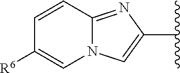 | CH₃ | 3 | 2 |
| 66 | 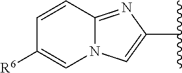 | CH₃ | 4 | 1 |
| 67 | 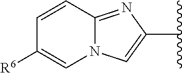 | Cl | 1 | 1 |
| 68 | 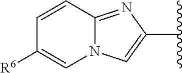 | Cl | 2 | 1 |
| 69 | 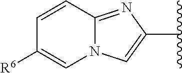 | Cl | 3 | 1 |
| 70 | 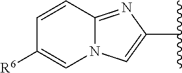 | Cl | 2 | 2 |
| 71 | 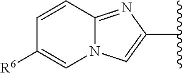 | Cl | 3 | 2 |
| 72 | 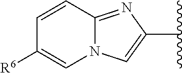 | Cl | 4 | 1 |
| 73 | 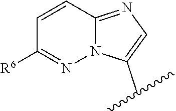 | CH₃ | 1 | 1 |
| 74 | 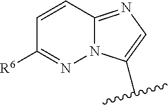 | CH₃ | 2 | 1 |
| 75 | 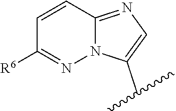 | CH₃ | 3 | 1 |
| 76 | 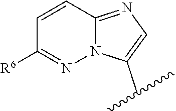 | CH₃ | 2 | 2 |
| 77 | 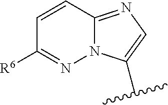 | CH₃ | 3 | 2 |
| 78 | 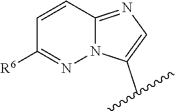 | CH₃ | 4 | 1 |
| 79 | 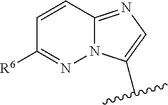 | Cl | 1 | 1 |
| 80 | 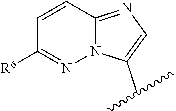 | Cl | 2 | 1 |
| 81 | 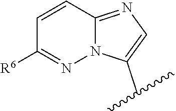 | Cl | 3 | 1 |
| 82 | 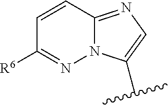 | Cl | 2 | 2 |
| 83 | 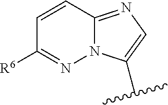 | Cl | 3 | 2 |
| 84 | 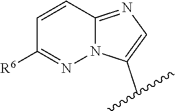 | Cl | 4 | 1 |
| 85 | 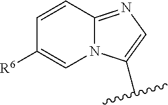 | CH₃ | 1 | 1 |
| 86 | 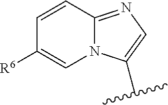 | CH₃ | 2 | 1 |
| 87 | 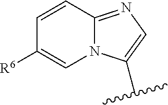 | CH₃ | 3 | 1 |
| 88 | 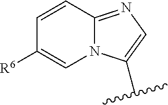 | CH₃ | 2 | 2 |
| 89 | 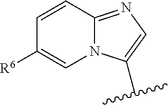 | CH₃ | 3 | 2 |
| 90 | 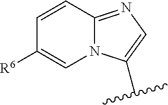 | CH₃ | 4 | 1 |
| 91 | 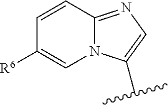 | Cl | 1 | 1 |
| 92 | 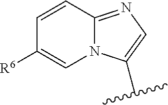 | Cl | 2 | 1 |
| 93 | 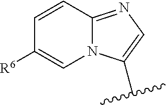 | Cl | 3 | 1 |
| 94 | 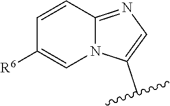 | Cl | 2 | 2 |
| 95 | 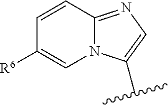 | Cl | 3 | 2 |
| 96 | 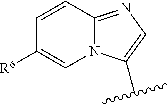 | Cl | 4 | 1 |
| 97 | 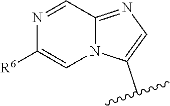 | CH₃ | 1 | 1 |
| 98 | 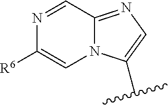 | CH₃ | 2 | 1 |
| 99 | 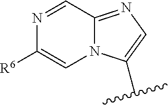 | CH₃ | 3 | 1 |
| 100 | 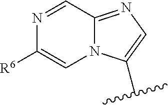 | CH₃ | 2 | 2 |
| 101 | 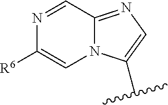 | CH₃ | 3 | 2 |
| 102 | 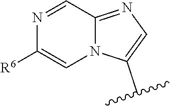 | CH₃ | 4 | 1 |
| 103 | 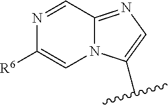 | Cl | 1 | 1 |
| 104 | 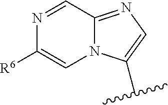 | Cl | 2 | 1 |
| 105 | 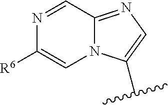 | Cl | 3 | 1 |
| 106 | 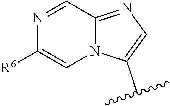 | Cl | 2 | 2 |
| 107 | 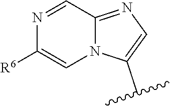 | Cl | 3 | 2 |
| 108 | 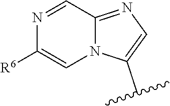 | Cl | 4 | 1 |
| 109 | 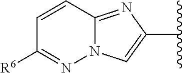 | CH₃ | 1 | 1 |
| 110 | 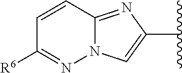 | CH₃ | 2 | 1 |
| 111 | 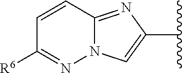 | CH₃ | 3 | 1 |
| 112 | 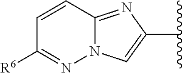 | CH₃ | 2 | 2 |
| 113 | 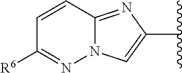 | CH₃ | 3 | 2 |
| 114 | 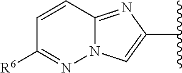 | CH₃ | 4 | 1 |
| 115 | 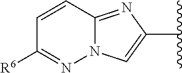 | Cl | 1 | 1 |
| 116 | 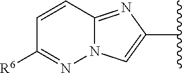 | Cl | 2 | 1 |
| 117 | 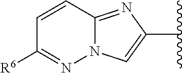 | Cl | 3 | 1 |
| 118 | 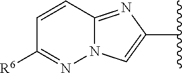 | Cl | 2 | 2 |
| 119 | 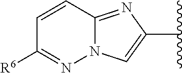 | Cl | 3 | 2 |
| 120 | 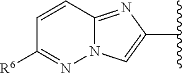 | Cl | 4 | 1 |
| 121 | 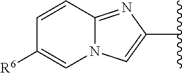 | CH₃ | 1 | 1 |
| 122 | 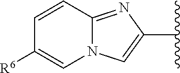 | CH₃ | 2 | 1 |
| 123 | 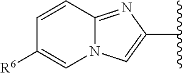 | CH₃ | 3 | 1 |
| 124 | 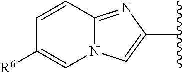 | CH₃ | 2 | 2 |
| 125 | 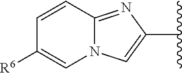 | CH₃ | 3 | 2 |
| 126 | 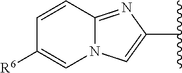 | CH₃ | 4 | 1 |
| 127 | 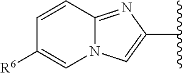 | Cl | 1 | 1 |
| 128 | 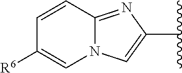 | Cl | 2 | 1 |
| 129 | 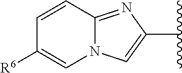 | Cl | 3 | 1 |
| 130 | 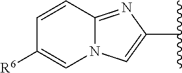 | Cl | 2 | 2 |
| 131 | 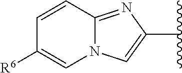 | Cl | 3 | 2 |
| 132 | 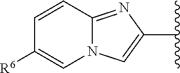 | Cl | 4 | 1 |
| 133 | 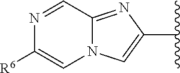 | CH₃ | 1 | 1 |
| 134 | 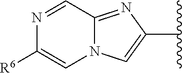 | CH₃ | 2 | 1 |
| 135 | 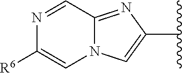 | CH₃ | 3 | 1 |
| 136 | 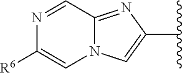 | CH₃ | 2 | 2 |
| 137 | 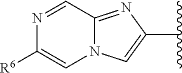 | CH₃ | 3 | 2 |
| 138 | 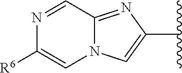 | CH₃ | 4 | 1 |
| 139 | 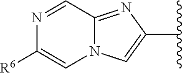 | Cl | 1 | 1 |
| 140 | 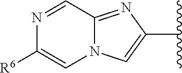 | Cl | 2 | 1 |
| 141 | 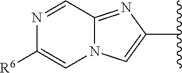 | Cl | 3 | 1 |
| 142 | 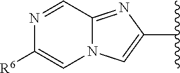 | Cl | 2 | 2 |
| 143 | 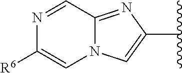 | Cl | 3 | 2 |
| 144 | 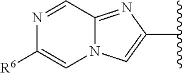 | Cl | 4 | 1 |

MNK inhibitors of the present disclosure include compounds having the formula (LIII) or a pharmaceutically acceptable salt form thereof:

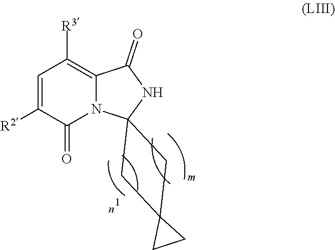

wherein non-limiting examples of R^{3′}, R^{2′}, n¹ and m are defined herein below in Table 3.

TABLE 3

| Entry | R^{3′} | R^{2′} | m | n¹ |
|---|---|---|---|---|
| 1 | CH₃ | 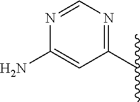 [SMILES: *c1cc(N)ncn1] | 1 | 1 |
| 2 | CH₃ | 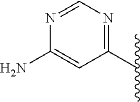 [SMILES: *c1cc(N)ncn1] | 2 | 1 |
| 3 | CH₃ | 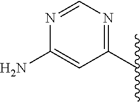 [SMILES: *c1cc(N)ncn1] | 3 | 1 |
| 4 | CH₃ | 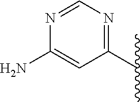 [SMILES: *c1cc(N)ncn1] | 2 | 2 |
| 5 | CH₃ | 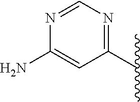 [SMILES: *c1cc(N)ncn1] | 3 | 2 |
| 6 | CH₃ | 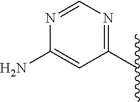 [SMILES: *c1cc(N)ncn1] | 4 | 1 |
| 7 | CH₃ | 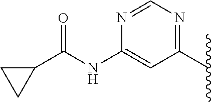 [SMILES: *c1cc(NC(=O)C2CC2)ncn1] | 1 | 1 |
| 8 | CH₃ | 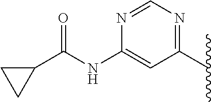 [SMILES: *c1cc(NC(=O)C2CC2)ncn1] | 2 | 1 |
| 9 | CH₃ | 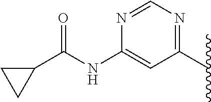 [SMILES: *c1cc(NC(=O)C2CC2)ncn1] | 3 | 1 |
| 10 | CH₃ | 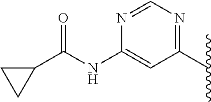 [SMILES: *c1cc(NC(=O)C2CC2)ncn1] | 2 | 2 |
| 11 | CH₃ | 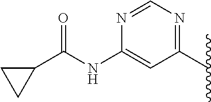 [SMILES: *c1cc(NC(=O)C2CC2)ncn1] | 3 | 2 |
| 12 | CH₃ | 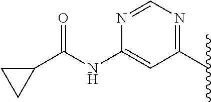 [SMILES: *c1cc(NC(=O)C2CC2)ncn1] | 4 | 1 |
| 13 | Cl | 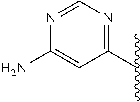 [SMILES: *c1cc(N)ncn1] | 1 | 1 |
| 14 | Cl | 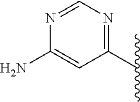 [SMILES: *c1cc(N)ncn1] | 2 | 1 |
| 15 | Cl | 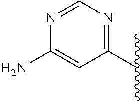 [SMILES: *c1cc(N)ncn1] | 3 | 1 |
| 16 | Cl | 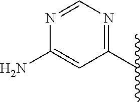 [SMILES: *c1cc(N)ncn1] | 2 | 2 |
| 17 | Cl | 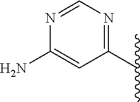 [SMILES: *c1cc(N)ncn1] | 3 | 2 |
| 18 | Cl | 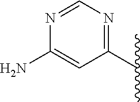 [SMILES: *c1cc(N)ncn1] | 4 | 1 |
| 19 | CH₃ | 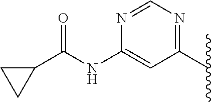 [SMILES: *c1cc(NC(=O)C2CC2)ncn1] | 1 | 1 |
| 20 | CH₃ | 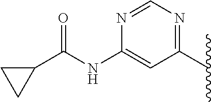 [SMILES: *c1cc(NC(=O)C2CC2)ncn1] | 2 | 1 |
| 21 | CH₃ | 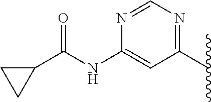 [SMILES: *c1cc(NC(=O)C2CC2)ncn1] | 3 | 1 |
| 22 | CH₃ | 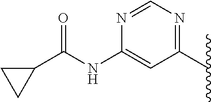 [SMILES: *c1cc(NC(=O)C2CC2)ncn1] | 2 | 2 |
| 23 | CH₃ | 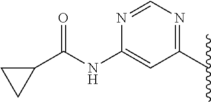 [SMILES: *c1cc(NC(=O)C2CC2)ncn1] | 3 | 2 |
| 24 | CH₃ | 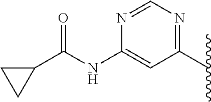 [SMILES: *c1cc(NC(=O)C2CC2)ncn1] | 4 | 1 |
| 25 | Br | 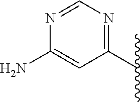 [SMILES: *c1cc(N)ncn1] | 1 | 1 |
| 26 | Br | 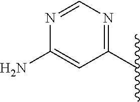 [SMILES: *c1cc(N)ncn1] | 2 | 1 |
| 27 | CH₂F | 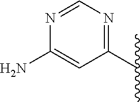 [SMILES: *c1cc(N)ncn1] | 1 | 1 |
| 28 | CH₂F | 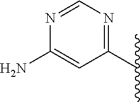 [SMILES: *c1cc(N)ncn1] | 2 | 1 |
| 29 | CH₂F | 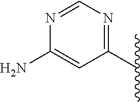 [SMILES: *c1cc(N)ncn1] | 3 | 1 |
| 30 | CH₂F | 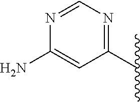 [SMILES: *c1cc(N)ncn1] | 2 | 2 |
| 31 | CH₂F | 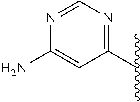 [SMILES: *c1cc(N)ncn1] | 3 | 2 |
| 32 | CH₂F | 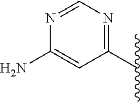 [SMILES: *c1cc(N)ncn1] | 4 | 1 |
| 33 | CH₂CH₃ | 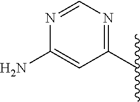 [SMILES: *c1cc(N)ncn1] | 1 | 1 |
| 34 | CH₂CH₃ | 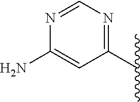 [SMILES: *c1cc(N)ncn1] | 2 | 1 |
| 35 | CH₂CH₃ | 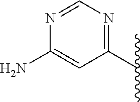 [SMILES: *c1cc(N)ncn1] | 3 | 1 |
| 36 | CH₂CH₃ | 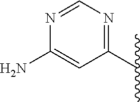 [SMILES: *c1cc(N)ncn1] | 2 | 2 |
| 37 | CH₂CH₃ | 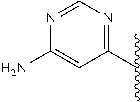 [SMILES: *c1cc(N)ncn1] | 3 | 2 |
| 38 | CH₂CH₃ | 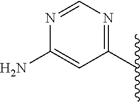 [SMILES: *c1cc(N)ncn1] | 4 | 1 |
| 39 | CH₂CH₂F | 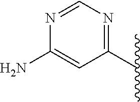 [SMILES: *c1cc(N)ncn1] | 1 | 1 |
| 40 | CH₂CH₂F | 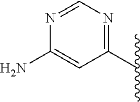 [SMILES: *c1cc(N)ncn1] | 2 | 1 |
| 41 | CH₂CH₂F | 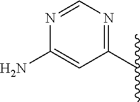 [SMILES: *c1cc(N)ncn1] | 3 | 1 |
| 42 | CH₂CH₂F | 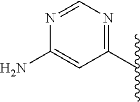 [SMILES: *c1cc(N)ncn1] | 2 | 2 |
| 43 | CH₂CH₂F | 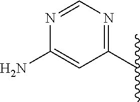 [SMILES: *c1cc(N)ncn1] | 3 | 2 |
| 44 | CH₂CH₂F | 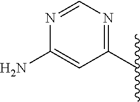 [SMILES: *c1cc(N)ncn1] | 4 | 1 |
| 45 | CF₂CH₃ | 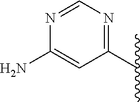 [SMILES: *c1cc(N)ncn1] | 1 | 1 |
| 46 | CF₂CH₃ | 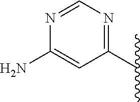 [SMILES: *c1cc(N)ncn1] | 2 | 1 |
| 47 | CF₂CH₃ | 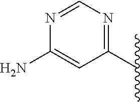 [SMILES: *c1cc(N)ncn1] | 3 | 1 |
| 48 | CF₂CH₃ | 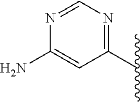 [SMILES: *c1cc(N)ncn1] | 2 | 2 |
| 49 | CF₂CH₃ | 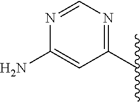 [SMILES: *c1cc(N)ncn1] | 3 | 2 |
| 50 | CF₂CH₃ | 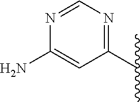 [SMILES: *c1cc(N)ncn1] | 4 | 1 |
| 51 | Br | 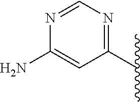 [SMILES: *c1cc(N)ncn1] | 3 | |
| 52 | Br | 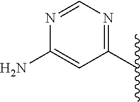 [SMILES: *c1cc(N)ncn1] | 4 | |

MNK inhibitors of the present disclosure include compounds having the formula (LIV) or a pharmaceutically acceptable salt form thereof:

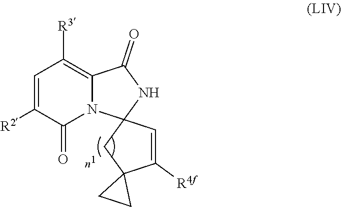

wherein non-limiting examples of R^{3′}, R^{4f}, and n¹ are defined herein below in Table 4.

TABLE 4

| Entry | R^{3′} | R^{4f} | n¹ | Entry | R^{3′} | R^{4f} | n¹ |
|---|---|---|---|---|---|---|---|
| 1 | CH₃ | H | 1 | 19 | Cl | H | 1 |
| 2 | CH₃ | CH ₃ | 1 | 20 | Cl | CH₃ | 1 |
| 3 | CH₃ | CH₂CH ₃ | 1 | 21 | Cl | CH₂CH₃ | 1 |
| 4 | CH₃ | CH(CH₃)₂ | 1 | 22 | Cl | CH(CH₃)₂ | 1 |
| 5 | CH₃ | CH₂F | 1 | 23 | Cl | CH₂F | 1 |
| 6 | CH₃ | CH₂CH₂F | 1 | 24 | Cl | CH₂CH₂F | 1 |
| 7 | CH₃ | H | 2 | 25 | Cl | H | 2 |
| 8 | CH₃ | CH₃ | 2 | 29 | Cl | CH₃ | 2 |
| 9 | CH₃ | CH₂CH ₃ | 2 | 27 | Cl | CH₂CH₃ | 2 |
| 10 | CH₃ | CH(CH₃)₂ | 2 | 28 | Cl | CH(CH ₃)₂ | 2 |
| 11 | CH₃ | CH₂F | 2 | 29 | Cl | CH₂F | 2 |
| 12 | CH₃ | CH₂CH₂F | 2 | 30 | Cl | CH₂CH₂F | 2 |
| 13 | CH₃ | H | 3 | 31 | Cl | H | 3 |
| 14 | CH₃ | CH₃ | 3 | 32 | Cl | CH₃ | 3 |
| 15 | CH₃ | CH₂CH₃ | 3 | 33 | Cl | CH₂CH ₃ | 3 |
| 16 | CH₃ | CH(CH₃)₂ | 3 | 34 | Cl | CH(CH₃)₂ | 3 |
| 17 | CH₃ | CH₂F | 3 | 35 | Cl | CH₂F | 3 |
| 18 | CH₃ | CH₂CH₂F | 3 | 36 | Cl | CH₂CH₂F | 3 |

In some embodiments, a MNK inhibitor is a compound selected from:
N-(6-((8″-methyl-1″,5″-dioxo-1″,5″-dihydro-2″H-dispiro[cyclopropane-1,1′-cyclohexane-3′,3″-imidazo[1,5-a]pyridin]-6″-yl)amino)pyrimidin-4-yl)cyclopropanecarboxamide;
6″-((6-Aminopyrimidin-4-yl)amino)-8″-methyl-2″H-dispiro[cyclopropane-1,1′-cyclohexane-3′,3″-imidazo[1,5-a]pyridine]-1″,5″-dione;
N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropanecarboxamide;
N-(6-((8″-methyl-1″,5″-dioxo-1″,5″-dihydro-2″H-dispiro[aziridine-2,1′-cyclohexane-4′,3″-imidazo[1,5-a]pyridin]-6″-yl)amino)pyrimidin-4-yl)cyclopropanecarboxamide;
N-(6-((8″-methyl-1″,5″-dioxo-1″,5″-dihydro-2′H-dispiro[cyclobutane-1,1′-cyclobutane-3′,3″-imidazo[1,5-a]pyridin]-6″-yl)amino)pyrimidin-4-yl)cyclopropanecarboxamide;
benzyl 6″-((6-(cyclopropanecarboxamido)pyrimidin-4-yl)amino)-8″-methyl-1″,5″-dioxo-1″,5″-dihydro-2′H-dispiro[aziridine-2,1′-cyclohexane-4′,3″-imidazo[1,5-a]pyridine]-1-carboxylate;
tert-butyl 6″-((6-(cyclopropanecarboxamido)pyrimidin-4-yl)amino)-8″-methyl-1″,5″-dioxo-1″,5″-dihydro-2″H-dispiro[azetidine-3,1′-cyclohexane-4′,3″-imidazo[1,5-a]pyridine]-1-carboxylate;
N-(6-((8″-methyl-1″,5″-dioxo-1″,5″-dihydro-2″H-dispiro[azetidine-3,1′-cyclohexane-4′,3″-imidazo[1,5-a]pyridin]-6″-yl)amino)pyrimidin-4-yl)cyclopropanecarboxamide;
6″-((6-((2-hydroxyethyl)amino)pyrimidin-4-yl)amino)-8″-methyl-2″H-dispiro[cyclopropane-1,1′-cyclohexa ne-4′,3″-imidazo[1,5-a]pyridine]-1″,5″-dione;
6″-((6-Aminopyrimidin-4-yl)amino)-8″-methyl-2′H-dispiro[cyclopropane-1,1′-cyclohexane-4′,3″-imidazo[1,5-a]pyridine]-1″,5″-d lone;
benzyl 6″-((6-aminopyrimidin-4-yl)amino)-8″-methyl-1″,5″-dioxo-1″,5″-dihydro-2″H-dispiro[aziridine-2,1′-cyclohexane-4′,3″-imidazo[1,5-a]pyridine]-1-carboxylate;
1-(aminomethyl)-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropane-1-carboxamide;
(1R,5S,6r)-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-3-azabicyclo[3.1.0]hexane-6-carboxamide;
N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-2-azaspiro[3.3]heptane-6-carboxamide;
2-methyl-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-2-azaspiro[3.3]heptane-6-carboxamide;
(1R,5S,6r)-3-methyl-N-(6-((8″-methyl-1″,5″-dioxo-1″,5″-dihydro-2″H-dispiro[cyclopropane-1,1′-cyclohexa ne-4′,3″-imidazo[1,5-a]pyridin]-6″-yl)amino)pyrimidin-4-yl)-3-azabicyclo[3.1.0]hexane-6-carboxamide;
N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-1-(methylsulfonamido methyl)cyclopropane-1-carboxamide;
1-((dimethylamino)methyl)-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropane-1-carboxamide;
6″-((6-aminopyrimidin-4-yl)amino)-8″-methyl-2′H-dispiro[cyclobutane-1,1′-cyclobuta ne-3′,3″-imidazo[1,5-a]pyridine]-1″,5″-d lone;
6″-((6-aminopyrimidin-4-yl)amino)-8″-methyl-2″H-dispiro[aziridine-2,1′-cyclohexane-4′,3″-imidazo[1,5-a]pyridine]-1″,5″-dione;
6″-((6-aminopyrimidin-4-yl)amino)-8″-methyl-2′H-dispiro[cyclopropane-1,1′-cyclopentane-3′,3″-imidazo[1,5-a]pyridine]-1″,5″-dione;
6″-((6-aminopyrimidin-4-yl)amino)-8″-methyl-2″H-dispiro[cyclopentane-1,1′-cyclopentane-3′,3″-imidazo[1,5-a]pyridine]-1″,5″-dione;
6″-((6-aminopyrimidin-4-yl)amino)-3,3-difluoro-8″-methyl-2″H-dispiro[cyclobuta ne-1,1′-cyclobutane-3′,3″-imidazo[1,5-a]pyridine]-1″,5″ dione;
6″-((6-aminopyrimidin-4-yl)amino)-8′-methyl-2′H-dispiro[cyclopentane-1,1′-cyclobutane-3′,3″-imidazo[1,5-a]pyridine]-1″,5″-dione;
6″-((6-aminopyrimidin-4-yl)amino)-8″-methyl-2′H-dispiro[cyclobutane-1,1′-cyclohexane-4′,3″-imidazo[1,5-a]pyridine]-1″,5″-d lone;
6″-((6-aminopyrimidin-4-yl)amino)-8″-methyl-2″H-dispiro[cyclohexane-1,1′-cyclobuta ne-3′,3″-imidazo[1,5-a]pyridine]-1″,5″-d lone;
ethyl 6″-((6-(cyclopropanecarboxamido)pyrimidin-4-yl)amino)-8″-methyl-1″,5″-dioxo-1″,5″-dihydro-2′H-dispiro[cyclopropane-1,1′-cyclohexane-4′,3″-imidazo[1,5-a]pyridine]-2-carboxylate;
tert-butyl (6″-((6-(cyclopropanecarboxamido)pyrimidin-4-yl)amino)-8″-methyl-1″,5″-dioxo-1″,5″-dihydro-2″H-dispiro[cyclopropane-1,1′-cyclohexane-4′,3″-imidazo[1,5-a]pyridin]-2-yl)carbamate;
N-(6-((2,2-difluoro-8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropanecarboxamide;
6″-((6-aminopyrimidin-4-yl)amino)-2,2-difluoro-8″-methyl-2″H-dispiro[cyclopropane-1,1′-cyclohexane-4′,3″-imidazo[1,5-a]pyridine]-1″,5″-dione;
6″-((6-Aminopyrimidin-4-yl)amino)-8″-methyl-2″H-dispiro[cyclopropane-1,1′-cycloheptane-4′,3″-imidazo[1,5-a]pyridine]-1″,5″-dione;
6″-((6-Aminopyrimidin-4-yl)amino)-8″-methyl-2″H-dispiro[cyclopropane-1,1′-cyclohexane-4′,3″-imidazo[1,5-a]pyridin]-2′-ene-1″,5″-dione;
   or a pharmaceutically acceptable salt thereof.

### MNK Inhibitors of Formula (IB)

In some embodiments, a MNK inhibitor is a compound of Formula (IB):

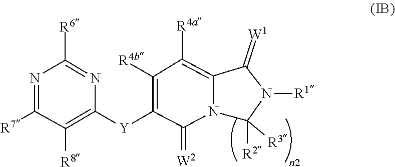

or a pharmaceutically acceptable salt thereof, wherein:
W¹ and W² are independently O, S or N—OR′, where R′ is lower alkyl;
Y is —N(R^{5″})—, —O—, —S—, —C(O)—, —S═O, —S(O)₂—, or —CHR⁹—;
R^{1″} is hydrogen, lower alkyl, cycloalkyl or heterocyclyl wherein any lower alkyl, cycloalkyl or heterocyclyl is optionally substituted with 1, 2 or 3 J groups;
n² is 1, 2 or 3;
R^{2″} and R^{3″} are each independently hydrogen, alkyl, alkenyl, alkynyl, aryl, araalkylene, heteroaryl, heteroarylalkylene, cycloalkyl, cycloalkylalkylene, heterocyclyl, or heterocyclylalkylene, wherein any alkyl, aryl, araalkylene, heteroaryl, heteroarylalkylene, cycloalkyl, cycloalkylalkylene, heterocyclyl, or heterocyclylalkylene, is optionally substituted with 1, 2 or 3 3 groups;
or R^{2″} and R^{3″} taken together with the carbon atom to which they are attached form a cycloalkyl or heterocyclyl, wherein any cycloalkyl or heterocyclyl is optionally substituted with 1, 2 or 3 J groups;
R^{4a″} and R^{4b″} are each independently hydrogen, halogen, hydroxyl, thiol, hydroxyalkylene, cyano, alkyl, alkoxy, acyl, thioalkyl, alkenyl, alkynyl, cycloalkyl, aryl, or heterocyclyl;
R^{5″} is hydrogen, cyano, or lower alkyl;
or R^{5″} and R⁸ taken together with the atoms to which they are attached form a fused heterocyclyl optionally substituted with 1, 2 or 3 J groups;
R^{6″}, R^{7″} and R⁸ are each independently hydrogen, hydroxy, halogen, cyano, amino, alkyl, alkenyl, alkynyl, alkoxy, cycloalkyl, cycloalkylalkylene, cycloalkylalkenylene, alkylaminyl, alkylcarbonylaminyl, cycloalkylcarbonylaminyl, cycloalkylaminyl, heterocyclylaminyl, heteroaryl, or heterocyclyl, and wherein any amino, alkyl, alkenyl, alkynyl, alkoxy, cycloalkyl, cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups;
or R^{7″} and R⁸ taken together with the atoms to which they are attached form a fused heterocyclyl or heteroaryl optionally substituted with 1, 2 or 3 3 groups;
J is —SH, —SR⁹, —S(O)R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)—NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene, NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl; or any two I groups bound to the same carbon or hetero atom may be taken together to form oxo; and
R⁹ is hydrogen, lower alkyl or —OH.

The following definitions apply to Formula (IB) and subgenera thereof:

“Amino” refers to the —NH₂ substituent.

“Aminocarbonyl” refers to the —C(O)NH₂ substituent.

“Carboxyl” refers to the —CO₂H substituent.

“Carbonyl” refers to a —C(O)— or —C(═O)— group.

“Cyano” refers to the —C≡N substituent.

“Cyanoalkylene” refers to the -(alkylene)C≡N substituent.

“Acetyl” refers to the —C(O)CH₃ substituent.

“Hydroxy” or “hydroxyl” refers to the —OH substituent.

“Hydroxyalkylene” refers to the -(alkylene)OH substituent.

“Oxo” refers to an oxygen of —O— substituent.

“Thio” or “thiol” refer to a —SH substituent.

“Alkyl” refers to a saturated, straight or branched hydrocarbon chain radical consisting solely of carbon and hydrogen atoms, having from one to twelve carbon atoms (C₁-C₁₂ alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like.

“Lower alkyl” has the same meaning as alkyl defined above but having from one to four carbon atoms (C₁-C₄ alkyl).

“Alkenyl” refers to an unsaturated alkyl group having at least one double bond and from two to twelve carbon atoms (C₂-C₁₂ alkenyl), from two to eight carbon atoms (C₂-C₈ alkenyl) or from two to six carbon atoms (C₂-C₆ alkenyl), and which is attached to the rest of the molecule by a single bond, e.g., ethenyl, propenyl, butenyl, pentenyl, hexenyl, and the like.

“Alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₆ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkenylene” refers to divalent alkene. Examples of alkenylene include without limitation, ethenylene (—CH═CH—) and all stereoisomer and conformational isomeric forms thereof. “Substituted alkenylene” refers to divalent substituted alkene. “Optionally substituted alkenylene” refers to alkenylene or substituted alkenylene.

“Alkynylene” refers to divalent alkyne. Examples of alkynylene include without limitation, ethynylene, propynylene. “Substituted alkynylene” refers to divalent substituted alkyne.

“Alkoxy” refers to a radical of the formula —OR, where Rₐ is an alkyl having the indicated number of carbon atoms as defined above. Examples of alkoxy groups include without limitation —O-methyl (methoxy), —O-ethyl (ethoxy), —O-propyl (propoxy), —O-isopropyl (iso propoxy) and the like.

“Acyl” refers to a radical of the formula —C(O)Rₐ where Rₐ is an alkyl having the indicated number of carbon atoms.

“Alkylaminyl” refers to a radical of the formula —NHRₐ or —NRₐRₐ where each Rₐ is, independently, an alkyl radical having the indicated number of carbon atoms as defined above.

“Cycloalkylaminyl” refers to a radical of the formula —NHRₐ where Rₐ is a cycloalkyl radical as defined herein.

“Alkylcarbonylaminyl” refers to a radical of the formula —NHC(O)Rₐ, where Rₐ is an alkyl radical having the indicated number of carbon atoms as defined herein.

“Cycloalkylcarbonylaminyl” refers to a radical of the formula —NHC(O)Rₐ, where Rₐ is a cycloalkyl radical as defined herein.

“Alkylaminocarbonyl” refers to a radical of the formula —C(O)NHRₐ or —C(O)NRₐRₐ, where each Rₐ is independently, an alkyl radical having the indicated number of carbon atoms as defined herein.

“Cyclolkylaminocarbonyl” refers to a radical of the formula —C(O)NHRₐ, where Rₐ is a cycloalkyl radical as defined herein.

“Aryl” refers to a hydrocarbon ring system radical comprising hydrogen, 6 to 18 carbon atoms and at least one aromatic ring. Exemplary aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted aryl” refers to an aryl group or a substituted aryl group.

“Arylene” denotes divalent aryl, and “substituted arylene” refers to divalent substituted aryl.

“Aralkyl” or “araalkylene” may be used interchangeably and refer to a radical of the formula —R_{b}—R_{c} where R_{b} is an alkylene chain as defined herein and R_{c} is one or more aryl radicals as defined herein, for example, benzyl, diphenylmethyl and the like.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, three to nine carbon atoms, three to eight carbon atoms, three to seven carbon atoms, three to six carbon atoms, three to five carbon atoms, a ring with four carbon atoms, or a ring with three carbon atoms. The cycloalkyl ring may be saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like.

“Cycloalkylalkylene” or “cycloalkylalkyl” may be used interchangeably and refer to a radical of the formula —R_{b}Rₑ where R_{b} is an alkylene chain as defined herein and Rₑ is a cycloalkyl radical as defined herein. In certain embodiments, R_{b} is further substituted with a cycloalkyl group, such that the cycloalkylalkylene comprises two cycloalkyl moieties. Cyclopropylalkylene and cyclobutylalkylene are exemplary cycloalkylalkylene groups, comprising at least one cyclopropyl or at least one cyclobutyl group, respectively.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo (bromine), chloro (chlorine), fluoro (fluorine), or iodo (iodine).

“Haloalkyl” refers to an alkyl radical having the indicated number of carbon atoms, as defined herein, wherein one or more hydrogen atoms of the alkyl group are substituted with a halogen (halo radicals), as defined above. The halogen atoms can be the same or different. Exemplary haloalkyls are trifluoromethyl, difluoromethyl, trichloromethyl, 2,2,2-trifluoroethyl, 1,2-difluoroethyl, 3-bromo-2-fluoropropyl, 1,2-dibromoethyl, and the like.

“Heterocyclyl”, heterocycle”, or “heterocyclic ring” refers to a stable 3-to 18-membered saturated or unsaturated radical which consists of two to twelve carbon atoms and from one to six heteroatoms, for example, one to five heteroatoms, one to four heteroatoms, one to three heteroatoms, or one to two heteroatoms selected from the group consisting of nitrogen, oxygen and sulfur. Exemplary heterocycles include without limitation stable 3-15 membered saturated or unsaturated radicals, stable 3-12 membered saturated or unsaturated radicals, stable 3-9 membered saturated or unsaturated radicals, stable 8-membered saturated or unsaturated radicals, stable 7-membered saturated or unsaturated radicals, stable 6-membered saturated or unsaturated radicals, or stable 5-membered saturated or unsaturated radicals.

Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, thietanyl, trithianyl, tetrahydropyranyl, thiomorpholinyl, thiamorpholinyl, 1-oxo-thiomorpholinyl, and 1,1-dioxo-thiomorpholinyl. Heterocyclyls include heteroaryls as defined herein, and examples of aromatic heterocyclyls are listed in the definition of heteroaryls below.

“Heterocyclylalkyl” or “heterocyclylalkylene” refers to a radical of the formula —R_{b}R_{f} where R_{b} is an alkylene chain as defined herein and R_{f} is a heterocyclyl radical as defined above, and if the heterocyclyl is a nitrogen-containing heterocyclyl, the heterocyclyl may be attached to the alkyl radical at the nitrogen atom.

“Heteroaryl” or “heteroarylene” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl).

“Heteroarylalkyl” or “heteroarylalkylene” refers to a radical of the formula —R_{b}R_{g} where R_{b} is an alkylene chain as defined above and R_{g} is a heteroaryl radical as defined above.

“Thioalkyl” refers to a radical of the formula —SRₐ where Rₐ is an alkyl radical as defined above containing one to twelve carbon atoms, at least 1-10 carbon atoms, at least 1-8 carbon atoms, at least 1-6 carbon atoms, or at least 1-4 carbon atoms.

“Heterocyclylaminyl” refers to a radical of the formula —NHR_{f} where R_{f} is a heterocyclyl radical as defined above.

“Thione” refers to a ═S group attached to a carbon atom of a saturated or unsaturated (C₃-C₈)cyclic or a (C₁-C₈)acyclic moiety.

“Sulfoxide” refers to a —S(O)— group in which the sulfur atom is covalently attached to two carbon atoms.

“Sulfone” refers to a —S(O)₂— group in which a hexavalent sulfur is attached to each of the two oxygen atoms through double bonds and is further attached to two carbon atoms through single covalent bonds.

The term “oxime” refers to a —C(Rₐ)═N—ORₐ radical where Rₐ is hydrogen, lower alkyl, an alkylene or arylene group as defined above.

The compound of the disclosure can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the disclosure can be in the form of an optical isomer or a diastereomer. Accordingly, the disclosure encompasses compounds of the disclosure and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, or via chemical separation of stereoisomers through the employment of optically active resolving agents.

In some embodiments, a MNK inhibitor is a compound selected from Table 5, or a pharmaceutically acceptable salt thereof.

TABLE 5

| No. | Compound |
|---|---|
| B1 | 7-(pyrimidin-4-ylamino)-3,4-dihydro-1H-pyrido[1,2-a]pyrazine-1,6(2H)-dione |
| B2 | 3,3-dimethyl-6-(pyrimidin-4-ylamino)-2,3-dihydroimidazo[1,5-a]pyridine-1,5-dione |
| B3 | 3-(4-fluorobenzyl)-3-methyl-6-(pyrimidin-4-ylamino)-2,3- |
| | dihydroimidazo[1,5-a]pyridine-1,5-dione |
| B4 | 3-(4-chlorobenzyl)-3-methyl-6-(pyrimidin-4-ylamino)-2,3- |
| | dihydroimidazo[1,5-a]pyridine-1,5-dione |
| B5 | 3-(3-fluorobenzyl)-3-methyl-6-(pyrimidin-4-ylamino)-2,3- |
| | dihydroimidazo[1,5-a]pyridine-1,5-dione |
| B6 | 6′-(pyrimidin-4-ylamino)-1'H-spiro[cyclopentane-1,3′-imidazo[1,5- |
| | alpyridine]-1′,5′(2′H)-dione |
| B7 | 3-methyl-6-(pyrimidin-4-ylamino)-3-(2,2,2-trifluoroethyl)-2,3- |
| | dihydroimidazo[1,5-a]pyridine-1,5-dione |
| B8 | 3-isopropyl-3-methyl-6-(pyrimidin-4-ylamino)-2,3-dihydroimidazo[1,5- |
| | alpyridine-1,5-dione |
| B9 | 3-cyclopentyl-3-methyl-6-(pyrimidin-4-ylamino)-2,3-dihydroimidazo[1,5- |
| | alpyridine-1,5-dione |
| B10 | N-(6-((3,3-dimethyl-1,5-dioxo-1,2,3,5-tetrahydroimidazo[1,5-a]pyridin-6- |
| | yl)amino) pyrimidin-4-yl)cyclopropanecarboxamide |
| B11 | 3-(4-fluorophenyl)-3-methyl-6-(pyrimidin-4-ylamino)-2,3- |
| | dihydroimidazo[1,5-a]pyridine-1,5-dione |
| B12 | 3-(3-fluorophenyl)-3-methyl-6-(pyrimidin-4-ylamino)-2,3- |
| | dihydroimidazo[1,5-a]pyridine-1,5-dione |
| B13 | 3-(4-chlorophenyl)-3-methyl-6-(pyrimidin-4-ylamino)-2,3- |
| | dihydroimidazo[1,5-a]pyridine-1,5-dione |
| B14 | 3-methyl-6-(pyrimidin-4-ylamino)-3-(trifluoromethyl)-2,3- |
| | dihydroimidazo[1,5-a]pyridine-1,5-dione |
| B15 | 3-(aminomethyl)-3-methyl-6-(pyrimidin-4-ylamino)-2,3- |
| | dihydroimidazo[1,5-a]pyridine-1,5-dione |
| B16 | 3-methyl-6-(pyrimidin-4-ylamino)-3-(thiophen-3-yl)-2,3- |
| | dihydroimidazo[1,5-a]pyridine-1,5-dione |
| B17 | 3-methyl-6-(pyrimidin-4-ylamino)-3-(thiazol-4-yl)-2,3- |
| | dihydroimidazo[1,5-a]pyridine-1,5-dione |
| B18 | 3-(3-chlorophenyl)-3-methyl-6-(pyrimidin-4-ylamino)-2,3- |
| | dihydroimidazo[1,5-a]pyridine-1,5-dione |
| B19 | 3-(3-chlorobenzyl)-3-methyl-6-(pyrimidin-4-ylamino)-2,3- |
| | dihydroimidazo[1,5-a]pyridine-1,5-dione |
| B20 | 6-(pyrimidin-4-ylamino)-1H-spiro[imidazo[1,5-a]pyridine-3,3′-piperidine]- |
| | 1,5(2H)-dione |
| B21 | N-[6-[(1,5-dioxospiro[2H-imidazo[1,5-a]pyridine-3,1′-cyclopentane]-6- |
| | yl)amino]pyrimidin-4-yl]cyclopropanecarboxamide |
| B22 | N-(6-((3-methyl-1,5-dioxo-3-(2,2,2-trifluoroethyl)-1,2,3,5- |
| | tetrahydroimidazo[1,5-a]pyridin-6-yl)amino)pyrimidin-4- |
| | yl)cyclopropanecarboxamide |
| B23 | N-[6-[(3-cyclopentyl-3-methyl-1,5-dioxo-2H-imidazo[1,5-a]pyridin-6- |
| | yl)amino]pyrimidin-4-yl]cyclopropanecarboxamide |
| B24 | N-[6-[[3-methyl-1,5-dioxo-3-(trifluoromethyl)-2H-imidazo[1,5-a]pyridin- |
| | 6-yllamino]pyrimidin-4-yl]cyclopropanecarboxamide |
| B25 | 3-methyl-3-((methylamino)methyl)-6-(pyrimidin-4-ylamino)-2,3- |
| | dihydroimidazo[1,5-a]pyridine-1,5-dione |
| B26 | 6-(pyrimidin-4-ylamino)-1H-spiro[imidazo[1,5-a]pyridine-3,3′- |
| | pyrrolidine]-1,5(2H)-dione |
| B27 | 3-(3,5-difluorophenyl)-3-methyl-6-(pyrimidin-4-ylamino)-2,3- |
| | dihydroimidazo[1,5-a]pyridine-1,5-dione |
| B28 | 3-(3-chloro-5-fluorophenyl)-3-methyl-6-(pyrimidin-4-ylamino)-2,3- |
| | dihydroimidazo[1,5-a]pyridine-1,5-dione |
| B29 | N-[6-[(3-isopropyl-3-methyl-1,5-dioxo-2H-imidazo[1,5-a]pyridin-6- |
| | yl)amino]pyrimidin-4-yl]cyclopropanecarboxamide |
| B30 | N-[6-[[3-(3-fluorophenyl)-3-methyl-1,5-dioxo-2H-imidazo[1,5-a]pyridin- |
| | 6- yllamino]pyrimidin-4-yl]cyclopropanecarboxamide |
| B31 | 6-[[6-[(E)-2-cyclopropylvinyl]pyrimidin-4-yl]amino]-3-methyl-3- |
| | (trifluoromethyl)-2H-imidazo[1,5-a]pyridine-1,5-dione |
| B32 | 3,3-dimethyl-6-(7H-pyrrolo[2,3-d]pyrimidin-7-yl)-2,3- |
| | dihydroimidazo[1,5-a]pyridine-1,5-dione |
| B33 | 3-(3,5-dichlorophenyl)-3-methyl-6-(pyrimidin-4-ylamino)-2,3- |
| | dihydroimidazo[1,5-a]pyridine-1,5-dione |
| B34 | 3-methyl-3-(pyridin-2-yl)-6-(pyrimidin-4-ylamino)-2,3- |
| | dihydroimidazo[1,5-a]pyridine-1,5-dione |
| B35 | 8-chloro-3-methyl-6-(pyrimidin-4-ylamino)-3-(trifluoromethyl)-2H- |
| | imidazo[1,5-a]pyridine-1,5-dione |
| B36 | 2′,2′-dimethyl-6-(pyrimidin-4-ylamino)spiro[2H-imidazo[1,5-a]pyridine- |
| | 3,1′-cyclopentane]-1,5-dione |
| B37 | 6-[(8-cyclopropyl-9H-purin-6-yl)amino]-3-methyl-3-(trifluoromethyl)-2H- |
| | imidazo[1,5-a]pyridine-1,5-dione |
| B38 | N-[6-[(8-chloro-1,5-dioxo-spiro[2H-imidazo[1,5-a]pyridine-3,1′- |
| | cyclopentane]-6-yl)amino\|pyrimidin-4-yl]cyclopropanecarboxamide |
| B39 | 8′-chloro-6′-(pyrimidin-4-ylamino)-2′H-spiro[cyclopentane-1,3′- |
| | imidazo[1,5-a]pyridine]-1′,5′-dione |
| B40 | 3,3-dimethyl-6-(5H-pyrrolo[2,3-d]pyrimidin-7(6H)-yl)-2,3- |
| | dihydroimidazo[1,5-a]pyridine-1,5-dione |
| B41 | 3-(2-aminoethyl)-3-methyl-6-(pyrimidin-4-ylamino)-2H-imidazo[1,5- |
| | alpyridine-1,5-dione |
| B42 | N-[6-[(8-chloro-3-cyclopentyl-3-methyl-1,5-dioxo-2H-imidazo[1,5- |
| | alpyridin-6-yl)amino]pyrimidin-4-yl]cyclopropanecarboxamide |
| B43 | N-[6-[(8-chloro-3,3-dimethyl-1,5-dioxo-2H-imidazo[1,5-a]pyridin-6- |
| | yl)amino]pyrimidin-4-yl]cyclopropanecarboxamide |
| B44 | N-(6-((8′-chloro-1′,5′-dioxo-1′,5′-dihydro-2′H-spiro[cyclobutane-1,3′- |
| | imidazo[1,5-a]pyridin]-6'-yl)amino)pyrimidin-4-yl)cyclopropanecarboxamide |
| B45 | N-(6-((8′-chloro-1′,5′-dioxo-1′,5′-dihydro-2′H-spiro[cyclohexane-1,3′- |
| | imidazo[1,5-a]pyridin]-6′-yl)amino)pyrimidin-4-yl)cyclopropanecarboxamide |
| B46 | N-[6-[[8-chloro-3-methyl-1,5-dioxo-3-(2,2,2-trifluoroethyl)-2H- |
| | imidazo[1,5-a]pyridin-6-yl]amino]pyrimidin-4-yllcyclopropanecarboxamide |
| B47 | 8-chloro-3,3-dimethyl-6-(pyrimidin-4-ylamino)-2,3-dihydroimidazo[1,5- |
| | alpyridine-1,5-dione |
| B48 | 8-chloro-3-cyclopentyl-3-methyl-6-(pyrimidin-4-ylamino)-2H- |
| | imidazo[1,5-a]pyridine-1,5-dione |
| B49 | N-(6-((8-chloro-3-methyl-1,5-dioxo-3-(trifluoromethyl)- 1,2,3,5- |
| | tetrahydroimidazo[1,5-a]pyridin-6-yl)amino)pyrimidin-4- |
| | yl)cyclopropanecarboxamide |
| B50 | N-[6-[(8-chloro-4′,4′-difluoro-1,5-dioxo-spiro[2H-imidazo[1,5-a]pyridine- |
| | 3,1′-cyclohexane]-6-yl)amino ]pyrimidin-4-yl]cyclopropanecarboxamide |
| B51 | N-[6-[[8-chloro-3-(3-chlorophenyl)-3-methyl-1,5-dioxo-2H-imidazo[1,5- |
| | a]pyridin-6-yl]amino]pyrimidin-4-yl]cyclopropanecarboxamide |
| B52 | N-[6-[[8-chloro-3-(3-fluorophenyl)-3-methyl-1,5-dioxo-2H-imidazo[1,5- |
| | alpyridin-6-yl]amino]pyrimidin-4-yl]cyclopropanecarboxamide |
| B53 | (3′S)-3′-amino-6-(pyrimidin-4-ylamino)spiro[2H-imidazo[1,5-a]pyridine- |
| | 3,1′-cyclohexane]-1,5-dione |
| B54 | 8-chloro-6-(pyrimidin-4-ylamino)spiro[2H-imidazo[1,5-a]pyridine-3,3′- |
| | piperidine]-1,5-dione |
| B55 | 8-chloro-3-methyl-6-(pyrimidin-4-ylamino)-3-(2,2,2-trifluoroethyl)-2H- |
| | imidazo[1,5-a]pyridine-1,5-dione |
| B56 | N-(6-((8-chloro-1,5-dioxo-1,5-dihydro-2H-spiro[imidazo[1,5-a]pyridine- |
| | 3,4′-piperidin]-6-yl)amino)pyrimidin-4-yl)cyclopropanecarboxamide |
| B57 | 8-chloro-3-(3-fluorophenyl)-3-methyl-6-(pyrimidin-4-ylamino)-2H- |
| | imidazo[1,5-a]pyridine-1,5-dione |
| B58 | 8-chloro-3-(3-chlorophenyl)-3-methyl-6-(pyrimidin-4-ylamino)-2H- |
| | imidazo[1,5-a]pyridine-1,5-dione |
| B59 | 6-((6-((1-cyclopropyl-2,2,2-trifluoroethyl)amino)pyrimidin-4-yl)amino)-3- |
| | methyl-3-(trifluoromethyl)-2,3-dihydroimidazo[1,5-a]pyridine-1,5-dione |
| B60 | N-(6-((8-chloro-1,5-dioxo-1,5-dihydro-2H-spiro[imidazo[1,5-a]pyridine- |
| | 3,3′-piperidin]-6-yl)amino)pyrimidin-4-yl)cyclopropanecarboxamide |
| B61 | N-(6-((8-chloro-1,5-dioxo-1,2′,3′,5,5′,6′-hexahydro-2H-spiro[imidazo[1,5- |
| | a]pyridine-3,4′-pyran]-6-yl)amino)pyrimidin-4-yl)cyclopropanecarboxamide |
| B62 | N-[6-[(8-chloro-1′,1′-difluoro-1,5-dioxo-spiro[2H-imidazo[1,5-a]pyridine- |
| | 3,3′-cyclobutane]-6-yl)amino]pyrimidin-4-yl]cyclopropanecarboxamide |
| B63 | 8-chloro-2′,2′-dimethyl-6-(pyrimidin-4-ylamino)spiro[2H-imidazo[1,5- |
| | alpyridine-3,1′-cyclopentane]-1,5-dione |
| B64 | 8-chloro-6-(pyrimidin-4-ylamino)-2′,3′,5′,6′-tetrahydro-2H- |
| | spiro[imidazo[1,5-a]pyridine-3,4′-pyran]-1,5-dione |
| B65 | N-(6-((8-chloro-1,5-dioxo-1,5-dihydro-2H-spiro[imidazo[1,5-a]pyridine- |
| | 3,3′-thietan]-6-yl)amino)pyrimidin-4-yl)cyclopropanecarboxamide |
| B66 | 8-chloro-2′-fluoro-6-(pyrimidin-4-ylamino)spiro[2H-imidazo[1,5- |
| | alpyridine-3,1′-cyclohexane]-1,5-dione |
| B67 | 3,3,8-trimethyl-6-(pyrimidin-4-ylamino)-2H-imidazo[1,5-a]pyridine-1,5-dione |
| B68 | 8-cyclopropyl-3,3-dimethyl-6-(pyrimidin-4-ylamino)-2H-imidazo[1,5- |
| | alpyridine-1,5-dione |
| B69 | 8-fluoro-3,3-dimethyl-6-(pyrimidin-4-ylamino)-2H-imidazo[1,5- |
| | alpyridine-1,5-dione |
| B70 | 6′-((6-aminopyrimidin-4-yl)amino)-8′-chloro-2'H-spiro[cyclohexane-1,3′- |
| | imidazo[1,5-a]pyridine]-1′,5′-dione |
| B71 | 8-ethyl-3,3-dimethyl-6-(pyrimidin-4-ylamino)-2H-imidazo[1,5-a]pyridine-1,5-dione |
| B72 | 8-chloro-3-methyl-3-(3-pyridyl)-6-(pyrimidin-4-ylamino)-2H-imidazo[1,5- |
| | alpyridine-1,5-dione |
| B73 | N-(6-((8-chloro-3-methyl-1,5-dioxo-3-(pyridin-4-yl)-1,2,3,5- |
| | tetrahydroimidazo[1,5-a]pyridin-6-yl)amino)pyrimidin-4- |
| | yl)cyclopropanecarboxamide |
| B74 | 8-chloro-3-methyl-3-(2-pyridyl)-6-(pyrimidin-4-ylamino)-2H-imidazo[1,5- |
| | alpyridine-1,5-dione |
| B75 | 8-chloro-3-methyl-3-(4-pyridyl)-6-(pyrimidin-4-ylamino)-2H-imidazo[1,5- |
| | alpyridine-1,5-dione |
| B76 | N-(6-((8-chloro-3-methyl-1,5-dioxo-3-(pyridin-2-yl)-1,2,3,5- |
| | tetrahydroimidazo[1,5-a]pyridin-6-yl)amino)pyrimidin-4-yl)cyclopropanecarboxamide |
| B77 | N-[6-[[8-chloro-3-methyl-1,5-dioxo-3-(3-pyridyl)-2H-imidazo[1,5- |
| | alpyridin-6-yllamino]pyrimidin-4-yl]cyclopropanecarboxamide |
| B78 | N-(6-((8′-chloro-2-fluoro-1′,5′-dioxo-1′,5′-dihydro-2′H-spiro[cyclohexane- |
| | 1,3′-imidazo[1,5-a]pyridin]-6′-yl)amino)pyrimidin-4-yl)cyclopropanecarboxamide |
| B79 | 3,3-dimethyl-6-(pyrimidin-4-ylamino)-8-vinyl-2H-imidazo[1,5-a]pyridine-1,5-dione |
| B80 | N-(6-((8-chloro-3-methyl-1,5-dioxo-3-(3-(trifluoromethyl)phenyl)- |
| | 1,2,3,5- tetrahydroimidazo[1,5-a]pyridin-6-yl)amino)pyrimidin-4- |
| | yl)cyclopropanecarboxamide |
| B81 | 8-methoxy-3,3-dimethyl-6-(pyrimidin-4-ylamino)-2H-imidazo[1,5- |
| | alpyridine-1,5-dione |
| B82 | 3,3-dimethyl-8-methylsulfanyl-6-(pyrimidin-4-ylamino)-2H-imidazo[1,5- |
| | alpyridine-1,5-dione |
| B83 | N-(6-((8-chloro-3-methyl-1,5-dioxo-3-(m-tolyl)-1,2,3,5-tetrahydroimidazo |
| | [1,5-a]pyridin-6-yl)amino)pyrimidin-4-yl)cyclopropanecarboxamide |
| B84 | N-[6-[(3-tert-butyl-8-chloro-3-methyl-1,5-dioxo-2H-imidazo[1,5- |
| | alpyridin-6-yl)amino]pyrimidin-4-yl]cyclopropanecarboxamide |
| B85 | N-[6-[[8-chloro-1,5-dioxo-3-(trifluoromethyl)-2,3-dihydroimidazo[1,5- |
| | alpyridin-6-yllamino]pyrimidin-4-yl]cyclopropanecarboxamide |
| B86 | N-[6-[(8-chloro-1, 5-dioxo-spiro [2H-imidazo [1, 5-a]pyridine-3,3′- |
| | azetidine]-6-yl)amino]pyrimidin-4-yl] cyclopropanecarboxamide |
| B87 | 6-[(6-aminopyrimidin-4-yl) amino]-8-chloro-3-(3-fluorophenyl)-3-methyl- |
| | 2H-imidazo[1,5-a]pyridine-1,5-dione |
| B88 | 6-[(6-aminopyrimidin-4-yl)amino]-8-chloro-3,3-dimethyl-2H-imidazo[1,5- |
| | alpyridine-1,5-dione |
| B89 | 8-chloro-3-(3-chlorophenyl)-3-methyl-6-[[6-(methylamino)pyrimidin-4- |
| | yllamino]-2H-imidazo[1,5-a]pyridine-1,5-dione |
| B90 | 8-chloro-3-methyl-3-(6-methyl-2-pyridy!)-6-(pyrimidin-4-ylamino)-2H- |
| | imidazo[1,5-a]pyridine-1,5-dione |
| B91 | N-[6-[[8-chloro-3-(3-fluorophenyl)-1,5-dioxo-2,3-dihydroimidazo[1,5- |
| | a]pyridin-6-yllamino]pyrimidin-4-yl ]cyclopropanecarboxamide |
| B92 | 6-[(6-aminopyrimidin-4-yl)amino]-3-(3-chlorophenyl)-3,8-dimethyl-2H- |
| | imidazo[1,5-a]pyridine-1,5-dione |
| B93 | 6-[(6-aminopyrimidin-4-yl)amino]-8-chloro-3-(3-chlorophenyl)-3-methyl- |
| | 2H-imidazo[1,5-a]pyridine-1,5-dione |
| B94 | 6-[(6-aminopyrimidin-4-yl)amino]-8-chloro-spiro[2H-imidazo[1,5- |
| | alpyridine-3,1′-cyclopentane]-1,5-dione |
| B95 | N-[6-[(8-chloro-1,1′,5-trioxo-spiro[2H-imidazo[1,5-a]pyridine-3,3′- |
| | thietane]-6-yl)amino]pyrimidin-4-yl]cyclopropanecarboxamide |
| B96 | 8-chloro-1′,l′-dimethyl-6-(pyrimidin-4-ylamino)spiro[2H-imidazo[1,5- |
| | alpyridine-3,2′-cyclohexane]-1,5-dione |
| B97 | N-(6-((8-chloro-3-methyl-1,5-dioxo-3-phenyl-1,2,3,5- |
| | tetrahydroimidazo[1,5-a]pyridin-6-yl)amino)pyrimidin-4- |
| | yl)cyclopropanecarboxamide |
| B98 | 6-[(6-aminopyrimidin-4-yl)amino]-8-methyl-spiro[2H-imidazo[1,5- |
| | alpyridine-3,1′-cyclopentane]-1,5-dione |
| B99 | 6-[(6-aminopyrimidin-4-yl)amino]-8-chloro-4',4'-difluoro-spiro[2H- |
| | imidazo[1,5-a]pyridine-3,1′-cyclohexane]-1,5-dione |
| B100 | 8-chloro-6-((7-cyclopropylpyrido[4,3-d]pyrimidin-4-yl)amino)-3,3- |
| | dimethyl-2,3-dihydroimidazo[1,5-a]pyridine-1,5-dione |
| B101 | 6-((6-aminopyrimidin-4-yl)amino)-3-(3-fluorophenyl)-3,8-dimethyl-2,3- |
| | dihydroimidazo[1,5-a]pyridine-1,5-dione |
| B102 | 6-((6-aminopyrimidin-4-yl)amino)-3,3,8-trimethyl-2,3- |
| | dihydroimidazo[1,5-a ]pyridine-1,5-dione |
| B103 | 6-[(6-aminopyrimidin-4-yl)amino]-4′,4′-difluoro-8-methyl-spiro[2H- |
| | imidazo[1,5-a]pyridine-3,1′-cyclohexane]-1,5-dione |
| B104 | 8-chloro-6-(pyrimidin-4-ylamino)spiro[2H-imidazo[1,5-a]pyridine-3,4′- |
| | piperidine]-1,5-dione |
| B105 | 6-[(6-aminopyrimidin-4-yl)amino]-3-tert-butyl-8-chloro-3-methyl-2H- |
| | imidazo[1,5-a]pyridine-1,5-dione |
| B106 | 6-((6-aminopyrimidin-4-yl)amino)-8-methyl-2H-spiro[imidazo[1,5- |
| | alpyridine-3,4′-piperidine]-1,5-dione |
| B107 | 6′-((6-aminopyrimidin-4-yl)amino)-8′-methyl-2′H-spiro[cyclohexane-1,3′- |
| | imidazo[1,5-a]pyridine]-1′,5′-dione |
| B108 | 6-((6-aminopyrimidin-4-yl)amino)-8-chloro-2H-spiro[imidazo[1,5- |
| | alpyridine-3,4′-piperidine]-1,5-dione |
| B109 | 8-chloro-6-(pyrimidin-4-ylamino)spiro[2H-imidazo[1,5-a]pyridine-3,1′- |
| | cyclohexane]-1,5-dione |
| B110 | 8-chloro-4′,4′-difluoro-6-(pyrimidin-4-ylamino)spiro[2H-imidazo[1,5- |
| | alpyridine-3,1′-cyclohexane]-1,5-dione |
| B111 | 6-[(6-aminopyrimidin-4-yl)amino]-6′,8-dichloro-spiro[2H-imidazo[1,5- |
| | alpyridine-3,1′-indane]-1,5-dione |
| B112 | 8-chloro-6-[(5-fluoropyrimidin-4-yl)amino]spiro[2H-imidazo[1,5- |
| | alpyridine-3,1′-cyclohexane]-1,5-dione |
| B113 | 8-chloro-6-(pyrimidin-4-ylamino)spiro[2H-imidazo[1,5-a]pyridine-3,2′- |
| | indane]-1,1′,5-trione |
| B114 | 6-((6-aminopyrimidin-4-yl)amino)-8-chloro-6′-fluoro-2′,3′-dihydro-2H- |
| | spiro[imidazo[1,5-a]pyridine-3,1′-indene]-1,5-dione |
| B115 | 8-chloro-6-[(5-chloropyrimidin-4-yl)amino]spiro[2H-imidazo[1,5- |
| | alpyridine-3,1′-cyclohexane]-1,5-dione |
| B116 | 8-chloro-6-[(6-methylpyrimidin-4-yl)amino]spiro[2H-imidazo[1,5- |
| | alpyridine-3,1′-cyclohexane]-1,5-dione |
| B117 | 6′-((7H-purin-6-yl)amino)-8′-chloro-2′H-spiro[cyclohexane-1,3′- |
| | imidazo[1,5-a]pyridine]-1′,5′-dione |
| B118 | 6-[(6-aminopyrimidin-4-yl)amino]-3-cyclopentyl-3-methyl-1,5-dioxo- |
| | imidazo[1,5-a]pyridine-2-carbonitrile |
| B119 | 6-[(6-aminopyrimidin-4-yl)amino]-8-chloro-3-(3-fluorophenyl)-2,3- |
| | dihydroimidazo[1,5-a ]pyridine-1,5-dione |
| B120 | 8′-chloro-2,2-dimethyl-6′-(pyrimidin-4-ylamino)-2′H-spiro[cyclobutane- |
| | 1,3′-imidazo[1,5-a]pyridine]-1′,5′-dione |
| B121 | 6′-((6-Aminopyrimidin-4-yl) amino)-8′-chloro-2,2-dimethyl-2′H- |
| | spiro[cyclobutane-1,3′-imidazo[1, 5-a ]pyridine]-1′,5′-dione |
| B122 | 6-[(6-amino-5-chloro-pyrimidin-4-yl)amino]-8-chloro-spiro[2H- |
| | imidazo[1,5-a]pyridine-3,1′-cyclohexane]- 1,5-dione |
| B123 | 6′-((7H-pyrrolo[2,3-d]pyrimidin-4-yl)amino)-8′-chloro-2′H- |
| | spiro[cyclohexane-1,3′-imidazo[1,5-a]pyridine]-1′,5′-dione |
| B124 | 8-chloro-3,3-dimethyl-6-(pyrido[4,3-d]pyrimidin-4-ylamino)-2,3- |
| | dihydroimidazo[1,5-a]pyridine-1,5-dione |
| B125 | 8′-chloro-6′-((2-methylpyrimidin-4-yl)amino)-2′H-spiro[cyclohexane-1,3′- |
| | imidazo[1,5-a]pyridine]-1′,5′-dione |
| B126 | 6-((6-aminopyrimidin-4-yl)amino)-8-chloro-2H-spiro[imidazo[1,5- |
| | alpyridine-3,6′-[1,4]diazepane]-1,5-dione |
| B127 | 6′-((1H-pyrazolo[3,4-d]pyrimidin-4-yl)amino)-8′-chloro-2′H- |
| | spiro[cyclohexane-1,3′-imidazo[1,5-a]pyridine]-1′,5′-dione |
| B128 | 6-[(6-aminopyrimidin-4-yl)amino]-8-chloro-1′-methyl-spiro[2H- |
| | imidazo[1,5-a]pyridine-3,4′-piperidine]-1,5-dione |
| B129 | 8-chloro-1′-methyl-6-(pyrimidin-4-ylamino)spiro[2H-imidazo[1,5- |
| | alpyridine-3,4′-piperidine]-1,5-dione |
| B130 | 1′,8-dimethyl-6-(pyrimidin-4-ylamino)-2H-spiro[imidazo[1,5-a]pyridine- |
| | 3,3′-piperidine]-1,5-dione |
| B131 | 6′-(pyrimidin-4-ylamino)-1′-thioxo-1′,2′-dihydro-5′H-spiro[cyclohexane- |
| | 1,3′-imidazo[1,5-a]pyridin]-5′-one |
| B132 | 8′-chloro-1′-(methoxyimino)-6′-(pyrimidin-4-ylamino)-1′,2′-dihydro-5′H- |
| | spiro[cyclohexane-1,3′-imidazo[1,5-a]pyridin]-5′-one |
| B133 | 8′-chloro-6′-(pyrimidin-4-ylamino)-5′-thioxo-2′H-spiro[cyclohexane-1,3′- |
| | imidazo[1,5-a]pyridin]-1′(5′H)-one |
| B134 | 8′-chloro-5′-(methoxyimino)-6′-(pyrimidin-4-ylamino)-2′H- |
| | spiro[cyclohexane-1,3′-imidazo[1,5-a]pyridin]-1′(5′H)-one |
| B135 | 8′-chloro-2′-cyclopropyl-6′-(pyrimidin-4-ylamino)-2′H-spiro[cyclohexane- |
| | 1,3′-imidazo[1,5-a]pyridine]-1′,5′-dione |
| B136 | 8′-chloro-2′-(pyridin-3-yl)-6′-(pyrimidin-4-ylamino)-2′H- |
| | spiro[cyclohexane-1,3′-imidazo[1,5-a]pyridine]-1′,5′-dione |
| B137 | 7′-fluoro-6′-(pyrimidin-4-ylamino)-2′H-spiro[cyclohexane-1,3′- |
| | imidazo[1,5-a]pyridine]-1′,5′-dione |
| B138 | 8-phenyl-6-(pyrimidin-4-ylamino)spiro[2H-imidazo[1,5-a]pyridine-3,1′- |
| | cyclohexane]-1,5-dione |
| B139 | 8′-(oxetan-2-yl)-6′-(pyrimidin-4-ylamino)-2′H-spiro[cyclohexane-1,3′- |
| | imidazo[1,5-a]pyridine]-1′,5′-dione |
| B140 | 8-chloro-3-methyl-6-(pyrimidin-4-ylamino)-3-vinyl-2,3- |
| | dihydroimidazo[1,5-a]pyridine-1,5-dione |
| B141 | 8-chloro-3-methyl-3-(prop-1-yn-1-yl)-6-(pyrimidin-4-ylamino)-2,3- |
| | dihydroimidazo[1,5-a]pyridine-1,5-dione |
| B142 | 4-((8′-chloro-1′,5′-dioxo-1′,5′-dihydro-2′H-spiro[cyclohexane-1,3′- |
| | imidazo[1,5-a]pyridin]-6′-yl)amino)pyrimidine-5-carbonitrile |
| B143 | 8′-chloro-6′-((5-(1-methyl-1H-pyrazol-4-yl)pyrimidin-4-yl)amino)-2′H- |
| | spiro[cyclohexane-1,3′-imidazo[1,5-a]pyridine]-1′,5′-dione |
| B144 | 8′-chloro-6′-((5-ethynylpyrimidin-4-yl)amino)-2′H-spiro[cyclohexane-1,3′- |
| | imidazo[1,5-a]pyridine]-1′,5′-dione |
| B145 | 6′-((1H-pyrazolo[3,4-d]pyrimidin-4-yl)amino)-8′-chloro-2′H- |
| | spiro[cyclohexane-1,3′-imidazo[1,5-a]pyridine]-1′,5′-dione |
| B146 | 8-chloro-6-(3H-triazolo[4,5-d]pyrimidin-7-ylamino)spiro[2H-imidazo[1,5- |
| | alpyridine-3,1′-cyclohexane]-1,5-dione |
| B147 | 8-((6-aminopyrimidin-4-yl)amino)-10-methyl-2,3,4,5- |
| | tetrahydropyrido[1,2-a][1,4]diazepine-1,7-dione |
| B148 | 3,3-di-tert-butyl-8-chloro-6-(pyrimidin-4-ylamino)-2,3- |
| | dihydroimidazo[1,5-a]pyridine-1,5-dione |
| B149 | 8-chloro-6-(pyrimidin-4-ylamino)-3,3-di(thiophen-2-yl)-2,3- |
| | dihydroimidazo[1,5-a ]pyridine-1,5-dione |
| B150 | 8-chloro-6-(pyrimidin-4-ylamino)-3,3-di(thiophen-3-yl)-2,3- |
| | dihydroimidazo[1,5-a]pyridine-1,5-dione |
| B151 | 8-chloro-3,3-dipropyl-6-(pyrimidin-4-ylamino)-2H-imidazo[1,5-a]pyridine- |
| | 1,5-dione |
| B152 | 3,3-bis(2-aminoethyl)-8-chloro-6-(pyrimidin-4-ylamino)-2,3- |
| | dihydroimidazo[1,5-a]pyridine-1,5-dione |
| B153 | 8-chloro-3,3-bis(2-hydroxyethyl)-6-(pyrimidin-4-ylamino)-2,3- |
| | dihydroimidazo[1,5-a]pyridine-1,5-dione |
| B154 | 3,3-bis(aminomethyl)-8-chloro-6-(pyrimidin-4-ylamino)-2,3- |
| | dihydroimidazo[1,5-a ]pyridine-1,5-dione |
| B155 | 8-chloro-1′-(2-hydroxyethyl)-6-(pyrimidin-4-ylamino)-2H- |
| | spiro[imidazo[1,5-a]pyridine-3,4′-piperidine]-1,5-dione |
| B156 | 8-chloro-1′-(2,2-difluoroethyl)-6-(pyrimidin-4-ylamino)spiro[2H- |
| | imidazo[1,5-a]pyridine-3,4′-piperidine]-1,5-dione |
| B157 | 8-chloro-6-(pyrimidin-4-ylamino)-1′-(2,2,2-trifluoroethyl)-2H- |
| | spiro[imidazo[1,5-a]pyridine-3,4′-piperidine]-1,5-dione |
| B158 | 8′-chloro-8-methyl-6′-(pyrimidin-4-ylamino)-2'H-8- |
| | azaspiro[bicyclo[3.2.1]octane-3,3′-imidazo[1,5-a]pyridine]-1′,5′-dione |
| B159 | 8′-chloro-6′-(pyrimidin-4-ylamino)-2′H-8-azaspiro[bicyclo[3.2.1 ]octane- |
| | 3,3′-imidazo[1,5-a]pyridine]-1′,5′-dione |
| B160 | 2-(8-chloro-1,5-dioxo-6-(pyrimidin-4-ylamino)-1,5-dihydro-2H- |
| | spiro[imidazo[1,5-a]pyridine-3,4′-piperidin]-1′-yl)acetonitrile |
| B161 | 8-chloro-1′-(pyrimidin-4-yl)-6-(pyrimidin-4-ylamino)-2H- |
| | spiro[imidazo[ 1,5-a]pyridine-3,4′-piperidine]-1,5-dione |
| B162 | 8-chloro-4′-(methylamino)-6-(pyrimidin-4-ylamino)spiro[2H-imidazo[1,5- |
| | alpyridine-3,1′-cyclohexane]-1,5-dione |
| B163 | 1′-acetyl-8-chloro-6-(pyrimidin-4-ylamino)-2H-spiro[imidazo[1,5- |
| | alpyridine-3,4′-piperidine]-1,5-dione |
| B164 | 8′-chloro-1′,5′-dioxo-6′-(pyrimidin-4-ylamino)-1′,5′-dihydro-2′H- |
| | spiro[cyclohexane-1,3′-imidazo[1,5-a]pyridine]-4-carbonitrile |
| B165 | 8-chloro-3,3-dimethyl-6-(pyrido[3,4-d]pyrimidin-4-ylamino)-2,3- |
| | dihydroimidazo[1,5-a]pyridine-1,5-dione |
| B166 | 8-chloro-3,3-dimethyl-6-(pyrimido[5,4-c]pyridazin-8-ylamino)-2,3- |
| | dihydroimidazo[1,5-a]pyridine-1,5-dione |
| B167 | 8-chloro-3,3-dimethyl-6-(pyrimido[5,4-d]pyrimidin-4-ylamino)-2,3- |
| | dihydroimidazo[1,5-a]pyridine-1,5-dione |
| B168 | (Z)-8-chloro-6-((6-(2-cyclopropyl-3,3,3-trifluoroprop-1-en-1-yl)pyrimidin- |
| | 4-yl)amino)-3-(3-fluorophenyl)-3-methyl-2,3-dihydroimidazo[1,5- |
| | alpyridine-1,5-dione |
| B169 | 6′-((6-amino-5-fluoropyrimidin-4-yl)amino)-8'-chloro-2′H- |
| | spiro[cyclohexane-1,3′-imidazo[1,5-a]pyridine]-1′,5′-dione |
| B170 | 6-[(6-aminopyrimidin-4-yl)amino]-1′,8-dimethyl-spiro[2H-imidazo[1,5- |
| | alpyridine-3,3′-piperidine]-1,5-dione |
| B171 | 8-chloro-6-(pyrimidin-4-ylamino)-2′,3′-dihydro-1′H,2H-spiro[imidazo[1,5- |
| | alpyridine-3,4′-isoquinoline]-1,5-dione |
| B172 | 6-((6-aminopyrimidin-4-yl)amino)-8-chloro-2′,3′-dihydro-1′H,2H- |
| | spiro[imidazo[1,5-a]pyridine-3,4′-isoquinoline]-1,5-dione |
| B173 | 8-chloro-6′-fluoro-6-(pyrimidin-4-ylamino)-2′,3′-dihydro-1′H,2H- |
| | spiro[imidazo[1,5-a]pyridine-3,4′-isoquinoline]-1,5-dione |
| B174 | 6-((6-aminopyrimidin-4-yl)amino)-8-chloro-6′-fluoro-2′,3′-dihydro- |
| | 1′H,2H-spiro[imidazo[1,5-a]pyridine-3,4′-isoquinoline]-1,5-dione |
| B175 | 8-chloro-6-(pyrimidin-4-ylamino)-2H-spiro[imidazo[1,5-a]pyridine-3,3′- |
| | indoline]-1,5-dione |
| B176 | 8-chloro-5′-fluoro-6-(pyrimidin-4-ylamino)-2H-spiro[imidazo[1,5- |
| | alpyridine-3,3′-indoline1-1,5-dione |
| B177 | 8-chloro-4′,4′-difluoro-6-(pyrimidin-4-ylamino)-2H-spiro[imidazo[1,5- |
| | alpyridine-3,3′-piperidine]-1,5-dione |
| B178 | 6-((6-aminopyrimidin-4-yl)amino)-8-chloro-4′,4′-difluoro-2H- |
| | spiro[imidazo[1,5-a]pyridine-3,3′-piperidine]-1,5-dione |
| B179 | 8′-chloro-2,2-dimethyl-6′-(pyrimidin-4-ylamino)-4,5-dihydro-2H,2′H- |
| | spiro[furan-3,3′-imidazo[1,5-a]pyridine]-1′,5′-dione |
| B180 | 6′-((6-aminopyrimidin-4-yl)amino)-8′-chloro-2,2-dimethyl-4,5-dihydro- |
| | 2H,2′H-spiro[furan-3,3′-imidazo[1,5-a ]pyridine]-1′,5′-dione |
| B181 | 3,3-dimethyl-1,5-dioxo-6-(pyrimidin-4-ylamino)-2H-imidazo[1,5- |
| | alpyridine-8-carbonitrile |
| B182 | 6′-((2-aminopyridin-4-yl)amino)-8′-methyl-2′H-spiro[cyclohexane-1,3′- |
| | imidazo[1,5-a]pyrazine]-1′,5′-dione |
| B183 | 3′-amino-6-((6-aminopyrimidin-4-yl)amino)-8-chloro-6′-fluoro-21,3′- |
| | dihydro-2H-spiro[imidazo[1,5-a]pyridine-3,1′-indene]-1,5-dione |
| B184 | N-(6-((8-chloro-3-(3-chlorophenyl)-3-methyl-1,5-dioxo-1,2,3,5- |
| | tetrahydroimidazo[1,5-a]pyridin-6-yl)amino)pyrimidin-4-yl)cyanamide |
| B185 | 8′-chloro-6′-(pyrimidin-4-ylamino)-2′H-spiro[bicyclo[2.2.1]heptane-7,3′- |
| | imidazo[1,5-a]pyridine]-1′,5′-dione |
| B186 | 6′-((6-aminopyrimidin-4-yl)amino)-8′-chloro-2′H- |
| | spiro[bicyclo[2.2.1]heptane-7,3′-imidazo[1,5-a]pyridine]-1′,5′-dione |
| B187 | 8-chloro-6-[(5-methoxypyrimidin-4-yl)amino]spiro[2H-imidazo[1,5- |
| | alpyridine-3,1′-cyclohexane]-1,5-dione |
| B188 | 6′-((6-amino-5-ethylpyrimidin-4-yl)amino)-8′-chloro-2′H- |
| | spiro[cyclohexane-1,3′-imidazo[ 1,5-a]pyridine]-1′,5′-dione |
| B189 | 6′-((6-amino-5-isopropylpyrimidin-4-yl)amino)-8′-chloro-2′H- |
| | spiro[cyclohexane-1,3′-imidazo[1,5-a]pyridine]-1′,5′-dione |
| B190 | 8′-chloro-6′-(7H-pyrrolo[2,3-d]pyrimidin-7-yl)-2′H-spiro[cyclohexane-1,3′- |
| | imidazo[1,5-a]pyridine]-1′,5′-dione |
| B191 | 8′-chloro-6′-(pyrimidin-4-ylamino)-2-(trifluoromethyl)-2′H- |
| | spiro[cyclohexane-1,3′-imidazo[1,5-a]pyridine]-1′,5′-dione |
| B192 | 8-chloro-3-methyl-3-(2-methylprop-1-enyl)-6-(pyrimidin-4-ylamino)-2H- |
| | imidazo[1,5-a]pyridine-1,5-dione |
| B193 | 6′-((6-amino-5-methylpyrimidin-4-yl)amino)-8′-chloro-2′H- |
| | spiro[cyclohexane-1,3′-imidazo[1,5-a]pyridine]-1′,5′-dione |
| B194 | 8′-chloro-6′-((5-ethylpyrimidin-4-yl)amino)-2′H-spiro[cyclohexane-1,3′- |
| | imidazo[1,5-a]pyridine]-1′,5′-dione |
| B195 | 6′-((6-amino-5-methoxypyrimidin-4-yl)amino)-8′-chloro-2′H- |
| | spiro[cyclohexane-1,3′-imidazo[1,5-a]pyridine]-1′,5′-dione |
| B196 | 8′-chloro-6′-((5-ethoxypyrimidin-4-yl)amino)-2′H-spiro[cyclohexane-1,3′- |
| | imidazo[1,5-a]pyridine]-1′,5′-dione |
| B197 | 8′-chloro-6′-((5-isopropoxypyrimidin-4-yl)amino)-2′H-spiro[cyclohexane- |
| | 1,3′-imidazo[1,5-a]pyridine]-1′,5′-dione |
| B198 | 8′-chloro-2′-cyclopentyl-6′-(pyrimidin-4-ylamino)-2′H-spiro[cyclohexane- |
| | 1,3′-imidazo[1,5-a]pyridine]-1′, 5′-dione |
| B199 | 6′-((6-aminopyrimidin-4-yl)amino)-8′-methyl-5′-thioxo-2′H- |
| | spiro[cyclopentane-1,3′-imidazo[1,5-a]pyridin]-1′(5′H)-one |
| B200 | 6′-((6-aminopyrimidin-4-yl)(methyl)amino)-8′-chloro-2′H- |
| | spiro[cyclopentane-1,3′-imidazo[1,5-a]pyridine]-1′,5′-dione |
| B201 | 6′-(pyrimidin-4-ylamino)-8′-(tetrahydro-2H-pyran-4-yl)-2′H- |
| | spiro[cyclohexane-1,3′-imidazo[1,5-a]pyridine]-1′,5′-dione |
| B202 | 6′-((6-amino-5-hydroxypyrimidin-4-yl)amino)-8′-methyl-2′H- |
| | spiro[cyclohexane-1,3′-imidazo[1,5-a]pyridine]-1',5′-dione |
| B203 | 6′-((6-amino-2-hydroxypyrimidin-4-yl)amino)-8′-methyl-2′H- |
| | spiro[cyclohexane-1,3′-imidazo[1,5-a]pyridine]-1′,5′-dione |
| B204 | 6′-((6-aminopyrimidin-4-yl)amino)-2-hydroxy-8′-methyl-2′H- |
| | spiro[cyclohexane-1,3′-imidazo[1,5-a]pyridine]-1′,5′-dione |
| B205 | 6′-((6-aminopyrimidin-4-yl)amino)-3-hydroxy-8'-methyl-2'H- |
| | spiro[cyclohexane-1,3′-imidazo[1,5-a]pyridine]-1',5'-dione |
| B206 | 6′-((6-aminopyrimidin-4-yl)amino)-4-hydroxy-8′-methyl-2′H- |
| | spiro[cyclohexane-1,3′-imidazo[1,5-a]pyridine]-1′,5′-dione |
| B207 | 6′-((6-aminopyrimidin-4-yl)amino)-8′-(hydroxymethyl)-2′H- |
| | spiro[cyclohexane-1,3′-imidazo[1,5-a]pyridine]-1′,5′-dione |
| B208 | 6′-((5-cyclopropylpyrimidin-4-yl)amino)-8′-methyl-2′H-spiro[cyclohexane- |
| | 1,3′-imidazo[1,5-a]pyridine]-1′,5′-dione |
| B209 | 8′-methyl-6′~(pyrimidin-4-yloxy)-2′H-spiro[cyclohexane-1,3′-imidazo[1,5- |
| | alpyridine]-1′,5′-dione |
| B210 | 8′-methyl-6′-(pyrimidin-4-ylthio)-2′H-spiro[cyclohexane-1,3′-imidazo[1,5- |
| | alpyridine]-1′,5′-dione |
| B211 | 8′-methyl-6′-(pyrimidine-4-carbonyl)-2′H-spiro[cyclohexane-1,3′- |
| | imidazo[1,5-a]pyridine]-1′,5′-dione |
| B212 | 8′-methyl-6′-(pyrimidin-4-ylmethyl)-2′H-spiro[cyclohexane-1,3′- |
| | imidazo[1,5-a]pyridine]-1′,5′-dione |
| B213 | 5-((6-aminopyrimidin-4-yl)amino)-3-chloro-1-isobutyl-6-oxo-1,6- |
| | dihydropyridine-2-carboxamide |
| B214 | 5-((6-aminopyrimidin-4-yl)amino)-3-chloro-6-oxo-1,6-dihydropyridine-2-carboxamide |
| B215 | 3′-(pyrimidin-4-ylamino)-4′H-spiro[cyclohexane-1,6′-imidazo[1,5- |
| | alpyrimidine]-4′,8′(7'H)-dione |
| B216 | 8′-methyl-6′-(pyrimidin-4-ylamino)-2′H-spiro[cyclohexane-1,3′- |
| | imidazo[1,5-a]pyrazine]-1′,5′-dione |
| B217 | 8-((6-aminopyrimidin-4-yl)amino)-10-methyl-2,3,4,5- |
| | tetrahydropyrido[1,2-a][1,4]diazepine-1,7-dione |
| B218 | 6-amino-4-((8'-methyl-1′,5′-dioxo-1′,5′-dihydro-2'H-spiro[cyclohexane- |
| | 1,3′-imidazo[1,5-a]pyridin]-6'-yl)amino)pyrimidine 1-oxide |
| B219 | 6-((6-aminopyrimidin-4-yl)amino)-1′,8-dimethyl-2H-spiro[imidazo[1,5- |
| | alpyridine-3,4′-piperidine]-1,5-dione |
| B220 | 6-((6-amino-5-methylpyrimidin-4-yl)amino)-1′,8-dimethyl-2H- |
| | spiro[imidazo[1,5-a]pyridine-3,4′-piperidine]-1,5-dione |
| B221 | 6′-((2-chloropyrimidin-4-yl)thio)-8′-methyl-2′H-spiro[cyclohexane-1,3′- |
| | imidazo[1,5-a]pyridine]-1′,5′-dione |
| B222 | 6′-((6-amino-5-methylpyrimidin-4-yl)amino)-8′-methyl-2′H- |
| | spiro[cyclohexane-1,3′-imidazo[1,5-a]pyridine]-1′,5′-dione |
| B223 | 6′-((6-amino-5-chloropyrimidin-4-yl)amino)-8′-methyl-2'H- |
| | spiro[cyclohexane-1,3′-imidazo[ 1,5-a]pyridine]-1′,5′-dione |
| B224 | 6′-((2-chloropyrimidin-4-yl)oxy)-8′-methyl-2′H-spiro[cyclohexane-1,3′- |
| | imidazo[1,5-a]pyridine]-1′,5′-dione |
| B225 | 2-(6-((6-aminopyrimidin-4-yl)amino)-8-methyl-1,5-dioxo-1,5-dihydro-2H- |
| | spiro[imidazo[1,5-a]pyridine-3,4′-piperidini-1′-yl)acetonitrile |
| B226 | 6-((6-aminopyrimidin-4-yl)amino)-1′-(2,2-difluoroethyl)-8-methyl-2H- |
| | spiro[imidazo[1,5-a]pyridine-3,4′-piperidine]-1,5-dione |
| B227 | 6-((5-methoxypyrimidin-4-yl)amino)-1′,8-dimethyl-2H-spiro[imidazo[1,5- |
| | alpyridine-3,4′-piperidine]-1,5-dione |
| B228 | 6-((6-amino-5-methoxypyrimidin-4-yl)amino)-1′,8-dimethyl-2H- |
| | spiro[imidazo[1,5-a]pyridine-3,4′-piperidine]-1,5-dione |
| B229 | 2-(6-((5-methoxypyrimidin-4-yl)amino)-8-methyl-1,5-dioxo-1,5-dihydro- |
| | 2H-spiro[imidazo[1,5-a]pyridine-3,4′-piperidin]-1′-yl)acetonitrile |
| B230 | 1′-(2,2-difluoroethyl)-6-((5-methoxypyrimidin-4-yl)amino)-8-methyl-2H- |
| | spiro[imidazo[1,5-a]pyridine-3,4′-piperidine]-1,5-dione |
| B231 | 6′-((6-amino-5-methoxypyrimidin-4-yl)amino)-8′-methyl-2′H- |
| | spiro[cyclohexane-1,3′-imidazo[ 1,5-a]pyridine]-1′,5′-dione |
| B232 | 6-((6-aminopyrimidin-4-yl)amino)-8-methyl-1′-(2,2,2-trifluoroethyl)-2H- |
| | spiro[imidazo[1,5-a]pyridine-3,4′-piperidine]-1,5-dione |
| B233 | 6-((6-amino-5-methylpyrimidin-4-yl)amino)-1′-(2,2-difluoroethyl)-8- |
| | methyl-2H-spiro[imidazo[1,5-a]pyridine-3,4′-piperidine]-1,5-dione |
| B234 | 6-((6-amino-5-methoxypyrimidin-4-yl)amino)-8-methyl-1′~(2,2,2- |
| | trifluoroethyl)-2H-spiro[imidazo[1,5-a]pyridine-3,4′-piperidine]-1,5-dione |
| B235 | 6-((5-methoxypyrimidin-4-yl)amino)-8-methyl-1′-(2,2,2-trifluoroethyl)- |
| | 2H-spiro[imidazo[1,5-a]pyridine-3,4′-piperidine]-1,5-dione |
| B236 | 6-((6-amino-5-methylpyrimidin-4-yl)amino)-8-methyl-1′-(2,2,2- |
| | trifluoroethyl)-2H-spiro[imidazo[1,5-a]pyridine-3,4′-piperidine]-1,5-dione |
| B237 | 6-((6-amino-5-chloropyrimidin-4-yl)amino)-1′,8-dimethyl-2H- |
| | spiro[imidazo[1,5-a]pyridine-3,4′-piperidine]-1,5-dione |
| B238 | 2-(6-((6-amino-5-chloropyrimidin-4-yl)amino)-8-methyl-1,5-dioxo-1,5- |
| | dihydro-2H-spiro[imidazo[1,5-a ]pyridine-3,4′-piperidin]-1′-yl)acetonitrile |
| B239 | 6-((6-amino-5-methylpyrimidin-4-yl)amino)-8-methyl-2′,3′,5′,6′- |
| | tetrahydro-2H-spiro[imidazo[1,5-a]pyridine-3,4′-pyran]-1,5-dione |
| B240 | 2-(6-((6-amino-5-methylpyrimidin-4-yl)amino)-8-methyl-1,5-dioxo-1,5- |
| | dihydro-2H-spiro[imidazo[1,5-a]pyridine-3,4′-piperidin]-1′-yl)acetonitrile |
| B241 | 6-((6-amino-5-chloropyrimidin-4-yl)amino)-1′-(2,2-difluoroethyl)-8- |
| | methyl-2H-spiro[imidazo[1,5-a]pyridine-3,4′-piperidine]-1,5-dione |
| B242 | 6-((6-amino-5-chloropyrimidin-4-yl)amino)-8-methyl-1′-(2,2,2- |
| | trifluoroethyl)-2H-spiro[imidazo[1,5-a]pyridine-3,4′-piperidine]-1,5-dione |
| B243 | 2-(6-((6-amino-5-methoxypyrimidin-4-yl)amino)-8-methyl-1,5-dioxo-1,5- |
| | dihydro-2H-spiro[imidazo[1,5-a]pyridine-3,4′-piperidin]-1′-yl)acetonitrile |
| B244 | 1′-(2,2-difluoroethyl)-6-((5-methoxypyrimidin-4-yl)amino)-8-methyl-2H- |
| | spiro[imidazo[1,5-a]pyridine-3,4′-piperidine]-1,5-dione |
| B245 | 3′-((6-aminopyrimidin-4-yl)amino)-1′-methylspiro[cyclohexane-1,5′- |
| | pyrrolo[3,4-b]pyridine]-4′,7′(1′H,6′H)-dione |
| B246 | 6′-((6-aminopyrimidin-4-yl)amino)-8′-methyl-2′H-spiro[cyclohexane-1,3′- |
| | indolizine]-1′,5′-dione |
| B247 | 6′-((6-amino-5-methylpyrimidin-4-yl)amino)-8′-methyl-2′H- |
| | spiro[cyclohexane-1,3′-indolizine]- 1′,5′-dione |

### Compositions and Methods

In another aspect, the invention provides a pharmaceutical composition comprising a type interferon inhibitor and one or more therapeutic agent for treating pain associated with Rheumatoid Arthritis in a patient, and/or one or more therapeutic agent for treating Rheumatoid Arthritis, in combination with a pharmaceutically-acceptable carrier, diluent or excipient.

For example, in a preferred embodiment the invention provides a pharmaceutical composition comprising an MNK inhibitor and one or more therapeutic agent for treating pain associated with Rheumatoid Arthritis in a patient, and/or one or more therapeutic agent for treating Rheumatoid Arthritis, in combination with a pharmaceutically-acceptable carrier, diluent or excipient. The MNK inhibitor may be a small molecule, or an antibody or part thereof; but is preferably selected from the group consisting of: eFT508; 4ET-03-053; BAY1143269; ETC-1907206; and derivatives thereof.

By “therapeutic agents for treating pain associated with RA in a patient”, we include the meaning of therapeutic agents that are known and/or clinically approved, therapeutic agents under clinical trial, or therapeutic agents being developed that are or will be used to treat pain associated with RA. In one embodiment, the therapeutic agent for treating pain associated with RA in a patient is any of the pain treatments listed herein.

Without being bound by theory, type I interferon inhibitors can complement therapeutic agents for treating pain associated with RA in a patient. As described herein, there are many drawbacks and side-effects of the currently approved pain treatments and therefore combination therapies with type I interferon inhibitors could be useful for treating pain associated with RA.

By “therapeutic agents for treating RA”, we include the meaning of therapeutic agents that are known and/or clinically approved, therapeutic agents under clinical trial, or therapeutic agents being developed that are or will be used to treat or prevent RA. A therapeutic agent is considered effective at treating RA when the patient enters the inactive RA inflammatory disease state and/or remission.

Therapeutic agents for treating RA may include but are not limited to ‘classical’ DMARDs such as methotrexate (also called amethopterin), leflunomide, hydroxychloroquine and/or sulfasalazine. As will be appreciated, RA patients are often given a combination of these as initial treatment. If RA disease is not controlled using classical DMARDs, methotrexate is often co-administered with ‘biological’ DMARDs, such as abatacept (Orencia), adalimumab (Humira), anakinra (Kineret), certolizumab (Cimzia), etanercept (Enbrel), golimumab (Simponi), infliximab (Remicade), rituximab (Rituxan), sarilumab (Kevzara), tocilizumab (Actemra), or a combination thereof. Furthermore, if both ‘classical’ and ‘biological’ DMARDs have been ineffective, then ‘targeted synthetic’ DMARDs may be used. Such ‘targeted synthetic’ DMARDs may include Janus kinases (JAKs) inhibitors such as baricitinib (Olumiant), tofacitinib (Xeljanz), upadacitinib (Rinvoq), or a combination thereof.

Without being bound by theory, type I interferon inhibitors can complement therapeutic agents for treating RA. As described herein, many of the therapeutic agents for treating RA are ineffective or sub-optimal at treating the pain component of RA despite their effectiveness and treating the inflammatory component of RA. Therefore, type I interferon inhibitors could be administered in combination with a therapeutic agent for treating RA to allow simultaneous management of pain and of the inflammatory disease activity.

The pharmaceutical composition in accordance with the invention may be administered with suitable pharmaceutically acceptable carriers, excipients, and other agents that are incorporated into formulations to provide improved transfer, delivery, tolerance, and the like.

By “pharmaceutically-acceptable”, we include that the formulation is sterile and pyrogen free. Suitable pharmaceutically acceptable carriers, excipients or diluents are well known in the art of pharmacy. The pharmaceutically acceptable carriers, excipients or diluents must be “acceptable” in the sense of being compatible with the agent of the invention and not deleterious to the recipients thereof. Typically, the pharmaceutically acceptable carriers, excipients or diluents will be water or saline which will be sterile and pyrogen free; however, other pharmaceutically acceptable carriers, excipients or diluents may be used.

Appropriate pharmaceutically-acceptable carrier, excipient or diluent materials that may be employed in compositions of the invention include relevant materials that, in the appropriate combination, are suitable (and/or approved) for pharmaceutical use and/or delivery, and are capable of maintaining their physical and/or chemical integrity, and/or do not affect the physical and/or chemical integrity of any active ingredients and/or any other ingredients that are or may be present in the composition under normal storage conditions.

By “pharmaceutically acceptable carriers”, we also include excipients or stabilisers that are non-toxic to the cell or mammal being exposed thereto at the dosages and concentrations employed. Often the pharmaceutically acceptable carrier is an aqueous pH buffered solution. Examples of pharmaceutically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or non-ionic surfactants such as TWEEN™, polyethylene glycol (PEG), and PLURONICS™ or polyethylene glycol (PEG).

By “diluent”, we include the meaning of one which is pharmaceutically acceptable (i.e. safe and non-toxic for administration to an individual, such as a human) and is useful for the preparation of a liquid formulation, such as a formulation reconstituted after lyophilisation. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

In a further aspect, the invention provides the pharmaceutical composition as defined herein for use in treating or preventing pain associated with Rheumatoid Arthritis in a patient. In a preferred embodiment of that aspect of the invention, the pharmaceutical composition is one that comprises an MNK inhibitor, such as an inhibitor selected from: eFT508; 4ET-03-053; BAY1143269; ETC-1907206; and derivatives thereof.

In yet another aspect, the invention provides a use of the pharmaceutical composition as defined herein in the manufacture of a medicament for treating or preventing pain associated with Rheumatoid Arthritis in a patient. In a preferred embodiment of that aspect of the invention, the pharmaceutical composition is one that comprises an MNK inhibitor, such as an inhibitor selected from: eFT508; 4ET-03-053; BAY1143269; ETC-1907206; and derivatives thereof.

In yet a further aspect, the invention provides a method of treating or preventing pain associated with Rheumatoid Arthritis in a patient, comprising the step of administering the pharmaceutical composition as defined herein to the patient. In a preferred embodiment of that aspect of the invention, the pharmaceutical composition is one that comprises an MNK inhibitor, such as an inhibitor selected from: eFT508; 4ET-03-053; BAY1143269; ETC-1907206; and derivatives thereof.

As will be appreciated, the various delivery systems which can be used to administer the pharmaceutical composition will be those as defined for the administering the type I interferon inhibitor to the patient.

A clinician can determine the most appropriate administrative regimen for a patient based on factors such as the patient's weight, age, gender, diagnosis or prognosis, and the half-life of the administered therapeutic molecule. However, in general it may be suitable to treat a patient with a single dose, or multiple doses, of an effective amount of a type I interferon inhibitor according to the aspects herein, or a pharmaceutical composition according to the aspects herein. Where multiple administrations are made, these may be made at a rate of, for example, once, twice, three times, four times or more often per day, week or month, and may be continued fora period of time necessary and effective to treat or prevent the pain associated with RA in the patient and thereby obtain a therapeutically or prophylactically beneficial effect. For example, treatment may continue for 1, 2, 3, 4, 5, 6, 7, 8, 9, 10, 11, 12 or more days, weeks, months or years, or even for the rest of the life of the patient. In the case of the use of the type I interferon inhibitor, then administration would most typically be made weekly, or once or twice per month, and continue for as long as is clinically beneficial.

Also included, is a method of treating or preventing pain associated with RA and treating or preventing RA itself in a patient, comprising the step of administering the pharmaceutical composition as defined herein to the patient. This would allow simultaneous management of the disease and pain using the pharmaceutical composition as defined herein. In a preferred embodiment of that aspect of the invention, the pharmaceutical composition is one that comprises an MNK inhibitor, such as an inhibitor selected from: eFT508; 4ET-03-053; BAY1143269; ETC-1907206; and derivatives thereof.

In another aspect, the invention provides a kit comprising a type I interferon inhibitor and one or more therapeutic agent for treating Rheumatoid Arthritis. In a preferred embodiment of that aspect of the invention, the type I interferon inhibitor is an MNK inhibitor, such as an inhibitor selected from: eFT508; 4ET-03-053; BAY1143269; ETC-1907206; and derivatives thereof.

In a further aspect, the invention provides a method for identifying a patient who has pain associated with Rheumatoid Arthritis and is in need of treatment with a type I interferon inhibitor, the method comprising the steps of:
(a) providing a test sample from a patient who has pain associated with Rheumatoid Arthritis;
(b) determining the level of type I interferon signalling in the test sample; and
(c) identifying the patient as one in need of treatment with a type I interferon inhibitor on the basis of the determination in Step (b).

By “a patient in need of treatment with a type I interferon inhibitor”, we include the meaning of a patient who would potentially benefit from type I interferon inhibitor therapy. For example, where an individual has RA, such type I interferon inhibitor would improve the health (for example, by reducing or stopping the symptoms of pain). In another embodiment, where the patient does yet have pain, such type I interferon inhibitor therapy would reduce the chance of the patient starting to experience pain.

The term “test sample” includes any biological sample from the patient, to be tested in the methods and uses of the invention. It will be appreciated that the test sample may comprise one or more tissue, cell and/or biological fluid taken from (such as isolated from) the patient (e.g., blood, skin, synovium, synovial fluid, sensory ganglion, serum, plasma, serum plasma, urine, saliva, intestinal cells, biopsy (such as muscle biopsies), stool).

It will also be appreciated that the methods and uses of the invention may be performed using tissues, cells and/or biological fluids when present within an individual. Accordingly, the detection method of the invention can be used to detect a virus infection in a test sample in vitro as well as in vivo. Preferably, the test sample is serum plasma, which has preferably been isolated from the individual.

By the “level of type I interferon signalling”, we include the meaning of type I interferon intracellular signalling, levels of type I interferons, activation of type I interferon receptors, expression of type I interferon-stimulated genes, expression of type I interferon-repressed genes. Methods for determining the levels are discussed herein.

A method of patient stratification of diagnosed SLE uses the level of type I interferon signalling in a test sample to predict patients (i) at risk of greater disease severity and (ii) at risk of developing Lupus Nephritis. DxCollect® micro collective devices (from DxTerity®) measure the relative expression of four messenger RNAs (mRNAs) of type 1 Interferon responsive genes by PCR and capillary electrophoresis. The patient's interferon status is then classified as high (above −0.5) or as low/normal (equal or below −0.5). In some embodiments, such a micro collective device may be used for identifying a patient who has pain associated with RA and is in need of treatment with a type I interferon inhibitor.

Preferably, the method as defined herein further comprises the step of administering a type I interferon inhibitor to the patient. In a preferred embodiment of that aspect of the invention, the type I interferon inhibitor is an MNK inhibitor, such as an inhibitor selected from: eFT508; 4ET-03-053; BAY1143269; ETC-1907206; and derivatives thereof.

In a further aspect, the invention provides a type I interferon inhibitor for use, or a use, or a method, or a composition, or a kit, substantially as described herein with reference to the accompanying description, examples, figures and/or claims.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the above description and the accompanying drawings. It should be understood, however, that the above description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The listing or discussion in this specification of an apparently prior-published document should not necessarily be taken as an acknowledgement that the document is part of the state of the art or is common general knowledge.

### Exemplary Embodiments

The following enumerated embodiments, while non-limiting, are exemplary of certain aspects of this disclosure:
1. A type I interferon inhibitor for use in treating or preventing pain associated with Rheumatoid Arthritis in a patient.
2. Use of a type I interferon inhibitor in the manufacture of a medicament for treating or preventing pain associated with Rheumatoid Arthritis in a patient.
3. A method of treating or preventing pain associated with Rheumatoid Arthritis in a patient, comprising the step of administering a type I interferon inhibitor to the patient.
4. The type I interferon inhibitor for use, or the use, or the method, according to any of embodiments 1 to 3, wherein the pain is dysfunctional pain, such as inflammatory joint pain.
5. The type I interferon inhibitor for use, or the use, or the method, according to any of embodiments 1 to 3, wherein the pain is not inflammatory pain.
6. The type I interferon inhibitor for use, or the use, or the method, according to any preceding embodiment wherein the pain is not neuropathic pain or neuroplastic pain.
7. The type I interferon inhibitor for use, or the use, or the method, according to any preceding embodiment, wherein the pain is chronic pain; for example, pain that is present for three months or more, or six months or more, or twelve months or more.
8. The type I interferon inhibitor for use, or the use, or the method, according to any preceding embodiment, wherein the pain is one or more selected from the group consisting of: pain hypersensitivity; allodynia; hyperalgesia; arthralgia.
9. The type I interferon inhibitor for use, or the use, or the method, according to any preceding embodiment, wherein the pain is associated with and/or caused by:
   systemic inflammation; and/or
   local inflammation; and/or
   clinical inflammation.
10. The type I interferon inhibitor for use, or the use, or the method, according to any preceding embodiment, wherein the pain is not associated with and/or caused by Rheumatoid Arthritis inflammatory disease activity.
11. The type I interferon inhibitor for use, or the use, or the method, according to any preceding embodiment, wherein the pain is at an affected joint and/or opposite parts of an affected joint and/or cephalic parts of an affected joint and/or caudal parts of an affected joint.
12. The type I interferon inhibitor for use, or the use, or the method, according to any preceding embodiment, wherein the type I interferon inhibitor does not prevent or treat an inflammatory disease with increased type I interferon signalling.
13. The type I interferon inhibitor for use, or the use, or the method, according to any preceding embodiment, wherein:
   the pain is present along with disease inflammation; and/or
   the pain is present after the remission of disease inflammation.
14. The type I interferon inhibitor for use, or the use, or the method, according to any preceding embodiment, wherein the patient has received, or is receiving, pain treatment, but the pain persists and/or recurs and/or progresses.
15. The type I interferon inhibitor for use, or the use, or the method, according to embodiment 14, wherein the pain treatment is selected from the group consisting of: a nonsteroidal anti-inflammatory drug (NSAID), such as celecoxib, diclofenac, etoricoxib, ibuprofen, naproxen; a steroid, such as corticosteroid, glucocorticoid; acetaminophen; an opioid, such as codeine, dextropropoxyphene, tramadol; an antidepressant, such as tricyclic antidepressant; an anticonvulsant; or a combination thereof.
16. The type I interferon inhibitor for use, or the use, or the method, according to any preceding embodiment, wherein the pain is associated with and/or caused by increased type I interferon signalling in the patient.
17. The type I interferon inhibitor for use, or the use, or the method, according to embodiment 16, wherein increased type I interferon signalling comprises:
   increased type I interferon intracellular signalling in the patient
   increased levels of type I interferons in the patient;
   increased activation of type I interferon receptors in the patient;
   increased expression of one or more type I interferon-stimulated gene in the patient; and/or
   reduced expression of one or more type I interferon-repressed gene in the patient.
18. The type I interferon inhibitor for use, or the use, or the method, according to any preceding embodiment, wherein the type I interferon is selected from the group comprising: Interferon-alpha; Interferon-beta.
19. The type I interferon inhibitor for use, or the use, or the method, according to any preceding embodiment, wherein the pain is associated with increased number and/or activity of one or more active sensory neuron in the patient; preferably increased number and/or activity of one or more active nociceptor of the patient.
20. The type I interferon inhibitor for use, or the use, or the method, according to embodiment 19, wherein the sensory neurons of the patient are TrkA-expressing sensory neurons; preferably TrkA-expressing nociceptors.
21. The type I interferon inhibitor for use, or the use, or the method, according to embodiments 19 or 20, wherein the sensory neurons of the patient are GFRa3-expressing sensory neurons; preferably GFRa3-expressing nociceptors.
22. The type I interferon inhibitor for use, or the use, or the method, according to any preceding embodiment, wherein the type I interferon inhibitor:
   prevents or reduces type I interferon intracellular signalling in the patient;
   prevents or reduces levels of type I interferons in the patient;
   prevents or reduces activation of type I interferon receptors in the patient;
   prevents or reduces expression of one or more type I interferon-stimulated gene in the patient; and/or
   induces and/or increases expression of one or more type I interferon-repressed gene in the patient.
23. The type I interferon inhibitor for use, or the use, or the method, according to any preceding embodiment, wherein the type I interferon inhibitor is selected from the group comprising: an IFNAR1 inhibitor, an IFNAR2 inhibitor, a TYK2 inhibitor, a type I interferon neutraliser, an MNK inhibitor (such as an MNK1 and/or MNK2 inhibitor), a eukaryotic translation initiation factor 4E (eIF4E) inhibitor.
24. The type I interferon inhibitor for use, or the use, or the method, according to any preceding embodiment, wherein the type I interferon inhibitor is selected from the group comprising: a small molecule, an antibody, an antibody part thereof, an antibody mimetic, a decoy receptor, a receptor body, a vaccine.
25. The type I interferon inhibitor for use, or the use, or the method, according to any preceding embodiment, wherein the type I interferon inhibitor is selected from the group comprising: deucravacitinib, anifrolumab, NDI-034858, NDI-031232, NDI-031301, NDI-031407, ESK-001, VTX-958, ICP-488, ropsacitinib, GLPG3667.
26. The type I interferon inhibitor for use, or the use, or the method, according to any of embodiments 1-24, wherein the type I interferon inhibitor is an MNK inhibitor, such as an MNK1 and/or MNK2 inhibitor.
27. The type I interferon inhibitor for use, or the use, or the method, according to Claim 26, wherein the MNK inhibitor is selected from the group consisting of: eFT508; 4ET-03-053; BAY1143269; ETC-1907206; and derivatives thereof.
28. A pharmaceutical composition comprising a type I interferon inhibitor and one or more therapeutic agent for treating pain associated with Rheumatoid Arthritis in a patient, and/or one or more therapeutic agent for treating Rheumatoid Arthritis, in combination with a pharmaceutically-acceptable carrier, diluent or excipient.
29. A pharmaceutical composition as defined in embodiment 28, for use in treating or preventing pain associated with Rheumatoid Arthritis in a patient.
30. Use of a pharmaceutical composition as defined in embodiment 28 in the manufacture of a medicament for treating or preventing pain associated with Rheumatoid Arthritis in a patient.
31. A method of treating or preventing pain associated with Rheumatoid Arthritis in a patient, comprising the step of administering a pharmaceutical composition as defined in embodiment 28 to the patient.
32. A kit comprising a type I interferon inhibitor and one or more therapeutic agent for treating Rheumatoid Arthritis.
33. A method for identifying a patient who has pain associated with Rheumatoid Arthritis and is in need of treatment with a type I interferon inhibitor, the method comprising the steps of:
   (a) providing a test sample from a patient who has pain associated with Rheumatoid Arthritis;
   (b) determining the level of type I interferon signalling in the test sample; and
   (c) identifying the patient as one in need of treatment with a type I interferon inhibitor on the basis of the determination in Step (b).
34. The method of embodiment 33, further comprising the step of administering a type I interferon inhibitor to the patient.
35. A type I interferon inhibitor for use, or a use, or a method, or a composition, or a kit, substantially as described herein with reference to the accompanying description, examples, figures and/or embodiments.

### EXAMPLES

### Example 1

### Example 1: Materials and Methods

### 1.1 Animals

All experiments were carried out in accordance with protocols approved by the Stockholm Ethical Committee for Animal Experiments (Stockholms Norra Djurforsoksetiska Namnd, Sweden, 9702-2018 and 10406-2020). Animals were provided with food and water ad libitum and maintained on a 12-hour light/dark cycle. Wild type C57BL/6N mice (adult, 8-9 wk) were ordered from Charles River (Scanbur AB). WntlCre (JAX #003829), Vglut3Cre (JAX #028534), Gfra3CreERT2 (JAX #029489), Rosa26RtdTomato (Ai14, JAX #007914), Rosa26RChR2-EYFP (Ai32, JAX #012569) and Rosa26RArchT-EGFP (Ai40D, JAX #021188) were ordered from The Jackson Laboratory. SstCre (generous gift from Jens Hjerling-Leffler, JAX #013044). MrgprDCre was ordered from Mutant Mouse Resource & Research Centers (MMRRC_036118) and TrkACreERT2 mice was generated in the lab as previously described (Furlan et al, 2016). All the strains crossed back to C57BL/6N wildtype mice at least for 3 passengers before using for breeding. The resulting strains from crosses as following: WntlCre, TrKACreERT2, SstCre, Vglut3Cre, Gfra3CreERT2, MrgprDCre mice were crossed to R26TOM and R26CHR2 for characterisation; while to R26CHR2 for gain-of-function behavioural experiments and TrkACreERT2 and Gfra3CreERT2 mice were crossed to R26ArchT for loss-of-function behavioural experiments. The resulting strains from crosses as following: WntlCre/+; R26RTOM/+(abbreviated Wnt1TOM), WntlCre/+; R26RChR2/+(abbreviated Wnt1ChR2), TrkACreERT2/+; R26RChR2/ChR2 (abbreviated TrkAChR2), TrkACreERT2/+; R26RArchT/ArchT (abbreviated TrkAArchT), MrgprDCre/+; R26RTOM/+(abbreviated MrgprDTOM), MrgprDCre/+; R26RChR2/+(abbreviated MrgprDChR2), SstCre/+; R26RTOM/+(abbreviated SstTOM), SstCre/+; R26RChR2/+(abbreviated SstChR2), Vglut3Cre/+; R26RTOM/+(abbreviated Vglut3TOM), Vglut3Cre/+; R26RChR2/+(abbreviated SstChR2), Gfra3CreERT2/CreERT2; R26RChR2/ChR2 (abbreviated Gfra3ChR2), Gfra3CreERT2/CreERT2; R26RArchT/ArchT (abbreviated Gfra3ArchT).

For TrkACreERT2 and Gfra3CreERT2 mice, Tamoxifen (Sigma, T5648) was dissolved in corn oil (Sigma, 8267) at a concentration of 20 mg/ml and delivered by intraperitoneal (i.p.) injection to one injection to P14 pups and then in adult for two consecutive days (140 mg/kg both pups and adults). Control groups of test mice were also received tamoxifen injections.

### 1.2 Antibody-Induced Arthritis Model

Arthritis was induced with intravenous injection of 6 mg cartilage antibody cocktail (Cab) containing 4 arthritogenic monoclonal antibodies (ACC1: anti-citrullinated C1 epitope of CII antibody; M2139: collagen type II antibody; L10D9: collagen type XI antibody; 15A: anti-Cartilage oligomeric matrix protein antibody) on day 0 followed by 25 μg lipopolysaccharide (LPS, 055:B5, Sigma) intraperitoneally on day 5 (Li et al, 2020). Control mice received 150 μl saline i.v. on day 0 while 100 μl saline or 25 μg LPS i.p on day 5.

The development of arthritis was checked in different time points by arthritis scoring. Briefly, each inflamed (both swollen and redness) digital was given score of 1 point and if dorsal side of the paw or wrist/ankle joint was inflamed, 2.5 points were given for moderate inflammation and 5 points for severe inflammation, resulting in a maximum 15 for each limb and in total 60 per mouse (Bas et al, 2012).

### 1.3 Light-Induced Response

A flexible optical fiber bundle monitored by power controller (DC2200, Thorlabs) was used to activate the channelrhodopsin2 (ChR2), and the withdrawal reflex were elicited using a pulsing laser (470 nm, 10 Hz, 50 ms ON/OFF) with intensities from low to high and applied onto the plantar surface of the hind paws. WntlCre-ChR2, TrkACreERT2-ChR2, SstCre-ChR2, Vglut3Cre-ChR2, Gfra3CreERT2-ChR2 and MrgprDCre-ChR2 mice were habituated for 1 hour on the mess floor, and a 20-second trial was conducted, alternating between the left and right hind paws with at least 10 minutes intervals.

For excitatory optogenetics, light threshold was determined as the lowest light power provoking a withdrawal response (for reflex) or nocifensive behaviour like shaking, lifting, licking and guarding (for coping) in one of hind paws. The percentage of withdrawal reflex responding mice in different strains is reported. In all experiments, subthreshold light stimulations (0.2% lower intensity than threshold) were applied simultaneously with below tests.

### 1.4 Behavioural Tests

For sensory behavioural tests, the mice were habituated to the test environment on two occasions before assessment of baseline. After two baseline recordings performed on different days, the animals were randomly assigned to saline control, LPS control and arthritis groups. Mechanical sensitivity was determined by assessment of paw withdrawal using von Frey filament (Stoelting) and the up-down method was applied as previously described (Presley et al, 1994). A series of filaments with a logarithmically incremental stiffness of 0.04, 0.07, 0.16, 0.4, 0.6, 1.0 and 2.0 (g) was applied to the plantar surface of the hind paw and held for 3 s. To avoid tissue-damage a cut-off of 2 g was applied. A brisk withdrawal of the paw was noted as a positive response. The 50% probability withdrawal threshold (force of the von Frey hair to which an animal reacts to 50% of the presentations) was calculated.

To assess heat sensitivity, a radiant heat source (IITC, Woodland Hills, CA, USA) was aimed at the plantar surface of the hind paw through a glass surface. Briefly, mice were placed in plexiglass cubicles on a glass surface. The thermal nociceptive stimulus originates from a projection bulb below the glass surface and the stimulus is delivered separately to one hind paw at a time. Latency was defined as the time required for the paw to show a brisk withdrawal. Each hind paw was tested three times and the average withdrawal latency calculated.

To quantitatively scale pain responses: mechanical stimulated response a 2.0 g von Frey filament was applied to both hind paws and coping episodes (paw shaking, lifting/guarding or licking) were measured; cold allodynia was measured by one drop of acetone was applied to both hind paws and the response of the mouse to acetone was recorded for 45 s and coping episodes was calculated; mechanical hyperalgesia (pinprick) was also tested with a safety pin (23 G needle, BD), and coping behaviours was recorded. Data from two hind paws were presented as median with interquartile range.

For gain-of-function study, different pain-like behaviour tests were detected to mechanical and thermal stimulation before measurement of light threshold; and then light threshold was determined as the lowest light power provoking a withdrawal (reflex) or nocifensive responses (coping) in one of the paws. Subthreshold light stimulations were then applied simultaneously with sensory stimuli: withdrawal reflex subthreshold for von Frey and Hargreaves tests and coping subthreshold for 2 g von Frey, Acetone and Pinprick tests.

In littermate control mice (Wnt1^{Cre/+}; TrkACre^{ERT2/+}; ChR^{2+/-} mice), optogenetic activation did not induce stimuli-associated response, neither withdrawal nor shaking behaviour. All of the three strains showed mechanical and cold hypersensitivity, however, no differences of sensitivities to mechanical or thermal stimuli were shown in these control mice compared to combination with blue led (data not shown).

### 1.4 Inhibitory Optogenetics

To test the effect of inhibition of TrkA and Gfra3 population, mechanical and thermal sensitivities were assessed before and after yellow light (563 nm, 30 min for TrkACreERT2-ArchT and 45 min for Gfra3CreERT2-ArchT mice) stimulation. A lab-made yellow-led plate (wave length: 563 nm; 0.44 mWatt/mm2) was applied under the testing floor. For mechanical sensitivity, after 1-hour habituation on a mesh floor, the plantar surface of the hind paws were stimulated with a series of calibrated monofilaments (Stoelting, IL, USA) with increasing force (0.07 g, 0.16 g, 0.4 g, 0.6 g, 1.0 g, 1.4 g and 2.0 g). Each filament was applied five times to both hind paws. The percentage of animals with withdrawal reaction was reported.

### 1.5 Single Cell Suspension Preparation

Cervical and lumbar DRGs were collected from C57BL/6N mice (8-10 wk, Charles River, Sweden) and were placed in 6 cm Petri-dish with DPBS (Sigma) on ice. Two male mice were included for each suspension experiment. The following single cell suspension was performed according to our previous protocol with modifications (Haring et al, 2020). Briefly, DRGs were chopped 1-2 times in 2 mL Papain (25 unit/mL, Worthington Biochemical) and then digested in a 37° C. incubator, with a mixture of digestion enzymes of papain/collagenase/dispase (Papain, 25 unit/mL, 4 ml; DNase I, 55 unit/mL, 0.5 mL, Worthington Biochemical; and Collagenase and Dispase 20 mg/ml, 800 ul, Worthington Biochemical) and then triturated up and down 10 times of every 10 min using glass Pasteur pipettes with decreasing diameter (pre-coated with 0.5% BSA). Cell suspension was filtered through a 30 μm cell strainer (CellTrics, Sysmex) and washed with additional 1.5 mL ACSF (modified ACSF: 87 mM NaCl, 2.5 mM KCl, NaH2PO4 1.25 mM, NaHCO₃26 mM, Sucrose 75 mM, Glucose 20 mM, CaCl₂) 0.5 mM, MgSO4 4 mM) and 0.5 mL DPBS. Cells were spun down with centrifugation (300 g′ 6 min, 4° C.) and resuspended with 1.5 mL cold ACSF with 0.5 mL DPBS. Cell suspension was carefully loaded on top of the same volume of OptiPrep density gradient medium (Sigma) and centrifuged with 700 g′ 10 min at 4° C. The cell pellet was resuspended with 3 mL cold ACSF. SYTOX Blue (Invitrogen, ThermoFisher Scientific) was added to stain dead cells. Then alive SYTOX Blue-negative cells were sorted with fluorescence activated cell sorting (FACS) cell sorters (BD FACSAria Fusion/BD FACSAria III) at 4° C. Cells were concentrated by centrifugation (300 g′ 5 min, 4° C.) and resuspended with proper volume (~1000 cells/μl) of ACSF solution.

### 1.6 Single Cell Gene Expression 3′ Sequencing

Sorted cells were loaded onto the 10′ Chromium chip G to yield single cell droplet with v3 or v3.1 kit (10′ genomics). Targeted cell recovery for neuronal atlas construction or SNI samples was settled to 5000 cells, whereas the samples from captured active ensembles were aiming for 1000 cells. Reverse transcription, cDNA amplification and library construction were performed according to the user guide provided by manufacture. Pooled libraries were sequenced on Illumina sequencing platform NovaSeq 6000 system on SP-100 flowcells with 91 bp sequenced into the 3′ end (5′ to 3′) of the mRNAs in National Genomics Infrastructure (SciLifeLab). Raw sequencing data were de-multiplexed, converted into fastq format, and aligned to mouse reference mm10 (modified by the addition of dsRed2-WPRE) using the STAR aligner to generate the gene-cell matrices.

### 1.7 Single Cell RNA-Sequencing Data Analysis

R (v.4.1.1) using Seurat (v.4.1.0) was used for the main scRNA-seq analysis. Individual count matrices created by CellRanger (v.5.0.1) were merged to a single Seurat object and all cells with more than 20% of counts originating from mitochondrial genes were discarded. A cut-off at more than 2000 detected genes was set for the primary data. These data were integrated using Harmony (v.0.1.0) and clustered using the default algorithm in Seurat. Putative neuronal clusters were identified using the neuronal marker gene Rbfox3. Nonneuronal clusters from control samples were extracted, integrated and clustered, and assigned cell labels based on gene markers from literature (Yim et al, 2022). The nonneuronal control data was then used to transfer labels (Seurat) to all remaining nonneuronal data. After this, all remaining original data with >999 detected genes were integrated, clustered and assigned labels from the primary data. All neurons from this secondary data were discarded to make sure that only high-quality neurons were used for the final analyses. Then, the primary (>2000 detected genes) and secondary (>999 detected genes) datasets were merged to produce the full working dataset. More granular identities for the immune cells in the data were assigned using a peripheral nerve immune cell atlas (Yim et al, 2022). For this, a mixture discriminant analysis (mda) based classifier (scPred, v.1.9.2) was built using these data and the cell type labels for the immune cells in the present data were learned using this model. All cells with a prediction score below 0.55 were discarded. For neurons, all cells labelled as neurons were extracted from the full working data, clustered and using iterative clustering steps removing all cells with less than 0.5 normalised counts of Rbfox3 and more than 2 normalised counts of Apoe. A classifier was then built as before, using data from Zeisel et al with Usoskin et al annotation and the cell type labels for the neuronal data were learned using the model and unassigned neurons discarded similarly as stated above. For a pseudobulk DE analysis, the neuron types were collapsed together and data from each individual timepoint after RA induction were compared against control (t0) using Wilcoxon Rank-Sum test with the Seurat function FindMarkers with adj.p.val cut-off set at 1×10-20. DE genes for each cell type between individual RA timepoint and control were defined in a similar fashion. Fcoex (v.1.10.0) was used to identify co-regulated gene modules in the dataset. For this, to reduce computational load, a random set (25%) of cells from each cell type-timepoint pair was sampled. Fcoex was run for the first 200 genes using “timepoint” as the target. The resulting set of modules was further filtered to contain only differentially expressed genes and modules that consisted of minimum 10 genes. A module score was calculated for modules and scaled to fall between 0 and 1. A gene enrichment analysis for gene modules was run using enrichR (v.3.0) with the “GO Biological Process 2021” database. For the perturbation analysis (Augur v.1.0.0), all genes situated on the Y-chromosome and non-protein-coding genes were first discarded. Following this, the analysis was run comparing each individual timepoint against control for each neuron type. The default minimum of 20 cells per type/timepoint was used; therefore, some neuron types were not compared for each timepoint.

Gene expression matrices were imported into R (4.1.0) and analysed with Seurat (4.0.6) with standard pipelines (Satijalab). For constructing spinal neuronal atlas, individual cells were filtered out from the dataset if they had fewer than 2,000 genes or more than 20% ratio of mitochondrial genes. Raw counts were normalised by a global-scaling normalisation method “Log Nomalize” that normalises the feature expression measurements for each cell by the total expression, multiplies this by a scale factor 10, 000 and natural log-transforms (log 1p) the results. Highly variable features were identified using FindVariableFeatures( ) function (2,000 features by default) for the following analysis. Counts were centered and scaled for each gene. The effects of total UMI and percent of mitochondrial genes in each cell were regressed out using a linear model in the Scaledata( ) function. The top 50 principal components (PCs) were retrieved with the RunPCA( ) function using default parameters. JackStraw( ) function and ElbowPlot( ) function were combined to determine the dimensionality of the dataset and for the following clustering. Clustering was done with FindClusters( ) function using the shared nearest neighbour (SNN) modularity optimization technique (Louvain algorithm by default). To avoid possible over clustering, we chose an approach where the cells were clustered from the highest level of separation through the adjustment of dimensionality and resolution and followed by merging of transcriptionally highly similar clusters or separating functionally hybrid clusters. Non-neuronal cells were filtered out. Finally, 27 clusters have been produced for spinal neuronal atlas with reduction=“pca”, dims=1:25, resolution=0.5. The non-linear dimensional reduction technique UMAP was used to visualise cell clusters. Cluster specific marker genes were identified with FindAllMarkers( ) function. Wilcoxon Rank Sum test was selected to identify differentially genes with at least 0.25 increasing log fc. threshold. Specific gene markers including canonical and new were selected from the list of differentially expressed genes for unbiased classified cell clusters.

For non-neuronal cells from SNI and control samples, individual cells were filtered out from the dataset (only protein coding genes) if they had fewer than 1,000 genes, less than 4000 UMIs, or more than 10% ratio of mitochondrial genes. The same pipeline described above was applied, where the dims (1:5) and resolution (0.4) were adjusted for oligodendrocyte and microglia.

### 1.8 Type I IFN Signalling Blocking

Cartilage antibody (Cab) injected mice received either a neutralising monoclonal antibody against IFNAR1 (1 mg/mouse, i.p., BioXCell) or an isotype of mouse IgG1 antibody (1 mg/mouse, i.p., BioXCell) 1 hour prior to arthritis induction (Day 0) and on day 22 and day 45 after Cab injection. Mechanical sensitivity was tested 12 hours (0.5 d), 36 hours (1.5 d), 60 hours (2.5 d) and 84 hours (3.5 d) after IFNAR1 or isotype injection (n=3-5). Tyk2 inhibitor (Deucravacitinib/MBS-986165, MCE) was orally administered for 9 days from day 13 after Cab injection in C57Bl/6N mice twice daily (8 am and 8 μm, 15 mg/kg, in EtOH:TPGS:PEG300 of 5:5:90)(Burke et al, 2019). Mechanical sensitivity of von Frey withdrawal threshold and 2 g von Frey coping behaviour was measured 2 hour after morning injection of TYK2 inhibitor or vehicle (EtOH:TPGS:PEG300 for 5:5:90) (n=6).

### 1.9 Endo S Treatment of Antibody

For the Fc N-glycan cleavage, GST-fused endoglycosidase S (Endo S) expressed by E. coli was used to incubate with Cartilage antibody cocktail at a ratio of 1:1000 (w/w) and 37° C. for 1 hour. All antibodies were purified by using Protein G GraviTrap Columns (VWR) according to the manufacturer's instructions.

### 1.10 Cytokines Measurement in Serum

Mice were deeply anesthetised with sodium pentobarbital (60 mg/kg) and blood was collected via the heart. After sitting at room temperature for 30 min, blood samples were centrifuged with 1000 g at 4° C., and serum samples were aliquoted from supernatant and stored at −80 degree until the following tests. Serum levels of interferon-alpha (IFNα) and interferon-beta (IFNb) as well as other 32 cytokines were measured by ELISA using IFN-alpha/IFN-beta 2-Plex Mouse ProcartaPlex™ Panel (Thermofisher) and Mouse ProcartaPlex™ Panel (Thermofisher), respectively.

1.11 Gene Expression in Arthritis DRGs (SYBR Green gPCR)

Total RNA was extracted from mouse cervical and lumbar DRGs using TRIzol Reagent (ThermoFisher) and Motorized Pestle Mixer (Argos Technologies) and cDNA was generated from 500 ng RNA using High-Capacity cDNA Reverse Transcription Kit (Applied Biosystems) as previous described (Zhang et al, 2018). Quantitative PCR (qPCR) reactions were performed using SYBR Green Master Mix (Thermo Fisher Scientific) on a QuantStudio5 System (Applied Biosystems). Primer pairs used in this study were listed: Ifna (pan primers, Forward: CCTGAGAA/GAGAAGAAACACAGCC; Reverse: GGCTCTCCAGAC/TTTCTGCTCTG); Ifnb (Forward: AGGGCGGACTTCAAGATC; Reverse: CTCATTCCACCCAGTGCT); Gapdh (Forward: AACTTTGGCATTGTGGAAGG; Reverse: ACACATTGGGGGTAGGAACA). Samples were collected in different time points after antibody injection: 1 hour, 12 hours, 3 days, 33 days and 63 days, 4 mice for each group. Naïve C57BL/6 N mice (n=8) used as control group. All assays were performed in duplicate for three independent experiments and the levels of transcripts were analysed by the comparative CT (2^{-DDct}) method relative to Gapdh.

### 1.12 Immunohistochemistry and Western Blotting

Mice were deeply anesthetised with sodium pentobarbital (60 mg/kg) and perfused transcardially with 20 ml of pre-warmed (37° C.) saline, followed by 20 ml of pre-warmed 4% paraformaldehyde containing 0.2% picric acid in 0.16 M phosphate buffer (pH 7.2-7.4) and 50 ml cold fixative. L4/L5 DRGs were dissected and post-fixed in the same fixative for 90 min at 4° C. After cryoprotection in 10% sucrose with 0.1 M phosphate buffer containing 0.01% sodium azide (VWR International) and 0.02% bacitracin (Sigma) for 48 h, the tissue was embedded with OCT (HistoLab), frozen with liquid carbon dioxide and sectioned on a CryoStar NX70 cryostat (Thermo Scientific) at 12 μm thickness.

Mounted sections were dried at RT for at least 30 min and then incubated with anti-) or anti-diluted in phosphate-buffered saline (PBS) containing 0.2% (wt/vol) BSA (Sigma) and 0.3% Triton X-100 (Sigma) in a humid chamber at 4° C. for 48 h. Immunoreactivities were visualised using the TSA Plus kit (PerkinElmer) as previously described. For double labelling, sections of mouse and human ganglion already stained with LPA1 by using TSA plus kit were rinsed with PBS and incubated with CGRP antibody (1:1000) in the humid chamber at 4° C. for 48 h. After washing, the CGRP staining was visualised with secondary IgG (H+L) antibody conjugated with carbocyanin 3 (Cy3, 1:150, Jackson ImmunoResearch Laboratories) at RT for 90 min. For IB4 staining, slides were rinsed in PBS for 20 min and incubated with IB4 (1:400) from Griffonia simplicifolia I (GSA I) (2.5 g/ml; Vector Laboratories, Burlingame, CA), followed by overnight incubation with a goat anti-GSA I antiserum (1:2,000; Vector Laboratories). Finally, the sections were incubated with a FITC-conjugated donkey anti-goat antibody at RT for 2 hr (1:200, Jackson Laboratories) to visualise the IB4 binding. Counterstaining was performed on single labelling sections with 0.001% propidium iodide (PI, Sigma) for 10 min at RT. Double labelling sections were counterstained with DAPI (Sigma) for 15 at RT. After rinse in PBS, the sections were mounted with fluorescence mounting medium (Agilent Dako). Western blotting was conducted on DRGs dissected from control and arthritis animals at indicated times and treatment using standard procedures and phospho-Stat1 (Ser-727), phospho-Mnk1 (Thr-197/202) and phospho-eIF4E (Ser-209) was detected using antibodies from Cell Signaling Technologies.

### 1.13 Ex Vivo Teased Tibial Nerve Recordings

Extracellular recordings from single cutaneous primary afferent axons in an isolated mouse glabrous skin-tibial nerve preparation were obtained following previously published procedures (Reeh P W, 1986; Walcher et al, 2018). In brief, around 3 months (day 85-day 98) after Cab or vehicle injection mice (both males and females) were euthanised by cervical dislocation and the glabrous skin from one hind paw with the tibial nerve attached was dissected and placed in a custom made two-compartment teflon recording chamber with the corium side down. The chamber containing the preparation was continuously superfused at a rate of 5 ml/min with oxygenated external solution consisting of: 107.8 mM NaCl, 26.2 mM NaHCO₃, 9.64 mM sodium gluconate, 7.6 mM sucrose, 5.55 mM glucose, 3.5 mM KCl, 1.67 mM NaH2PO4, 1.53 mM CaCl₂) and 0.69 mM MgSO4, which was adjusted to pH 7.4 by continuously gassing with 95% O2-5% CO2. Temperature of the bathing solution was maintained at 33±1° C. using a heat exchanger connected to a thermostat (Zimmermann et al, 2009). The tibial nerve was placed into an adjacent chamber of the bath filled with mineral oil and then teased into small bundles that were individually placed on a gold wire electrode. A reference electrode was positioned inside the recording chamber dipped into the aqueous solution. Input signals were amplified through a high gain AC differential amplifier (Neurolog NL104A; Digitimer), digitised (PowerLab 8?; ADInstruments) at 25 kHz and stored in the hard drive of a PC for off-line analysis. LabChart software package (ADlnstruments) was used for recording and off-line analysis. Mechanically responsive receptive fields were identified by probing the skin flap with a blunt glass rod. Once a suitable fiber was found a mechanical stimulator consisting of a tension/length feedback controller (300C-I; Aurora Scientific) was used to apply mechanical stimuli. Two different force protocols were used to characterise mechanical responses. Threshold and firing frequencies were measured during continuous force ramps from 0 to 100 mN (ramp duration 10 seconds). Firing frequencies were also recorded during static force applications from 0 to 5, 10, 20, 40, 50, 75, 150, and 200 mN (step duration 10 seconds; 50 seconds interforce interval). Only mechanically responsive C fibers [conduction velocity<1.2 m/s (Koltzenburg et al, 1997)] were used in these experiments. The experimenter was blinded to genotype until data analysis was complete.

### 1.14 Statistics

Clinical score was shown as mean±standard error of mean (SEM), and behaviour data for von Frey filament test (non-continuous data) was presented as median with interquartile range and was assessed by Mann-Whitney test for non-parameters. Heat hypersensitivity behavioural data is presented as mean with SEM. For coping behavioural tests 2 g von Frey test and acetone cold allodynia, Kruskal-Wallis test, followed by Dunn's multiple. Cytokine expression result of p values of less than 0.05 were considered significant. Data was analysed with Prism 9.0 (GraphPad software).

### Example 2

### Example 2: Results

2.1 Pain Associated with Arthritis is Associated with Persistent Molecular Alterations of Transcription.

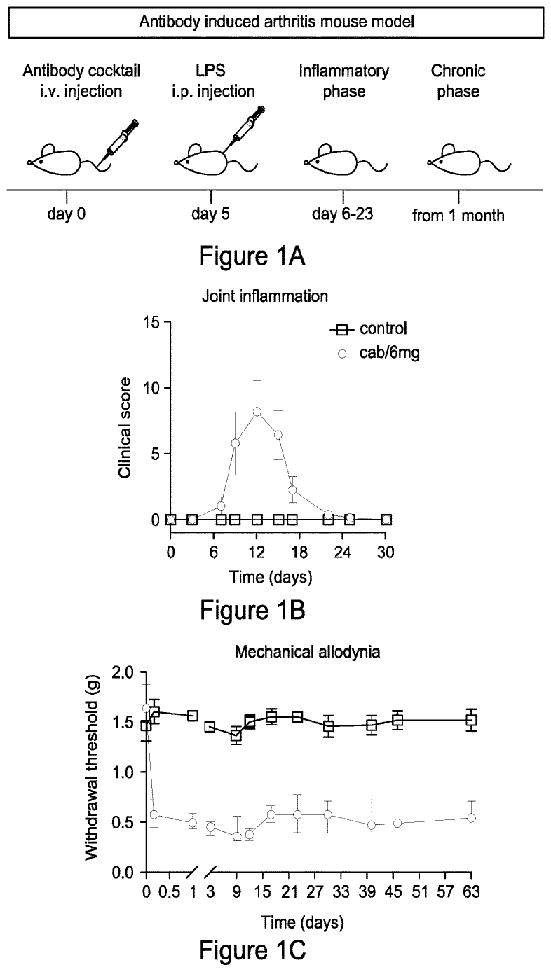

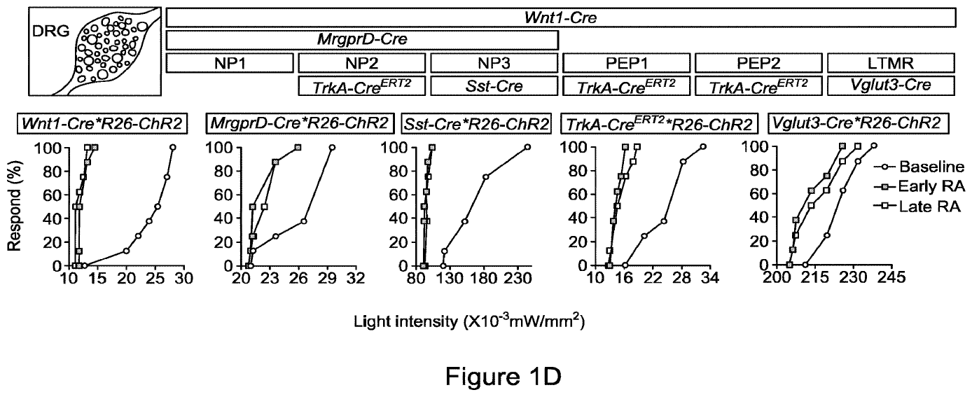

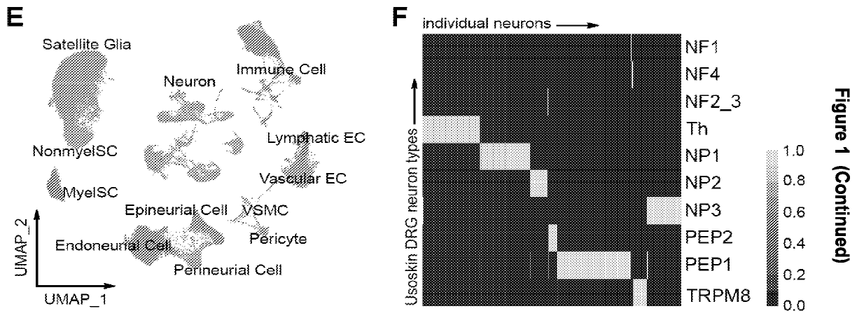

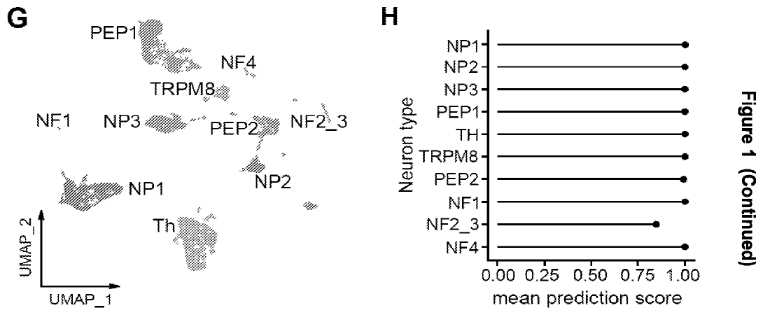

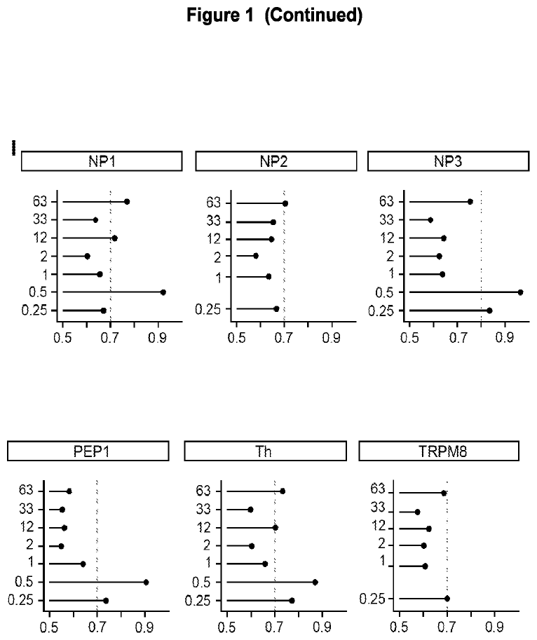

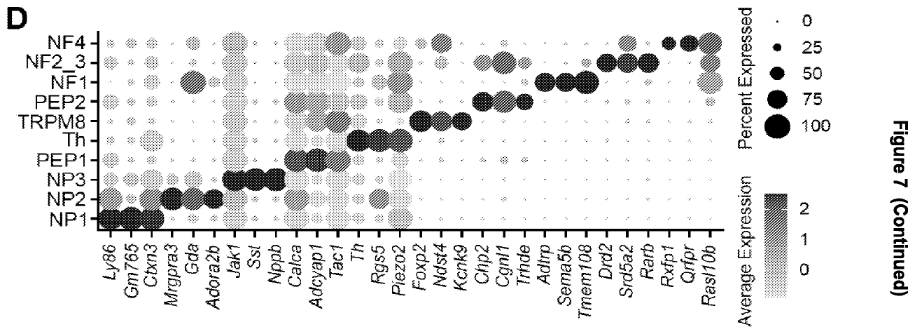

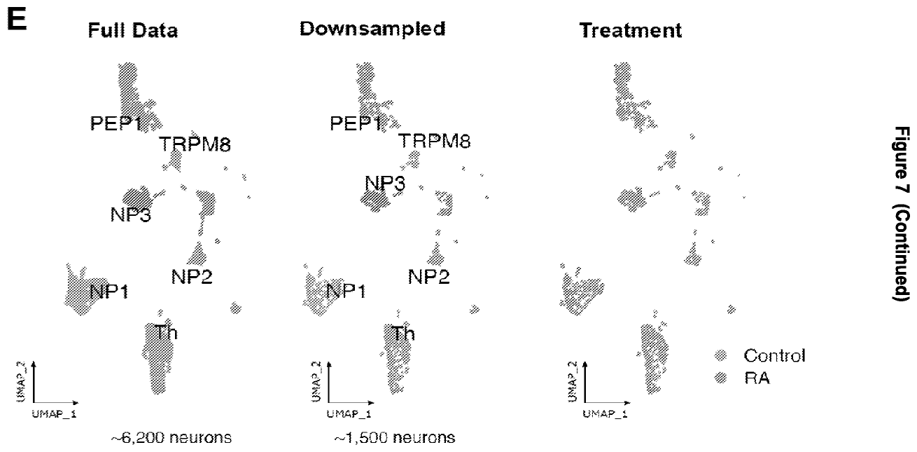

Monoclonal antibodies directed towards proteins and post-translationally modified proteins targeted by autoantibodies present in the blood and synovial fluid of early rheumatoid patients, such as anti-citrullinated collagen type II, collagen type II, collagen type XI and cartilage oligomeric matrix protein, initiate arthritis in the mouse (Krishnamurthy et al, 2016; Li et al, 2020; Wigerblad et al, 2016). Similar to patients, joint inflammation, epitope spreading of the autoimmune response and eventually bone erosion is observed. Injecting a cocktail of autoreactive cartilage-binding antibodies (Li et al, 2020) (FIG. 1A) led to macroscopic clinical arthritis such as swelling and redness observed between day 6 and 23 after antibody injection (FIG. 1B). The mice developed allodynia within 4 hours with reduced withdrawal threshold to von Frey hairs before any inflammation (4 h, d1, d3), during inflammation (d9, d12, d17, d23) as well as after inflammation had resolved (d30, d40, d46, d63) (FIG. 1C, FIG. 6A). The mice also showed an increased pain behaviour to pricking pain and cold hyperalgesia both during inflammation (early phase) and after inflammation had resolved (late phase) (FIG. 6A). Thus, pain hypersensitivity was seen before any inflammation was observed and persisted after inflammation had resolved, similar what can be observed in patients. We thereafter used the following Cre mouse lines driving expression in neuron types defined by scRNA-seq using the Usoskin et al., 2015 nomenclature of sensory neuron types (Usoskin et al, 2015): Wnt1-Cre with expression in all sensory neurons (Peng et al, 2017); MrgprD-Cre mice in pruriceptors (NP1, NP2 and NP3 neurons) (Warwick et al, 2021); TrkACreERT2 mice with expression in C-polymodal nociceptors (PEP1), Ad-heat nociceptors (PEP2), Ad-high threshold mechanoreceptor (PEP3) and Mrgpra3+ pruriceptors (NP2 neurons) (Furlan et al, 2016); Sst-Cre mice with expression in NP3 pruriceptors (Huang et al, 2019); and Vglut3-Cre mice with expression in low threshold mechanoreceptors (Lou et al, 2013) (FIG. 1D). We confirmed patterns of cell type-specific recombination consistent with previous characterisations of these mouse lines by crossing the driver lines onto a ROSA26Tomato reporter strain (FIG. 6B) and thereafter crossed them to Ai32 mice with a conditional ChR2 allele, hereafter referred to as Wnt1Chr2, MrgprDChr2, TrkAChr2, SstChr2 and Vglut3Chr2 mice. To assess sensitisation during arthritis, an increasing intensity of light was applied to the paw of the ChR2 expressing mice and the light-induced withdrawal threshold was recorded. In control mice, a striking difference was observed in the threshold between different sensory neuron types, with Wnt1Chr2, TrkAChr2 and MrgprDChr2 mice being most sensitive (16-21×10−3 mW/mm2) while SStChr2 and Vglut3Chr2 mice required more than 100×10⁻³ mW/mm2. Following cartilage antibody-induced pain, all neuron types were sensitised, and the sensitisation persisted both during inflammation and after remission of inflammation (FIG. 1D).

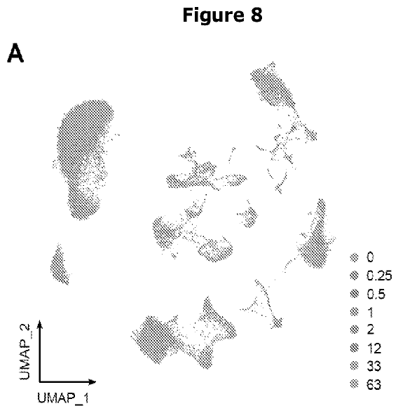

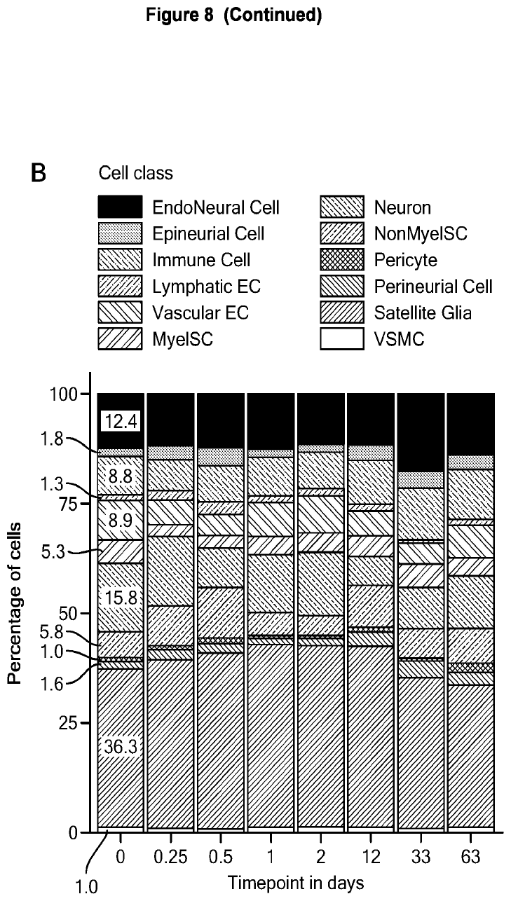

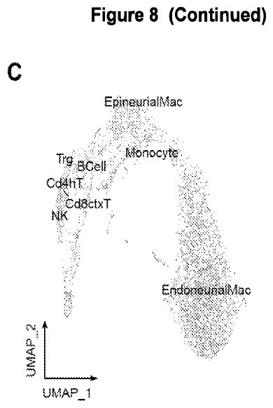

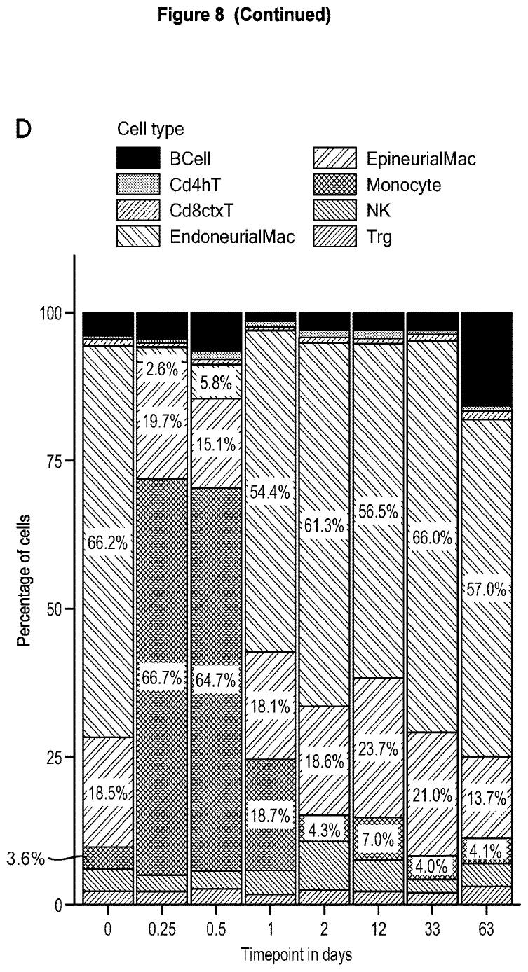

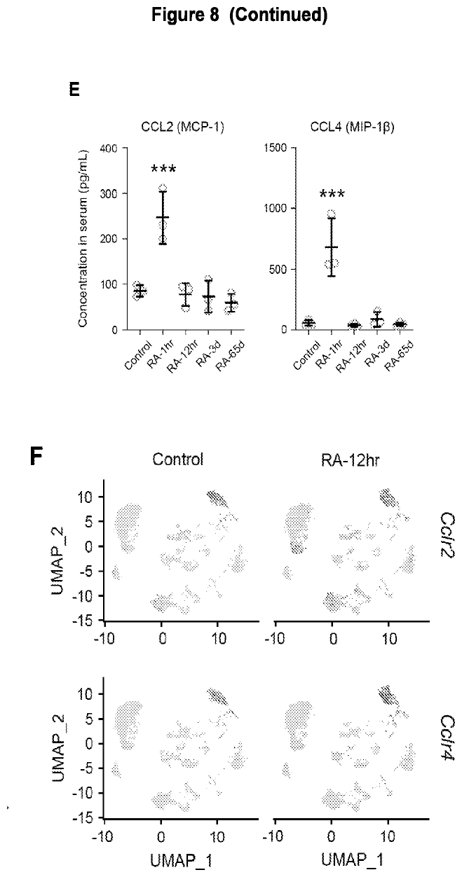

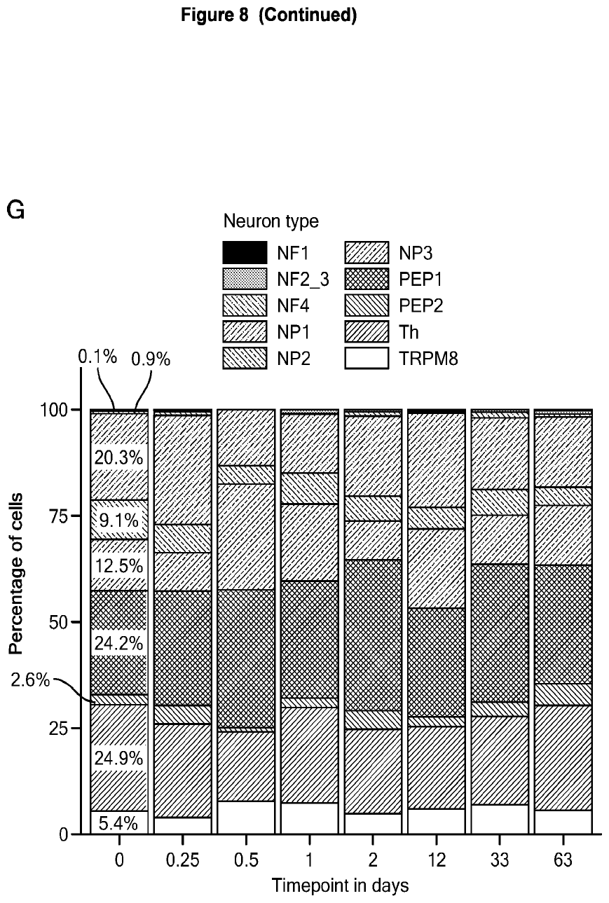

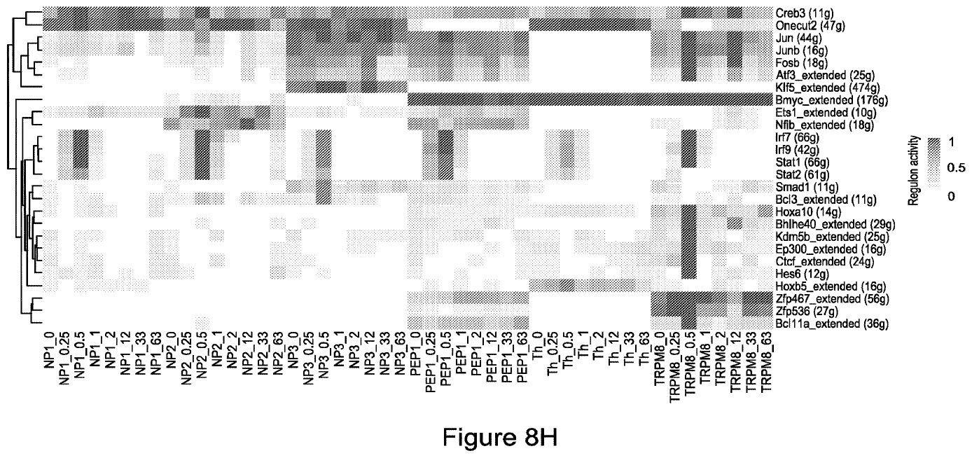

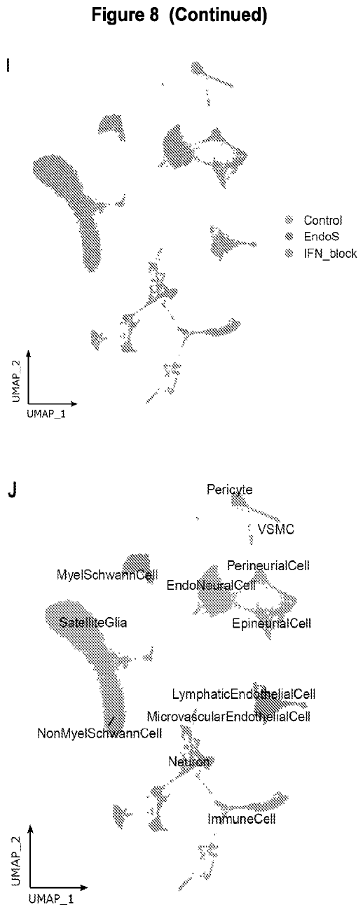

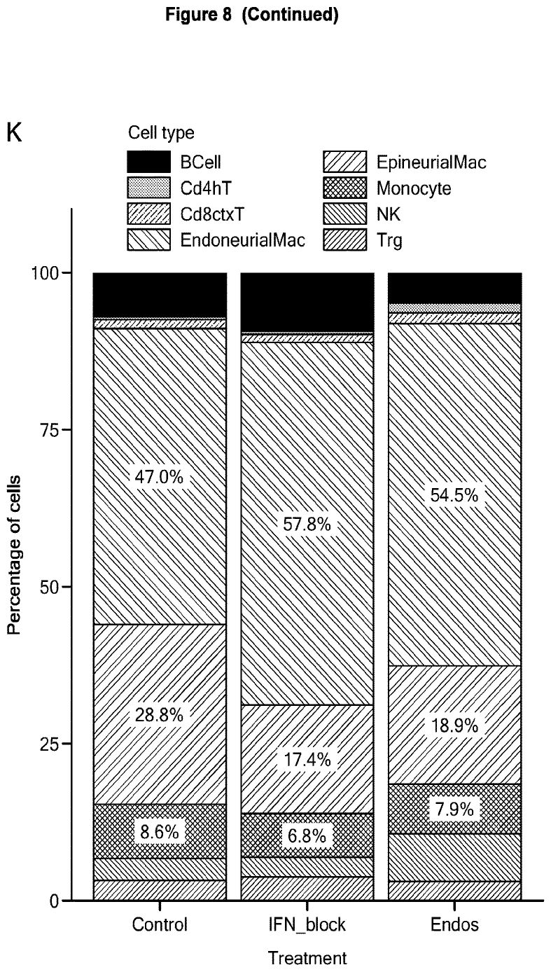

The rapid and persistent sensitisation indicated a pain mechanism independent of disease inflammation. To gain insight into the molecular underpinning sensitisation, we administered the autoreactive cartilage antibodies and collected dorsal root ganglia (DRG) at 6 and 12 hours (i.e. 0.25 and 0.5 days) and day 1, 2, 12, 33 and 63 for single cell RNA sequencing (scRNA-seq) (FIG. 7A-E). A total of about 86 000 cells were sequenced, including nerve sheet associated cells (perineural, epineural and endoneural fibroblasts), vessel associated endothelial cells and lymphatic endothelial cells, pericytes, vascular smooth muscle cells, and various types of Schwann cells (satellite, non-myelinating and myelinating) as well as immune cells and neurons (FIG. 1E). All cell types identified by clustering in controls (time point 0) were observed at all analysed timepoints and furthermore, we did not observe any overt alterations in proportion of cell types across timepoints after arthritis was induced (FIG. 8A, B). However, a more detailed analysis of immune cell types in the DRG led to identification of B, CD4T-helper, Cd8T-cytotoxic, NK, T-reg, endoneurial macrophage, epineurial macrophage and monocyte cells (FIG. 8C) and analysis of these revealed a nearly 20-fold increase in monocytes in the DRG at 6 and 12 hours (from ~3.5% to ~65%) which dropped back to baseline by day 2. Simultaneously, the proportion of endoneurial macrophages decreased from ~65% to ~3% and back to ~60% in the same window of 2 days (FIG. 8D). The upregulation of expression in DRG cells and rapid systemic increase in serum at one hour of chemokines CCL2 and CCL4 and expression of the receptors in immune cells and endoneurial macrophages (FIG. 8E, F) is consistent with the CCL-dependent recruitment of CCR2/4-expressing monocytes (Mogil et al, 2017). Thus, we interpret this to represent a turnover of endoneurial macrophages from a new set of monocytes. We assigned the sequenced neurons to sensory neuron types using the nomenclature of Usoskin et al., 2015. The heat map of the prediction score revealed a one-to-one assignment without any ambiguity (FIG. 1F) and thus, led to annotation of sequenced cells to sensory neuron types (FIG. 1G). Examination of the proportion of sequenced neurons across all the timepoints did not reveal any overt alterations (FIG. 8G). Intriguingly, the prediction score for identifying the neuron types using machine learning was close to 1 for all neurons analysed, including those in arthritis (FIG. 1H). This shows that arthritis does not affect the variable features defining each of the different neuron types. We next tested if a machine learning module trained to identify experimental versus control cells could reliably identify sensory neurons from mice with arthritis. By this, a perturbation score explaining the biological effect across the different sensory neuron types was obtained. Among the sensory neuron types, NP1, NP3 and PEP1 were mostly perturbed, with the greatest perturbation at 12 h (FIG. 1I). These results evidence a major perturbation in sensory neurons during arthritis which does not involve the cell-type defining features.

2.2 Arthritis is Associated with Transcription of an Interferon-Induced Co-Expressed Gene Program in Sensory Neurons.

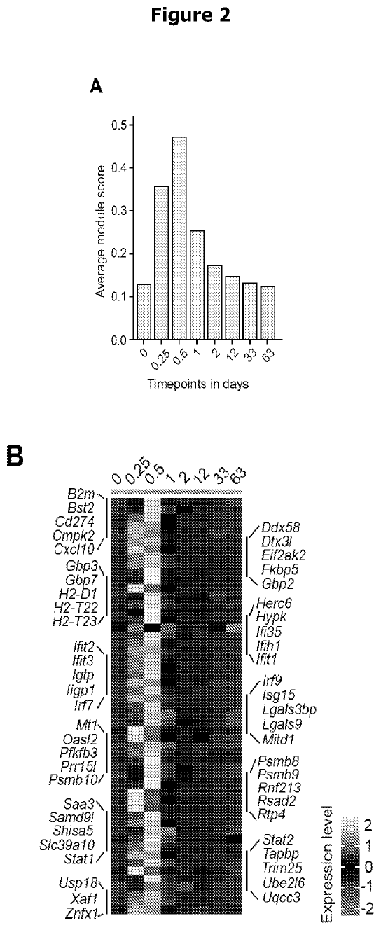

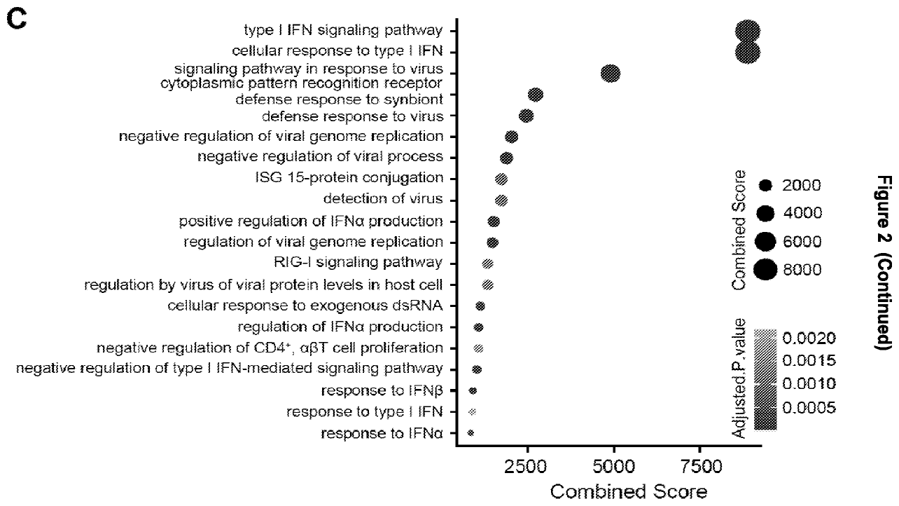

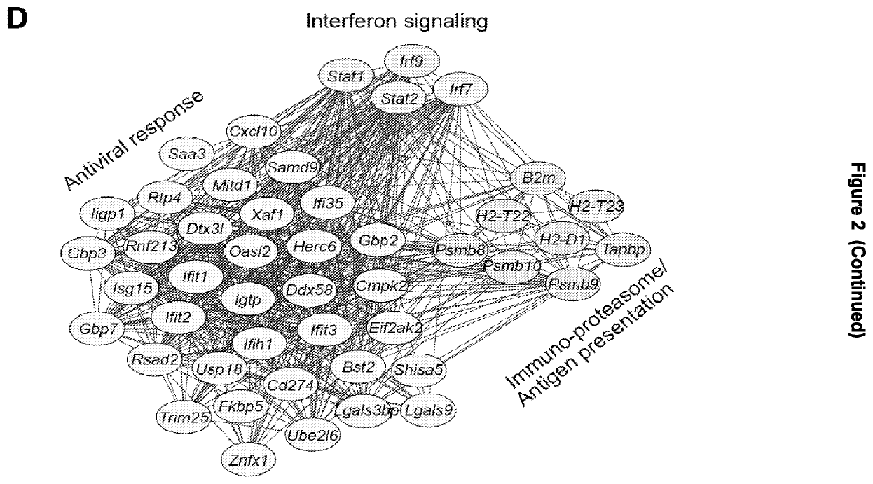

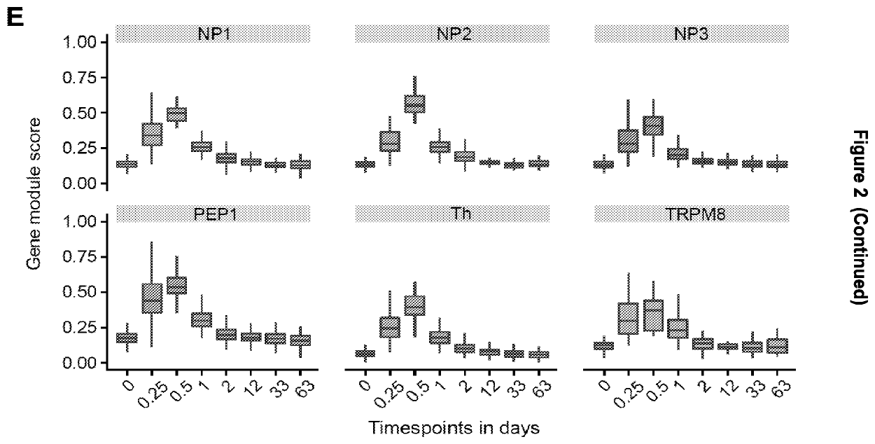

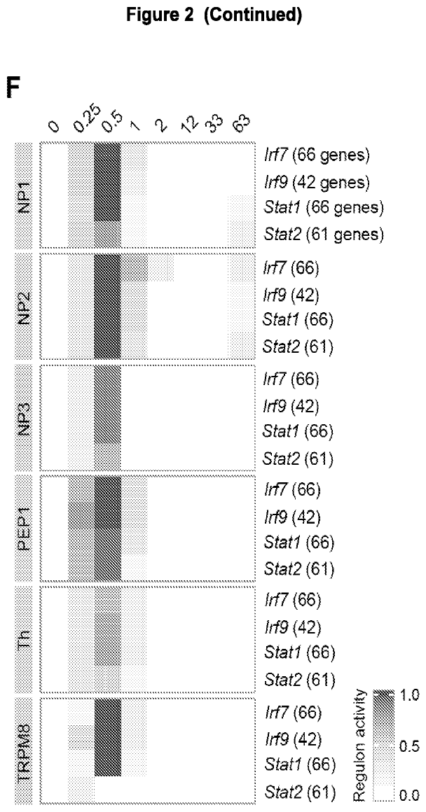

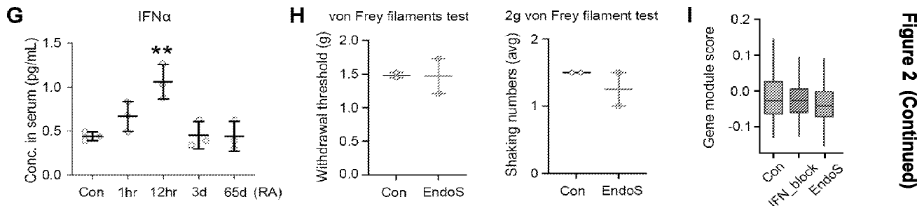

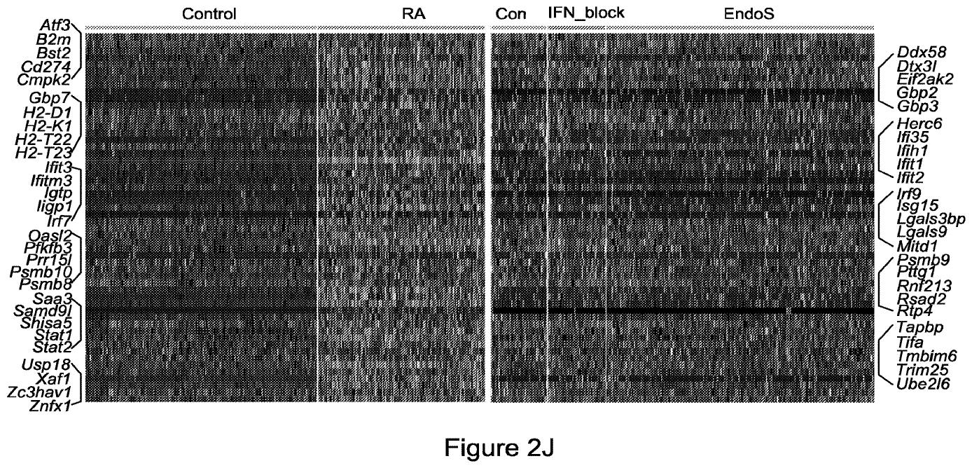

To obtain insights into the regulated gene expression caused by arthritis we examined co-regulated genes at all timepoints (0 h, 6 h, 12 h, 1 d, 2 d, 12 d, and 63 d) in a dataset with neuron types conflated, thus, a pseudobulk analysis of all DRG neurons. This led to the identification of a gene expression module of co-regulated genes consisting of 54 genes. Examining the gene expression score of these 54 genes revealed a rapid increase already at 6 h, peak at 12 h which had returned to normal at 2 days (FIG. 2A, B). Top odds ratio in gene ontology analysis of the co-expressed module were (i) interferon signalling pathway and (ii) response to type I interferons (FIG. 2C). Co-expressed genes analysed by protein association network included interferon signalling genes (IRF7, IRF9, STAT1, STAT2, ATF3), interferon-induced effectors such as virus translation and replication inhibition proteins belonging to the “IFN-induced proteins with tetratricopeptide repeats” IFIT1, IFIT3 and UBE2L6, TRIM25, USP18, TRIM30a, inhibition of virus budding such as RSAD2, BST2 (Aibar et al, 2017) and immunoproteasome/antigen presentation (Psmb8,9,10 and MHC class I genes (FIG. 2D), collectively known as interferon stimulated genes (ISGs). Analysis of the expression score of co-regulated genes in each of the different neuron types showed that all types responded similarly, with a robust increase at 6 h, peak at 12 h and downregulation at 2 days (FIG. 2E).

Activation of type I IFN receptor (IFNAR1/IFNAR2 heterodimer) leads to STAT1 and STAT2 phosphorylation and recruitment of IRF7 and IRF9, and these together form the IFN-stimulated gene factor 3 (ISGF3) that activates ISGs (Antonczyk et al, 2019). We used SCENIC (Aibar et al, 2017) to identify gene regulatory networks formed from master transcription factors and their gene targets (i.e. regulons) in all DRG cell types at all time points (FIG. 2F, FIG. 8H). Only one regulon associated with arthritis was identified and included IFN-regulatory factor 7 (IRF7), IRF9, STAT1 and STAT2.

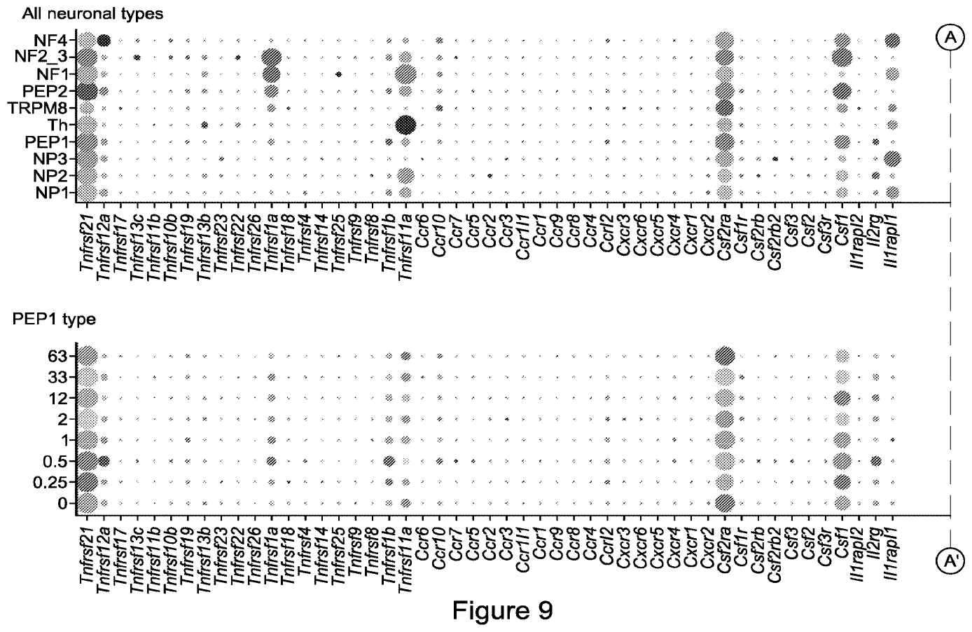

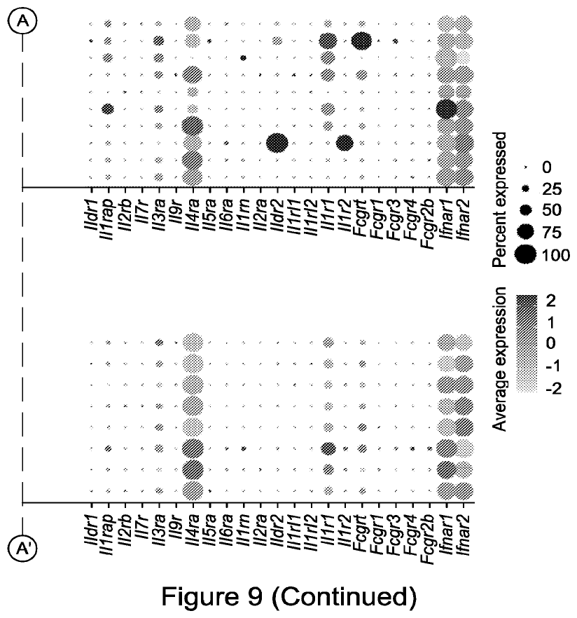

The above results implicated type I interferons as the major mediator of transcriptional alterations in sensory neurons during arthritis. Measurement of circulating IFNa and IFNb levels revealed a rapid increase during the first 12 h, thereafter returning to baseline (FIG. 2G). An effect on all sensory neuron types was consistent with expression of type I interferon receptors INFRA1 and INFRA2 in all types of the mouse and non-human primate, while autoantibody immune complex FC gamma receptors (FCGR1A, FCGR1B, FCGR2A, FCGR2B, FCGR3A) as well as most cytokine and chemokine receptors were absent or only in some sensory neuron types (FIG. 9). These results suggest an induction of ISGs through interferon a/b activation of type I interferon receptors rather than through cytosolic sensors and pathways or toll-like receptors. However, to exclude that the ISGs were induced by DNA, RNA or pathogen contaminants in the antibody mix used to induce arthritis, we used the endoglycosidase from the human pathogen Streptococcus pyogenes, EndoS, which hydrolyses different glycoforms from the Fc-glycosylation site on antibodies (Sjogren et al, 2015). Fc-glycosylation is critical for high-affinity Fc-receptor affinity, and hence, receptor recognition of Fc glycan is a major factor for high affinity binding of IgG-FC to its cognate FC-receptors. We treated the cocktail of autoreactive cartilage-binding antibodies with EndoS, repurified the antibodies, and thereafter administered them to the mice at the same concentration as used in the previous experiments. Animals administered EndoS-treated autoantibodies did not develop allodynia or hyperalgesia (FIG. 2H). DRG from EndoS-treated and control mice were collected and scRNA-seq at the 12 h peak timepoint for ISG expression. Clustering revealed all cell types previously identified in the DRG to be present in both control and experimental mice, including nerve sheath associated cells, vessel associated cell types, various types of Schwann cell glia, neurons and immune cells (FIG. 8I-K), including immune cell types (FIG. 8K). Mice administered EndoS treated autoantibody did not show any induction of ISGs and had no monocyte infiltration (FIG. 2I, FIG. 8I-K), nor were any differentially expressed genes identified between control and experimental mice (FIG. 2J). This shows that expression of ISGs is the only transcriptional change occurring during arthritis in sensory neurons and that gene regulation as well as hyperalgesia is caused by functional autoantibodies. To establish whether the gene expression changes can be directly ascribed to type I interferons, we administered an IFNAR1 inhibiting antibody and 1 hour later induced arthritis. Sensory neurons were isolated for scRNA-seq at the peak of differential gene expression. Similar to the EndoS control experiment, mice with IFN block displayed all previously identified cell types in the DRG, had an absence of monocyte infiltration and ISGs as well as a complete lack of any differentially expressed genes (FIG. 2I, J, FIG. 8I-K). Thus, we conclude that arthritis autoantibodies relying on post-translational FC-glycan modifications induce interferon expression in unknown cells resulting in type I interferon release and activation of interferon receptors expressed by sensory neurons, resulting in ISG expression.

### 2.3 the Neural Basis for Hyperalgesia and Pain in Arthritis

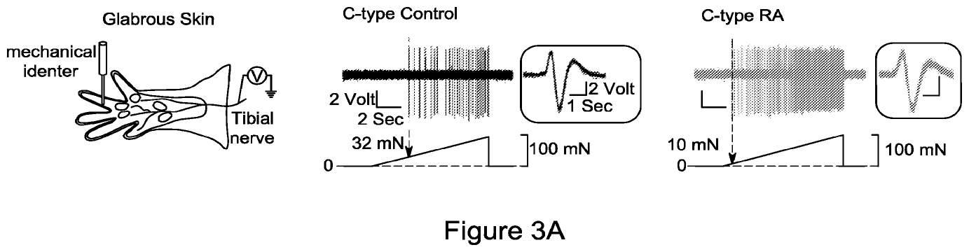

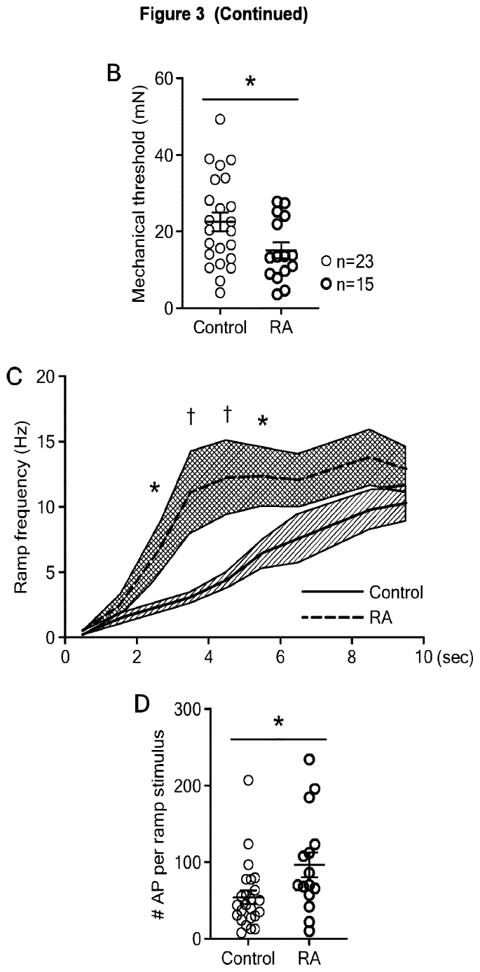

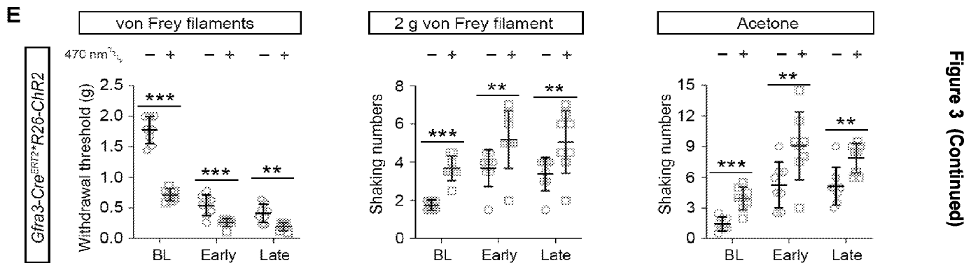

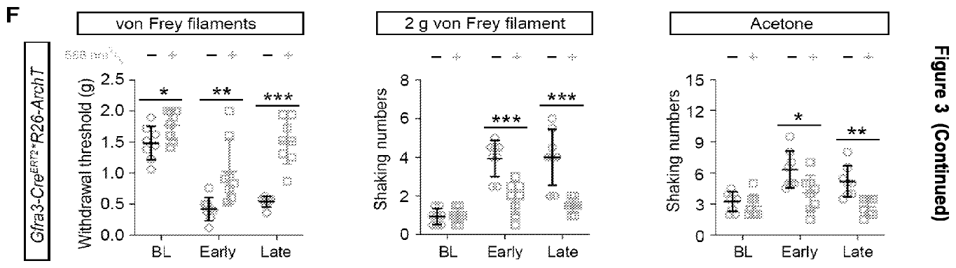

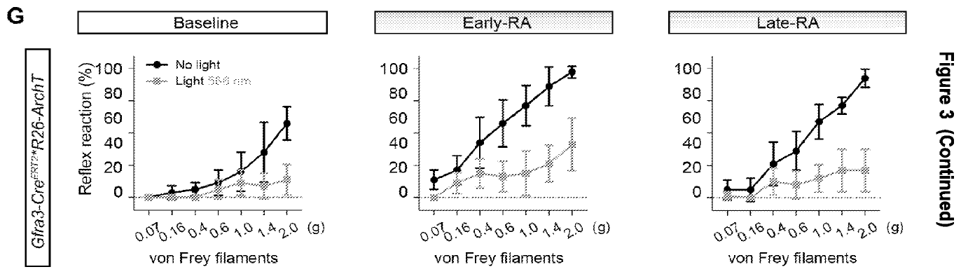

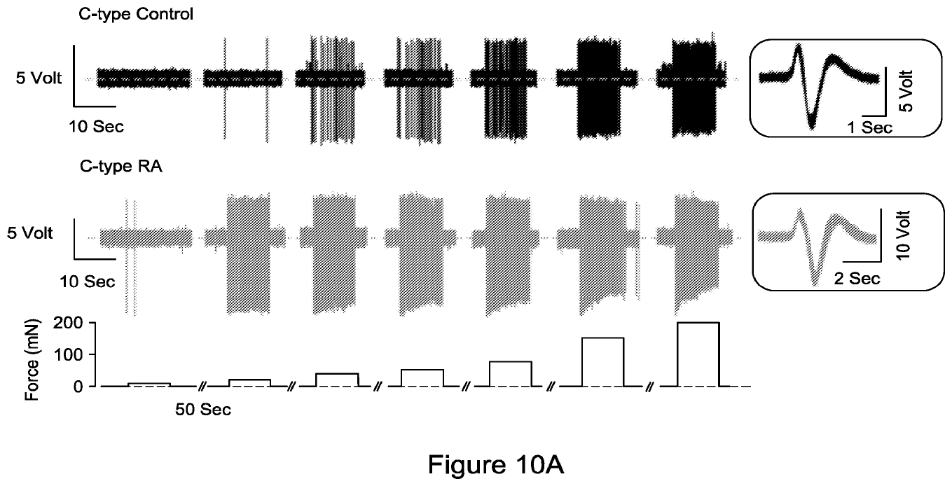

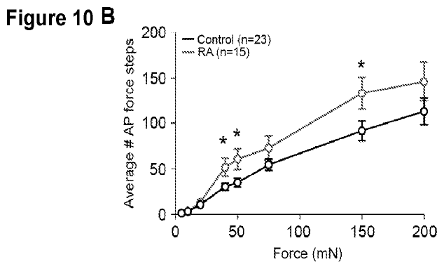

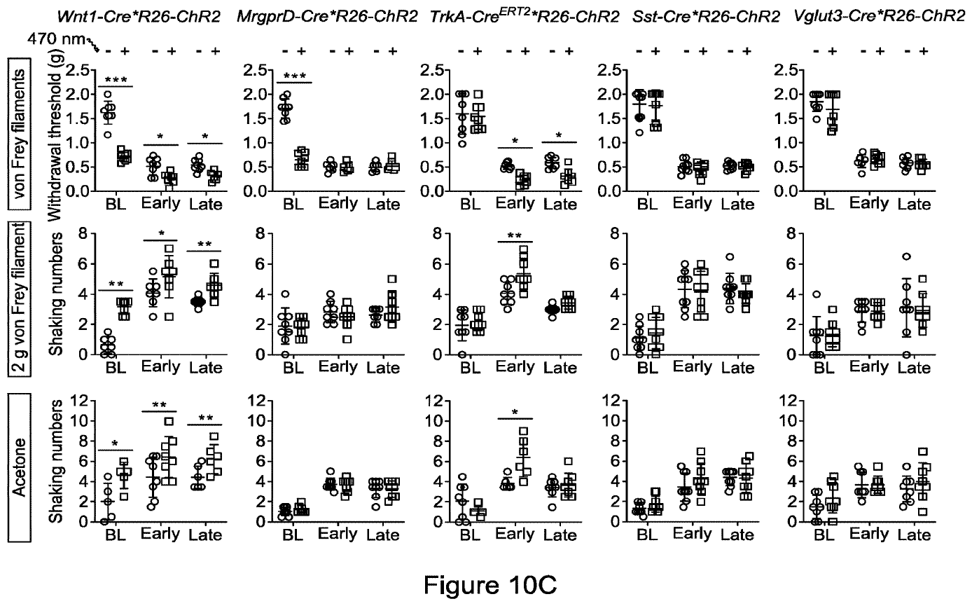

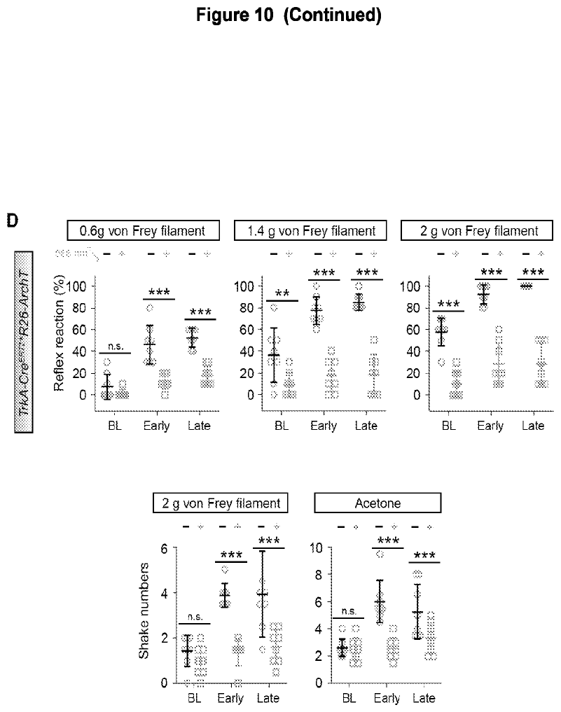

The increased behavioural response in mice following light activation of primary sensory neurons (FIG. 1D) suggests that primary sensory neurons sensitise during autoantibody-induced arthritis. To directly assess sensitisation, we performed single nerve recordings of mechanosensitive C-fibers in mice using the skin-nerve preparation (FIG. 3A). C-fibers and their receptive fields were identified and a force ramp from 0-100 mN (10 sec) was applied to find the mechanical threshold and to determine the firing frequency during the ramp in control mice and autoantibody-treated mice. Thereafter 10 s force steps were applied with the forces: 5, 10, 20, 40, 50, 75, 150, 200 mN (FIG. 10A). The firing frequency (action potentials, APs, per second) during the force steps and the total number of APs during each step was measured. The mechanical threshold was significantly reduced in mice with autoantibody-induced arthritis (FIG. 3B) and firing frequency was markedly increased during the ramp, starting already during relatively low forces as compared to control mice (FIG. 3C). Consistently, measuring average mechanically induced APs during the force ramp application revealed a significant increase starting already at 30 mN force (FIG. 3D, FIG. 10B). Thus, nociceptors become sensitised during autoantibody-induced arthritis.

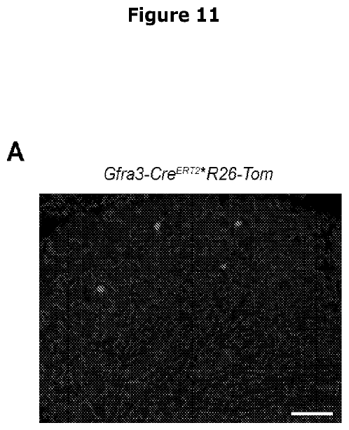

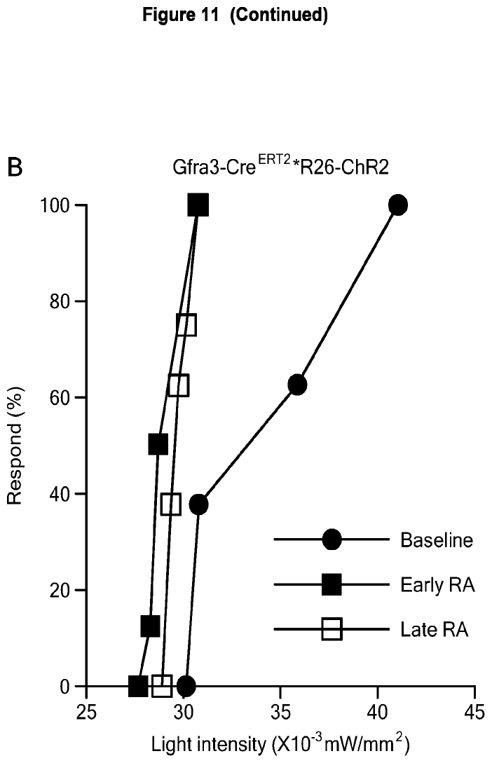

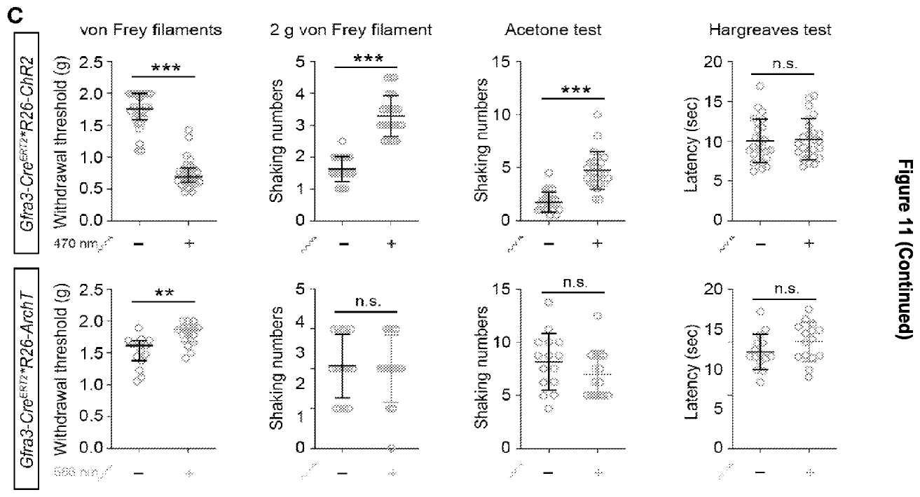

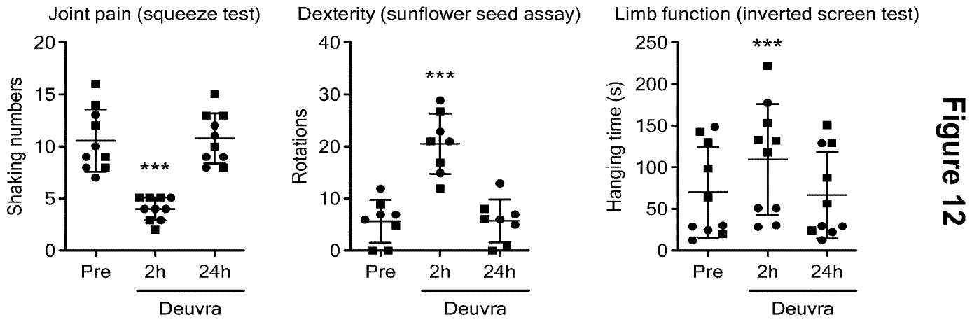

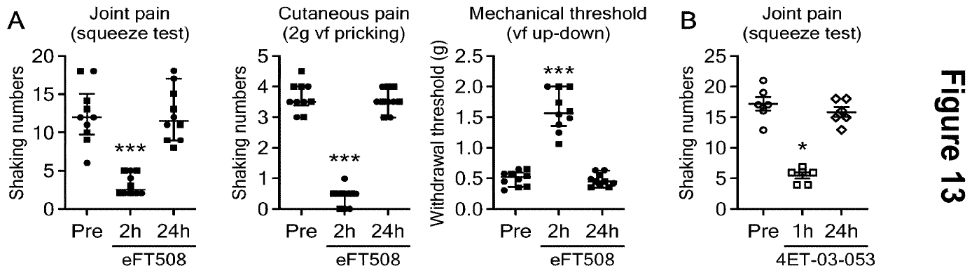

Because all C-fiber sensory neuron types induced ISG expression, our analyses did not help to identify exactly which neuron type(s) is the cause for pain associated with arthritis. To identify the neurons responsible for allodynia and hyperalgesia to mechanical and cold pain we combined subthreshold light activation of Wnt1Chr2, MrgprDChr2, TrkAChr2, SStChr2 and Vglut3Chr2 mice with von Frey threshold measurements, 2 g von Frey pricking and cold (acetone) to see if we could potentiate coping behaviour that may serve to soothe suffering (Huang et al, 2019) in animals with arthritis. We measured basal response prior to arthritis, during inflammation (early) and after resolution of inflammation (late) with and without subthreshold light. Arthritis led to allodynia and hyperalgesia in all mouse strains, however, subthreshold light only potentiated allodynia, pricking and cold pain in the TrkAChr2 mice (FIG. 10C). The critical role of TRKA expressing sensory neurons led us to cross the TrkACreERT2 mice to Ai40 mice carrying a conditional ArchT allele (TrkAArchT mice) in order to optogenetically silence TrkA expressing neurons. Silencing resulted in a complete reversal of allodynia, pricking pain and cold hyperalgesia both during and after resolution of inflammation (FIG. 10D). We next set out to establish whether the C-fiber polymodal sensory neuron type PEP1 among the TrkA expressing sensory neuron types (PEP1, PEP2, PEP3 and NP2) is responsible for pain associated with arthritis. Gfra3 is expressed exclusively in PEP1 sensory neurons (Usoskin et al, 2015). We generated Gfra3CreERT2 mice carrying a conditional reporter, ChR2 or ArchT allele (Gfra3Tomato, Gfra3ChR2 and Gfra3ArchT mice) and induced arthritis in the latter two strains of mice. Gfra3Tomato mice confirmed recombination in the DRG (FIG. 11A) and C-polymodal nociceptors in Gfra3ChR2 mice were sensitised, as shown by reduced light-induced withdrawal thresholds which persisted both during inflammation and after remission of inflammation (FIG. 11B). In Gfra3ChR2 mice, subthreshold light markedly potentiated allodynia, pricking and cold pain behaviour (FIG. 3E) and silencing the Gfra3 neurons in Gfra3ArchT mice reversed arthritis-induced allodynia, pricking and cold pain behaviour both during inflammation and after remission while keeping a normal tactile sensitivity (FIG. 3F, G). While the contribution of Trpv1 and Calca (CGRP) expressing sensory neurons to normal pain behaviour has previously been studied, these genes mark several sensory neuron types including C-polymodal nociceptors, Ab-nociceptors as well as several types of pruriceptors and hence, the normal function of C-polymodal nociceptors is unknown. To establish if polymodal C-nociceptors change function during arthritis, we first examined their role in the naïve animal. In naïve Gfra3ChR2 mice, subthreshold light combined with naturalistic stimuli revealed a contribution of Gfra3+ nociceptors to mechanical threshold detection, mechanical pain behaviour and cold pain behaviour while there no effect was seen on threshold detection to heat (FIG. 11C). Silencing Gfra3+ polymodal nociceptors in Gfra3ArchT mice revealed only a small shift in mechanical threshold detection (FIG. 11C). This shows a gain of function of Gfra3+ C-polymodal nociceptors responsible for pain associated with arthritis. Thus, silencing of this neuron type in naïve animals does not affect pain behavioural responses but is sufficient to completely revoke pain associated with arthritis.

2.4 Chronic Pain Associated with Arthritis is Caused by a Sustained Interferon Signalling that is Reversible.

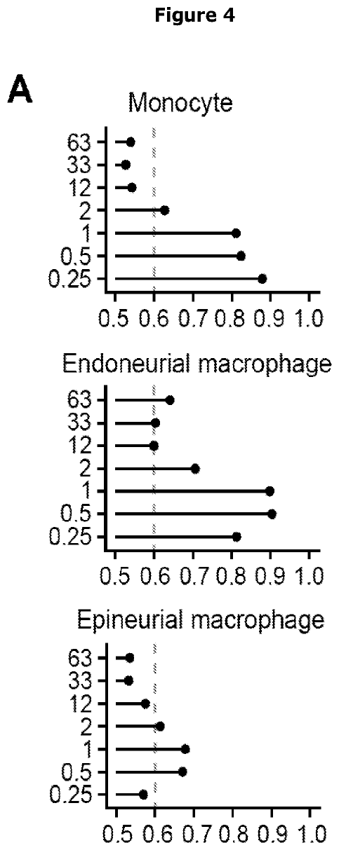

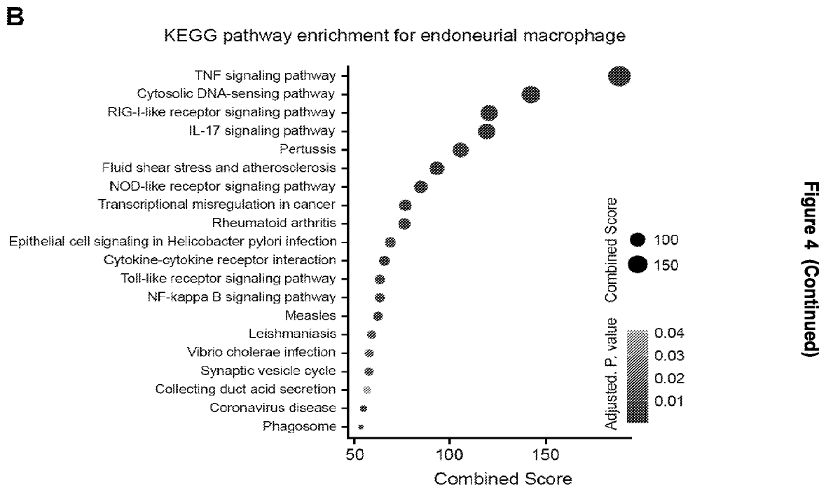

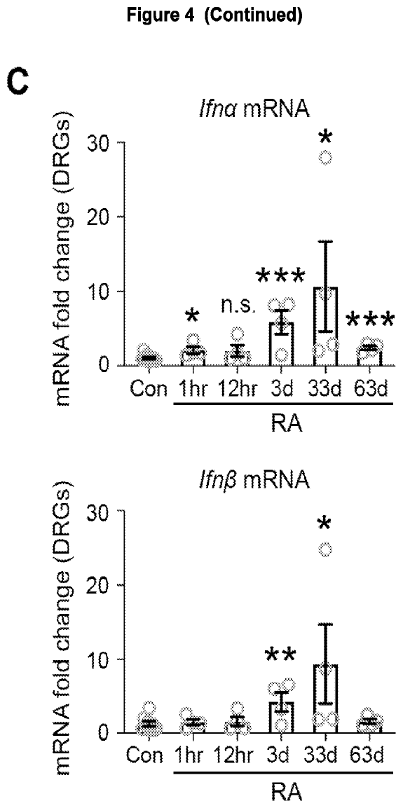

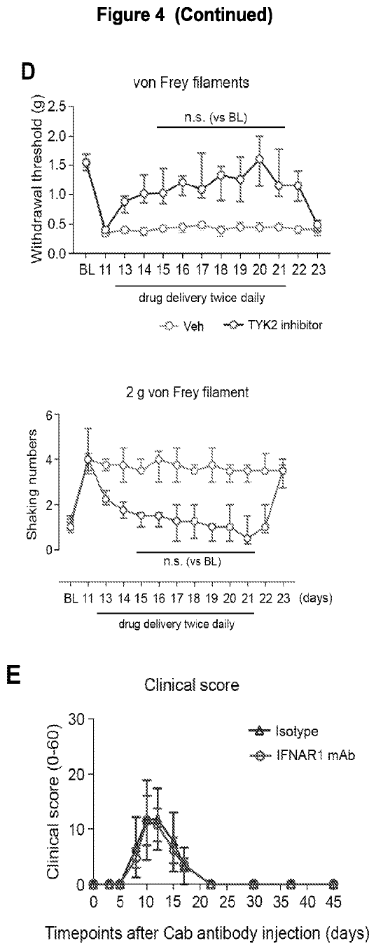

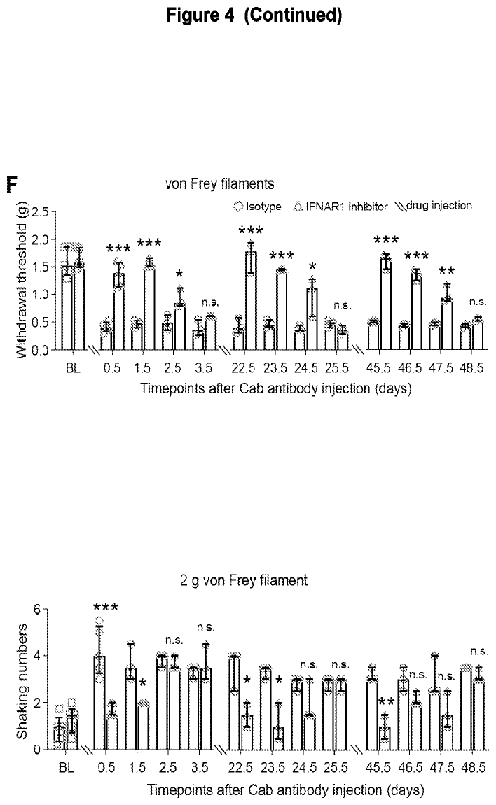

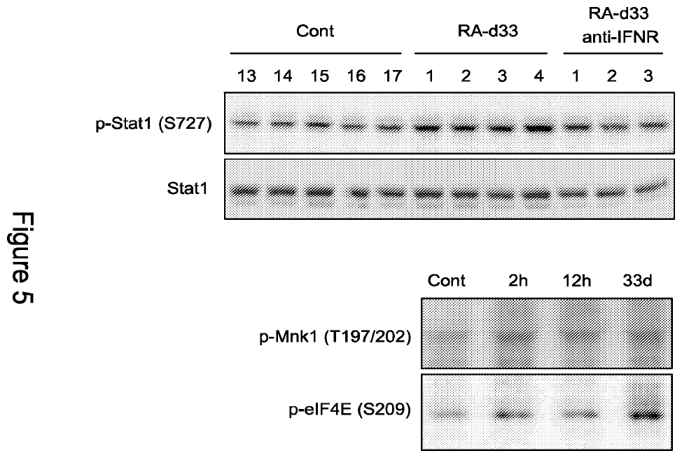

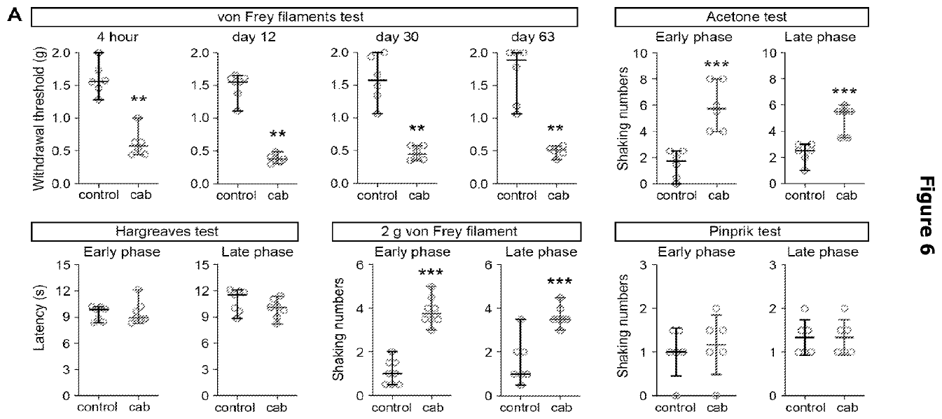

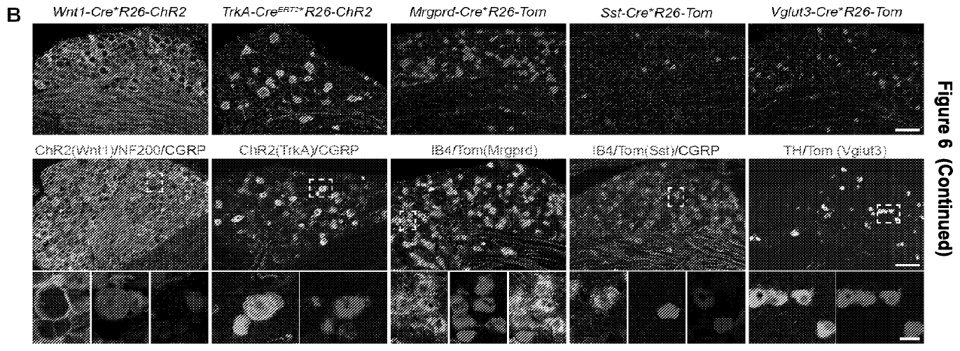

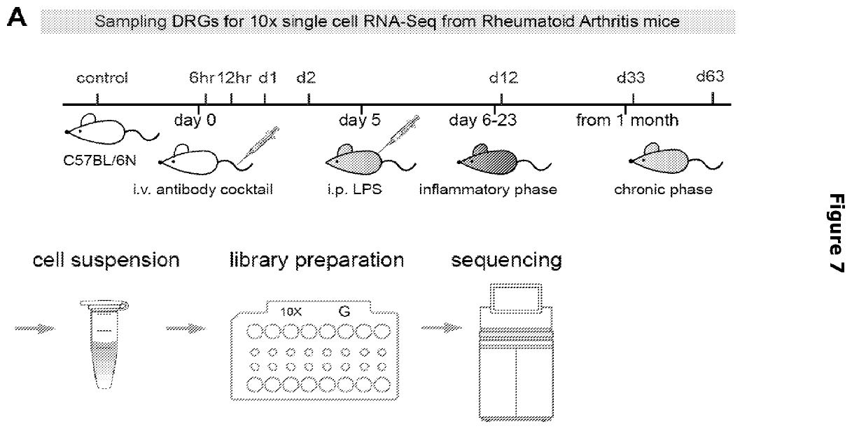

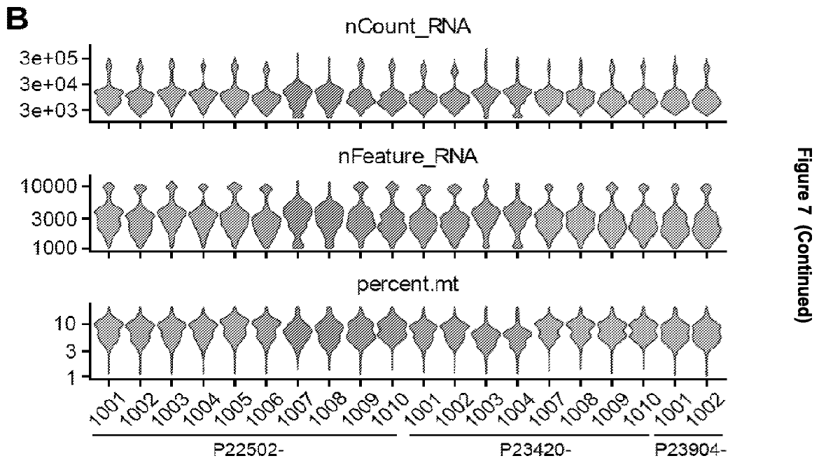

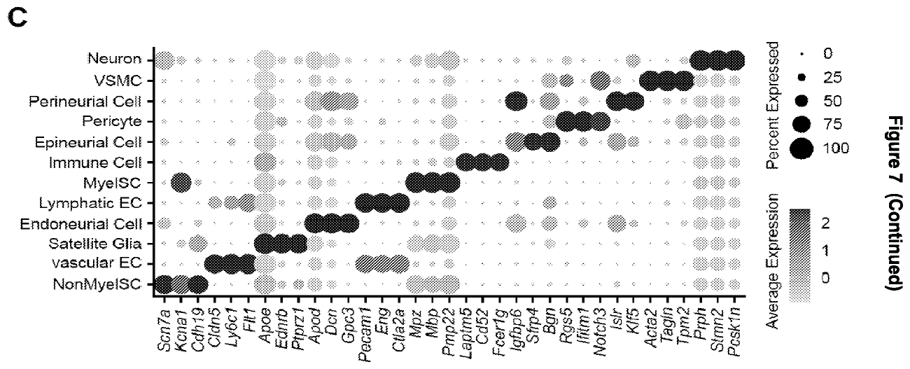

While Gfra3+ polymodal C-nociceptors were critical for pain associated with arthritis, it remained unclear if interferon signalling is transient or sustained and whether a putative sustained signalling is the cause for arthritis allodynia and hyperalgesia. Because systemic IFN levels were transient, a persistent IFN-dependent sensitisation must arise from local inflammation and IFNs production. Analysis of perturbation in all DRG cell types revealed alterations in non-neuronal DRG cells, with the greatest perturbation at 12 h in monocytes, endoneurial macrophages and epineurial macrophages, however, endoneurial macrophages showed a persistent perturbation all through 63 days (FIG. 4A). Top odds ratio for gene ontology were TNF signalling and terms related to inflammation (FIG. 4B). Because type I interferons are not polyadenylated, we were unable to identify expression in the scRNA-seq data and instead performed quantitative PCR for all 14 IFNa combined and for IFNb. Arthritis led a sustained local transcription of IFNs in the DRG (FIG. 4C). Thus, pain in arthritis is associated first with transient systemic IFNs and thereafter local IFN transcription associated with inflammation in the DRG. To test if IFNs are the cause for allodynia and hyperalgesia we first used the allosteric tyrosine kinase 2 (TYK2) inhibitor deucravacitinib that is a modulator of cytokines relying on TYK2 signalling (IFN-α, IL-12, IL-23). Deucravacitinib administered daily during 10 days potently reversed allodynia and hyperalgesia (FIG. 4D). To establish if IFNs were responsible for the initiation of pain, an IFNAR1 antagonist antibody was injected 1 h before inducing arthritis in mice. Blocking IFNAR1 did not significantly affect inflammation (FIG. 4E) yet prevented mechanical allodynia and hyperalgesia during the first two days after the blocking antibody was administered, after which allodynia and hyperalgesia developed (FIG. 4F). Blocking IFNAR1 in animals with already established arthritis during inflammation at day 22.5 and after resolution of inflammation at day 45.5 significantly reversed mechanical allodynia and mechanical hyperalgesia (FIG. 4F). Phospho-STAT1 (5727), phospho-MNK1 (T197/202) and phospho-eIF4E was persistently increased in DRG of mice with induced arthritis as compared to control mice and the increase depended on an ongoing interferon signalling since the IFNAR1 antagonist antibody reversed the increase (FIG. 5). Thus, blocking type I interferon signalling can both prevent and reverse pain associated with arthritis.

We propose that a systemic burst by immune or other cell types initiates pain and thereafter locally produced type I interferons has a causative role for pain in arthritis. Type I interferons interact with sensory neurons inducing ISGs, sensitisation, allodynia and hyperalgesia. A continuous interferon signalling is predicted because preventive therapy does not have a sustained effect and already established chronic pain can be overcome by inhibiting ongoing type I interferon receptor signalling. The systemic interferon signature in our animal model is in agreement with data from patients with active rheumatoid arthritis (Rodriguez-Carrio et al, 2015, Van der Pouw Kraan et al, 2007). The synovial joints are innervated by unmyelinated peptide-rich sensory C-fibers and thinly myelinated A6 fibers and with the latter mainly present in the capsule, ligaments and meniscus (Grigg, 2001). Optogenetic control of excitation and inhibition of the different sensory neuron types reveal pain associated with arthritis to be caused by a molecularly unique polymodal C-fiber nociceptor marked by expression of Gfra3. Thus, we find that the molecular mechanism for pain is different from the main drivers of synovial inflammation during arthritis. This could explain why some painful rheumatoid arthritis patients fail to respond to therapies suppressing joint inflammation (Sun et al, 2018). Our results suggest therapies based on inhibiting type I interferon signalling to be efficacious for pain relief during rheumatoid arthritis.

### Example 3

### Materials and Methods

### Autoantibody-Induced Arthritis Model

Arthritis was induced by intravenous (i.v.) injection of 6 mg cartilage autoantibody cocktail containing 4 arthritogenic monoclonal antibodies (ACC1: anti-citrullinated C1 epitope of collagen type II (COL2) antibody; M2139: COL2 antibody; L10D9: collagen type XI antibody; 15A: anti-cartilage oligomeric matrix protein antibody) on day 0 followed by 25 μg lipopolysaccharide (LPS, 055:B5, Sigma) intraperitoneally (i.p.) on day 5. Control mice received 150 μL saline i.v. on day 0 while 100 μL saline or 25 μg LPS i.p on day 5.

### Pharmacological Blocking

TYK2 inhibitor (Deucravacitinib/MBS-986165, MCE) was orally administrated for 7 times from day 43 after autoantibody injection in C57BL/6N mice twice daily (8 AM and 8 PM, 15 mg/kg, in EtOH:TPGS:PEG300 of 5:5:90). Mechanical sensitivity of von Frey withdrawal threshold and 2 g von Frey coping behavior was measured 2 h after morning injection of TYK2 inhibitor or vehicle (EtOH:TPGS:PEG300 for 5:5:90) (n=10).

A single i.p. injection of MNK1/2 inhibitor, Tomivosertib (eFT508/HY-100022, MCE) on day 48 after antibody injection in C57BL/6N mice (1 mg/kg, in DMSO:PEG300:Tween-80:Saline of 5:40:5:50), and then mechanical sensitivity of von Frey withdrawal threshold and 2 g von Frey coping behavior was measured 1 h and 24 h later while clip squeeze test was measured 2 h and 24 h after Tomivosertib administration (n=10). Another MNK1/2 inhibitor, 4ET-03-053, was orally administrated (1 mg/kg, in PEG300:Saline of 50:50, on day 51) into arthritis mice. Joint pain (squeeze test) was measured 1 h and 24 h after 4ET-03-053 delivery (n=6).

eIF4E/eIF4G interaction inhibitor, 4EGI-1 (324517, Sigma) was i.p. injected into antibody-induced arthritis C57BL/6N mice (15 mg/kg, in DMSO:PEG300:Tween-80:Saline of 5:40:5:50, on day 56). Mechanical sensitivity (von Frey up-down and 2 g von Frey coping tests) as well as clip squeeze test were checked (n=5).

For testing local blockage of MNK inhibitor, intraplantar administration of eFT508 (2 mg/kg, DMSO:PEG300:Saline of 10:40:50) 15 min prior to IFNA3 injection (3000/10 ul, intraplantar) on left hind paw (ipsi-). Mechanical sensitivity (von Frey up-down and 2 g von Frey coping tests) were tested 1 h, 3 h, 24 h, 3 d and 6 d after IFN administration on both hind paws (n=5).

### Behavioral Tests

Joint pain: Clip squeeze test was used for checking joint pain, after 1 hour of incubation in the Hargreaves' box (IITC), a toothless clip (420 G) was applied to squeeze the proximal interphalangeal (PIP) joint and extension of the metatarsal-phalangeal (MTP) joint of one hind paw for 5 sec; then coping episodes (shaking numbers) was analyzed for 4 min after clip removal.

Dexterity test: To test the dexterity of forepaws, sunflower seed assay was used. Animal was habituation to separated test box (animal enclosure, IITC) placed on the grey matte acrylic floor. After habituation, 2-3 sunflower seeds (provided by KM-B, Karolinska Institutet) were applied to the floor for 20 min for 3 consecutive days (1 round of training). Only the activated mice (completely deshelling the seeds, around 60%) after 2 rounds of training were used for further study.

Two days prior to testing (habituation day 1 and day 2) and during testing day (testing on day 3), animals were transferred from their home cage and placed in the test box and allowed to explore the environment for 10 min. And then 2 seeds were placed on the floor and seed eating activity was recorded for 20 min. The episodes of rotation were calculated: the act of manipulating shell orientation rotating for 180 degree within the forepaws.

Overall limb behavior test: To perform limb's function, inverted screen test was measured: the mouse was placed in the center of the wire screen (width 7 mm and diameter 2 mm for wire, GMC500) and rotate the screen to an inverted position over 2 sec, with the mouse's head declining first. Hold the screen steadily 45-50 cm above a soft material padded surface. Record the time mouse falls off (hanging time); animal will be removed from screen when it reaches the cutoff point (6 min).

Mechanical sensitivity: For sensory behavioral tests, the mice were habituated to the test environment on two occasions before assessment of baseline. After two baseline recordings performed on different days, the animals were randomly assigned to saline control and arthritis groups. Mechanical sensitivity was determined by assessment of paw withdrawal using von Frey filament (Stoelting). A series of filaments with a logarithmically incremental stiffness of 0.04, 0.07, 0.16, 0.4, 0.6, 1.0 and 2.0 (g) was applied to the plantar surface of the hind paw and held for 3 sec according to the up-down method. A brisk withdrawal of the paw was noted as a positive response. The 50% probability of withdrawal threshold (force of the von Frey hair to which an animal reacts to 50% of the presentations) was calculated as threshold. To avoid any potential tissue damage, a cut-off value of 2.0 g was applied. The average withdrawal threshold of two hind paws was used. Coping episodes (paw shaking, lifting/guarding or licking) were measured as a quantitative scale of pain responses to a 2.0 g von Frey filament applied to both hind paws. The average shaking numbers of two hind paws were used.

### Western Blot

Human L5 DRGs of normal donors and RA patients were ordered from (Anabios). Tissues were homogenized using TissuelyserII system (Qiagen) and the total protein was extracted from DRG tissue using N-PER neuronal protein extraction reagent (87792, ThermoFisher) containing Protease inhibitor cocktail (G6251, Promega) and Halt Phosphatase inhibitor cocktail (78428, ThermoFisher). The protein concentration was determined with a BCA Protein Assay Kit (Pierce) and 30 μg of denatured protein was separated by electrophoresis on a NuPAGE 4-12% Bis-Tris gel and then transferred by iBlot2 Dry Blot system (Life Technologies). Membranes were then blocked for 1 h in 5% non-fat milk in TBS.T (0.1% Tween 20) at room temperature and then incubated over night with primary antibodies against IFN alpha (ThermoFisher), pSTAT1 (S727, Cell Signaling) overnight at 4° C. After washing, membranes were probed with secondary antibodies conjugated to horseradish peroxidase, which were then detected with SuperSignal West Femto reagents (Thermo) and imaged with a ChemiDoc™ MP system (Bio-Rad Laboratories). Membranes were stripped with Re-Blot Solution (Life Technologies) and re-probed with betaIII-tubulin (Promega, neuronal marker) and Actin (Abcam, housekeeping). Band intensity was quantified using ImageJ software.

### Patch-Clamp Electrophysiology

Cell cultures for electrophysiology patch-clamp were prepared from adult C57BL/6N mice (both sexes, 7-10 weeks). Briefly, all level of cervical and lumbar DRGs were dissected and digested in Papain/Collagenase/Dispase mixture, then triturated by glass Pasteur pipettes. Single cell suspended in L-15 medium (Liebovitz, L1518, Merck) contained 10% FBS, NaHCO₃, Glucose, penicillin/streptomycin (1X) and Floxuridine (PHR2589, Merck) and plated on poly-D-lysine (A-003-E, Merck) and laminin ([2020, Merck) pre-coated coverslip. The following day, changes in neuronal excitability in nociceptors (small-sized, diameter≤20 μm) were tested after 1 hour stimulation of recombinant mouse IFN-alpha A protein (12100-1, 3000/mL, B&D systems), PBS containing 0.1% BSA (Sigma) stimulation in neurons served as controls. To test the effects of MNK1/2 inhibition on IFN-stimulated nociceptors, neurons was first incubated with 10 μM eFT508 (in L-15 medium) or vehicle (DMSO, 0.1% v/v) for 1 hour and IFNA3 (3000/mL) was added into the same well stimulated for 1 hour.

Whole cell patch-clamp experiments were performed using a MultiClamp 700B (Molecular Devices) patch-clamp amplifier and low-pass filtered at 10 kHz. Glass electrodes pulled with a micropipette puller (P-1000, Sutter Instruments) from borosilicate glass (Hilgenberg) filled with intracellular solution (containing (in mM): 120 K-gluconate, 5 KCl, 10 HEPES, 4 Mg2ATP, 0.3 Na4GTP, 10 Na-phosphocreatine with pH 7.4 adjusted with KOH and an osmolarity of 275 mOsm. Data were analyzed using Clampfit 10 (Molecular Devices). Data are from six independent mice cultured on separate days. The purpose of this experimental protocol was to consider both biological and experimental variability, potentially coming from the culturing process. All neurons included in the analysis had a resting membrane potential more negative than −40 mV. The RMP was recorded 1-3 min after achieving whole-cell configuration. In current-clamp mode, cells were held at −60 mV and action potentials were elicited by injecting slow ramp currents from 100 to 700 pA with A200 pA over 1 s to mimic slow depolarization. Only cells that responded to the ramp depolarization; at least one spike at the maximum 700 pA, were considered for further analysis.

### Experiments and Results

As discussed in the Examples above, monoclonal antibodies directed towards proteins and post-translationally modified proteins targeted by autoantibodies present in the blood and synovial fluid of early rheumatoid patients, such as anti-citrullinated collagen type II, collagen type II, collagen type XI and cartilage oligomeric matrix protein, initiate arthritis in the mouse 15 (Krishnamurthy et al, 2016; Li et al, 2020; Wigerblad et al, 2016). Similar to patients, joint inflammation, epitope spreading of the autoimmune response and eventually bone erosion is observed. Injecting a cocktail of autoreactive cartilage-binding antibodies (Li et al, 2020) (FIG. 1A) led to macroscopic clinical arthritis such as swelling and redness observed between day 6 and 23 after antibody injection (FIG. 1B). The 20 mice developed allodynia within 4 hours with reduced withdrawal threshold to von Frey hairs before any inflammation (4 h, d1, d3), during inflammation (d9, d12, d17, d23) as well as after inflammation had resolved (d30, d40, d46, d63) (FIG. 1C, FIG. 6A). The mice also showed an increased pain behaviour to pricking pain and cold hyperalgesia both during inflammation (early phase) and after inflammation had resolved (late 25 phase) (FIG. 6A). Thus, pain hypersensitivity was seen before any inflammation was observed and persisted after inflammation had resolved, similar what can be observed in patients.

Arthritis led a sustained local transcription of IFNs in the DRG (FIG. 4C).

To test if IFNs are the cause for allodynia and hyperalgesia we first used the allosteric tyrosine kinase 2 (TYK2) inhibitor deucravacitinib that is a modulator of cytokines relying on TYK2 signalling (IFN-α, IL-10 12, IL-23). Deucravacitinib administered daily during 10 days potently reversed allodynia and hyperalgesia (FIG. 4D). To establish if IFNs were responsible for the initiation of pain, an IFNAR1 antagonist antibody was injected 1 h before inducing arthritis in mice. Blocking IFNAR1 did not significantly affect inflammation (FIG. 4E) yet prevented mechanical allodynia and hyperalgesia during the first two days after 15 the blocking antibody was administered, after which allodynia and hyperalgesia developed (FIG. 4F). Blocking IFNAR1 in animals with already established arthritis during inflammation at day 22.5 and after resolution of inflammation at day 45.5 significantly reversed mechanical allodynia and mechanical hyperalgesia (FIG. 4F). Phospho-STAT1 (5727), phospho-MNK1 (T197/202) and phospho-eIF4E was 20 persistently increased in DRG of mice with induced arthritis as compared to control mice and the increase depended on an ongoing interferon signalling since the IFNAR1 antagonist antibody reversed the increase (FIG. 5). Thus, blocking type I interferon signalling can both prevent and reverse pain associated with arthritis.

In the present Example, to test if IFNs are the cause for joint pain, loss of dexterous use of paws and overall paw function we first used the allosteric tyrosine kinase 2 (TYK2) inhibitor deucravacitinib that is a modulator of cytokines relying on TYK2 signalling (IFN-α, IL-10 12, IL-23). Deucravacitinib administered twice daily during 4 days potently reversed joint pain, dexterity and limb function, and the effect was washed out at 24 h after the last injection (FIG. 12).

To test if blocking IFNs activation of MNK1/2 are the cause for allodynia, joint pain and loss in dexterity, we used the specific MNK inhibitors eFT508 and 4ET-03-053. Systemic injection (i.p.) of MNK1/2 inhibitor (eFT508, i.p., 1 mg/kg) in mice in the chronic phase of arthritis, potently reversed joint pain, dexterity and overall limb function, an effect that was washed out with the compound at 24 h (FIG. 13A). Per oral administration of another MNK inhibitor, 4ET-03-053, in the chronic phase of arthritis potently reversed joint pain, an effect that washed out with the compound at 24 h (FIG. 13B).

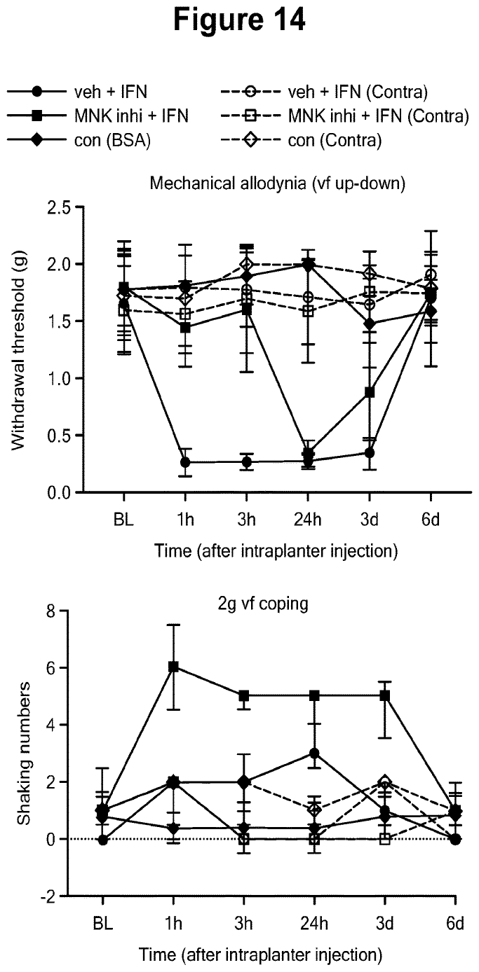

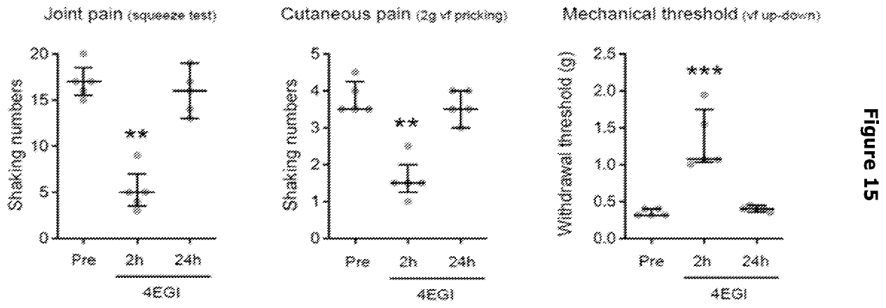

Intraplantar injection of type I interferon (IFNA3, 3000/10microliter) injection led to a marked sensitization and mechanical allodynia as well as increased mechanical pain for up to 3 days with a reversal at 6 d. Intraplantar injection of MNK1/2 inhibitor eFT508 prevented allodynia and pain in animals receiving intraplantar injection of interferon (FIG. 14).

To test if blocking IFN signalling by inhibition of eIF4E, the specific inhibitor 4EGI (15 mg/kg, i.p.) was administered in mice with arthritis. 4EGI reversed joint pain, arthritis induced cutaneous pain and allodynia (FIG. 15).

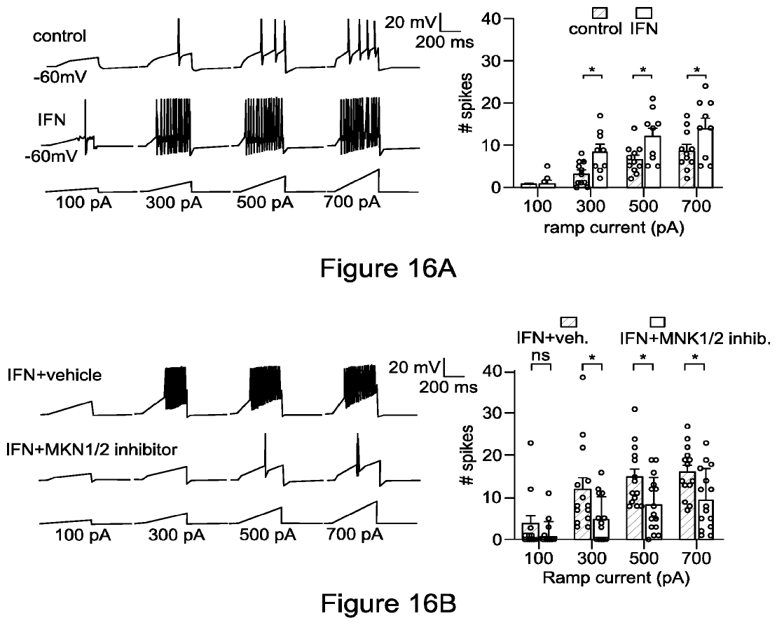

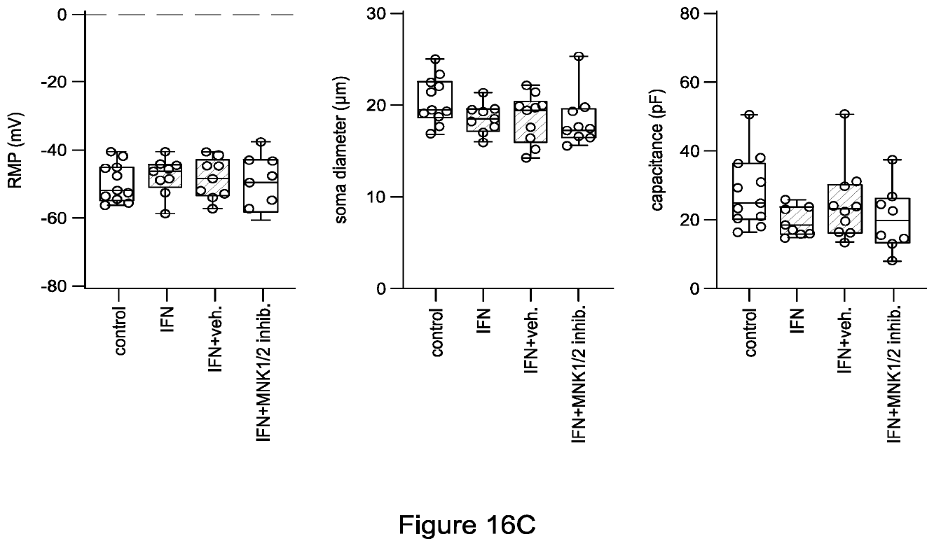

To test if joint pain, loss of dexterity and disabilities in limb function is caused by sensitization and hyperexcitability of sensory neurons by type I interferon and if so, whether MNK1/2 inhibition can reverse sensitization, we performed patch clamp recordings on mouse sensory neurons. Type I interferons markedly increased firing of sensory neurons and decreased the threshold of initiation of the first action potential (FIG. 16A). The reduction in action potential threshold and increased firing by type I interferon was reversed by the MNK1/2 inhibitor eFT508 (FIG. 16B).

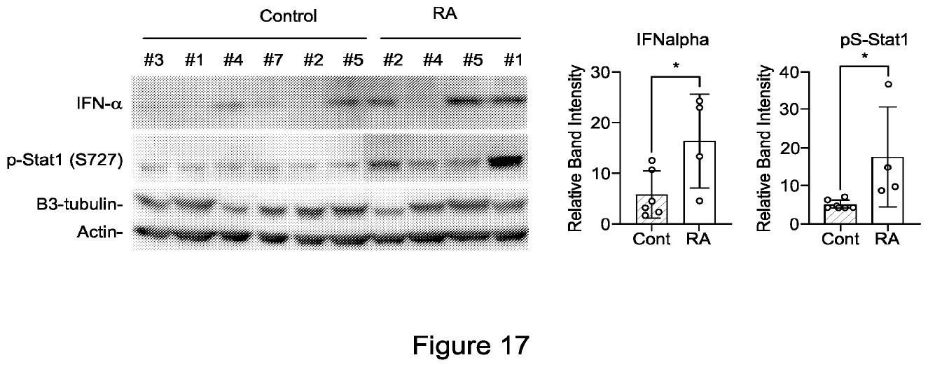

Similar to mouse dorsal root ganglion with arthritis, type I interferon (IFNalpha) protein levels were increased in donor DRGs from patients with rheumatoid arthritis and joint pain as compared to healthy donor tissue without rheumatoid arthritis and pain (FIG. 17A). Thus, patients with rheumatoid arthritis have an ongoing production of type I interferons that sensitize the neurons through interferon signalling. Interferon signaling as revealed by phospho-STAT (S727) was also increased in patients with RA and pain as compared to healthy controls (FIG. 17B).

### Discussion

The mechanisms causing persistent pain in inflammation have been ascribed to an observed increase in excitability of sensory neurons residing in dorsal root ganglia (Cao et al., 2020; McWilliams and Walsh, 2017). Activity in primary sensory neurons is essential, because local anesthesia relieves not only acute (Nagi et al., 2011) but also chronic pain (Elsaman et al., 2021). Therefore, hypersensitivity of sensory neurons may account for chronic inflammatory pain. Recent and compelling evidence shows that a broad-spectrum cytokine inhibitor baricitinib (JAK1/2 inhibitor), targeting multiple cytokine pathways, lessens pain associated with rheumatoid arthritis, as demonstrated in the Phase 3 clinical trial RA-BEAM (NCT01710358). This finding highlights the important role of cytokines, directly or indirectly, in mediating pain in arthritis (Simon et al., 2021). However, the disconnect between inflammatory remission and pain indicates the importance of cytokines beyond those causing arthritis, and their identity remains unknown.

We find unexpectedly that Type I interferons interact with sensory neurons inducing, sensitisation, allodynia and hyperalgesia and that such sensitization relies on activation of a signaling pathway including TYK2 with increased STAT1 phospho-Ser272 and in phospho-MNK1/2 and phospho-eIF4E. Thus, the interferon signalling involves an unconventional type I interferon signalling pathway, different from the canonical antiviral pathway that relies on STAT1, STAT2 and IRF9. Sensory neuron hypersensitivity caused by activating this pathway by interferons involves a lowered threshold for initiation of an action potential as well as increased firing of action potentials once they are initiated.

There is a requirement of the MNK1/2 and eIF4E pathway because we find that inhibiting MNK1/2 prevented interferon induced hypersensitization of sensory neurons. Thus, MNK1/2 creates the hypersensitivity state in sensory neurons which leads to decreased threshold for action potentials to be initiated and increased action potential firing once the threshold is reached. In animals with arthritis, inhibiting MNK1/2 or eIF4E completely reverses joint pain and restored paw function in animals with arthritis. This shows that MNK1/2 and eIF4E are required for pain in arthritis.

Thus, we find that the molecular mechanism for pain is different from the main drivers of synovial inflammation during arthritis. This could explain why some painful rheumatoid arthritis patients fail to respond to therapies suppressing joint inflammation (Smolen et al., 2018).

Our results suggest therapies based on inhibiting type I interferon signalling, including the signalling pathway components, such as MNK1/2 and eIF4E, to be efficacious for pain relief during rheumatoid arthritis.

### REFERENCES

Alunno, F. Carubbi, R. Giacomelli, R. Gerli, Cytokines in the pathogenesis of rheumatoid arthritis: new players and therapeutic targets. BMC Rheumatology 1, 3 (2017).
N. Selim, B. Ahmet Merih, in New Developments in the Pathogenesis of Rheumatoid Arthritis, I. S. Lazaros, Ed. (IntechOpen, Rijeka, 2017), pp. Ch. 3.
L. A. Ridgley, A. E. Anderson, A. G. Pratt, What are the dominant cytokines in early rheumatoid arthritis? Current Opinion in Rheumatology 30, (2018).
P. Vergne-Salle et al, The burden of pain in rheumatoid arthritis: Impact of disease activity and psychological factors. European journal of pain (London, England) 24, 1979-1989 (2020)
L. Day, J. R. Curtis, Opioid use in rheumatoid arthritis: trends, efficacy, safety, and best practices. Curr Opin Rheumatol 31, 264-270 (2019)
J. Bullock et al, Rheumatoid Arthritis: A Brief Overview of the Treatment. Medical Principles and Practice 27, 501-507 (2018)
Y. Cao, D. Fan, Y. Yin, Pain Mechanism in Rheumatoid Arthritis: From Cytokines to Central Sensitization. Mediators of Inflammation 2020, U.S. Pat. No. 2,076,328 (2020)
J. M. Cunha, F. Q. Cunha, S. Poole, S. H. Ferreira, Cytokine-mediated inflammatory hyperalgesia limited by interleukin-1 receptor antagonist. British journal of pharmacology 130, 1418-1424 (2000)
S. Poole, F. Q. Cunha, S. Selkirk, B. B. Lorenzetti, S. H. Ferreira, Cytokine-mediated inflammatory hyperalgesia limited by interleukin-10. British journal of pharmacology 115, 684-688 (1995)
R. Amann, R. Schuligoi, G. Herzeg, J. Donnerer, Intraplantar injection of nerve growth factor into the rat hind paw: local edema and effects on thermal nociceptive threshold. Pain 64, 323-329 (1996)
S. Iyengar, M. H. Ossipov, K. W. Johnson, The role of calcitonin gene-related peptide in peripheral and central pain mechanisms including migraine. Pain 158, 543-559 (2017)
F. Kim, G. Moalem-Taylor, Interleukin-17 contributes to neuroinflammation and neuropathic pain following peripheral nerve injury in mice. The journal of pain 12, 370-383 (2011)
K. M. Lee et al, IL-23 in arthritic and inflammatory pain development in mice. Arthritis research & therapy 22, 123 (2020)
J. Ji et al, IL-23 Enhances C-Fiber-Mediated and Blue Light-Induced Spontaneous Pain in Female Mice. Frontiers in immunology 12, 787565 (2021).
P. Barragan-Iglesias et al, Type I Interferons Act Directly on Nociceptors to Produce Pain Sensitization: Implications for Viral Infection-Induced Pain. The Journal of neuroscience: the official journal of the Society for Neuroscience 40, 3517-3532 (2020)
Achuthan et al, Granulocyte macrophage colony-stimulating factor induces CCL17 production via IRF4 to mediate inflammation. The Journal of clinical investigation 126, 3453-3466 (2016)
R. Raoof, H. Willemen, N. Eijkelkamp, Divergent roles of immune cells and their mediators in pain. Rheumatology (Oxford, England) 57, 429-440 (2018)
L. S. Simon et al, The Jak/STAT pathway: A focus on pain in rheumatoid arthritis. Seminars in arthritis and rheumatism 51, 278-284 (2021)
F. Seif et al, The role of JAK-STAT signaling pathway and its regulators in the fate of T helper cells. Cell Communication and Signaling 15, 23 (2017)
Heisler A C, Song J, Dunlop D D et al. Association of pain centralization and patient-reported pain in active rheumatoid arthritis. Arthritis Care Res (Hoboken) 2020; 72:1122-9
Daniel F McWilliams, Divya Thankaraj, Julie Jones-Diette, Rheinallt Morgan, Onosi S Ifesemen, Nicholas G Shenker, David A Walsh. The efficacy of systemic glucocorticosteroids for pain in rheumatoid arthritis: a systematic literature review and meta-analysis. Rheumatology, Volume 61, Issue 1, January 2022, Pages 76-89.
Rheumatoid Arthritis (update): Final Analgesics in Rheumatoid Arthritis. National Institute for Health and Care Excellence. Rheumatoid arthritis in adults: diagnosis and management. Evidence review G Analgesics NICE guideline NG100 Evidence review July 2018.
Joharatnam N, McWilliams D F, Wilson D et al. A cross-sectional study of pain sensitivity, disease-activity assessment, mental health, and fibromyalgia status in rheumatoid arthritis. Arthritis Res Ther 2015; 17:11.
Thomas Karonitsch, Lusine Yeghiazaryan, Angelika Lackner, Hans Peter Brezinsek, Tanja A Stamm, Franz Konig, Daniel Aletaha, Josef S Smolen. Targeting type I interferon (IFN) signalling in patients with RA with a high type I IFN gene signature. RMD Open 2022; 8:e002525.
Castañeda-Delgado, Bastián-Hernandez, Macias-Segura,Santiago-Algarra, Castillo-Ortiz, Alemán-Navarro, Martinez-Tejada, Enciso-Moreno, Garcia-De Lira, Olguín-Calderón, Trouw, Ramos-Remus and Enciso-Moreno. Type I Interferon Gene Response Is Increased in Early and Established Rheumatoid Arthritis and Correlates with Autoantibody Production. Frontiers in Immunology. 2017; 8(285): 1-12.
Furlan A, La Manno G, Lubke M, Haring M, Abdo H, Hochgerner H, et al. Visceral motor neuron diversity delineates a cellular basis for nipple- and pilo-erection muscle control. Nat Neurosci. 2016; 19(10):1331-40.
Li Y, Tong D, Liang P. Lonnblom E, Viljanen J, Xu B, et al. Cartilage-binding antibodies initiate joint inflammation and promote chronic erosive arthritis. Arthritis Res Ther. 2020; 22(1):120.
Bas D B, Su J, Sandor K, Agalave N M, Lundberg J, Codeluppi S, et al. Collagen antibody-induced arthritis evokes persistent pain with spinal glial involvement and transient prostaglandin dependency. Arthritis Rheum. 2012; 64(12): 3886-96).
Presley R W, Menetrey D, Levine J D, and Basbaum A I. Systemic morphine suppresses noxious stimulus-evoked Fos protein-like immunoreactivity in the rat spinal cord. J Neurosci. 1990; 10(1):323-35).
Haring M, Fatt M, and Kupari J. Protocol to Prepare Single-Cell Suspensions from Mouse Vagal Sensory Ganglia for Transcriptomic Studies. STAR Protoc. 2020; 1(1):100030).
Yim A K Y, Wang P L, Bermingham J R, Jr., Hackett A, Strickland A, Miller T M, et al. Disentangling glial diversity in peripheral nerves at single-nuclei resolution. Nat Neurosci. 2022; 25(2):238-51).
Zhang M D, Su J, Adori C, Cinquina V, Malenczyk K, Girach F, et al. Ca2+-binding protein NECAB2 facilitates inflammatory pain hypersensitivity. J Clin Invest. 2018; 128(9):3757-68).
Reeh P W. Sensory receptors in mammalian skin in an in vitro preparation. Neurosci Lett. 1986; 66(2):141-6;
Walcher J, Ojeda-Alonso J, Haseleu J, Oosthuizen M K, Rowe A H, Bennett N C, et al. Specialized mechanoreceptor systems in rodent glabrous skin. J Physiol. 2018; 596(20):4995-5016).
Zimmermann K, Hein A, Hager U, Kaczmarek J S, Turnquist B P, Clapham D E, et al. Phenotyping sensory nerve endings in vitro in the mouse. Nat Protoc. 2009; 4(2):174-96).
Koltzenburg M, Stucky C L, and Lewin G R. Receptive properties of mouse sensory neurons innervating hairy skin. J Neurophysiol. 1997; 78(4):1841 50.
Burke J R, Cheng L, Gillooly K M, Strnad 3, Zupa-Fernandez A, Catlett I M, et al. Autoimmune pathways in mice and humans are blocked by pharmacological stabilization of the TYK2 pseudokinase domain. Sci Transl Med. 2019; 11(502).
Krishnamurthy et al., Identification of a novel chemokine-dependent molecular mechanism underlying rheumatoid arthritis-associated autoantibody-mediated bone loss. Ann Rheum Dis 75, 721-729 (2016).
G. Wigerblad et al., Autoantibodies to citrullinated proteins induce joint pain independent of inflammation via a chemokine-dependent mechanism. Ann Rheum Dis 75, 730-738 (2016).
Usoskin et al., Unbiased classification of sensory neuron types by large-scale single-cell RNA sequencing. Nat Neurosci 18, 145-153 (2015).
Peng et al., miR-183 cluster scales mechanical pain sensitivity by regulating basal and neuropathic pain genes. Science 356, 1168-1171 (2017).
Warwick et al., MrgprdCre lineage neurons mediate optogenetic allodynia through an emergent polysynaptic circuit. Pain 162, 2120-2131 (2021).
J. Huang et al., Circuit dissection of the role of somatostatin in itch and pain. Nat Neurosci 21, 707-716 (2018).
S. Lou, B. Duan, L. Vong, B. B. Lowell, Q. Ma, Runxl controls terminal morphology and mechanosensitivity of VGLUT3-expressing C-mechanoreceptors. J Neurosci 33, 870-882 (2013).
J. S. Mogil, Sources of Individual Differences in Pain. Annu Rev Neurosci 44, 1-25 (2021).
Aibar et al., SCENIC: single-cell regulatory network inference and clustering. Nat Methods 14, 1083-1086 (2017).
Peter C. Taylor, Yvonne C. Lee, Roy Fleischmann, Tsutomu Takeuchi, Elizabeth L. Perkins, Bruno Fautrel, Baojin Zhu, Amanda K. Quebe, Carol L. Gaich, Xiang Zhang, Christina L. Dickson, Douglas E. Schlichting, Himanshu Patel, Frederick Durand and Paul Emery. Achieving Pain Control in Rheumatoid Arthritis with Baricitinib or Adalimumab Plus Methotrexate: Results from the RA-BEAM Trial. J. Clin. Med. 2019, 8(6), 831.
Elsaman, A.M., Maaty, A., and Hamed, A. (2021). Genicular nerve block in rheumatoid arthritis: a randomized clinical trial. Clinical rheumatology 40, 4501-4509.
McWilliams, D. F., and Walsh, D.A. (2017). Pain mechanisms in rheumatoid arthritis. Clinical and experimental rheumatology 35 Suppl 107, 94-101.
Nagi, S.S., Rubin, T.K., Chelvanayagam, D.K., Macefield, V. G., and Mahns, D.A. (2011). Allodynia mediated by C-tactile afferents in human hairy skin. The Journal of physiology 589, 4065-4075.
Smolen, J.S., Aletaha, D., Barton, A., Burmester, G.R., Emery, P., Firestein, G.S., Kavanaugh, A., McInnes, I.B., Solomon, D.H., Strand, V., et al. (2018). Rheumatoid arthritis. Nature reviews Disease primers 4, 18001.

1. A method of treating or preventing pain associated with Rheumatoid Arthritis in a patient, comprising a step of administering a type I interferon inhibitor to the patient.

2. The method according to claim 1, wherein the pain is dysfunctional pain, such as inflammatory joint pain.

3. The method according to claim 1, wherein the pain is not inflammatory pain.

4. The method according to any preceding claim, wherein the pain is not neuropathic pain or neuroplastic pain.

5. The method according to any preceding claim, wherein the pain is chronic pain; for example, pain that is present for three months or more, or six months or more, or twelve months or more.

6. The method according to any preceding claim, wherein the pain is one or more selected from the group consisting of: pain hypersensitivity; allodynia; hyperalgesia; and arthralgia.

7. The method according to any preceding claim, wherein the pain is associated with and/or caused by:
systemic inflammation; and/or
local inflammation; and/or
clinical inflammation.

8. The method according to any preceding claim, wherein the pain is not associated with and/or caused by Rheumatoid Arthritis inflammatory disease activity.

9. The method according to any preceding claim, wherein the pain is at an affected joint and/or opposite parts of an affected joint and/or cephalic parts of an affected joint and/or caudal parts of an affected joint.

10. The method according to any preceding claim, wherein the type I interferon inhibitor does not prevent or treat an inflammatory disease with increased type I interferon signalling.

11. The method according to any preceding claim, wherein:
the pain is present along with disease inflammation; and/or
the pain is present after the remission of disease inflammation.

12. The method according to any preceding claim, wherein the patient has received, or is receiving, pain treatment, but the pain persists and/or recurs and/or progresses.

13. The method according to claim 12, wherein the pain treatment is selected from the group consisting of: a nonsteroidal anti-inflammatory drug (NSAID), such as celecoxib, diclofenac, etoricoxib, ibuprofen, naproxen; a steroid, such as corticosteroid, glucocorticoid; acetaminophen; an opioid, such as codeine, dextropropoxyphene, tramadol; an antidepressant, such as tricyclic antidepressant; an anticonvulsant; or a combination thereof.

14. The method according to any preceding claim, wherein the pain is associated with and/or caused by increased type I interferon signalling in the patient.

15. The method according to claim 14, wherein increased type I interferon signalling comprises:
increased type I interferon intracellular signalling in the patient
increased levels of type I interferons in the patient;
increased activation of type I interferon receptors in the patient;
increased expression of one or more type I interferon-stimulated gene in the patient; and/or
reduced expression of one or more type I interferon-repressed gene in the patient.

16. The method according to any preceding claim, wherein the type I interferon is selected from the group comprising: Interferon-alpha; Interferon-beta.

17. The method according to any preceding claim, wherein the pain is associated with increased number and/or activity of one or more active sensory neuron in the patient; preferably increased number and/or activity of one or more active nociceptor of the patient.

18. The method according to claim 17, wherein the sensory neurons of the patient are TrkA-expressing sensory neurons; preferably TrkA-expressing nociceptors.

19. The method according to claims 17 or 18, wherein the sensory neurons of the patient are GFRa3-expressing sensory neurons; preferably GFRa3-expressing nociceptors.

20. The method according to any preceding claim, wherein the type I interferon inhibitor:
prevents or reduces type I interferon intracellular signalling in the patient;
prevents or reduces levels of type I interferons in the patient;
prevents or reduces activation of type I interferon receptors in the patient;
prevents or reduces expression of one or more type I interferon-stimulated gene in the patient; and/or
induces and/or increases expression of one or more type I interferon-repressed gene in the patient.

21. The method according to any preceding claim, wherein the type I interferon inhibitor is selected from the group consisting of: an MNK inhibitor (such as an MNK1 and/or MNK2 inhibitor), an IFNAR1 inhibitor, an IFNAR2 inhibitor, a TYK2 inhibitor, a type I interferon neutraliser, and a eukaryotic translation initiation factor 4E (eIF4E) inhibitor.

22. The method according to any preceding claim, wherein the type I interferon inhibitor is selected from the group consisting of: a small molecule, an antibody, an antibody part thereof, an antibody mimetic, a decoy receptor, a receptor body, and a vaccine.

23. The method according to any preceding claim, wherein the type I interferon inhibitor is selected from the group consisting of: deucravacitinib, anifrolumab, NDI-034858, NDI-031232, NDI-031301, NDI-031407, ESK-001, VTX-958, ICP-488, ropsacitinib, and GLPG3667.

24. The method, according to any of claims 1-22, wherein the type I interferon inhibitor is an MNK inhibitor, such as an MNK1 and/or MNK2 inhibitor.

25. The method, according to claim 24, wherein the MNK inhibitor is selected from the group consisting of: eFT508; 4ET-03-053; BAY1143269; ETC-1907206; and derivatives thereof.

26. The method according to claim 24, wherein the MNK inhibitor is a compound of Structure (II): 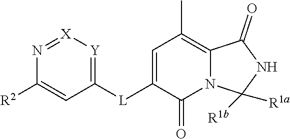
or a pharmaceutically acceptable salt thereof, wherein
R^{1a} is C₁-C₆ alkyl or aryl;
R^{1b} is C₁-C₆ alkyl or aryl,
or R^{1a} and R^{1b}, together with the carbon to which they are both attached, join to form cycloalkyl, cycloalkenyl, heterocyclyl, aryl, or heteroaryl;
R² is —NHR^{3a}, —NHC(═O)R^{3b}, —NHC(═S)R^{3b}, or —C(═O)R^{3c};
R^{3a} is hydrogen, C₁-C₆ alkyl, or C₃-C₆ cycloalkyl, each of which is optionally substituted with one or more substituents selected from the group consisting of hydroxyl, C₃-C₆ cycloalkyl, —NHS(O)₂CH₃, heterocyclyl, —C(═O)OH, —C(═O)N(R^{3d})R^{3d}, or —N(R^{3d})R^{3d};
R^{3b} is C₁-C₆ alkyl, C₃-C₆ cycloalkyl, or heterocyclyl each of which is optionally substituted with one or more substituents selected from the group consisting of hydroxyl, halo, C₁-C₆ alkyl, C₃-C₆ cycloalkyl, —NHS(O)₂CH₃, —N(R^{3d})R^{3d}, heterocyclyl, —C(═O)OH, —C(═O)N(R^{3d})R^{3d}, —NHC(═O)CH₃, —CH₂C(═O)OH,
R^{3c} is —N(R^{3d})R^{3d} or heterocyclyl;
R^{3d} is, at each occurrence, independently hydrogen, C₁-C₆ alkyl, or C₃-C₆ cycloalkyl;
L is —NH— or —CH₂NH—; and
X is N and Y is CH or X is CH and Y is N.

27. The method of claim 26, wherein when R^{1a} and R^{1b} are both —CH₃ or when R^{1a} and R join to form a 5- or 6-membered cycloalkyl or heterocyclyl, then R² does not have the following structure:

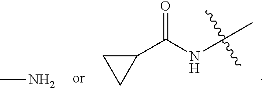

28. The method of claim 26, wherein the MNK inhibitor is selected from Table 1.

29. The method of claim 24, wherein the MNK inhibitor is a compound of Formula (I′): 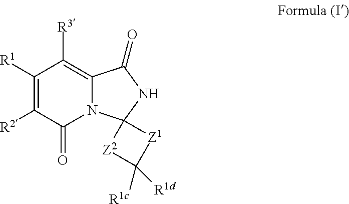
or a pharmaceutically acceptable salt thereof, wherein:
R¹ is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl:
R^{2′} is selected from the group consisting of 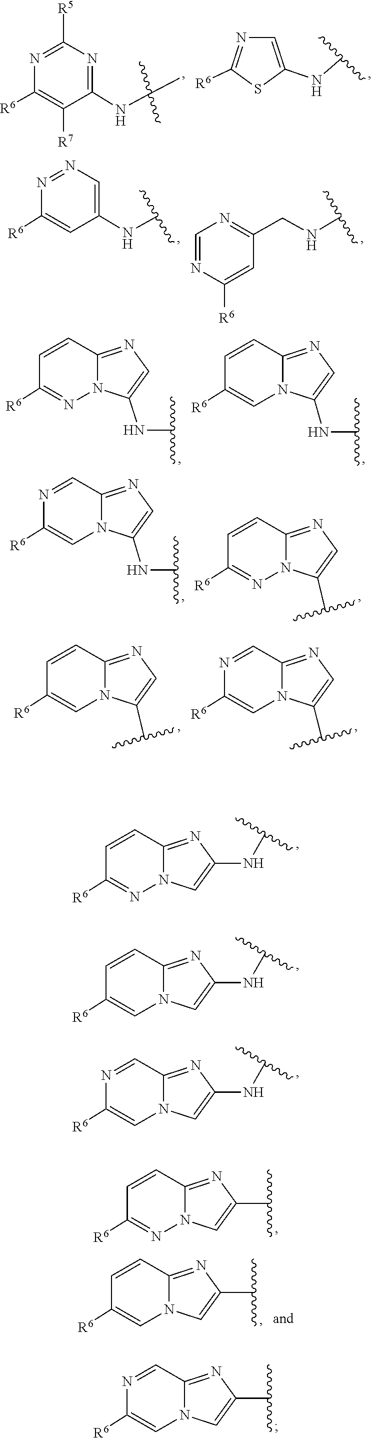
R^{3′} is selected from the group consisting of hydrogen, halogen, C₁₋₆ alkyl, C₃₋₇ branched alkyl, C₁₋₆ haloalkyl, C₃₋₇ branched haloalkyl, C₁₋₆ hydroxyalkyl, C₃₋₇ branched hydroxyalkyl, cyano, C₁₋₆ alkoxyl, C₃₋₇ branched alkoxy, hydroxy, and C₃₋₆ cycloalkyl that is optionally substituted with 1 to 3 substituents selected from the groups consisting of halogen, C₁₋₆ alkyl, C₁₋₆ haloalkyl, and C₁₋₆ hydroxyalkyl;
R^{1c} and R^{1d} are taken together to form a 3- to 7-membered ring having 0-2 heteroatoms selected from the group consisting of N, O and S, wherein the 3- to 7-membered ring may be further optionally substituted with one or more substituents selected from the group consisting of halo, oxo, C₁₋₆ alkyl, R⁸, and —C(═O)OR⁹;
Z¹ and Z² are each independently a direct bond or —{C(R^{4a})(R^{4b})}ₚ—Y¹—; wherein p is 0, 1, 2, 3, 4, or 5, Y¹ is a direct bond, —O—, or —N(R⁸)—;
R^{4a} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl); or two R⁴⁸ attached to two adjacent carbons to form a direct bond;
R^{4b} is at each occurrence independently selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ alkoxyl, C₃₋₇ branched alkoxy, NHCO(C₁₋₆alkyl), NHCO(C₃₋₇ branched alkyl), NHCO(C₃₋₇ cycloalkyl), NHSO₂(C₁₋₆alkyl), NHSO₂(C₃₋₇ branched alkyl), and NHSO₂(C₃₋₇ cycloalkyl);
R⁵ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxy:
R⁶ is selected from the group consisting of hydrogen, NH₂, NHR⁶a, NHCH₂CH₂OH, NHCH₂CH₂NHSO₂Me, C₁₋₆ alkoxyl, C₃₋₇ branched alkoxy, and hydroxy;
R^{6a} is selected from the group consisting of —(CO)C₁₋₆ alkyl, —(CO)C₃₋₇ branched alkyl, —(CO)C₁₋₆ hydroxyalkyl, 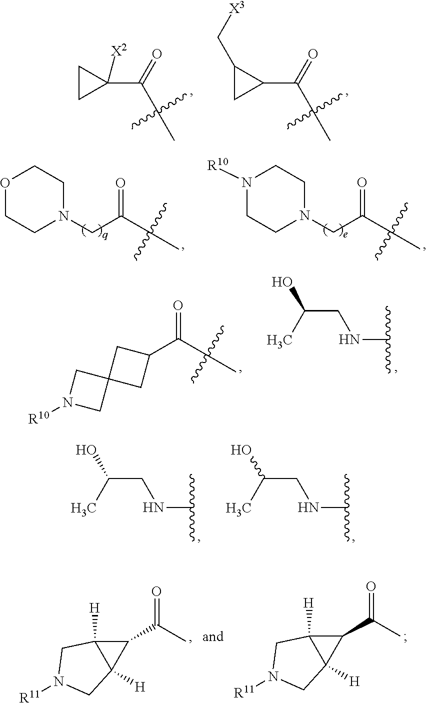
q is 1, 2, 3, 4, 5, or 6;
e is 1, 2, 3, 4, 5, or 6;
X² is selected from the group consisting of hydrogen, halogen, C₁₋₆alkyl, C₃₋₇ branched alkyl, C₁₋₆haloalkyl, C₃₋₇ branched haloalkyl, hydroxy, C₁₋₆hydroxyalkyl, C₃₋₇ branched hydroxyalkyl, C₁₋₆alkoxy, C₃₋₇ branched alkoxy, C₁₋₆haloalkoxy, C₃₋₇ branched haloalkoxy, NH₂, NH(C₁₋₆alkyl), N(C₁₋₆alkyl)₂, C₁₋₅(COOH), C₁₋₆(NHSO₂Me);
X³ is selected from the group consisting of hydrogen, halogen, C₁₋₆ alkyl, C₃₋₇ branched alkyl, C₁₋₆ haloalkyl, C₃₋₇ branched haloalkyl, hydroxy, C₁₋₆ hydroxyalkyl, C₃₋₇ branched hydroxyalkyl, C₁₋₆ alkoxy, C₃₋₇ branched alkoxy, C₁₋₆ haloalkoxy, C₃₋₇ branched haloalkoxy, NH₂, NH(C₁₋₆ alkyl), N(C₁₋₆ alkyl)₂, COOH, C₁₋₅(COOH), NHSO₂Me, C₁₋₅(NHSO₂Me);
R⁷ is selected from the group consisting of hydrogen, halogen, C₁₋₆ alkyl, C₃₋₇ branched alkyl, C₁₋₆ haloalkyl, C₃₋₇ branched haloalkyl, C₁₋₆ alkoxyl, C₃₋₇ branched alkoxy, and hydroxyl;
R⁸ is selected from the group consisting of C₁₋₆ alkyl, C₁₋₆ haloalkyl, C₃₋₇ branched haloalkyl, C₁₋₆ hydroxyalkyl, C₃₋₇ branched hydroxyalkyl, C₁₋₆ alkoxyl, C₃₋₇ branched alkoxy, CO(C₁₋₆alkyl), CO(C₃₋₇ branched alkyl), SO₂(C₁₋₆alkyl), and SO₂(C₃₋₇ branched alkyl); and
R⁹ is selected from the group consisting of hydrogen, C₁₋₆ alkyl, and aralkyl.

30. The method of claim 29, wherein the MNK inhibitor is a compound selected from:
N-(6-((8″-methyl-1″,5″-dioxo-1″,5″-dihydro-2″H-dispiro[cyclopropane-1,1′-cyclohexane-3′,3″-imidazo[1,5-a]pyridin]-6″-yl)amino)pyrimidin-4-yl)cyclopropanecarboxamide;
6″-((6-Aminopyrimidin-4-yl)amino)-8″-methyl-2″H-dispiro[cyclopropane-1,1′-cyclohexane-3′,3″-imidazo[1,5-a]pyridine]-1″,5″-dione;
N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropanecarboxamide;
N-(6-((8″-methyl-1″,5″-dioxo-1″,5″-dihydro-2″H-dispiro[aziridine-2,1′-cyclohexane-4′,3″-imidazo[1,5-a]pyridin]-6″-yl)amino)pyrimidin-4-yl)cyclopropanecarboxamide;
N-(6-((8″-methyl-1″,5″-dioxo-1″,5″-dihydro-2′H-dispiro[cyclobutane-1,1′-cyclobutane-3′,3″-imidazo[1,5-a]pyridin]-6″-yl)amino)pyrimidin-4-yl)cyclopropanecarboxamide;
benzyl 6″-((6-(cyclopropanecarboxamido)pyrimidin-4-yl)amino)-8″-methyl-1″,5″-dioxo-1″,5″-dihydro-2″H-dispiro[aziridine-2,1′-cyclohexane-4′,3″-imidazo[1,5-a]pyridine]-1-carboxylate;
tert-butyl 6″-((6-(cyclopropanecarboxamido)pyrimidin-4-yl)amino)-8″-methyl-1″,5″-dioxo-1″,5″-dihydro-2″H-dispiro[azetidine-3,1′-cyclohexane-4′,3″-imidazo[1,5-a]pyridine]-1-carboxylate;
N-(6-((8″-methyl-1″,5″-dioxo-1″,5″-dihydro-2″H-dispiro[azetidine-3,1′-cyclohexane-4′,3″-imidazo[1,5-a]pyridin]-6″-yl)amino)pyrimidin-4-yl)cyclopropanecarboxamide;
6″-((6-((2-hydroxyethyl)amino)pyrimidin-4-yl)amino)-8″-methyl-2″H-dispiro[cyclopropane-1,1′-cyclohexane-4′,3″-imidazo[1,5-a]pyridine]-1″,5″ d lone;
6″-((6-Aminopyrimidin-4-yl)amino)-8″-methyl-2″H-dispiro[cyclopropane-1,1′-cyclohexane-4′,3″-imidazo[1,5-a]pyridine]-1″,5″-dione;
benzyl 6″-((6-aminopyrimidin-4-yl)amino)-8″-methyl-1″,5″-dioxo-1″,5″-dihydro-2″H-dispiro[aziridine-2,1′-cyclohexane-4′,3″-imidazo[1,5-a]pyridine]-1-carboxylate;
1-(aminomethyl)-N-(6-((8″-methyl-1″,5″-dioxo-1″,5″-dihydro-2′H-dispiro[cyclopropane-1,1′-cyclohexane-4′,3″-imidazo[1,5-a]pyridin]-6″-yl)amino)pyrimidin-4-yl)cyclopropane-1-carboxamide;
(1R,5S,6r)-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-3-azabicyclo[3.1.0]hexane-6-carboxamide;
N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-2-azaspiro[3.3]heptane-6-carboxamide;
2-methyl-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-2-azaspiro[3.3]heptane-6-carboxamide;
(1R,5S,6r)-3-methyl-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-3-azabicyclo[3.1.0]hexane-6-carboxamide;
N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)-1-(methylsulfonamido methyl)cyclopropane-1-carboxamide;
1-((dimethylamino)methyl)-N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropane-1-carboxamide;
6″-((6-aminopyrimidin-4-yl)amino)-8″-methyl-2′H-dispiro[cyclobutane-1,1′-cyclobuta ne-3′,3″-imidazo[1,5-a]pyridine]-1″,5″-d lone;
6″-((6-aminopyrimidin-4-yl)amino)-8″-methyl-2″H-dispiro[aziridine-2,1′-cyclohexa ne-4′,3″-imidazo[1,5-a]pyridine]-1″,5″-dione;
6″-((6-aminopyrimidin-4-yl)amino)-8″-methyl-2″H-dispiro[cyclopropane-1,1′-cyclopentane-3′,3″-imidazo[1,5-a]pyridine]-1″,5″-dione;
6″-((6-aminopyrimidin-4-yl)amino)-8″-methyl-2′H-dispiro[cyclopentane-1,1′-cyclopentane-3′,3″-imidazo[1,5-a]pyridine]-1″,5″-dione;
6″-((6-aminopyrimidin-4-yl)amino)-3,3-difluoro-8″-methyl-2″H-dispiro[cyclobuta ne-1,1′-cyclobutane-3′,3″-imidazo[1,5-a]pyridine]-1″,5″ d lone;
6″-((6-aminopyrimidin-4-yl)amino)-8″-methyl-2″H-dispiro[cyclopentane-1,1′-cyclobuta ne-3′,3″-imidazo[1,5-a]pyridine]-1″,5″-d lone;
6″-((6-aminopyrimidin-4-yl)amino)-8″-methyl-2″H-dispiro[cyclobutane-1,1′-cyclohexane-4′,3″-imidazo[1,5-a]pyridine]-1″,5″-dione;
6″-((6-aminopyrimidin-4-yl)amino)-8″-methyl-2′H-dispiro[cyclohexane-1,1′-cyclobuta ne-3′,3″-imidazo[1,5-a]pyridine]-1″,5″-d lone;
ethyl 6″-((6-(cyclopropanecarboxamido)pyrimidin-4-yl)amino)-8″-methyl-1″,5″-dioxo-1″,5″-dihydro-2″H-dispiro[cyclopropane-1,1′-cyclohexa ne-4′,3″-imidazo[1,5-a]pyridine]-2-carboxylate;
tert-butyl (6″-((6-(cyclopropanecarboxamido)pyrimidin-4-yl)amino)-8″-methyl-1″,5″-dioxo-1″,5″-dihydro-2″H-dispiro[cyclopropane-1,1′-cyclohexane-4′,3″-imidazo[1,5-a]pyridin]-2-yl)carbamate;
N-(6-((2,2-difluoro-8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropanecarboxamide;
6″-((6-aminopyrimidin-4-yl)amino)-2,2-difluoro-8″-methyl-2″H-dispiro[cyclopropane-1,1′-cyclohexane-4′,3″-imidazo[1,5-a]pyridine]-1″,5″-dione;
6″-((6-Aminopyrimidin-4-yl)amino)-8″-methyl-2″H-dispiro[cyclopropane-1,1′-cycloheptane-4′,3″-imidazo[1,5-a]pyridine]-1″,5″-dione;
6″-((6-Aminopyrimidin-4-yl)amino)-8″-methyl-2″H-dispiro[cyclopropane-1,1′-cyclohexane-4′,3″-imidazo[1,5-a]pyridin]-2′-ene-1″,5″-dione;
or a pharmaceutically acceptable salt thereof.

31. The method of claim 24, wherein the MNK inhibitor is a compound of Formula IB: 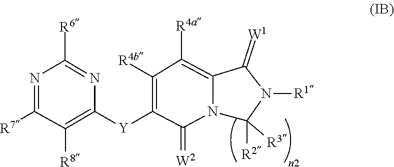
or a pharmaceutically acceptable salt thereof, wherein:
W¹ and W² are independently O, S or N—OR′, where R′ is lower alkyl;
Y is —N(R^{5″})—, —O—, —S—, —C(O)—, —S═O, —S(O)₂—, or —CHR⁹—;
R^{1″} is hydrogen, lower alkyl, cycloalkyl or heterocyclyl wherein any lower alkyl, cycloalkyl or heterocyclyl is optionally substituted with 1, 2 or 3 3 groups;
n² is 1, 2 or 3;
R^{2″} and R^{3″} are each independently hydrogen, alkyl, alkenyl, alkynyl, aryl, araalkylene, heteroaryl, heteroarylalkylene, cycloalkyl, cycloalkylalkylene, heterocyclyl, or heterocyclylalkylene, wherein any alkyl, aryl, araalkylene, heteroaryl, heteroarylalkylene, cycloalkyl, cycloalkylalkylene, heterocyclyl, or heterocyclylalkylene, is optionally substituted with 1, 2 or 3 J groups;
or R^{2″} and R^{3″} taken together with the carbon atom to which they are attached form a cycloalkyl or heterocyclyl, wherein any cycloalkyl or heterocyclyl is optionally substituted with 1, 2 or 3 J groups;
R^{4a″} and R^{4b″} are each independently hydrogen, halogen, hydroxyl, thiol, hydroxyalkylene, cyano, alkyl, alkoxy, acyl, thioalkyl, alkenyl, alkynyl, cycloalkyl, aryl, or heterocyclyl;
R^{5″} is hydrogen, cyano, or lower alkyl;
or R^{5″} and R⁸ taken together with the atoms to which they are attached form a fused heterocyclyl optionally substituted with 1, 2 or 3 J groups;
R^{6″}, R^{7″} and R⁸ are each independently hydrogen, hydroxy, halogen, cyano, amino, alkyl, alkenyl, alkynyl, alkoxy, cycloalkyl, cycloalkylalkylene, cycloalkylalkenylene, alkylaminyl, alkylcarbonylaminyl, cycloalkylcarbonylaminyl, cycloalkylaminyl, heterocyclylaminyl, heteroaryl, or heterocyclyl, and wherein any amino, alkyl, alkenyl, alkynyl, alkoxy, cycloalkyl, cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups;
or R^{7″} and R⁸ taken together with the atoms to which they are attached form a fused heterocyclyl or heteroaryl optionally substituted with 1, 2 or 3 3 groups;
J is —SH, —SR⁹, —S(O)R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)—NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene, NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl; or any two J groups bound to the same carbon or hetero atom may be taken together to form oxo; and
R⁹ is hydrogen, lower alkyl or —OH.

32. The method of claim 31, wherein the MNK inhibitor is a compound selected from Table 5, or a pharmaceutically acceptable salt thereof.

33. A pharmaceutical composition comprising a type I interferon inhibitor and one or more therapeutic agent for treating pain associated with Rheumatoid Arthritis in a patient, and/or one or more therapeutic agent for treating Rheumatoid Arthritis, in combination with a pharmaceutically-acceptable carrier, diluent or excipient.

34. A method of treating or preventing pain associated with Rheumatoid Arthritis in a patient, comprising a step of administering a pharmaceutical composition as defined in claim 33 to the patient.

35. A method of treating or preventing pain associated with Rheumatoid Arthritis in a patient, comprising administering to the patient a type I interferon inhibitor and one or more therapeutic agent for treating pain associated with Rheumatoid Arthritis in a patient, and/or one or more therapeutic agent for treating Rheumatoid Arthritis.

36. A kit comprising a type I interferon inhibitor and one or more therapeutic agent for treating Rheumatoid Arthritis.

37. A method for identifying a patient who has pain associated with Rheumatoid Arthritis and is in need of treatment with a type I interferon inhibitor, the method comprising the steps of:
(a) providing a test sample from a patient who has pain associated with Rheumatoid Arthritis;
(b) determining the level of type I interferon signalling in the test sample; and
(c) identifying the patient as one in need of treatment with a type I interferon inhibitor on the basis of the determination in Step (b).

38. The method of claim 37, further comprising the step of administering a type I interferon inhibitor to the patient.


SEQUENCE LISTING (6 sequences)

SEQ ID NO: 1 - DNA, 24 bp, synthetic construct
  misc_feature 1..24: note = Description of sequence: Ifna pan primer Forward
  source 1..24: mol_type = other DNA
cctgagaagagaagaaacacagcc

SEQ ID NO: 2 - DNA, 23 bp, synthetic construct
  misc_feature 1..23: note = Description of sequence: Ifna pan primer Reverse
  source 1..23: mol_type = other DNA
ggctctccagactttctgctctg

SEQ ID NO: 3 - DNA, 18 bp, synthetic construct
  misc_feature 1..18: note = Description of sequence: Ifnb pan primer Forward
  source 1..18: mol_type = other DNA
agggcggacttcaagatc

SEQ ID NO: 4 - DNA, 18 bp, synthetic construct
  misc_feature 1..18: note = Description of sequence: Ifnb pan primer Reverse
  source 1..18: mol_type = other DNA
ctcattccacccagtgct

SEQ ID NO: 5 - DNA, 20 bp, synthetic construct
  misc_feature 1..20: note = Description of sequence: Gapdh pan primer Forward
  source 1..20: mol_type = other DNA
aactttggcattgtggaagg

SEQ ID NO: 6 - DNA, 20 bp, synthetic construct
  misc_feature 1..20: note = Description of sequence: Gapdh pan primer Reverse
  source 1..20: mol_type = other DNA
acacattgggggtaggaaca